SEQUENCE LISTING (150 sequences)

SEQ ID NO: 1 - protein, 473 aa, Artificial Sequence
  Description of Artificial Sequence Synthetic polypeptide
MDPKGSLSWRILLFLSLAFELSYGEVKLVESGGGLVQPGGSLRLSCAASGFTFRTYAMSWVRQAPGKGLEWVATIGSDRRHTYYPDSVKGRFTISRDNAKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 473 aa, Artificial Sequence
  Description of Artificial Sequence Synthetic polypeptide
MDPKGSLSWRILLFLSLAFELSYGEVQLVESGGGLVQPGGSLRLSCAASGFTFRTYAMSWVRQAPGKGLEWVATIGSDRRHTYYPDSVKGRFTISRDNSKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 473 aa, Artificial Sequence
  Description of Artificial Sequence Synthetic polypeptide
MDPKGSLSWRILLFLSLAFELSYGEVKLVQSGAEVKKPGASVKVSCKASGFTFRTYAMSWVRQAPGQRLEWVATIGSDRRHTYYPDKFQGRVTITRDNAKNTLYMELSSLRSEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 473 aa, Artificial Sequence
  Description of Artificial Sequence Synthetic polypeptide
MDPKGSLSWRILLFLSLAFELSYGEVQLVESGGGLVKPGGSLRLSCAASGFTFSRYGMSWVRQAPGKGLEWVATISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 473 aa, Artificial Sequence
  Description of Artificial Sequence Synthetic polypeptide
MDPKGSLSWRILLFLSLAFELSYGEVQLVESGGGLVKPGGSLRLSCAASGFTFSRYGMSWVRQAPGKGLEWVSTISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 473 aa, Artificial Sequence
  Description of Artificial Sequence Synthetic polypeptide
MDPKGSLSWRILLFLSLAFELSYGEVQLVESGGGLVQPGRSLRLSCTASGFTFSRYGMSWVRQAPGKGLEWVATISSGGSYTYYTDSVKGRFTISRDNAKNILYLQMNSLKTEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 239 aa, Artificial Sequence
  Description of Artificial Sequence Synthetic polypeptide
METDTLLLWVLLLWVPGSTGDVVMTQSPLSLPVTLGQPASISCRSSQSLLDSDGKTFLNWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 239 aa, Artificial Sequence
  Description of Artificial Sequence Synthetic polypeptide
METDTLLLWVLLLWVPGSTGDVVMTQSPLSLPVTLGQPASISCRSSQSLLDSDGKTFLNWLQQRPGQSPRRLIYLVSKRDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 239 aa, Artificial Sequence
  Description of Artificial Sequence Synthetic polypeptide
METDTLLLWVLLLWVPGSTGDVVMTQSPDSLAVSLGERATINCKSSQSLLDSDGKTFLNWLQQKPGQPPKRLIYLVSKLDSGVPDRFSGSGSGTDFTLTISSLQAEDVAVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 233 aa, Artificial Sequence
  Description of Artificial Sequence Synthetic polypeptide
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 233 aa, Artificial Sequence
  Description of Artificial Sequence Synthetic polypeptide
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYYCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 233 aa, Artificial Sequence
  Description of Artificial Sequence Synthetic polypeptide
METDTLLLWVLLLWVPGSTGDIVMTQSPATLSLSPGERATLSCRASQDISNYLNWYQQKPGQAVRLLIYYTSRLHSGIPARFSGSGSGTDYTLTISSLEPEDFAVYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 473 aa, Artificial Sequence
  Description of Artificial Sequence Synthetic polypeptide
  MOD_RES (27)..(27): Any amino acid, such as Q or K
  MOD_RES (30)..(30): Any amino acid, such as E or Q
  MOD_RES (33)..(33): Any amino acid, such as G or A
  MOD_RES (34)..(34): Any amino acid, such as G or E
  MOD_RES (35)..(35): Any amino acid, such as L or V
  MOD_RES (36)..(36): Any amino acid, such as V or K
  MOD_RES (37)..(37): Any amino acid, such as Q or K, or absent
  MOD_RES (40)..(40): Any amino acid, such as R, G or A
  MOD_RES (42)..(42): Any amino acid, such as L or V
  MOD_RES (43)..(43): Any amino acid, such as R or K
  MOD_RES (44)..(44): Any amino acid, such as L or V
  MOD_RES (47)..(47): Any amino acid, such as T, A or K
  MOD_RES (54)..(54): Any amino acid, such as S or R, or absent
  MOD_RES (55)..(55): Any amino acid, such as R or T, or absent
  MOD_RES (57)..(57): Any amino acid, such as G or A, or absent
  MOD_RES (67)..(67): Any amino acid, such as K or Q
  MOD_RES (68)..(68): Any amino acid, such as G or R
  MOD_RES (73)..(73): Any amino acid, such as A or S
  MOD_RES (76)..(76): Any amino acid, such as S or G, or absent
  MOD_RES (78)..(78): Any amino acid, such as G or D, or absent
  MOD_RES (79)..(79): Any amino acid, such as G or R, or absent
  MOD_RES (80)..(80): Any amino acid, such as S or R, or absent
  MOD_RES (81)..(81): Any amino acid, such as Y or H, or absent
  MOD_RES (85)..(85): Any amino acid, such as T or P, or absent
  MOD_RES (87)..(87): Any amino acid, such as S or K
  MOD_RES (88)..(88): Any amino acid, such as V or F
  MOD_RES (89)..(89): Any amino acid, such as K or Q
  MOD_RES (92)..(92): Any amino acid, such as F or V
  MOD_RES (95)..(95): Any amino acid, such as S or T
  MOD_RES (99)..(99): Any amino acid, such as A or S
  MOD_RES (102)..(102): Any amino acid, such as I, S or T
  MOD_RES (105)..(105): Any amino acid, such as L or M
  MOD_RES (106)..(106): Any amino acid, such as Q or E
  MOD_RES (107)..(107): Any amino acid, such as M or L
  MOD_RES (108)..(108): Any amino acid, such as N or S
  MOD_RES (111)..(111): Any amino acid, such as K or R
  MOD_RES (112)..(112): Any amino acid, such as T, A or S
  MOD_RES (121)..(121): Any amino acid, such as E or V, or absent
  MOD_RES (122)..(122): Any amino acid, such as R or G, or absent
  MOD_RES (123)..(123): Any amino acid, such as H or P, or absent
  MOD_RES (124)..(124): Any amino acid, such as G or Y, or absent
  MOD_RES (125)..(125): Any amino acid, such as G or D, or absent
  MOD_RES (126)..(126): Any amino acid, such as D or G, or absent
  MOD_RES (127)..(127): Any amino acid, such as G or Y, or absent
  MOD_RES (129)..(129): Any amino acid, such as W or G, or absent
  MOD_RES (130)..(130): Any amino acid, such as Y or E, or absent
  MOD_RES (133)..(133): Any amino acid, such as V or Y, or absent
  MOD_RES (139)..(139): Any amino acid, such as M or L, or absent
MDPKGSLSWRILLFLSLAFELSYGEVXLVXSGXXXXXPGXSXXXSCXASGFTFXXYXMSWVRQAPGXXLEWVXTIXSXXXXTYYXDXXXGRXTIXRDNXKNXLYXXXXSLXXEDTAVYYCXXXXXXXYXXFDXWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 239 aa, Artificial Sequence
  Description of Artificial Sequence Synthetic polypeptide
  MOD_RES (22)..(22): Any amino acid, such as I or V, or absent
  MOD_RES (23)..(23): Any amino acid, such as V or Q
  MOD_RES (29)..(29): Any amino acid, such as A, S, D or L, or absent
  MOD_RES (30)..(30): Any amino acid, such as T or S
  MOD_RES (32)..(32): Any amino acid, such as S, A or P
  MOD_RES (33)..(33): Any amino acid, such as L, A or V
  MOD_RES (34)..(34): Any amino acid, such as S or T
  MOD_RES (35)..(35): Any amino acid, such as P, V or L
  MOD_RES (37)..(37): Any amino acid, such as E, D or Q, or absent
  MOD_RES (38)..(38): Any amino acid, such as R or P
  MOD_RES (39)..(39): Any amino acid, such as A or V
  MOD_RES (40)..(40): Any amino acid, such as T or S
  MOD_RES (41)..(41): Any amino acid, such as L or I
  MOD_RES (42)..(42): Any amino acid, such as S, T or N
  MOD_RES (44)..(44): Any amino acid, such as R or K
  MOD_RES (45)..(45): Any amino acid, such as A or S, or absent
  MOD_RES (48)..(48): Any amino acid, such as D or S, or absent
  MOD_RES (49)..(49): Any amino acid, such as I or L, or absent
  MOD_RES (50)..(50): Any amino acid, such as L or absent
  MOD_RES (51)..(51): Any amino acid, such as D or absent
  MOD_RES (52)..(52): Any amino acid, such as S or absent
  MOD_RES (53)..(53): Any amino acid, such as D or absent
  MOD_RES (54)..(54): Any amino acid, such as G or absent
  MOD_RES (55)..(55): Any amino acid, such as S or K, or absent
  MOD_RES (56)..(56): Any amino acid, such as N or T, or absent
  MOD_RES (57)..(57): Any amino acid, such as Y or F, or absent
  MOD_RES (61)..(61): Any amino acid, such as Y or L, or absent
  MOD_RES (64)..(64): Any amino acid, such as K or R
  MOD_RES (67)..(67): Any amino acid, such as Q or K
  MOD_RES (68)..(68): Any amino acid, such as A, P or S
  MOD_RES (69)..(69): Any amino acid, such as V or P, or absent
  MOD_RES (70)..(70): Any amino acid, such as R or K, or absent
  MOD_RES (71)..(71): Any amino acid, such as L or R, or absent
  MOD_RES (75)..(75): Any amino acid, such as Y or L, or absent
  MOD_RES (76)..(76): Any amino acid, such as T or V, or absent
  MOD_RES (78)..(78): Any amino acid, such as R or K, or absent
  MOD_RES (79)..(79): Any amino acid, such as L or R
  MOD_RES (80)..(80): Any amino acid, such as H or D, or absent
  MOD_RES (83)..(83): Any amino acid, such as I or V
  MOD_RES (85)..(85): Any amino acid, such as A, S or D
  MOD_RES (96)..(96): Any amino acid, such as Y or F, or absent
  MOD_RES (99)..(99): Any amino acid, such as T or K
  MOD_RES (102)..(102): Any amino acid, such as S or R
  MOD_RES (103)..(103): Any amino acid, such as L or V
  MOD_RES (104)..(104): Any amino acid, such as E or Q, or absent
  MOD_RES (105)..(105): Any amino acid, such as P or A, or absent
  MOD_RES (108)..(108): Any amino acid, such as F or V, or absent
  MOD_RES (109)..(109): Any amino acid, such as A or G
  MOD_RES (110)..(110): Any amino acid, such as V or T
  MOD_RES (112)..(112): Any amino acid, such as F or Y
  MOD_RES (114)..(114): Any amino acid, such as Q or W, or absent
  MOD_RES (117)..(117): Any amino acid, such as N or T, or absent
  MOD_RES (118)..(118): Any amino acid, such as P or H, or absent
  MOD_RES (119)..(119): Any amino acid, such as F or absent
  MOD_RES (120)..(120): Any amino acid, such as L or P, or absent
  MOD_RES (121)..(121): Any amino acid, such as R or Y, or absent
  MOD_RES (125)..(125): Any amino acid, such as G or Q, or absent
  MOD_RES (129)..(129): Any amino acid, such as V or L, or absent
METDTLLLWVLLLWVPGSTGDXXMTQSPXXLXXXXGXXXXXXCXXSQXXXXXXXXXXLNWXQQXPGXXXXXLIYXXSXXXSGXPXRFSGSGSGTDXTLXISXXXXEDXXXYXCXQGXXXXXTFGXGTKXEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 16 - protein, 330 aa, Homo sapiens [sequence omitted]

SEQ ID NO: 17 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 18 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 19 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 20 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 22 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 23 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 473 aa, Artificial Sequence
  Description of Artificial Sequence Synthetic polypeptide
  MOD_RES (27)..(27): K or Q
  MOD_RES (30)..(30): Q or E
  MOD_RES (33)..(33): A or G
  MOD_RES (34)..(34): E or G
  MOD_RES (35)..(35): V or L
  MOD_RES (36)..(36): K or V
  MOD_RES (37)..(37): K or Q
  MOD_RES (40)..(40): A or G
  MOD_RES (42)..(42): V or L
  MOD_RES (43)..(43): K or R
  MOD_RES (44)..(44): V or L
  MOD_RES (47)..(47): K or A
  MOD_RES (67)..(67): Q or K
  MOD_RES (68)..(68): R or G
  MOD_RES (87)..(87): K or S
  MOD_RES (88)..(88): F or V
  MOD_RES (89)..(89): Q or K
  MOD_RES (92)..(92): V or F
  MOD_RES (95)..(95): T or S
  MOD_RES (99)..(99): A or S
  MOD_RES (105)..(105): M or L
  MOD_RES (106)..(106): E or Q
  MOD_RES (107)..(107): L or M
  MOD_RES (108)..(108): S or N
  MOD_RES (112)..(112): S or A
MDPKGSLSWRILLFLSLAFELSYGEVXLVXSGXXXXXPGXSXXXSCXASGFTFRTYAMSWVRQAPGXXLEWVATIGSDRRHTYYPDXXXGRXTIXRDNXKNTLYXXXXSLRXEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 239 aa, Artificial Sequence
  Description of Artificial Sequence Synthetic polypeptide
  MOD_RES (29)..(29): D or L
  MOD_RES (32)..(32): A or P
  MOD_RES (34)..(34): S or T
  MOD_RES (37)..(37): E or Q
  MOD_RES (38)..(38): R or P
  MOD_RES (40)..(40): T or S
  MOD_RES (42)..(42): N or S
  MOD_RES (44)..(44): K or R
  MOD_RES (64)..(64): K or R
  MOD_RES (68)..(68): P or S
  MOD_RES (70)..(70): K or R
  MOD_RES (79)..(79): L or R
  MOD_RES (99)..(99): T or K
  MOD_RES (102)..(102): S or R
  MOD_RES (103)..(103): L or V
  MOD_RES (104)..(104): Q or E
  MOD_RES (109)..(109): A or G
METDTLLLWVLLLWVPGSTGDVVMTQSPXSLXVXLGXXAXIXCXSSQSLLDSDGKTFLNWLQQXPGQXPXRLIYLVSKXDSGVPDRFSGSGSGTDFTLXISXXXAEDVXVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 473 aa, Artificial Sequence
  Description of Artificial Sequence Synthetic polypeptide
  MOD_RES (37)..(37): Q or K
  MOD_RES (40)..(40): R or G
  MOD_RES (47)..(47): T or A
  MOD_RES (73)..(73): A or S
  MOD_RES (102)..(102): I or S
  MOD_RES (111)..(111): K or R
  MOD_RES (112)..(112): T or A
MDPKGSLSWRILLFLSLAFELSYGEVQLVESGGGLVXPGXSLRLSCXASGFTFSRYGMSWVRQAPGKGLEWVXTISSGGSYTYYTDSVKGRFTISRDNAKNXLYLQMNSLXXEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 233 aa, Artificial Sequence
  Description of Artificial Sequence Synthetic polypeptide
  MOD_RES (23)..(23): V or Q
  MOD_RES (29)..(29): A or S
  MOD_RES (30)..(30): T or S
  MOD_RES (33)..(33): L or A
  MOD_RES (35)..(35): P or V
  MOD_RES (37)..(37): E or D
  MOD_RES (39)..(39): A or V
  MOD_RES (41)..(41): L or I
  MOD_RES (42)..(42): S or T
  MOD_RES (62)..(62): Q or K
  MOD_RES (65)..(65): R or K
  MOD_RES (78)..(78): I or V
  MOD_RES (80)..(80): A or S
  MOD_RES (99)..(99): E or Q
  MOD_RES (105)..(105): V or T
  MOD_RES (107)..(107): F or Y
METDTLLLWVLLLWVPGSTGDIXMTQSPXXLSXSXGXRXTXXCRASQDISNYLNWYQQKPGXAVXLLIYYTSRLHSGXPXRFSGSGSGTDYTLTISSLXPEDFAXYXCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 13 aa, Artificial Sequence
  Description of Artificial Sequence Synthetic peptide
WATCAANNNNTTR

SEQ ID NO: 29 - protein, 90 aa, Escherichia coli
MNKTQLIDVIAEKAELSKTQAKAALESTLAAITESLKEGDAVQLVGFGTFKVNHRAERTGRNPQTGKEIKIAAANVPAFVSGKALKDAVK

SEQ ID NO: 30 - protein, 90 aa, Escherichia coli
MNKSQLIDKIAAGADISKAAAGRALDAIIASVTESLKEGDDVALVGFGTFAVKERAARTGRNPQTGKEITIAAAKVPSFRAGKALKDAVN

SEQ ID NO: 31 - protein, 21 aa, Haemophilus influenzae
NFELRDKSSRPGRNPKTGDVV

SEQ ID NO: 32 - protein, 21 aa, Haemophilus influenzae
SLHHRQPRLGRNPKTGDSVNL

SEQ ID NO: 33 - protein, 6 aa, Artificial Sequence
  Description of Artificial Sequence Synthetic peptide
GPSLKL

SEQ ID NO: 34 - protein, 4 aa, Artificial Sequence
  Description of Artificial Sequence Synthetic peptide
GPSL

SEQ ID NO: 35 - protein, 4 aa, Artificial Sequence
  Description of Artificial Sequence Synthetic peptide
PSLK

SEQ ID NO: 36 - protein, 5 aa, Artificial Sequence
  Description of Artificial Sequence Synthetic peptide
GPSLK

SEQ ID NO: 37 - protein, 4 aa, Artificial Sequence
  Description of Artificial Sequence Synthetic peptide
SLKL

SEQ ID NO: 38 - protein, 60 aa, Artificial Sequence
  Description of Artificial Sequence Synthetic polypeptide
  MOD_RES (7)..(7): Any amino acid
  MOD_RES (21)..(40): Any amino acid
  MISC_FEATURE (21)..(40): This region may encompass 1-20 residues
  MOD_RES (55)..(55): Any amino acid
RPGRNPXTGDVVPVSARRVVXXXXXXXXXXXXXXXXXXXXFSLHHRQPRLGRNPXTGDSV

SEQ ID NO: 39 - protein, 60 aa, Artificial Sequence
  Description of Artificial Sequence Synthetic polypeptide
  MOD_RES (21)..(40): Any amino acid
  MISC_FEATURE (21)..(40): This region may encompass 1-20 residues
RPGRNPKTGDVVPVSARRVVXXXXXXXXXXXXXXXXXXXXFSLHHRQPRLGRNPKTGDSV

SEQ ID NO: 40 - protein, 44 aa, Artificial Sequence
  Description of Artificial Sequence Synthetic polypeptide
RPGRNPKTGDVVPVSARRVVGPSLFSLHHRQPRLGRNPKTGDSV

SEQ ID NO: 41 - protein, 60 aa, Artificial Sequence
  Description of Artificial Sequence Synthetic polypeptide
  MOD_RES (21)..(40): Any amino acid
  MISC_FEATURE (21)..(40): This region may encompass 1-20 residues
FLEEIRLSLESGQDVKLSGFXXXXXXXXXXXXXXXXXXXXTLSAKEIENMVKDILEFISQ

SEQ ID NO: 42 - protein, 6 aa, Artificial Sequence
  Description of Artificial Sequence Synthetic peptide
GGSGGS

SEQ ID NO: 43 - protein, 6 aa, Artificial Sequence
  Description of Artificial Sequence Synthetic peptide
GPSLKL

SEQ ID NO: 44 - protein, 3 aa, Artificial Sequence
  Description of Artificial Sequence Synthetic peptide
GGG

SEQ ID NO: 45 - protein, 4 aa, Artificial Sequence
  Description of Artificial Sequence Synthetic peptide
GPSL

SEQ ID NO: 46 - protein, 3 aa, Artificial Sequence
  Description of Artificial Sequence Synthetic peptide
GPS

SEQ ID NO: 47 - protein, 4 aa, Artificial Sequence
  Description of Artificial Sequence Synthetic peptide
PSLK

SEQ ID NO: 48 - protein, 5 aa, Artificial Sequence
  Description of Artificial Sequence Synthetic peptide
GPSLK

SEQ ID NO: 49 - protein, 4 aa, Artificial Sequence
  Description of Artificial Sequence Synthetic peptide
SLKL

SEQ ID NO: 50 - protein, 44 aa, Artificial Sequence
  Description of Artificial Sequence Synthetic polypeptide
FLEEIRLSLESGQDVKLSGFGPSLTLSAKEIENMVKDILEFISQ

SEQ ID NO: 51 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 52 - protein, 215 aa, Artificial Sequence
  mHMGB1 (C45S)
MGKGDPKKPRGKMSSYAFFVQTCREEHKKKHPDASVNFSEFSKKSSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 53 - protein, 215 aa, Artificial Sequence
  mHMGB1 (C23S)
MGKGDPKKPRGKMSSYAFFVQTSREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 54 - protein, 215 aa, Artificial Sequence
  mHMGB1 (C106S)
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFSSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 55 - protein, 215 aa, Artificial Sequence
  mHMGB1 (C23S, C45S - double mutant)
MGKGDPKKPRGKMSSYAFFVQTSREEHKKKHPDASVNFSEFSKKSSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 56 - protein, 215 aa, Artificial Sequence
  mHMGB1 (C23S, C106S - double mutant)
MGKGDPKKPRGKMSSYAFFVQTSREEHKKKHPDASVNFSEFSKKCSERWKTMSAKEKGKFEDMAKADKARYEREMKTYIPPKGETKKKFKDPNAPKRPPSAFFLFSSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 57 - protein, 215 aa, Artificial Sequence
  mHMGB1 (C45S, C106S - double mutant)
MGKGDPKKPRGKMSSYAFFVQTCREEHKKKHPDASVNFSEFSKKSSERWKTMSAKEKGKFEDMAKADKARYEREMKTYIPPKGETKKKFKDPNAPKRPPSAFFLFSSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 58 - protein, 215 aa, Artificial Sequence
  mHMGB1 (C23S, C45S, C106S - triple mutant)
MGKGDPKKPRGKMSSYAFFVQTSREEHKKKHPDASVNFSEFSKKSSERWKTMSAKEKGKFEDMAKADKARYEREMKTYIPPKGETKKKFKDPNAPKRPPSAFFLFSSEYRPKIKGEHPGLSIGDVAKKLGEMWNNTAADDKQPYEKKAAKLKEKYEKDIAAYRAKGKPDAAKKGVVKAEKSKKKKEEEEGEEDEEDEEEEEDEEDEDEEEDDDDE

SEQ ID NO: 59 - DNA, 645 bp, Homo sapiens
atgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 60 - DNA, 645 bp, Artificial Sequence
  mHMGB1 (C23S)
atgggcaaaggagatcctaagaagccgagaggcaaaatgtcatcatatgcattttttgtgcaaactagtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 61 - DNA, 645 bp, Artificial Sequence
  mHMGB1 (C45S)
atgggcaaaggagatcctaagaagccgagaggcaaaatgtcatcatatgcattttttgtgcaaacttgtcgggaggagcataagaagaagcacccagatgcttcagtcaacttctcagagttttctaagaagagttcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 62 - DNA, 645 bp, Artificial Sequence
  mHMGB1 (C106S)
atgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttcagctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 63 - DNA, 645 bp, Artificial Sequence
  mHMGB1 (C23S, C45S - double mutant)
atgggcaaaggagatcctaagaagccgagaggcaaaatgtcatcatatgcattttttgtgcaaactagtcgggaggagcataagaagaagcacccagatgcttcagtcaacttctcagagttttctaagaagagttcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 64 - DNA, 645 bp, Artificial Sequence
  mHMGB1 (C23S, C106S - double mutant)
atgggcaaaggagatcctaagaagccgagaggcaaaatgtcatcatatgcattttttgtgcaaactagtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttcagctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 65 - DNA, 645 bp, Artificial Sequence
  mHMGB1 (C45S, C106S - double mutant)
atgggcaaaggagatcctaagaagccgagaggcaaaatgtcatcatatgcattttttgtgcaaacttgtcgggaggagcataagaagaagcacccagatgcttcagtcaacttctcagagttttctaagaagagttcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttcagctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 66 - DNA, 645 bp, Artificial Sequence
  mHMGB1 (C23S, C45S, C106S - triple mutant)
atgggcaaaggagatcctaagaagccgagaggcaaaatgtcatcatatgcattttttgtgcaaactagtcgggaggagcataagaagaagcacccagatgcttcagtcaacttctcagagttttctaagaagagttcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttcagctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaaggtgaggaagatgaagaggatgaggaggaggaggaagatgaagaagatgaagatgaagaagaagatgatgatgatgaa

SEQ ID NO: 67 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 68 - protein, 209 aa, Artificial Sequence
  mHMGB2 (C23S)
MGKGDPNKPRGKMSSYAFFVQTSREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 69 - protein, 209 aa, Artificial Sequence
  mHMGB2 (C45S)
MGKGDPNKPRGKMSSYAFFVQTCREEHKKKHPDSSVNFAEFSKKSSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 70 - protein, 209 aa, Artificial Sequence
  mHMGB2 (C106S)
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFSSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 71 - protein, 209 aa, Artificial Sequence
  mHMGB2 (C23S, C45S - double mutant)
MGKGDPNKPRGKMSSYAFFVQTSREEHKKKHPDSSVNFAEFSKKSSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 72 - protein, 209 aa, Artificial Sequence
  mHMGB2 (C23S, C106S - double mutant)
MGKGDPNKPRGKMSSYAFFVQTSREEHKKKHPDSSVNFAEFSKKCSERWKTMSAKEKSKFEDMAKSDKARYDREMKNYVPPKGDKKGKKKDPNAPKRPPSAFFLFSSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 73 - protein, 209 aa, Artificial Sequence
  mHMGB2 (C45S, C106S - double mutant)
MGKGDPNKPRGKMSSYAFFVQTCREEHKKKHPDSSVNFAEFSKKSSERWKTMSAKEKSKFEDMAKSDKARYDREMKNYVPPKGDKKGKKKDPNAPKRPPSAFFLFSSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 74 - protein, 209 aa, Artificial Sequence
  mHMGB2 (C23S, C45S, C106S - triple mutant)
MGKGDPNKPRGKMSSYAFFVQTSREEHKKKHPDSSVNFAEFSKKSSERWKTMSAKEKSKFEDMAKSDKARYDREMKNYVPPKGDKKGKKKDPNAPKRPPSAFFLFSSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 75 - DNA, 630 bp, Homo sapiens
atgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 76 - DNA, 630 bp, Artificial Sequence
  mHMGB2 (C23S)
atgggtaaaggagaccccaacaagccgcggggcaaaatgtcctcgtacgccttcttcgtgcagaccagtcgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 77 - DNA, 630 bp, Artificial Sequence
  mHMGB2 (C45S)
atgggtaaaggagaccccaacaagccgcggggcaaaatgtcctcgtacgccttcttcgtgcagacctgccgggaagagcacaagaagaaacacccggactcttccgtcaatttcgcggaattctccaagaagagttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 78 - DNA, 630 bp, Artificial Sequence
  mHMGB2 (C106S)
atgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgtttagctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 79 - DNA, 630 bp, Artificial Sequence
  mHMGB2 (C23S, C45S - double mutant)
atgggtaaaggagaccccaacaagccgcggggcaaaatgtcctcgtacgccttcttcgtgcagaccagtcgggaagagcacaagaagaaacacccggactcttccgtcaatttcgcggaattctccaagaagagttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 80 - DNA, 630 bp, Artificial Sequence
  mHMGB2 (C23S, C106S - double mutant)
atgggtaaaggagaccccaacaagccgcggggcaaaatgtcctcgtacgccttcttcgtgcagaccagtcgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgtttagctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 81 - DNA, 630 bp, Artificial Sequence
  mHMGB2 (C45S, C106S - double mutant)
atgggtaaaggagaccccaacaagccgcggggcaaaatgtcctcgtacgccttcttcgtgcagacctgccgggaagagcacaagaagaaacacccggactcttccgtcaatttcgcggaattctccaagaagagttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgtttagctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 82 - DNA, 630 bp, Artificial Sequence
  mHMGB2 (C23S, C45S, C106S - triple mutant)
atgggtaaaggagaccccaacaagccgcggggcaaaatgtcctcgtacgccttcttcgtgcagaccagtcgggaagagcacaagaagaaacacccggactcttccgtcaatttcgcggaattctccaagaagagttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgtttagctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataa

SEQ ID NO: 83 - protein, 200 aa, Homo sapiens
MAKGDPKKPKGKMSAYAFFVQTCREEHKKKNPEVPVNFAEFSKKCSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 84 - protein, 200 aa, Artificial Sequence
  mHMGB3(C23S)
MAKGDPKKPKGKMSAYAFFVQTSREEHKKKNPEVPVNFAEFSKKCSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 85 - protein, 200 aa, Artificial Sequence
  mHMGB3 (C45S)
MAKGDPKKPKGKMSAYAFFVQTCREEHKKKNPEVPVNFAEFSKKSSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 86 - protein, 200 aa, Artificial Sequence
  mHMGB3 (C104S)
MAKGDPKKPKGKMSAYAFFVQTCREEHKKKNPEVPVNFAEFSKKCSERWKTMSGKEKSKFDEMAKADKVRYDREMKDYGPAKGGKKKKDPNAPKRPPSGFFLFSSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 87 - protein, 200 aa, Artificial Sequence
  mHMGB3 (C23S, C45S - double mutant)
MAKGDPKKPKGKMSAYAFFVQTSREEHKKKNPEVPVNFAEFSKKSSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 88 - protein, 200 aa, Artificial Sequence
  mHMGB3 (C23S, C104S - double mutant)
MAKGDPKKPKGKMSAYAFFVQTSREEHKKKNPEVPVNFAEFSKKCSERWKTMSGKEKSKFDEMAKADKVRYDREMKDYGPAKGGKKKKDPNAPKRPPSGFFLFSSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 89 - protein, 200 aa, Artificial Sequence
  mHMGB3 (C45S, C104S - double mutant)
MAKGDPKKPKGKMSAYAFFVQTCREEHKKKNPEVPVNFAEFSKKSSERWKTMSGKEKSKFDEMAKADKVRYDREMKDYGPAKGGKKKKDPNAPKRPPSGFFLFSSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 90 - protein, 200 aa, Artificial Sequence
  mHMGB3 (C23S, C45S, C104S - triple mutant)
MAKGDPKKPKGKMSAYAFFVQTSREEHKKKNPEVPVNFAEFSKKSSERWKTMSGKEKSKFDEMAKADKVRYDREMKDYGPAKGGKKKKDPNAPKRPPSGFFLFSSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 91 - DNA, 604 bp, Homo sapiens
atggctaaaggtgaccccaagaaaccaaagggcaagatgtccgcttatgccttctttgtgcagacatgccagagaagaacataagaagaaaaacccagaggtccctgtcaattttgcggaattttccaagaagtgctctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttctgttcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 92 - DNA, 604 bp, Artificial Sequence
  mHMGB3 (C23S)
atggctaaaggtgaccccaagaaaccaaagggcaagatgtccgcttatgccttctttgtgcagacaagtcagagaagaacataagaagaaaaacccagaggtccctgtcaattttgcggaattttccaagaagtgctctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttctgttcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 93 - DNA, 604 bp, Artificial Sequence
  mHMGB3 (C45S)
atggctaaaggtgaccccaagaaaccaaagggcaagatgtccgcttatgccttctttgtgcagacatgccagagaagaacataagaagaaaaacccagaggtccctgtcaattttgcggaattttccaagaagagttctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttctgttcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 94 - DNA, 604 bp, Artificial Sequence
  mHMGB3 (C104S)
atggctaaaggtgaccccaagaaaccaaagggcaagatgtccgcttatgccttctttgtgcagacatgccagagaagaacataagaagaaaaacccagaggtccctgtcaattttgcggaattttccaagaagtgctctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttcagctcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 95 - DNA, 604 bp, Artificial Sequence
  mHMGB3 (C23S, C45S - double mutant)
atggctaaaggtgaccccaagaaaccaaagggcaagatgtccgcttatgccttctttgtgcagacaagtcagagaagaacataagaagaaaaacccagaggtccctgtcaattttgcggaattttccaagaagagttctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttctgttcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 96 - DNA, 604 bp, Artificial Sequence
  mHMGB3 (C23S, C104S - double mutant)
atggctaaaggtgaccccaagaaaccaaagggcaagatgtccgcttatgccttctttgtgcagacaagtcagagaagaacataagaagaaaaacccagaggtccctgtcaattttgcggaattttccaagaagtgctctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttcagctcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 97 - DNA, 604 bp, Artificial Sequence
  mHMGB3 (C45S, C104S - double mutant)
atggctaaaggtgaccccaagaaaccaaagggcaagatgtccgcttatgccttctttgtgcagacatgccagagaagaacataagaagaaaaacccagaggtccctgtcaattttgcggaattttccaagaagagttctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttcagctcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 98 - DNA, 604 bp, Artificial Sequence
  mHMGB3 (C23S, C45S, C104S - triple mutant)
atggctaaaggtgaccccaagaaaccaaagggcaagatgtccgcttatgccttctttgtgcagacaagtcagagaagaacataagaagaaaaacccagaggtccctgtcaattttgcggaattttccaagaagagttctgagaggtggaagacgatgtccgggaaagagaaatctaaatttgatgaaatggcaaaggcagataaagtgcgctatgatcgggaaatgaaggattatggaccagctaagggaggcaagaagaagaaggatcctaatgctcccaaaaggccaccgtctggattcttcctgttcagctcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaagaggaggaggaagaagaggaggaggaggaggaggaggatgaataa

SEQ ID NO: 99 - protein, 186 aa, Homo sapiens
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQCNARKKYRMSARNRCRGKRVRQS

SEQ ID NO: 100 - protein, 186 aa, Artificial Sequence
  mHMGB4 (C45S)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQCNARKKYRMSARNRCRGKRVRQS

SEQ ID NO: 101 - protein, 186 aa, Artificial Sequence
  mHMGB4 (C104S)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQCNARKKYRMSARNRCRGKRVRQS

SEQ ID NO: 102 - protein, 186 aa, Artificial Sequence
  mHMGB4 (C164S)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQSNARKKYRMSARNRCRGKRVRQS

SEQ ID NO: 103 - protein, 186 aa, Artificial Sequence
  mHMGB4 (C178S)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQCNARKKYRMSARNRSRGKRVRQS

SEQ ID NO: 104 - protein, 186 aa, Artificial Sequence
  mHMGB4 (C45S, C104S - double mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQCNARKKYRMSARNRCRGKRVRQS

SEQ ID NO: 105 - protein, 186 aa, Artificial Sequence
  mHMGB4 (C45S, C164S - double mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQSNARKKYRMSARNRCRGKRVRQS

SEQ ID NO: 106 - protein, 186 aa, Artificial Sequence
  mHMGB4 (C45S, C178S - double mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQCNARKKYRMSARNRSRGKRVRQS

SEQ ID NO: 107 - protein, 186 aa, Artificial Sequence
  mHMGB4 (C104S, C164S - double mutant)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQSNARKKYRMSARNRCRGKRVRQS

SEQ ID NO: 108 - protein, 186 aa, Artificial Sequence
  mHMGB4 (C104S, C178S - double mutant)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQCNARKKYRMSARNRSRGKRVRQS

SEQ ID NO: 109 - protein, 186 aa, Artificial Sequence
  mHMGB4 (C164S, C178S - double mutant)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQSNARKKYRMSARNRSRGKRVRQS

SEQ ID NO: 110 - protein, 186 aa, Artificial Sequence
  mHMGB4 (C45S, C104S, C164S - triple mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQSNARKKYRMSARNRCRGKRVRQS

SEQ ID NO: 111 - protein, 186 aa, Artificial Sequence
  mHMGB4 (C45S, C104S, C178S - triple mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQCNARKKYRMSARNRSRGKRVRQS

SEQ ID NO: 112 - protein, 186 aa, Artificial Sequence
  mHMGB4 (C45S, C164S, C178S - triple mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFCQDHYAQLKRENPNWSVVQVAKATGKMWSTATDLEKHPYEQRVALLRAKYFEELELYRKQSNARKKYRMSARNRSRGKRVRQS

SEQ ID NO: 113 - protein, 186 aa, Artificial Sequence
  mHMGB4 (C104S, C164S, C178S) - triple mutant)
MGKEIQLKPKANVSSYVHFLLNYRNKFKEQQPNTYVGFKEFSRKCSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQSNARKKYRMSARNRSRGKRVRQS

SEQ ID NO: 114 - protein, 186 aa, Artificial Sequence
  mHMGB4 (C45S, C104S, C164S, C178S - quadruple mutant)
MGKEIQLKPKANVSSYVHFLLNYRNKFKEQQPNTYVGFKEFSRKSSEKWRSISKHEKAKYEALAKLDKARYQEEMMNYVGKRKKRRKRDPQEPRRPPSSFLLFSQDHYAQLKRENPNWSVVQVAKATGKMWSTATDLEKHPYEQRVALLRAKYFEELELYRKQSNARKKYRMSARNRSRGKRVRQS

SEQ ID NO: 115 - DNA, 560 bp, Homo sapiens
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggtgcagagggaaaagagtcaggcagagctga

SEQ ID NO: 116 - DNA, 560 bp, Artificial Sequence
  mHMGB4 (C45S)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggtgcagagggaaaagagtcaggcagagctga

SEQ ID NO: 117 - DNA, 560 bp, Artificial Sequence
  mHMGB4 (C104S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggtgcagagggaaaagagtcaggcagagctga

SEQ ID NO: 118 - DNA, 560 bp, Artificial Sequence
  mHMGB4 (C164S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggtgcagagggaaaagagtcaggcagagctga

SEQ ID NO: 119 - DNA, 560 bp, Artificial Sequence
  mHMGB4 (C178S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggagcagagggaaaagagtcaggcagagctga

SEQ ID NO: 120 - DNA, 560 bp, Artificial Sequence
  mHMGB4 (C45S, C104S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggtgcagagggaaaagagtcaggcagagctga

SEQ ID NO: 121 - DNA, 560 bp, Artificial Sequence
  mHMGB4 (C45S, C164S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggtgcagagggaaaagagtcaggcagagctga

SEQ ID NO: 122 - DNA, 560 bp, Artificial Sequence
  mHMGB4 (C45S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggagcagagggaaaagagtcaggcagagctga

SEQ ID NO: 123 - DNA, 560 bp, Artificial Sequence
  mHMGB4 (C104S, C164S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggtgcagagggaaaagagtcaggcagagctga

SEQ ID NO: 124 - DNA, 560 bp, Artificial Sequence
  mHMGB4 (C104S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggagcagagggaaaagagtcaggcagagctga

SEQ ID NO: 125 - DNA, 560 bp, Artificial Sequence
  mHMGB4 (C164S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggagcagagggaaaagagtcaggcagagctga

SEQ ID NO: 126 - DNA, 560 bp, Artificial Sequence
  mHMGB4 (C45S, C104S, C164S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggtgcagagggaaaagagtcaggcagagctga

SEQ ID NO: 127 - DNA, 560 bp, Artificial Sequence
  mHMGB4 (C45S, C104S, C178S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacatgtaatgccaggaagaagtaccgaatgtcagctagaaaccggagcagagggaaaagagtcaggcagagctga

SEQ ID NO: 128 - DNA, 560 bp, Artificial Sequence
  mHMGB4 (C45S, C164S, C178S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggagcagagggaaaagagtcaggcagagctga

SEQ ID NO: 129 - DNA, 560 bp, Artificial Sequence
  mHMGB4 (C104S, C164S, C178S) - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggagcagagggaaaagagtcaggcagagctga

SEQ ID NO: 130 - DNA, 560 bp, Artificial Sequence
  mHMGB4 (C45S, C104S, C164S, C178S - quadruple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaagtaatgccaggaagaagtaccgaatgtcagctagaaaccggagcagagggaaaagagtcaggcagagctga

SEQ ID NO: 131 - protein, 10 aa, Artificial Sequence
  A peptide linker
PPKGETKKKF

SEQ ID NO: 132 - protein, 176 aa, Mus musculus
MGKGDPKKPRRKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAEKLKEKYEKDIAAYRAKGKPDAAKKGVV

SEQ ID NO: 133 - protein, 30 aa, Artificial Sequence
  A C box polypeptide
EEEEDEEDEEDEEEEEDEEDEDEEEDDDDE

SEQ ID NO: 134 - protein, 8 aa, Artificial Sequence
  T2A
  MISC_FEATURE (2)..(2): V or I
  misc_feature (4)..(4): Xaa can be any naturally occurring amino acid
DXEXNPGP

SEQ ID NO: 135 - DNA, 561 bp, Homo sapiens
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggtgcagagggaaaagagtcaggcagagctga

SEQ ID NO: 136 - DNA, 561 bp, Artificial Sequence
  mHMGB4 (C45S)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggtgcagagggaaaagagtcaggcagagctga

SEQ ID NO: 137 - DNA, 561 bp, Artificial Sequence
  mHMGB4 (C104S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggtgcagagggaaaagagtcaggcagagctga

SEQ ID NO: 138 - DNA, 561 bp, Artificial Sequence
  mHMGB4 (C164S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggtgcagagggaaaagagtcaggcagagctga

SEQ ID NO: 139 - DNA, 561 bp, Artificial Sequence
  mHMGB4 (C178S)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggagcagagggaaaagagtcaggcagagctga

SEQ ID NO: 140 - DNA, 561 bp, Artificial Sequence
  mHMGB4 (C45S, C104S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggtgcagagggaaaagagtcaggcagagctga

SEQ ID NO: 141 - DNA, 561 bp, Artificial Sequence
  mHMGB4 (C45S, C164S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggtgcagagggaaaagagtcaggcagagctga

SEQ ID NO: 142 - DNA, 561 bp, Artificial Sequence
  mHMGB4 (C45S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggagcagagggaaaagagtcaggcagagctga

SEQ ID NO: 143 - DNA, 561 bp, Artificial Sequence
  mHMGB4 (C104S, C164S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggtgcagagggaaaagagtcaggcagagctga

SEQ ID NO: 144 - DNA, 561 bp, Artificial Sequence
  mHMGB4 (C104S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggagcagagggaaaagagtcaggcagagctga

SEQ ID NO: 145 - DNA, 561 bp, Artificial Sequence
  mHMGB4 (C164S, C178S - double mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggagcagagggaaaagagtcaggcagagctga

SEQ ID NO: 146 - DNA, 561 bp, Artificial Sequence
  mHMGB4 (C45S, C104S, C164S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggtgcagagggaaaagagtcaggcagagctga

SEQ ID NO: 147 - DNA, 561 bp, Artificial Sequence
  mHMGB4 (C45S, C104S, C178S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaatgtaatgccaggaagaagtaccgaatgtcagctagaaaccggagcagagggaaaagagtcaggcagagctga

SEQ ID NO: 148 - DNA, 561 bp, Artificial Sequence
  mHMGB4 (C45S, C164S, C178S - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttctgccaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggagcagagggaaaagagtcaggcagagctga

SEQ ID NO: 149 - DNA, 561 bp, Artificial Sequence
  mHMGB4 (C104S, C164S, C178S) - triple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagtgttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggagcagagggaaaagagtcaggcagagctga

SEQ ID NO: 150 - DNA, 561 bp, Artificial Sequence
  mHMGB4 (C45S, C104S, C164S, C178S - quadruple mutant)
atgggaaaagaaatccagctaaagcctaaggcaaatgtctcttcttacgttcactttttgctgaattacagaaacaaattcaaggagcagcagccaaatacctatgttggctttaaagagttctctagaaagagttcggaaaaatggagatccatctcaaagcatgaaaaggccaaatatgaagccctggccaaactcgacaaagcccgataccaggaagaaatgatgaattatgttggcaagaggaagaaacggagaaagcgggatccccaggaacccagacggcctccatcatccttcctactcttcagtcaagaccactatgctcagctgaagagggagaacccgaactggtcggtggtgcaggtggccaaggccacagggaagatgtggtcaacagcgacagacctggagaagcacccttatgagcaaagagtggctctcctgagagctaagtacttcgaggaacttgaactctaccgtaaacaaagtaatgccaggaagaagtaccgaatgtcagctagaaaccggagcagagggaaaagagtcaggcagagctga

(19) United States
(12) Patent Application Publication
(10) Pub. No.: US 2023/0272054 A1
(43) Pub. Date: Aug. 31, 2023

(54) COMBINATION THERAPIES FOR THE TREATMENT AND PREVENTION OF BIOFILMS

(71) Applicant: RESEARCH INSTITUTE AT NATIONWIDE CHILDREN'S HOSPITAL, Columbus, OH (US)

(72) Inventors: Steven D. Goodman, Columbus, OH (US); Lauren O. Bakaletz, Columbus, OH (US)

(21) Appl. No.: 18/014,980

(22) PCT Filed: Jul. 6, 2021
(86) PCT No.: PCT/US2021/040576

§ 371 Date: Jan. 6, 2023

Related U.S. Application Data
(60) Provisional application No. 63/049,065, filed on Jul. 7, 2020.
(60) Provisional application No. 63/175,487, filed on Apr. 15, 2021.

(51) Int. Cl. C07K 16/12; A61P 31/04; C07K 16/24; A61K 39/40

(52) U.S. Cl. CPC C07K 16/12; A61P 31/04; C07K 16/24; A61K 39/40; C07K 2317/565; A61K 38/00

(57) ABSTRACT

Provided herein are compositions and combinations for the therapeutic and diagnostic use in treating and preventing biofilms and associated disorders using a high mobility group box protein (HMGB) polypeptide, mutant and/or fragment thereof and an anti-DNABII antibody, fragment or variant thereof. The polypeptide and antibody can be administered in the same or separate compositions.

### CROSS-REFERENCE TO RELATED APPLICATIONS

This application claims priority under 35 U.S.C. § 119(e) of U.S. Provisional Application Nos. 63/049,065 and 63/175,487, filed Jul. 7, 2020 and Apr. 15, 2021, respectively, the contents of each of which is incorporated by reference in its entireties into the present application.

### STATEMENT OF GOVERNMENT SUPPORT

This invention was made with government support under Grant No. DC011818 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 2, 2021, is named 106887-7960_ST25.txt and is 233,163 bytes in size.

### BACKGROUND

The DNABII family of proteins are naturally found outside of the bacterial cell and contribute to biofilm formation. At least one protein from the DNABII family is found in all known eubacteria. While these proteins elicit a strong innate and acquired immune response, host subjects fail to naturally produce immunoprotective antibodies to family members as a result of infection. The major problem with bacterial biofilms is the inability of the host immune system and/or antibiotics and other antimicrobials to gain access to the bacteria protected within the biofilm.

Bacteria within a human host prefer a community architecture, also known as a biofilm. Biofilms form when free-living (planktonic) bacteria adhere to each other (aggregate biofilms) or adhere to a surface (attached biofilm) and initiate a developmental program that includes alterations in gene expression, intercellular communication, and importantly production of a self-made extracellular matrix (extracellular polymeric substances or EPS). The resident bacteria within each of these community architectures are recalcitrant to the host immune system and antimicrobials (Slinger et al., Diagn Microbiol Infect Dis, 2006. 56(3): p. 247-53; and Starner et al., Antimicrob Agents Chemother, 2008. 52(1): p. 137-45), which enables the bacteria to persist and serve as reservoirs to maintain chronic and recurrent infections. Hence, there is a critical need to develop targeted strategies to resolve bacterial biofilms.

Biofilms are present in an industrial setting as well. For example, biofilms are implicated in a wide range of petroleum process problems, from the production field to the gas station storage tank. In the field, sulfate reducing biofilm bacteria produce hydrogen sulfide (soured oil). In the process pipelines, biofilm activity develops slimes which impede filters and orifices. Biofilm and biofilm organisms also cause corrosion of pipeline and petroleum process equipment. These problems can be manifested throughout an oil or gas production facility, to the point where fouling and corrosive biofilm organisms have even been found on the surfaces of final product storage tanks.

Biofilms are implicated in a wide range of water processes, both domestic and industrial. They can grow on the surface of process equipment and impede the performance of the equipment, such as degradation of heat transfer or plugging of filters and membranes. Biofilms growing on a cooling tower fill can add enough weight to cause collapse of the fill. Biofilms cause corrosion of even highly specialized stainless steels. Biofilms in a water process can degrade the value of a final product such as biofilm contamination in a paper process or the attachment of even a single cell on a silicon chip. Biofilms growing in drinking water distribution systems can harbor potential pathogenic organisms, corrosive organisms or bacteria that degrade the aesthetic quality of the water. In the home, biofilms are found in or on any surface that supports microbial growth, e.g., in drains, on food preparation surfaces, in toilets, and in swimming pools and spas.

Thus, a need exists to break through the protective barrier of biofilms to treat or kill the associated bacterial infections and clear them from surfaces and in water systems. This disclosure satisfies this need and provides related advantages as well.

### SUMMARY

Provided herein is a combination of an HMG-box polypeptide (a non-limiting example of such comprises the HMGB1 polypeptide) and an anti-DNABII antibody or an antigen binding fragment thereof (such as an antibody specifically recognizing and binding a tip or chimeric tip domain of the DNABII) or an equivalent of each thereof. Additionally provided are polynucleotides, vectors, and host cells expressing the elements of the combination alone or in combination with each other. Yet additionally provided are compositions, combinations and kits comprising, or consisting essentially of, or yet further consisting of the combination, or the polynucleotide or vector or host cell expressing the combination, or both, along with methods of producing or using the same. Without wishing to be bound by the theory, the combination as disclosed shows synergistic effect in preventing the formation of or disrupting a biofilm.

In one aspect, provided herein is a composition or combination comprising, or consisting essentially of, or yet further consisting of:
(a) a high mobility group box protein (HMGB) polypeptide, optionally a HMGB1 polypeptide, further optionally comprising one or more mutations (i.e., a mutant HMGB1 (mHMGB1) polypeptide), or a fragment thereof optionally comprising, or consisting essentially of, or consisting of a B box or an A box or both (e.g., an AB box) of the HMGB optionally a HMGB1 polypeptide, optionally wherein the HMGB polypeptide or HMGB1 polypeptide or fragment thereof is isolated or engineered or both; and
(b) an anti-DNABII antibody or an antigen-binding fragment thereof comprising, or alternatively consisting essentially of, or yet further consisting of:
   (i) a heavy chain (HC) immunoglobulin variable domain comprising, or alternatively consisting essentially of, or yet further consisting of amino acid (aa) 25 to aa 144 of any one of SEQ ID NO: 13, 24 or 26, or an equivalent of each thereof, and/or
   (ii) a light chain (LC) immunoglobulin variable domain comprising, or alternatively consisting essentially of, or yet further consisting of: aa 21 to aa 132 of SEQ ID NO: 14 or 25, or aa 21 to aa 126 of SEQ ID NO: 27, or an equivalent of each thereof.

In a further aspect, provided is a composition or combination comprising, or consisting essentially of, or yet further consisting of:
(a) a high mobility group box protein (HMGB) polypeptide optionally a HMGB1 polypeptide, further optionally comprising one or more mutations (e.g., a mutant HMGB1 (mHMGB1) polypeptide), or a fragment thereof optionally comprising, or consisting essentially of, or consisting of a B box or an A box or both (e.g., an AB box) of the HMGB polypeptide, optionally wherein the HMGB1 polypeptide or fragment thereof is isolated or engineered or both; and
(b) an anti-DNABII antibody or an antigen-binding fragment thereof comprising, or alternatively consisting essentially of, or yet further consisting of:
   (i) a heavy chain (HC) comprising, or alternatively consisting essentially of, or yet further consisting of: any one or more of SEQ ID NOs: 13, 24 or 26, or an equivalent of each thereof, and/or
   (ii) a light chain (LC) comprising, or alternatively consisting essentially of, or yet further consisting of: any one or more of SEQ ID NOs: 14, 25, or 27, or an equivalent of each thereof.

In yet a further aspect, provided is a composition or combination comprising, or consisting essentially of, or yet further consisting of:
(a) a high mobility group box protein (HMGB) polypeptide, optionally a HMGB1 polypeptide optionally comprising one or more mutations (i.e., a mutant HMGB1 (mHMGB1) polypeptide (or corresponding mutations in HMGB2, HMGB3, or HMGB4), or a fragment thereof optionally comprising, or consisting essentially of, or consisting of a B box or an A box or both (e.g., an AB box) of the HMGB1 polypeptide, optionally wherein the HMGB1 polypeptide or fragment thereof is isolated or engineered or both; and
(b) an anti-DNABII antibody or an antigen-binding fragment thereof comprising, or alternatively consisting essentially of, or yet further consisting of one or two or three or four or five or all six of the following (i) to (vi):
   (i) a heavy chain complementarity-determining region 1 (CDRH1) comprising, or alternatively consisting essentially of, or yet further consisting of any one or more of: GFTFXXY (amino acid (aa) 50 to aa 56 of SEQ ID NO: 13), GFTFRTY (aa 50 to aa 56 of SEQ ID NO: 1 or 2 or 3 or 24), or GFTFSRY (aa 50 to aa 56 of SEQ ID NO: 4 or 5 or 6 or 26), wherein X is any amino acid or an amino acid at the aligned aa position of a sequence selected from SEQ ID NOs: 1-6;
   (ii) a heavy chain complementarity-determining region 2 (CDRH2) comprising, or alternatively consisting essentially of, or yet further consisting of any one or more of: XSXXXX (amino acid (aa) 76 to aa 81 of SEQ ID NO: 13), GSDRRH (aa 76 to aa 81 of SEQ ID NO: 1 or 2 or 3 or 24), or SSGGSY (aa 76 to aa 81 of SEQ ID NO: 4 or 5 or 6 or 26), wherein X is any amino acid or an amino acid at the aligned aa position of a sequence selected from SEQ ID NOs: 1-6; (iii) a heavy chain complementarity-determining region 3 (CDRH3) comprising, or alternatively consisting essentially of, or yet further consisting of any one or more of: XXXXXXXYXXFDX (amino acid (aa) 121 to aa 133 of SEQ ID NO: 13), VGPYDGYYGEFDY (aa 121 to aa 133 of SEQ ID NO: 1 or 2 or 3 or 24), or ERHGGDGYWYFDV (aa 121 to aa 133 of SEQ ID NO: 4 or 5 or 6 or 26), wherein X is any amino acid or an amino acid at the aligned aa position of a sequence selected from SEQ ID NOs: 1-6; (iv) a light chain complementarity-determining region 1 (CDRL1) comprising, or alternatively consisting essentially of, or yet further consisting of any one or more of: QXXXXXXXXXX (aa 47 to aa 57 of SEQ ID NO: 14), QXXXXX (aa 47 to aa 52 of SEQ ID NO: 14), QSLLDSDGKTF (aa 47 to aa 57 of SEQ ID NO: 7 or 8 or 9 or 25), or QDISNY (aa 47 to aa 52 of SEQ ID NO: 10 or 11 or 12 or 27), wherein X is any amino acid or an amino acid at the aligned aa position of a sequence selected from SEQ ID NOs: 7-12;
(v) a light chain complementarity-determining region 2 (CDRL2) comprising, or alternatively consisting essentially of, or yet further consisting of any one or more of: XXS (aa 75 to aa 77 of SEQ ID NO: 14), LVS (aa 75 to aa 77 of SEQ ID NO: 7 or 8 or 9 or 25), or YTS (aa 70 to aa 72 of SEQ ID NO: 10 or 11 or 12 or 27), wherein X is any amino acid or an amino acid at the aligned aa position of a sequence selected from SEQ ID NOs: 7-12; and (vi) a light chain complementarity-determining region 3 (CDRL3) comprising, or alternatively consisting essentially of, or yet further consisting of any one or more of: XQGXXXXXT (aa 114 to aa 122 of SEQ ID NO: 14), WQGTHFPYT (aa 114 to aa 122 of SEQ ID NO: 7 or 8 or 9 or 25), or QQGNPLRT (aa 109 to aa 116 of SEQ ID NO: 10 or 11 or 12 or 27), wherein X is any amino acid or an amino acid at the aligned aa position of a sequence selected from SEQ ID NOs: 7-12.

In one aspect, provided is a composition or combination comprising, or consisting essentially of, or yet further consisting of:
(a) a high mobility group box protein (HMGB) polypeptide (HMGB) polypeptide, optionally a HMGB1 polypeptide, further optionally comprising one or more mutations (i.e., a mutant HMGB1 (mHMGB1) polypeptide or corresponding mutations in HMGB2, HMGB3, or HMGB4), or a fragment thereof optionally comprising, or consisting essentially of, or consisting of a B box or an A box or both (e.g., an AB box) of the HMGB1 polypeptide, optionally wherein the HMGB1 polypeptide or fragment thereof is isolated or engineered or both; and
(b) an anti-DNABII antibody or an antigen-binding fragment thereof comprising, or alternatively consisting essentially of, or yet further consisting of one or two or three or four or five or all six of the following (i) to (vi):
   (i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
   (ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
   (iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
   (iv) a light chain complementarity-determining region 1 (CDRL1) comprising, or alternatively consisting essentially of, or yet further consisting of QSLLDSDGKTF (aa 47 to aa 57 of SEQ ID NO: 7 or 8 or 9 or 25);
   (v) a light chain complementarity-determining region 2 (CDRL2) comprising, or alternatively consisting essentially of, or yet further consisting of LVS (aa 75 to aa 77 of SEQ ID NO: 7 or 8 or 9 or 25); and
   (vi) a light chain complementarity-determining region 3 (CDRL3) comprising, or alternatively consisting essentially of, or yet further consisting of WQGTHFP (aa 114 to aa 120 of SEQ ID NO: 7 or 8 or 9 or 25).

In another aspect, provided is a composition or combination comprising, or consisting essentially of, or yet further consisting of:
(a) a high mobility group box protein HMGB polypeptide, optionally a HMGB1 polypeptide, or a fragment thereof comprising, or consisting essentially of, or yet further consisting of a B box or an A box or an AB box thereof, and
(b) an anti-DNABII antibody or an antigen-binding fragment thereof comprising, or consisting essentially of, or yet further consisting of:
   (i) a heavy chain complementarity-determining region 1 (CDRH1) comprising, or consisting essentially of, or yet further consisting of GFTFRTY (aa 50 to aa 56 of SEQ ID NO: 1 or 2 or 3 or 24);
   (ii) a heavy chain complementarity-determining region 2 (CDRH2) comprising, or consisting essentially of, or yet further consisting of GSDRRH (aa 76 to aa 81 of SEQ ID NO: 1 or 2 or 3 or 24);
   (iii) a heavy chain complementarity-determining region 3 (CDRH3) comprising, or consisting essentially of, or yet further consisting of VGPYDGYYGEFDY (aa 121 to aa 133 of SEQ ID NO: 1 or 2 or 3 or 24);
   (iv) a light chain complementarity-determining region 1 (CDRL1) comprising, or consisting essentially of, or yet further consisting of QSLLDSDGKTF (aa 47 to aa 57 of SEQ ID NO: 7 or 8 or 9 or 25);
   (v) a light chain complementarity-determining region 2 (CDRL2) comprising, or consisting essentially of, or yet further consisting of LVS (aa 75 to aa 77 of SEQ ID NO: 7 or 8 or 9 or 25); and
   (vi) a light chain complementarity-determining region 3 (CDRL3) comprising, or consisting essentially of, or yet further consisting of WQGTHFPYT (aa 114 to aa 122 of SEQ ID NO: 7 or 8 or 9 or 25).

In yet another aspect, provided is a composition or combination comprising, or consisting essentially of, or yet further consisting of:
(a) a high mobility group box protein (HMGB) polypeptide, optionally a HMGB1 polypeptide or a fragment thereof comprising, or consisting essentially of, or consisting of a B box or an A box or an AB box thereof, and
(b) an antibody or an antigen-binding fragment thereof specifically recognizing and binding a tip domain of a DNABII protein, with the proviso that
   (i) the composition or combination does not comprise SEQ ID NO: 52, or
   (ii) the antigen-binding fragment does not comprise an Fab optionally an Fab of polyclonal antibodies or the antibody that does not comprise polyclonal antibodies, or
   both (i) and (ii).

In one aspect, provided is a composition or combination comprising, or alternatively consisting essentially of, or yet further consisting of:
(a) a high mobility group box protein (HMGB) polypeptide optionally a HMGB1 polypeptide optionally comprising one or more mutations (i.e., a mutant HMGB1 (mHMGB1) polypeptide or corresponding mutations in HMGB2, HMGB3, or HMGB4, or a fragment thereof optionally comprising, or consisting essentially of, or consisting of a B box or an A box or both (e.g., an AB box) of the HMGB1 polypeptide, optionally wherein the HMGB1 polypeptide or fragment thereof is isolated or engineered or both; and
(b) an anti-DNABII antibody or an antigen-binding fragment thereof comprising, or alternatively consisting essentially of, or yet further consisting of:
   (i) CDRs 1-3 of any one of SEQ ID NO: 1-6, 13, 24 or 26, or an equivalent of each thereof; and/or
   (ii) CDRs 1-3 of any one of SEQ ID NO: 7-12, 14, 25 or 27, or an equivalent of each thereof.

In a further aspect, provided is a composition or combination comprising, or alternatively consisting essentially of, or yet further consisting of:
(a) a high mobility group box protein (HMGB) polypeptide optionally a HMGB1 polypeptide optionally comprising one or more mutations (i.e., a mutant HMGB1 (mHMGB1) polypeptide), or a fragment thereof optionally comprising, or consisting essentially of, or consisting of a B box or an A box or both (e.g., an AB box) of the HMGB1 polypeptide, optionally wherein the HMGB polypeptide, optionally the HMGB1 polypeptide or fragment thereof is isolated or engineered or both; and
(b) an anti-DNABII antibody or an antigen-binding fragment thereof that competes for binding to an epitope with an antibody or an antigen-binding fragment thereof as disclosed herein.

In yet a further aspect, provided is a composition or combination comprising, or alternatively consisting essentially of, or yet further consisting of:
(a) a high mobility group box protein (HMGB) polypeptide, optionally a HMGB1 polypeptide optionally comprising one or more mutations (e.g., a mutant HMGB1 (mHMGB1) polypeptide or corresponding mutations in the HMGB2, HMGB3, or HMGB4 polypeptides), or a fragment thereof optionally comprising, or consisting essentially of, or consisting of a B box or an A box or both (e.g., an AB box) of the HMGB1 polypeptide, optionally wherein the HMGB1 polypeptide or fragment thereof is isolated or engineered or both; and
(b) a polypeptide comprising a complementarity-determining region (CDR) as disclosed herein. In some embodiments, the combination is separately formulated to be administered in combination. In some embodiments, the CDR comprises, or consists essentially of, or yet further consists of any one or more of heavy chain (HC) CDR 1 (CDRH1), HC CDR 2 (CDRH2), HC CDR 3 (CDRH3), light chain (LC) CDR 1 (CDRL1), LC CDR 2 (CDRL2), or LC CDR 3 (CDRL3).

In one aspect, provided is a composition or combination comprising, or alternatively consisting essentially of, or yet further consisting of:
(a) a high mobility group box protein (HMGB) polypeptide, optionally a HMGB1 polypeptide, further optionally comprising one or more mutations (e.g., a mutant HMGB1 (mHMGB1) polypeptide or corresponding mutations in the HMGB2, HMGB3, or HMGB4 polypeptides), or a fragment thereof optionally comprising, or consisting essentially of, or consisting of a B box or an A box or both (e.g., the AB box) of the HMGB1 polypeptide, optionally wherein the HMGB1 polypeptide or fragment thereof is isolated or engineered or both; and
(b) a complementarity-determining region (CDR) comprising, or alternatively consisting essentially of, or yet further consisting of any one or more of the following:
   (i) a heavy chain complementarity-determining region 1 (CDRH1) comprising, or alternatively consisting essentially of, or yet further consisting of any one of: GFTFXXY (amino acid (aa) 50 to aa 56 of SEQ ID NO: 13, wherein X is any amino acid or an amino acid at the aligned aa position of a sequence selected from SEQ ID NOs: 1-6), GFTFRTY (aa 50 to aa 56 of SEQ ID NO: 1 or 2 or 3 or 24), GFTFSRY (aa 50 to aa 56 of SEQ ID NO: 4 or 5 or 6 or 26), GFTFRTYA (aa 50 to aa 57 of SEQ ID NO: 1 or 2 or 3 or 24), aASGFTFRTYAMS (aa 47 to aa 59 of SEQ ID NO: 24: wherein the small letter a is A, i.e., aa 47 to aa 59 of SEQ ID NO: 1 or 2, or wherein the small letter a is K, i.e., aa 47 to aa 59 of SEQ ID NO: 3), GFTFSRYG (aa 50 to aa 57 of SEQ ID NO: 4 or 5 or 6 or 26), or aASGFTFSRYGMS (aa 47 to aa 59 of SEQ ID NO: 26: wherein the small letter a is A, i.e., aa 47 to aa 59 of SEQ ID NO: 4 or 5, or wherein the small letter a is T, i.e., aa 47 to aa 59 of SEQ ID NO: 6);
   (ii) a heavy chain complementarity-determining region 2 (CDRH2) comprising, or alternatively consisting essentially of, or yet further consisting of any one of: XSXXXX (amino acid (aa) 76 to aa 81 of SEQ ID NO: 13, wherein X is any amino acid or an amino acid at the aligned aa position of a sequence selected from SEQ ID NOs: 1-6), GSDRRH (aa 76 to aa 81 of SEQ ID NO: 1 or 2 or 3 or 24), SSGGSY (aa 76 to aa 81 of SEQ ID NO: 4 or 5 or 6 or 26), IGSDRRHT (aa 75 to aa 82 of SEQ ID NO: 1 or 2 or 3 or 24), IGSDRRHTY (aa 75 to aa 83 of SEQ ID NO: 1 or 2 or 3 or 24), TIGSDRRHTY (aa 74 to aa 83 of SEQ ID NO: 1 or 2 or 3 or 24), WVATIGSDRRHTYYP (aa 71 to aa 85 of SEQ ID NO: 1 or 2 or 3 or 24), ISSGGSYT (aa 75 to aa 82 of SEQ ID NO: 4 or 5 or 6 or 26), or TISSGGSYTY (aa 74 to aa 83 of SEQ ID NO: 4 or 5 or 6 or 26);
   (iii) a heavy chain complementarity-determining region 3 (CDRH3) comprising, or alternatively consisting essentially of, or yet further consisting of any one of: XXXXXXXYXXFDX (amino acid (aa) 121 to aa 133 of SEQ ID NO: 13, wherein X is any amino acid or an amino acid at the aligned aa position of a sequence selected from SEQ ID NOs: 1-6), VGPYDGYYGEFDY (aa 121 to aa 133 of SEQ ID NO: 1 or 2 or 3 or 24), ERHGGDGYWYFDV (aa 121 to aa 133 of SEQ ID NO: 4 or 5 or 6 or 26), VGPYDGYYGEFDY (aa 121 to aa 133 of SEQ ID NO: 1 or 2 or 3 or 24), or ER (aa 121 to aa 122 of SEQ ID NO: 4 or 5 or 6 or 26);
   (iv) a light chain complementarity-determining region 1 (CDRL1) comprising, or alternatively consisting essentially of, or yet further consisting of any one of: QXXXXXXXXXX (aa 47 to aa 57 of SEQ ID NO: 14, wherein X is any amino acid or an amino acid at the aligned aa position of a sequence selected from SEQ ID NOs: 7-12), QXXXXX (aa 47 to aa 52 of SEQ ID NO: 14, wherein X is any amino acid or an amino acid at the aligned aa position of a sequence selected from SEQ ID NOs: 7-12), QSLLDSDGKTF (aa 47 to aa 57 of SEQ ID NO: 7 or 8 or 9 or 25), QDISNY (aa 47 to aa 52 of SEQ ID NO: 10 or 11 or 12 or 27), rSSQSLLDSDGKTFLN (aa 44 to aa 59 of SEQ ID NO: 25: wherein the smaller letter r is R, i.e., aa 44 to aa 59 of SEQ ID NO: 7 or 8, or wherein the smaller letter r is K, i.e., aa 44 to aa 59 of SEQ ID NO: 9, or RASQDISNYLN (aa 44 to aa 54 of SEQ ID NO: 10 or 11 or 12 or 27);
   (v) a light chain complementarity-determining region 2 (CDRL2) comprising, or alternatively consisting essentially of, or yet further consisting of any one of: XXS (aa 75 to aa 77 of SEQ ID NO: 14, wherein X is any amino acid or an amino acid at the aligned aa position of a sequence selected from SEQ ID NOs: 7-12), LVS (aa 75 to aa 77 of SEQ ID NO: 7 or 8 or 9 or 25), YTS (aa 70 to aa 72 of SEQ ID NO: 10 or 11 or 12 or 27), LVSKlDS (aa 75 to aa 81 of SEQ ID NO: 25: wherein the smaller letter l is L, i.e., aa 75 to aa 81 of SEQ ID NO: 7 or 9, or wherein the smaller letter l is R, i.e., aa 75 to aa 81 of SEQ ID NO: 8), YLVSKlDS (aa 74 to aa 81 of SEQ ID NO: 25: wherein the smaller letter l is L, i.e., aa 74 to aa 81 of SEQ ID NO: 7 or 9, or wherein the smaller letter l is R, i.e., aa 74 to aa 81 of SEQ ID NO: 8), LVSKlDSG (aa 75 to aa 82 of SEQ ID NO: 25: wherein the smaller letter l is L, i.e., aa 75 to aa 82 of SEQ ID NO: 7 or 9, or wherein the smaller letter l is R, i.e., aa 75 to aa 82 of SEQ ID NO: 8), YLVSKlDSGV (aa 74 to aa 83 of SEQ ID NO: 25: wherein the smaller letter l is L, i.e., aa 74 to aa 83 of SEQ ID NO: 7 or 9, or wherein the smaller letter l is R, i.e., aa 74 to aa 83 of SEQ ID NO: 8), RLIYLVSKlDSGVPD (aa 71 to aa 85 of SEQ ID NO: 25: wherein the smaller letter l is L, i.e., aa 71 to aa 85 of SEQ ID NO: 7 or 9, or wherein the smaller letter l is R, i.e., aa 71 to aa 85 of SEQ ID NO: 8), YTSRLHS (aa 70 to aa 76 of SEQ ID NO: 10 or 11 or 12 or 27); or YYTSRLHS (aa 69 to aa 76 of SEQ ID NO: 10 or 11 or 12 or 27); and
   (vi) a light chain complementarity-determining region 3 (CDRL3) comprising, or alternatively consisting essentially of, or yet further consisting of any one of: XQGXXXXXT (aa 114 to aa 122 of SEQ ID NO: 14, wherein X is any amino acid or an amino acid at the aligned aa position of a sequence selected from SEQ ID NOs: 7-12), WQGTHFPYT (aa 114 to aa 122 of SEQ ID NO: 7 or 8 or 9 or 25), QQGNPLRT (aa 109 to aa 116 of SEQ ID NO: 10 or 11 or 12 or 27), WQGTHFP (aa 114 to aa 120 of SEQ ID NO: 7 or 8 or 9 or 25), WQGTHFPY (aa 114 to aa 121 of SEQ ID NO: 7 or 8 or 9 or 25), WQGTHFPYT (aa 114 to aa 122 of SEQ ID NO: 7 or 8 or 9 or 25), or QQ (aa 109 to aa 110 of SEQ ID NO: 10 or 11 or 12 or 27).

In a further aspect, provided is a composition or combination comprising, or alternatively consisting essentially of, or yet further consisting of:
(a) a high mobility group box protein (HMGB) polypeptide, optionally the HMGB1 polypeptide, further optionally comprising one or more mutations (i.e., a mutant HMGB1 (mHMGB1) polypeptide or corresponding mutations in the HMGB2, HMGB3, or HMGB4 polypeptides), or a fragment thereof optionally comprising, or consisting essentially of, or consisting of a B box or an A box or both (e.g., an AB box) of the HMGB1 polypeptide, optionally wherein the HMGB1 polypeptide or fragment thereof is isolated or engineered or both; and
(b) an isolated polypeptide comprising, or consisting essentially of, or yet further consisting of any one or more of SEQ ID NOs: 1-14 or 24-27, or an equivalent of each thereof.

In yet a further aspect, provided is a composition or combination comprising, or alternatively consisting essentially of, or yet further consisting of:
(a) an isolated polynucleotide encoding the HMGB polypeptide, optionally HMGB1 polypeptide, or fragment thereof as disclosed herein, or an equivalent of each thereof, and optionally operatively linked to a regulatory sequence directing the expression thereof, such as a promoter or an enhancer or both; and
(b) an isolated polynucleotide encoding the antibody or antigen-binding fragment thereof as disclosed herein, or an equivalent of each thereof, and optionally operatively linked to a regulatory sequence directing the expression thereof, such as a promoter or an enhancer or both.

In one aspect, provided is a polypeptide comprising, or consisting essentially of, or yet further consisting of:
(a) a high mobility group box protein (HMGB) polypeptide, optionally HMGB1 polypeptide, or a fragment thereof comprising, or consisting essentially of, or consisting of a B box or an A box or an AB box thereof, and
(b) an anti-DNABII antibody or an antigen-binding fragment thereof as disclosed herein, such as the antibody or antigen-binding fragment thereof comprising, or consisting essentially of, or yet further consisting of:
   (i) a heavy chain complementarity-determining region 1 (CDRH1) comprising, or consisting essentially of, or yet further consisting of GFTFRTY (aa 50 to aa 56 of SEQ ID NO: 1 or 2 or 3 or 24);
   (ii) a heavy chain complementarity-determining region 2 (CDRH2) comprising, or consisting essentially of, or yet further consisting of GSDRRH (aa 76 to aa 81 of SEQ ID NO: 1 or 2 or 3 or 24);
   (iii) a heavy chain complementarity-determining region 3 (CDRH3) comprising, or consisting essentially of, or yet further consisting of VGPYDGYYGEFDY (aa 121 to aa 133 of SEQ ID NO: 1 or 2 or 3 or 24);
   (iv) a light chain complementarity-determining region 1 (CDRL1) comprising, or consisting essentially of, or yet further consisting of QSLLDSDGKTF (aa 47 to aa 57 of SEQ ID NO: 7 or 8 or 9 or 25);
   (v) a light chain complementarity-determining region 2 (CDRL2) comprising, or consisting essentially of, or yet further consisting of LVS (aa 75 to aa 77 of SEQ ID NO: 7 or 8 or 9 or 25); and
   (vi) a light chain complementarity-determining region 3 (CDRL3) comprising, or consisting essentially of, or yet further consisting of WQGTHFPYT (aa 114 to aa 122 of SEQ ID NO: 7 or 8 or 9 or 25).

In a further aspect, provided is a polypeptide comprising, or consisting essentially of, or yet further consisting of:
(a) a high mobility group box protein (HMGB) polypeptide, optionally a HMGB1 polypeptide, or a fragment thereof comprising, or consisting essentially of, or consisting of a B box or an A box or an AB box thereof, and
(b) an antibody or an antigen-binding fragment thereof specifically recognizing and binding a tip domain of a DNABII protein, with the proviso that
   (i) the polypeptide does not comprise SEQ ID NO: 52, or
   (ii) the antigen-binding fragment that does not comprise an Fab optionally an Fab of polyclonal antibodies or the antibody that does not comprise polyclonal antibodies, or
   both (i) and (ii).

In one aspect, provided is a polynucleotide encoding:
(a) a high mobility group box protein (HMGB) polypeptide, optionally a HMGB1 polypeptide, or a fragment thereof comprising, or consisting essentially of, or consisting of a B box or an A box or an AB box thereof, and
(b) an anti-DNABII antibody or an antigen-binding fragment thereof as disclosed herein, such as the antibody or antigen-binding fragment thereof comprising, or consisting essentially of, or yet further consisting of:
   (i) a heavy chain complementarity-determining region 1 (CDRH1) comprising, or consisting essentially of, or yet further consisting of GFTFRTY (aa 50 to aa 56 of SEQ ID NO: 1 or 2 or 3 or 24);
   (ii) a heavy chain complementarity-determining region 2 (CDRH2) comprising, or consisting essentially of, or yet further consisting of GSDRRH (aa 76 to aa 81 of SEQ ID NO: 1 or 2 or 3 or 24);
   (iii) a heavy chain complementarity-determining region 3 (CDRH3) comprising, or consisting essentially of, or yet further consisting of VGPYDGYYGEFDY (aa 121 to aa 133 of SEQ ID NO: 1 or 2 or 3 or 24);
   (iv) a light chain complementarity-determining region 1 (CDRL1) comprising, or consisting essentially of, or yet further consisting of QSLLDSDGKTF (aa 47 to aa 57 of SEQ ID NO: 7 or 8 or 9 or 25);
   (v) a light chain complementarity-determining region 2 (CDRL2) comprising, or consisting essentially of, or yet further consisting of LVS (aa 75 to aa 77 of SEQ ID NO: 7 or 8 or 9 or 25); and
   (vi) a light chain complementarity-determining region 3 (CDRL3) comprising, or consisting essentially of, or yet further consisting of WQGTHFPYT (aa 114 to aa 122 of SEQ ID NO: 7 or 8 or 9 or 25).
or a polynucleotide complementary thereto.

In a further aspect, provided is a polynucleotide encoding:
(a) a high mobility group box protein (HMGB) polypeptide, optionally a HMGB1 polypeptide, or a fragment thereof comprising, or consisting essentially of, or consisting of a B box or an A box or an AB box thereof, and
(b) an antibody or an antigen-binding fragment thereof specifically recognizing and binding a tip domain of a DNABII protein, or a polynucleotide complementary thereto, with the proviso that
   (i) the polynucleotide does not encode SEQ ID NO: 52, or
   (ii) the antigen-binding fragment that does not comprise an Fab optionally an Fab of polyclonal antibodies or the antibody that does not comprise polyclonal antibodies, or
   both (i) and (ii).

In one aspect, provided is a vector comprising, or alternatively consisting essentially of, or yet further consisting of a polynucleotide as disclosed herein. In some embodiments, the vector is a non-viral vector (such as a plasmid) or a viral vector. In further embodiments, the viral vector is selected from a group consisting of a retroviral vector, a lentiviral vector, an adenoviral vector, or an adeno-associated viral vector. Additionally or alternatively, the vector further comprises a regulatory sequence directing the expression of the polynucleotide.

A host cell is provided herein, comprising one or more of: a composition or combination as described herein, a polypeptide as disclosed herein, a polynucleotide as disclosed herein, or a vector as disclosed herein.

In one aspect, provided is a method for inhibiting or competing with the binding of a DNABII polypeptide or protein to a microbial DNA. The method comprises, or alternatively consists essentially of, or yet further consists of contacting the DNABII polypeptide or protein with a combination or composition as disclosed herein.

In another aspect, provided is a method to disrupt a biofilm. The method comprises, or alternatively consists essentially of, or yet further consists of contacting the biofilm with a composition or combination as disclosed herein.

In yet another aspect, provided is a method to prevent the formation of or to disrupt a biofilm on a surface. The method comprises, or alternatively consists essentially of, or yet further consists of contacting the biofilm with a combination or composition as disclosed herein, or treating the surface susceptible to or containing a biofilm with a combination or composition as disclosed herein.

In a further aspect, provided is a method to prevent the formation of or disrupt a biofilm in a subject. The method comprises, or alternatively consists essentially of, or yet further consists of administering to the subject a composition or a combination as disclosed herein.

In yet a further aspect, provided is a method for inhibiting, preventing or treating a microbial infection that produces a biofilm in a subject. The method comprises, or alternatively consists essentially of, or yet further consists of administering to the subject a combination or composition as disclosed herein.

In one aspect, provided is a method to treat a condition characterized by the formation of a biofilm in a subject. The method comprises, or alternatively consists essentially of, or yet further consists of administering to the subject a composition or combination as disclosed herein.

In one aspect, provided is a method for one or more of the following: (A) preventing the formation of or disrupting a biofilm in vitro or ex vivo, (B) preventing the formation of or disrupting a biofilm in a subject, (C) inhibiting, preventing or treating a microbial infection that produces a biofilm in a subject, or (D) treating a condition characterized by the formation of a biofilm in a subject. The method comprises, or alternatively consists essentially of, or yet further consists of administering to the subject:
(a) a high mobility group box protein (HMGB) polypeptide, optionally a HMGB1 polypeptide or a fragment thereof comprising, or consisting essentially of, or yet further consisting of a B box or an A box or an AB box thereof, and
(b) an anti-DNABII antibody or an antigen-binding fragment thereof as disclosed herein.

In one aspect, provided is a method for inducing or increasing the formation of a neutrophil extracellular trap (NET) immediately adjacent to a biofilm in a subject, and disrupting the biofilm optionally without inducing a pro-inflammatory response. The method comprises, or alternatively consists essentially of, or yet further consists of administering to the subject:
(a) a high mobility group box 1 protein (HMGB1) polypeptide comprising the amino acid sequence of SEQ ID NO: 52, or a fragment thereof comprising, or consisting essentially of, or yet further consisting of a B box or an A box or an AB box thereof, and
(b) an anti-DNABII antibody or an antigen-binding fragment thereof as disclosed herein.

In one aspect, provided is a method for one or more of the following: (A) preventing the formation of or disrupting a biofilm in vitro or ex vivo, (B) preventing the formation of or disrupting a biofilm in a subject, (C) inhibiting, preventing or treating a microbial infection that produces a biofilm in a subject, or (D) treating a condition characterized by the formation of a biofilm in a subject. The method comprises, or consists essentially of, or yet further consists of administering to the subject one or more of: a composition or combination as disclosed herein, a polypeptide as disclosed herein, a polynucleotide as disclosed herein, a vector as disclosed herein, or a host cell as disclosed herein.

In one aspect, provided is a method for inducing or increasing the formation of a neutrophil extracellular trap (NET) immediately adjacent to a biofilm in a subject, and disrupting the biofilm optionally without inducing a pro-inflammatory response. The method comprises, or consists essentially of, or yet further consists of administering to the subject one or more of: a composition or combination as disclosed herein, a polypeptide as disclosed herein, a polynucleotide as disclosed herein, a vector as disclosed herein, or a host cell as disclosed herein, with the proviso that the HMGB1 polypeptide comprises, or consists essentially of, or yet further consists of SEQ ID NO: 52.

Additionally provided is a kit for use in a method as disclosed herein. The kit comprises, or consists essentially of, or yet further consists of instructions for use and one or more of: a composition or combination as disclosed herein, a polypeptide as disclosed herein, a polynucleotide as disclosed herein, a vector as disclosed herein, or a host cell as disclosed herein.

### BRIEF DESCRIPTION OF THE DRAWINGS

FIG. 1 provides a model of HMGB1-mediated control of the eDNA-dependent bacterial biofilm matrix.

FIGS. 2A-2E provide composite of images that depict the zone between the biofilm and the abundance of PMNs elicited to the site of infection. FIG. 2A is a representative low magnification light micrograph of an H&E stained frozen section of a 17-day biofilm produced by nontypeable Haemophilus influenzae (NTHI) in the middle ear of the chinchilla during experimental otitis media. Area of dense PMN infiltration primarily located in top right-hand corner region, whereas NTHI-induced biofilm occupies the lower left-hand region of image. Zone where these two regions meet is demarked by dashed lines. Bar=100 μm. FIG. 2B is a representative image of a serial section of the in situ biofilm shown in FIG. 2A, where the PMN-rich area intersects with the NTHI biofilm, immunolabeled with antibodies directed against elastase to demark the PMNs and also antibodies directed NTHI outer membrane proteins to demark the NTHI-induced biofilm, as well as the area of intersection where both fluorochromes are visible as admixed. Bar=100 μm. High magnification immunolabeled confocal image of an 11-day old NTHI biofilm recovered from the chinchilla middle ear: FIG. 2C shows NTHI biofilm nearly exclusively labeled with antibodies to the DNABII protein HU where HU labeling is detected on strands of bacterial eDNA; FIG. 2D provides an area where NTHI-induced biofilm intersects with PMN-rich region where now both anti-DNABII (HU) labeling is evident, as well as labeling with anti-HMGB1; d1 and d2 are consecutive 1 μm Z-plane images of the inset, which demonstrate that there is no physical overlap of DNABII and HMGB1 labeling FIG. 2E provides a PMN-rich area wherein the labeling is exclusively for anti-HMGB1. Bars in panels C-E=5 μm.

FIGS. 3A-3B show that HMGB1 variants disrupted biofilms formed by diverse high priority human pathogens. FIG. 3A shows that indicated isoform of HMGB1 (200 nM unless otherwise indicated) were added to 24 h biofilms, in vitro for 16 h. For each pathogen tested, five bars are presented, and they, from left to right, represent data obtained from control, α-IHF_{Ec}, rHMGB1, mHMGB1, and nHMGB1. Exceptions were: 800 nM rHMGB1 or 200 nM mHMGB1 for S. aureus (S as indicated in ESKAPE); 800 nM rHMGB1, 800 nM mHMGB1, 3.3 mM α-IHF_{Ec} IgG for E. faecium (E as indicated in ESKAPE) and for only 1 h to avoid potential degradation by E. faecium produced proteases. Biofilms were stained with LIVE/DEAD® stain and visualized via confocal laser scanning microscope (CLSM) and analyzed by COMSTAT to calculate average thickness. Percent change in biofilm thickness compared to control was plotted. Bars represent the SEM. *P<0.05, **P<0.01, ***P<0.001, ****P<0.0001 via unpaired t tests. FIG. 3B provides representative images of UPEC biofilms incubated with the indicated concentrations of rHMGB1. Data collectively show that rHMGB1, nHMGB1 and mHMGB1 significantly disrupted bacterial biofilms formed by diverse human pathogens and further show that rHMGB1 induced dose-dependent disruption of a UPEC biofilm.

FIGS. 4A-4B show that HMGB1 disrupted biofilms and released biofilm-resident bacteria into the planktonic state. 24 h UPEC biofilms were incubated with rHMGB1 (200 nM) for 16 h, then bacteria in the planktonic (conditioned media) versus biofilm state (adherent bacteria) were enumerated. Total CFU (planktonic+biofilm) was plotted in FIG. 4A. Relative percent of total bacteria in the planktonic versus the biofilm state was plotted in FIG. 4B. Bars represent the SEM. *P<0.05 via paired t test. Note that rHMGB1 had no bactericidal effect, however it induced the partitioning of bacteria from biofilm residence into the planktonic state.

FIGS. 5A-5B show that rHMGB1 synergized with antibiotics in vitro in the eradication of planktonic and biofilm-resident bacteria. 24 h NTHI biofilms were incubated with rHMGB1 (200 nM) alone or in combination with ampicillin (32 μg/ml) or amoxicillin-clavulanate (1 μg/ml) for 16 h, then bacteria in the planktonic (conditioned media) versus biofilm state (adherent bacteria) were enumerated. Biofilm CFU (FIG. 5A) and planktonic CFU (FIG. 5B) were plotted. Bars represent the SEM. *P<0.05, **P<0.01 via unpaired t test. Note that rHMGB1 had no bactericidal effect, however when delivered in combination with antibiotics, rHMGB1 promoted killing of both planktonic and biofilm-resident bacteria.

FIGS. 6A-6C show that oxidation of rHMGB1 negatively affected its antibiofilm activity (FIG. 6A) and acetylation and phosphorylation of rHMGB1 did not affect the antibiofilm activity of rHMGB1 (FIGS. 6B & 6C). In FIG. 6A, 24 h biofilms formed in vitro by UPEC was incubated with ox-rHMGB1 (200 nM) for 16 hours. Biofilms were stained with LIVE/DEAD® stain and visualized via confocal laser scanning microscope (CLSM) and analyzed by COMSTAT to calculate average thickness. Bars represent the SEM. *P<0.05, **P<0.01, ***P<0.001, ****P<0.0001 as assessed by unpaired t tests. Note that the anti-biofilm function of HMGB1 was significantly reduced upon oxidation of rHMGB1. In FIG. 6B, the acetylated or phosphorylated forms were confirmed by triton acetic acid urea gel (TAU gel; top) and Western blot with α-acetyl-lysine (α-Ac-Lys; bottom). In FIG. 6C, 24 h biofilms formed in vitro by: (Bc—B. cenocepacia; E—Enterobacter spp, or K—K. pneumoniae) were significantly disrupted by both Ac- and PrHMGB1 compared to control. Bars represent the SEM. *P<0.05, **P<0.01, ***P<0.001, ****P<0.0001 as assessed by unpaired t tests. Note that the anti-biofilm function of HMGB1 remained unchanged regardless of either acetylation or phosphorylation.

FIG. 7 shows that engineered single amino acid variant mHMGB1 retained its ability to bind to HJ DNA. 5′ end labeled 6-carboxyfluorescein HJ DNA (20 nM) was incubated with increasing concentrations (50-500 nM) of IHF, rHMGB1 or mHMGB1, then resolved by non-denaturing PAGE. Arrows indicate the HJ DNA-protein complex. Note that mHMGB1 maintained its ability to bind HJ DNA.

FIGS. 8A-8C show that HMGB1 bound to HJ DNA but was unable to stabilize the HJ DNA. 5′ end-labeled ³²P HJ DNA was incubated with increasing concentrations (25-500 nM) of IHF (FIG. 8A), rHMGB1 (FIG. 8B) or RuvA (FIG. 8C) either at room temperature (RT) or at 55° C. for 10 min, then resolved on a 6% non-denaturing PAGE. Asterisk indicates melted oligos and arrows indicate DNA-protein complex. Whereas the DNA-HMGB1 complex was stable at RT, it was unstable at 55° C., which resulted in the increase in abundance of the melted constituent oligos. This outcome contrasts with that observed with both IHF and the prototypic HJ DNA binding protein, RuvA.

FIG. 9 shows that HMGB1 isoforms disrupted the lattice-like eDNA network within K. pneumoniae biofilms, in vitro. 24 h K. pneumoniae biofilms were incubated with the indicated protein (200 nM) for 16 h. Unfixed biofilms were incubated with α-dsDNA monoclonal antibody, then incubated with goat α-mouse IgG conjugated to AlexaFluor 488 (bottom panels). K. pneumoniae were stained with FilmTracer FM 4-64 (top panels). Biofilms were visualized by CLSM. Note the intertwined web-like structure in the control and the disruption of the web-like structure by rHMGB1 and mHMGB1. Scale bars represent 10 μm.

FIGS. 10A-10B show that HMGB1 disrupted biofilms via its ability to bind to HJ-like structures within the biofilm extracellular matrix. FIG. 10A shows that 24 h biofilms formed in vitro by UPEC were incubated with mHMGB1 (200 nM) in the presence or absence of each of the indicated proteins for 16 hours. FIG. 10B shows that 24 h biofilms formed in vitro by UPEC were incubated with rHMGB1 (200 nM) or NEM-rHMGB1 (200 nM) for 16 hours. Biofilms were stained with LIVE/DEAD® stain, visualized via confocal laser scanning microscope (CLSM) and analyzed by COMSTAT to calculate average thickness. Bars represent the SEM. *P<0.05, **P<0.01, ***P<0.001, ****P<0.0001 as assessed by unpaired t tests. Note that the anti-biofilm function of HMGB1 was lost in the presence of competitors that bind directly to eDNA (HU, RuvA) and therefore compete with HMGB1 for binding to eDNA or upon modification of rHMGB1 that directly affects its ability to bind to HJ DNA.

FIGS. 11A-11B show that NEM-rHMGB1 does not bind to HJ DNA. FIG. 11A provides that NEM-modified rHMGB1 was confirmed by triton acetic acid urea gel (TAU gel). As shown in FIG. 11B, 5′ end-labeled 6-carboxyfluorescein HJ DNA (20 nM) was incubated with increasing concentrations (250-500 nM) of IHF, rHMGB1 or NEM-rHMGB1, then resolved by non-denaturing PAGE. Arrows indicate the HJ DNA-protein complex. Note that NEM-rHMGB1 lost its ability to bind HJ DNA.

FIG. 12 shows that HMGB1 isoforms induced varied degrees of neutrophil-mediated NETs formation, in vitro. Neutrophils were incubated with the indicated protein (200 nM) for 3.5 h. Neutrophils were fixed, then incubated with α-dsDNA monoclonal antibody and α-neutrophil elastase antibody, then incubated with goat α-mouse IgG conjugated to AlexaFluor 488 and goat α-rabbit IgG conjugated to AlexaFluor 594. Neutrophils were stained with wheat germ agglutinin (WGA) conjugated to AlexaFluor 350. NETs were visualized by CLSM. Note the intertwined NETs specifically formed upon incubation of neutrophils with rHMGB1, mHMGB1 and NEM-rHMGB1 albeit to variable degrees. Scale bars represent 10 μm.

FIGS. 13A-13F show that HMGB1 promotes clearance of B. cenocepacia aggregates from the murine lung. FIG. 13A provides a representative IF image of a lung section of lung recovered from a mouse infected with B. cenocepacia. C57BL/6 mice were challenged i.t. with 10⁷ CFU and either simultaneously (prevention), or 24 h later (treatment), received 0.2 nmol of the indicated HMGB1 variant. Bronchoalveolar lavage (BAL) was collected at 18 hpi (prevention, FIG. 13B) or at 72 hpi (treatment, FIG. 13C) then analyzed for CFU. FIG. 13D provides representative images stained with H&E (10× and 40× magnification). As shown in FIG. 13E, cells in BAL were stained with α-CD45, CD11b, and Ly-6G and analyzed by flow cytometry to measure relative neutrophil influx (FIG. 13F). Bars represent SD. *P<0.05, **P<0.01 as assessed by Mann-Whitney test. HMGB1 treatment significantly decreased CFU of B. cenocepacia in the murine lung and treatment with the engineered C45S mutation within mHMGB1 eliminated the pro-inflammatory activity.

FIGS. 14A-14C show that mHMGB1 cleared B. cenocepacia from the murine lungs despite reduced phagocytosis and exhibited an attenuated neutrophil recruitment to the peritoneal cavity. FIG. 14A provides that C57BL/6 mice were challenged i.t. with 10⁷ CFU B. cenocepacia and treated 24 hours later with 0.2 nmol of rHMGB1 or mHMGB1. 48 h after treatment, mice were sacrificed, and sections of lung were labeled with E. coli α-EF-Tu monoclonal antibody to label B. cenocepacia and with DAPI to detect neutrophils. FIG. 14B shows that macrophages were treated with 5 μg/ml rHMGB1, 5 μg/ml mHMGB1 or 10 μM cytochalasin D for 2 h, then pHrodo red E. coli bioparticles were added and incubated for 2 h. Any excess bioparticles were removed by washing the cells with HBSS, the phagocyted bioparticles were measured by plate reader (560/585 nm). FIG. 14C shows that C57BL/6 mice were injected i.p. with the indicated HMGB1 isoform, then 24 h later, influx of neutrophils into the peritoneal cavity was determined by flow cytometry with α-CD45, CD11b, and Ly-6G antibodies. n=3. Bars represent the SD. *P<0.05. Whereas treatment with rHMGB1 induced significant migration of neutrophils into the peritoneal cavity, treatment with mHMGB1 significantly attenuated this pro-inflammatory response albeit with a modest reduction in phagocytosis.

FIG. 15 shows that rHMGB1 used to treat biofilms in vivo did not induce dysregulated host response to infection associated with septic shock in mice. Mice were injected i.p. with either 0.2 nmol endotoxin-free HMGB1, 5 mg/kg LPS, or both then monitored for 24 h for signs of septic shock. Serum TNF-α was measured by ELISA after 24 h. Bars represent SD. LoD: limit of detection. Note that rHMGB1 at the same concentration that was used to treat in vivo biofilms did not induce septic shock as indicated by an increase in TNF-α.

FIGS. 16A-16J show that mHMGB1 mediated clearance of biofilm-resident NTHI, eradication of established mucosal biofilms and resolution of experimental disease, an outcome that was enhanced upon co-delivery with antibody fragments directed against the immunoprotective domains of a DNABII protein (tip-chimer Fabs). FIG. 16A provides a study timeline to assess the relative ability of rHMGB1 or mHMGB1 to resolve NTHI biofilms already established in the chinchilla middle ear. FIG. 16B provides relative quantity of NTHI resident within mucosal biofilms and adherent to the middle ear mucosa 1-day after completion of therapy. As shown in FIG. 16C, rubric used to qualitatively assess the amount of middle ear mucosal biofilm that remained 1-day after completion of treatment. FIG. 16D provides relative amount of mucosal biofilm within each middle ear per cohort. In FIG. 16E, rubric used to qualitatively assess the amount of middle ear mucosal inflammation 1-day after completion of treatment. FIG. 16F provides relative amount of mucosal inflammation within each middle ear per cohort. FIG. 16G provides representative image of middle ears from each cohort to demonstrate relative presence/clearance of mucosal biofilm and inflamed/non-inflamed state. FIG. 16H is a study timeline to assess additive potential of mHMGB1 co-delivered with tip-chimer Fabs to resolve NTHI biofilms already established in the chinchilla middle ear. FIG. 16I provides relative quantity of NTHI resident within mucosal biofilms and adherent to the middle ear mucosa 24 h after 1 or 2 treatment doses. FIG. 16J provides relative amount of mucosal biofilm within each middle ear per cohort 24 h after 1 or 2 treatment doses. Whereas both rHMGB1 and mHMGB1 induced rapid clearance of biofilm-resident NTHI and eradication of established mucosal biofilms, only mHMGB1 induced limited mucosal inflammation. Moreover, co-delivery of mHMGB1 with tip-chimer Fab fragments was highly efficacious to eradicate NTHI and associated biofilms from the middle ear.

FIG. 17 shows that pro-inflammatory cytokines IL-1β and IL-17A were significantly more abundant in middle ear fluids recovered from chinchillas treated with rHMGB1, whereas anti-inflammatory cytokines predominated in the mHMGB1-treated cohort. Six days after NTHI challenge (one day after completion of treatment), middle ear fluids were recovered and screened for relative quantity of a panel of pro-inflammatory and anti-inflammatory cytokines by cytometric bead array. Each data point represents an individual middle ear fluid and the mean for each cohort shown. Note significantly elevated concentration of pro-inflammatory cytokines IL-1β and IL-17A in rHMGB1-treated chinchillas (p<0.05), whereas a significantly elevated concentration of the anti-inflammatory cytokine IL-10 was observed in the cohort treated with mHMGB1 (P<0.01).

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. All nucleotide sequences provided herein are presented in the 5′ to 3′ direction. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, levels, rates, dose forms, etc. disclosed herein intend that said component, range, level, rate, dose form, etc. is suitable for the disclosed purpose.

As used herein, comparative terms as used herein, such as higher, lower, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

A “biofilm” intends an organized community of microorganisms that at times adhere to the surface of a structure, that may be organic or inorganic, together with the polymers such as DNA that they secrete, release and/or become available in the extracellular milieu due to bacterial lysis. The biofilms are very resistant to microbiotics and antimicrobial agents. They live on gingival tissues, teeth and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control) estimate that over 65% of nosocomial (hospital-acquired) infections are caused by biofilms. They cause chronic vaginal infections and lead to life-threatening systemic infections in people with hobbled immune systems. Biofilms also are involved in numerous diseases. For instance, cystic fibrosis patients have Pseudomonas infections that often result in antibiotic resistant biofilms. In one embodiment, the biofilm comprises a DNABII polypeptide or protein. In a further embodiment, the biofilm comprises an IHF and/or an HU. In yet a further embodiment, the biofilm comprises an IHFA and/or an IHFB.

The term “neutrophil” refers to a type of granulocytes, a type of white blood cells, and a type of phagocyte in mammals and are first-responders of inflammatory cells. Neutrophils are granule-containing, polymorphonuclear leukocytes that develop in the bone marrow from myeloid precursors. They play a central role in the innate immune response by destroying foreign particles either intracellularly in phagosomes or extracellularly by releasing neutrophil extracellular traps (NETs), and promoting acute inflammation. In humans, neutrophils are the most abundant circulating leukocyte, accounting for 50-70% of white blood cells, while 10-25% of circulating mouse leukocytes are neutrophils. Although neutrophils can be visually identified based on the shape of their nuclei and cytoplasmic granularity, they can also be identified based on their expression of certain marker(s). Mouse neutrophils are commonly identified based on the cell surface expression of Ly-6G and CD11b/Integrin alpha M. Since mouse granulocytic myeloid-derived suppressor cells can also express these markers, neutrophils are frequently distinguished from these cells in mice based on their lack of expression of M-CSF R/CD115 and CD244/SLAMF4, along with an absence of immunosuppressive properties. In humans, neutrophils are distinguished from eosinophils and monocytes based on the expression of both CD15 and CD16/Fc gamma RIII on human neutrophils, along with the lack of expression of CD14. In addition, CD66b/CEACAM-8, CD11b/Integrin alpha M, CD33, and the cytoplasmic marker, myeloperoxidase, are other common markers that are used to identify human neutrophils.

As used herein, a neutrophil extracellular trap (NET) refers to networks of extracellular fibers, primarily composed of DNA from neutrophils, which bind pathogens. NETs allow neutrophils to kill extracellular pathogens while minimizing damage to the host cells. High-resolution scanning electron microscopy has shown that NETs consist of stretches of DNA and globular protein domains with diameters of 15-17 nm and 25 nm, respectively. These aggregate into larger threads with a diameter of 50 nm. However, under flow conditions, NETs can form much larger structures, reaching hundreds of nanometers in length and width. Analysis by immunofluorescence corroborated that NETs contain proteins from azurophilic granules (neutrophil elastase, cathepsin G and myeloperoxidase), specific granules (lactoferrin), tertiary granules (gelatinase), and the cytoplasm; however, CD63, actin, tubulin and various other cytoplasmatic proteins are not present in NETs. In some embodiments, the NET can be measured by evaluating one or more of its components (including but not limited to neutrophil elastase) for example by immunofluorescence. NET activation and release is referred to herein as NETosis, which is a dynamic process that can come in two forms, suicidal and vital NETosis.

As used herein, the phrase “immediately adjacent” refers to a position directly connected, without a gap or spacing, to a reference position or structure.

In some embodiments, the term “disrupt” intends a reduction in the formation of the DNA/protein matrix that is a component of a microbial biofilm. Additionally or alternatively, the term “disrupt” intends a reduction of a formed biofilm, such as dispersing the biofilm partially or completely. Such reduction can be shown in various parameters. For example, the biomass or the bacterial load of a biofilm or both can be evaluated prior to and after a treatment, and the reduction thereof after the treatment can be used to show the efficacy of the treatment. Another example of the parameter is relative mucosal biofilm score or biomass score given by a blinded evaluator. Other suitable parameters are shown in the Examples as disclosed herein. In some embodiments, a treatment reduces a biofilm to at least about 90% (including but not limited to at least about 85%, or at least about 80%, or at least about 75%, or at least about 70%, or at least about 65%, or at least about 60%, or at least about 55%, or at least about 50%, or at least about 45%, or at least about 40%, or at least about 35%, or at least about 30%, or at least about 25%, or at least about 20%, or at least about 15%, or at least about 10%, or at least about 9%%, or at least about 8%, or at least about 7%, or at least about 6%, or at least about 5%, or at least about 4%, or at least about 3%, or at least about 2%, or at least about 1%, or less than 1%, or about 0%) of the biofilm prior to the treatment. In certain embodiments, disrupting a biofilm refers to dispersing the biofilm (completely or partially), releasing microorganisms from the DNA/protein matrix of the biofilm, and optionally allowing killing the microorganisms by host immune effectors and/or antibiotics.

A “DNABII polypeptide or protein” intends a DNA-binding protein or polypeptide that is composed of DNA-binding domains and thus have a specific or general affinity for microbial DNA. In one aspect, they bind DNA in the minor grove. Non-limiting examples of DNABII proteins are an integration host factor (IHF) protein and a histone-like protein from E. coli strain U93 (HU). Other DNA binding proteins that may be associated with the biofilm include DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP-418813).

An “integration host factor” or “IHF” protein is a bacterial protein that is used by bacteriophages to incorporate their DNA into the host bacteria. They also bind extracellular microbial DNA. The genes that encode the IHF protein subunits in E. coli are himA (Genbank Accession No.: POA6X7.1) and himD (POA6Y1.1) genes. Homologs for these genes are found in other organisms. In certain embodiments, the term “IHF” refers to one or both of the two IHF subunits: integration host factor subunit alpha (IHFA or IhfA) and integration host factor subunit beta (IHFB or IhfB).

“HU” or “histone-like protein from E. coli strain U93” refers to a class of heterodimeric proteins typically associate with E. coli. HU proteins are known to bind DNA junctions. Related proteins have been isolated from other microorganisms. The complete amino acid sequence of E. coli HU was reported by Laine et al. (1980) Eur. J. Biochem 103(3)447-481. Antibodies to the HU protein are commercially available from Abeam. The genes that encode the HU protein subunits in E. coli are hupA and hupB corresponding to SEQ ID NOs: 29 and 30, respectively. Homologs for these genes are found in other organisms, and peptides corresponding to these genes from other organisms can be found in Table 10 of WO 2011/123396.

The term “surface antigens” or “surface proteins” refers to proteins or peptides on the surface of cells such as bacterial cells. Examples of outer membrane proteins such as OMP P5 (Genbank Accession No.: YP-004139079.1), OMP P2 (Genbank Accession No.: ZZX87199.1) and OMP P26 (Genbank Accession No.: YP-665091.1) whereas examples of surface antigens are rsPilA or recombinant soluble PilA (Genbank Accession No.: EFU96734.1) and Type IV Pilin (Genbank Accession No.: Yp-003864351.1).

The term “Haemophilus influenzae” refers to pathogenic bacteria that can cause many different infections such as, for example, ear infections, eye infections, and sinusitis. Many different strains of Haemophilus influenzae have been isolated and have an IhfA, ihfB and hupA genes or protein. Some non-limiting examples of different strains of Haemophilus influenzae include Rd KW20, 86-028NP, R2866, PittGG, PittEE, R2846, and 2019.

“Microbial DNA” intends single or double stranded DNA from a microorganism that is incorporated into a biofilm.

“Inhibiting, preventing or disrupting” a biofilm intends the prophylactic or therapeutic reduction in the structure of a biofilm.

A “bent polynucleotide” intends a double strand polynucleotide that contains a small loop on one strand which does not pair with the other strand. In some embodiments, the loop is from 1 base to about 20 bases long, or alternatively from 2 bases to about 15 bases long, or alternatively from about 3 bases to about 12 bases long, or alternatively from about 4 bases to about 10 bases long, or alternatively has about 4, 5, or 6, or 7, or 8, or 9, or 10 bases.

“Polypeptides that compete with DNABII binding, such as IHF in DNA binding” intend proteins or peptides that compete with DNABII (e.g., IHF) in binding bent or distorted DNA structures but do not form a biofilm with the DNA. Examples, without limitation, include fragments of IHF that include one or more DNA binding domains of the IHF, or the biological equivalents thereof.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “C-terminal polypeptide” intends at least the 10, or alternatively at least the 15, or alternatively at least 20, or at least the 25 C-terminal amino acids or alternatively half of a polypeptide. In another aspect, for polypeptides containing 90 amino acids, the C-terminal polypeptide would comprise amino acids 46 through 90. In one aspect, the term intends the C-terminal 20 amino acids from the carboxyl terminus.

A “tip fragment” of a DNABII polypeptide intends a DNABII polypeptide that, using IHFalpha and IHFbeta as examples, forms the two arms of the proteins. Non-limiting examples of such include IhfA, A tip fragment: NFELRDKSSRPGRNPKTGDVV, SEQ ID NO: 31, and IhfB, B tip fragment: SLHHRQPRLGRNPKTGDSVNL, SEQ ID NO: 32, as well as the amino acid sequences of the DNABII polypeptide from another species (such as another bacterium) aligned to SEQ ID NO: 31 or 32 as well as peptides containing two tip domains or modified domains to provide the necessary confirmation, e.g., the mIhFB4 and the IhfA tip domains, exemplified below.

A “tail fragment” of a DNABII polypeptide intends a region of the protein that is both exposed to the bulk medium and not occluded by DNA or other polypeptides.

As used herein, the ESKAPE pathogens include Enterococcus faecium, Staphylococcus aureus, Klebsiella pneumoniae, Acinetobacter baumannii, Pseudomonas aeruginosa, and Enterobacter species. These pathogens are the leading cause of nosocomial infections throughout the world.

An “HMG domain,” “high mobility group (HMG) box domain,” or “HMGB” refers to an amino acid sequence that is involved in binding DNA (Stros et al., Cell Mol Life Sci. 64(19-20):2590-606 (2007)). In one embodiment, the structure of the HMG-box domain consists of three helices in an irregular array. In another embodiment, an HMG-box domain enables a protein to bind non-B-type DNA conformations (kinked or unwound) with high affinity. HMG-box domains can be found in high mobility group proteins, which are involved in the regulation of DNA-dependent processes such as transcription, replication and DNA repair, all of which require changing the conformation of chromatin (Thomas (2001) Biochem. Soc. Trans. 29(Pt 4):395-401).

HMG-box proteins are found in a variety of eukaryotic organisms and can be broadly divided into two groups, based on sequence-dependent and sequence-independent DNA recognition; the former usually contain one HMG-box motif, while the latter can contain multiple HMG-box motifs. Non-limiting examples of polypeptides comprising an HMG-box domain include HMG1(HMGB1), HMG2(HMGB2), HMGB3 and HMGB4 non-histone components of chromatin; SRY (sex determining region Y protein) involved in differential gonadogenesis; the SOX family of transcription factors (Harley et al. (2003) Endocr. Rev. 24(4):466-87); sequence-specific LEF1 (lymphoid enhancer binding factor 1) and TCF-1 (T-cell factor 1) involved in regulation of organogenesis and thymocyte differentiation (Labbe et al. (2000) Proc. Natl. Acad. Sci. USA 97(15):8358-63); structure-specific recognition protein SSRP involved in transcription and replication; MTF1 mitochondrial transcription factor; nucleolar transcription factors UBF 1/2 (upstream binding factor) involved in transcription by RNA polymerase I; Abf2 yeast ARS-binding factor (Cho et al. (2001) Biochim. Biophys. Acta. 1522(3):175-86); yeast transcription factors lxrl, Rox1, Nhp6b and Spp41; mating type proteins (MAT) involved in the sexual reproduction of fungi (Barve et al. (2003) Fungal Genet. Biol. 39(2):151-67); and the YABBY plant-specific transcription factors.

“HMGB1” is a high mobility group box (HMGB) 1 protein that is reported to bind to and distort the minor groove of DNA. Recombinant or isolated protein and polypeptide are commercially available from Atgenglobal, ProSpecBio, Protein1 and Abnova. An HMGB1 polypeptide is utilized herein, referring to an HMGB1 protein or an equivalent thereof. In some embodiments, the HMGB1 protein comprises, or consists essentially of, or yet further consists of SEQ ID NO: 51. In some embodiments, the HMGB1 equivalent comprises, or consists essentially of, or yet further consists of an HMG-box protein, such as those as disclosed herein. In some embodiments, the HMGB1 equivalent comprises, or consists essentially of, or yet further consists of an HMG-box domain, such as those as disclosed herein. In further embodiments, the HMGB1 equivalent comprises, or consists essentially of, or yet further consists of one or more of HMGB2, HMGB3, or HMGB4. Additionally or alternatively, the HMGB1 equivalent comprises, or consists essentially of, or yet further consists of one or more mutations as disclosed herein. In some embodiments, the HMGB1 equivalent comprises, or consists essentially of, or yet further consists of any one or more of SEQ ID NOs: 52-58, 68-74, 84-90 or 100-114, or an equivalent of each thereof.

Exemplary sequences of polypeptides comprising an HMG-box domain include NP_002119 (human HMGB1), NP_001124160 (human HMGB2), NP_005333 (human HMGB3) and NP_660206 (human HMGB4). Amino acid residues from about 9 to about 76 of the human HMGB1, for example, form an HMG-box domain and amino acid residues from about 90 to about 138 form another HMG-box domain. An HMGB1 fragment that contains either of these two HMG-box domains, for example, also constitutes a polypeptide comprising an HMG-box domain, within the meaning of the present disclosure. In the examples described herein, a recombinant HMGB1 (derived from a human and recombinantly expressed and purified in E. coli) is used as a comparator to mHMGB1 (C45S) and has the sequence:

(SEQ ID NO: 51)
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEGEEDEEDEEEE
EDEEDEDEEEDDDDE.

As used herein, an amino acid mutation can be identified by two letters separated by a number. The first letter refers to the original amino acid residue, the number indicates the position of the mutation in a reference sequence, and the second letter indicates the mutated amino acid residue. For example, the HMGB1 polypeptide comprising a C45S mutation indicates that cysteine (C) that is the 45^{th} amino acid residue of HMGB1 is mutated to serine (S). In further embodiments, a full-length sequence, such as SEQ ID NO: 51, can be used as a reference, for example when identifying an amino acid position in a fragment. One example is that, a B box fragment of a HMGB1 polypeptide can comprise a C106S mutation, wherein the number 106 refers to the amino acid residue position in the full-length HMGB1 polypeptide instead of the B box fragment. In some embodiments, the reference is SEQ ID NO: 51. In some embodiments, when referring to a position, the second letter may be omitted.

As used herein, an amino acid position in a sequence are identified by a letter followed by a number. The letter refers to the amino acid residue at the position, while the number indicates the position of the mutation in a reference sequence. For example, C45 indicates the 45^{th} amino acid residue of HMGB1, which is cysteine (C). In further embodiments, a full-length sequence, such as SEQ ID NO: 51, can be used as a reference, for example when identifying an amino acid position in a fragment. One example is that, a B box fragment of a HMGB1 polypeptide can comprise a C106S mutation, wherein the number 106 refers to the amino acid residue position in the full-length HMGB1 polypeptide instead of the B box fragment. In some embodiments, the reference is SEQ ID NO: 51.

The term “modified high mobility group-box 1 domain” and mHMGB1 as used herein refer to an HMGB1 that has been mutated, e.g., via substitutions of the cysteine residues at positions 23, 45, and/or 106 based on the consensus sequence polypeptide of HMGB1 derived from human: SEQ ID NO: 51.

Non-limiting exemplary sequences of modified high mobility group-box 1 domains (i.e., mHMGB1) include but are not limited to: mHMGB1 (C23S) which is SEQ ID NO: 51 further comprising one mutation of C23S, i.e., SEQ ID NO: 53; mHMGB1 (C45S) is SEQ ID NO: 51 further comprising one mutation of C45S, i.e., SEQ ID NO: 52 consisting of MGKGDPKKPRGKMSSYAFFVQTCREEHKKKHPDASVNFSEFSKKSSERWKTMSAKE KGKFEDMAKADKARYEREMKTYIPPKGETKKKFKDPNAPKRPPSAFFLFCSEYRPKI KGEHPGLSIGDVAKKLGEMWNNTAADDKQPYEKKAAKLKEKYEKDIAAYRAKGKP DAAKKGVVKAEKSKKKKEEEEGEEDEEDEEEEEDEEDEDEEEDDDDE (SEQ ID NO: 52); mHMGB1 (C106S) which is SEQ ID NO: 51 further comprising one mutation of C106S, i.e., SEQ ID NO: 54; mHMGB1 (C23S, C45S—double mutant) which is SEQ ID NO: 51 further comprising two mutations of C23S and C45S, i.e., SEQ ID NO: 55; mHMGB1 (C23S, C106S—double mutant) which is SEQ ID NO: 51 further comprising two mutations of C23S and C106S, i.e., SEQ ID NO: 56; mHMGB1 (C45S, C106S—double mutant) which is SEQ ID NO: 51 further comprising two mutations of C45S and C106S, i.e., SEQ ID NO: 57; and mHMGB1 (C23S, C45S, C106S—triple mutant) which is SEQ ID NO: 51 further comprising three mutations of C23S, C45S and C106S, i.e., SEQ ID NO: 58.

An exemplified nucleic acid sequence encoding a human HMGB1 is provided below:

(SEQ ID NO: 59)
ATGGGCAAAGGAGATCCTAAGAAGCCGAGAGGCAAAATGTCATCATATGC
ATTTTTTGTGCAAACTTGTCGGGAGGAGCATAAGAAGAAGCACCCAGATG
CTTCAGTCAACTTCTCAGAGTTTTCTAAGAAGTGCTCAGAGAGGTGGAAG
ACCATGTCTGCTAAAGAGAAAGGAAAATTTGAAGATATGGCAAAGGCGGA
CAAGGCCCGTTATGAAAGAGAAATGAAAACCTATATCCCTCCCAAAGGGG
AGACAAAAAAGAAGTTCAAGGATCCCAATGCACCCAAGAGGCCTCCTTCG
GCCTTCTTCCTCTTCTGCTCTGAGTATCGCCCAAAAATCAAAGGAGAACA
TCCTGGCCTGTCCATTGGTGATGTTGCGAAGAAACTGGGAGAGATGTGGA
ATAACACTGCTGCAGATGACAAGCAGCCTTATGAAAAGAAGGCTGCGAAG
CTGAAGGAAAAATACGAAAAGGATATTGCTGCATATCGAGCTAAAGGAAA
GCCTGATGCAGCAAAAAAGGGAGTTGTCAAGGCTGAAAAAAGCAAGAAAA
AGAAGGAAGAGGAGGAAGGTGAGGAAGATGAAGAGGATGAGGAGGAGGAG
GAAGATGAAGAAGATGAAGATGAAGAAGAAGATGATGATGATGAA.

Nucleic acid sequences encoding these modified high mobility group-box 1 domains (i.e., mHMGB1) are also provided: mHMGB1 (C23S) which is SEQ ID NO: 59 further comprising an AGT encoding the C23S, i.e., SEQ ID NO: 59 further comprising one mutation from T to A at nucleotide residue 67 of SEQ ID NO: 59, see, SEQ ID NO: 60; mHMGB1 (C45S) which is SEQ ID NO: 59 further comprising an AGT encoding the C45S, i.e., SEQ ID NO: 59 further comprising one mutation from T to A at nucleotide residue 133 of SEQ ID NO: 59 and one mutation from C to T at nucleotide residue 135 of SEQ ID NO: 59, see, SEQ ID NO: 61; mHMGB1 (C106S) which is SEQ ID NO: 59 further comprising an AGC encoding the C106S, i.e., SEQ ID NO: 59 further comprising one mutation from T to A at nucleotide residue 316 of SEQ ID NO: 59, see, SEQ ID NO: 62; mHMGB1 (C23S, C45S—double mutant) which is SEQ ID NO: 59 further comprising an AGT encoding the C23S and an AGT encoding the C45S, i.e., SEQ ID NO: 59 further comprising one mutation from T to A at nucleotide residue 67 of SEQ ID NO: 59, one mutation from T to A at nucleotide residue 133 of SEQ ID NO: 59 and one mutation from C to T at nucleotide residue 135 of SEQ ID NO: 59, see, SEQ ID NO: 63; mHMGB1 (C23S, C106S—double mutant) which is SEQ ID NO: 59 further comprising an AGT encoding the C23S and an AGC encoding the C106S, i.e., SEQ ID NO: 59 further comprising one mutation from T to A at nucleotide residue 67 of SEQ ID NO: 59 and one mutation from T to A at nucleotide residue 316 of SEQ ID NO: 59, see, SEQ ID NO: 64; mHMGB1 (C45S, C106S—double mutant) which is SEQ ID NO: 59 further comprising an AGT encoding the C45S and an AGC encoding the C106S, i.e., SEQ ID NO: 59 further comprising one mutation from T to A at nucleotide residue 133 of SEQ ID NO: 59, one mutation from C to T at nucleotide residue 135 of SEQ ID NO: 59, and one mutation from T to A at nucleotide residue 316 of SEQ ID NO: 59, see, SEQ ID NO: 65; and mHMGB1 (C23S, C45S, C106S—triple mutant) which is SEQ ID NO: 59 further comprising an AGT encoding the C23S, an AGT encoding the C45S and an AGC encoding the C106S, i.e., SEQ ID NO: 59 further comprising one mutation from T to A at nucleotide residue 67 of SEQ ID NO: 59, one mutation from T to A at nucleotide residue 133 of SEQ ID NO: 59, one mutation from C to T at nucleotide residue 135 of SEQ ID NO: 59, and one mutation from T to A at nucleotide residue 316 of SEQ ID NO: 59, see, SEQ ID NO: 66.

It is appreciated that the term “modified high mobility group-box 1 domain” further encompasses those equivalents which have at least about 60%, or at least about 65%, or at least about 70%, or at least about 75%, or at least about 80%, or at least about 85%, or at least about 90%, or at least about 95%, or at least about 98% or at least about 99% identical to the HMGB1 consensus sequence or any one of SEQ ID NOs: 51-58 and comprise the same substitutions at corresponding positions in the equivalent sequences based on alignment against the HMGB1 consensus sequence or any one of SEQ ID NOs: 51-58.

It is appreciated that among the equivalents to a modified high mobility group-box 1 domain are modified high mobility group-box 2, modified high mobility group-box 3, and modified high mobility group-box 4 comprising cysteine to serine substitutions at one or more amino acid positions corresponding to C23S, C45S, and C106S of a modified high mobility group box 1 or an equivalent thereof. As it would be understood by one of skill in the art, mutations in more than one HMG-box proteins (such as HMGB1, HMGB2, HMGB3 or HMGB4) are considered equivalent if the mutated positions in the more than one HMG-box proteins are aligned with each other in a sequence alignment of the more than one HMG-box proteins. Additionally and optionally, the original amino acid residues to be mutated are the same in the equivalent mutations. Further additionally and optionally, the amino acid residues mutated to are the same in the equivalent mutations. Accordingly, an equivalent of the HMGB1 polypeptide can be a wild type HMGB1 mutated at one or more positions, such as C23S, C45S, C106S or any combination thereof as disclosed herein. Further, an equivalent of the HMGB1 polypeptide can be a HMG-box protein other than HMGB1, such as HMGB2, HMGB3 or HMGB4, comprising the equivalent mutations. One non-limiting example is HMGB1 C45S mutant is an equivalent of HMGB2 C45S mutant, as well as HMGB3 C45S or HMGB4 C45S. Alternatively, the C to S mutations as specified herein can be considered as equivalents to each other no matter where their positions are, since without being bound by theory, Applicant believes that the modified HMGB1 fragments as disclosed herein will perform similarly.

Corresponding amino acid substitutions to yield a modified high mobility group-box 2 can be made at the cysteine residues at positions 23, 45, and/or 106 based on the consensus sequence polypeptide of HMGB2 derived from a human:

(SEQ ID NO: 67)
MGKGDPNKPRGKMSSYAFFVQTCREEHKKKHPDSSVNFAEFSKKCSERWK
TMSAKEKSKFEDMAKSDKARYDREMKNYVPPKGDKKGKKKDPNAPKRPPS
AFFLFCSEHRPKIKSEHPGLSIGDTAKKLGEMWSEQSAKDKQPYEQKAAK
LKEKYEKDIAAYRAKGKSEAGKKGPGRPTGSKKKNEPEDEEEEEEEEDED
EEEEDEDEE.

Non-limiting exemplary sequences a modified high mobility group-box 2 domains include but are not limited to: mHMGB2 (C23S) which is SEQ ID NO: 67 further comprising one mutation of C23S, i.e., SEQ ID NO: 68; mHMGB2 (C45S) which is SEQ ID NO: 67 further comprising one mutation of C45S, i.e., SEQ ID NO: 69; mHMGB2 (C106S) which is SEQ ID NO: 67 further comprising one mutation of C106S, i.e., SEQ ID NO: 70; mHMGB2 (C23S, C45S—double mutant) which is SEQ ID NO: 67 further comprising two mutations of C23S and C45S, i.e., SEQ ID NO: 71; mHMGB2 (C23S, C106S—double mutant) which is SEQ ID NO: 67 further comprising two mutations of C23S and C106S, i.e., SEQ ID NO: 72; mHMGB2 (C45S, C106S—double mutant) which is SEQ ID NO: 67 further comprising two mutations of C45S and C106S, i.e., SEQ ID NO: 73; and mHMGB2 (C23S, C45S, C106S—triple mutant) which is SEQ ID NO: 67 further comprising three mutations of C23S, C45S and C106S, i.e., SEQ ID NO: 74.

An exemplified nucleic acid sequence encoding a human HMGB2 is provided below:

(SEQ ID NO: 75)
ATGGGTAAAGGAGACCCCAACAAGCCGCGGGGCAAAATGTCCTCGTACGC
CTTCTTCGTGCAGACCTGCCGGGAAGAGCACAAGAAGAAACACCCGGACT
CTTCCGTCAATTTCGCGGAATTCTCCAAGAAGTGTTCGGAGAGATGGAAG
ACCATGTCTGCAAAGGAGAAGTCGAAGTTTGAAGATATGGCAAAAAGTGA
CAAAGCTCGCTATGACAGGGAGATGAAAAATTACGTTCCTCCCAAAGGTG
ATAAGAAGGGGAAGAAAAAGGACCCCAATGCTCCTAAAAGGCCACCATCT
GCCTTCTTCCTGTTTTGCTCTGAACATCGCCCAAAGATCAAAAGTGAACA
CCCTGGCCTATCCATTGGGGATACTGCAAAGAAATTGGGTGAAATGTGGT
CTGAGCAGTCAGCCAAAGATAAACAACCATATGAACAGAAAGCAGCTAAG
CTAAAGGAGAAATATGAAAAGGATATTGCTGCATATCGTGCCAAGGGCAA
AAGTGAAGCAGGAAAGAAGGGCCCTGGCAGGCCAACAGGCTCAAAGAAGA
AGAACGAACCAGAAGATGAGGAGGAGGAGGAGGAAGAAGAAGATGAAGAT
GAGGAGGAAGAGGATGAAGATGAAGAATAA.

Nucleic acid sequences encoding these modified high mobility group-box 2 domains are also provided: mHMGB2 (C23S) which is SEQ ID NO: 75 further comprising an AGT encoding the C23S, i.e., SEQ ID NO: 75 further comprising one mutation from T to A at nucleotide residue 67 of SEQ ID NO: 75 and one mutation from C to T at nucleotide residue 69 of SEQ ID NO: 75, see, SEQ ID NO: 76; mHMGB2 (C45S) which is SEQ ID NO: 75 further comprising an AGT encoding the C45S, i.e., SEQ ID NO: 75 further comprising one mutation from T to A at nucleotide residue 133 of SEQ ID NO: 75, see, SEQ ID NO: 77; mHMGB2 (C106S) which is SEQ ID NO: 75 further comprising an AGC encoding the C106S, i.e., SEQ ID NO: 75 further comprising one mutation from T to A at nucleotide residue 316 of SEQ ID NO: 75, see, SEQ ID NO: 78; mHMGB2 (C23S, C45S—double mutant) which is SEQ ID NO: 75 further comprising an AGT encoding the C23S and an AGT encoding the C45S, i.e., SEQ ID NO: 75 further comprising one mutation from T to A at nucleotide residue 67 of SEQ ID NO: 75, one mutation from C to T at nucleotide residue 69 of SEQ ID NO: 75, and one mutation from T to A at nucleotide residue 133 of SEQ ID NO: 75, see, SEQ ID NO: 79; mHMGB2 (C23S, C106S—double mutant) which is SEQ ID NO: 75 further comprising an AGT encoding the C23S and an AGC encoding the C106S, i.e., SEQ ID NO: 75 further comprising one mutation from T to A at nucleotide residue 67 of SEQ ID NO: 75, one mutation from C to T at nucleotide residue 69 of SEQ ID NO: 75, and one mutation from T to A at nucleotide residue 316 of SEQ ID NO: 75, see, SEQ ID NO: 80; mHMGB2 (C45S, C106S—double mutant) which is SEQ ID NO: 75 further comprising an AGT encoding the C45S and an AGC encoding the C106S, i.e., SEQ ID NO: 75 further comprising one mutation from T to A at nucleotide residue 133 of SEQ ID NO: 75 and one mutation from T to A at nucleotide residue 316 of SEQ ID NO: 75, see, SEQ ID NO: 81; and mHMGB2 (C23S, C45S, C106S—triple mutant) which is SEQ ID NO: 75 further comprising an AGT encoding the C23S, an AGT encoding the C45S and an AGC encoding the C106S, i.e., SEQ ID NO: 75 further comprising one mutation from T to A at nucleotide residue 67 of SEQ ID NO: 75, one mutation from C to T at nucleotide residue 69 of SEQ ID NO: 75, one mutation from T to A at nucleotide residue 133 of SEQ ID NO: 75, and one mutation from T to A at nucleotide residue 316 of SEQ ID NO: 75, see, SEQ ID NO: 82.

Corresponding amino acid substitutions to yield a modified high mobility group-box 3 (mHMGB3) can be made at the cysteine residues at positions 23, 45, and/or 104 based on the consensus sequence polypeptide of HMGB3 derived from a human: MAKGDPKKPKGKMSAYAFFVQTCREEHKKKNPEVPVNFAEFSKKCSERWKTMSGK EKSKFDEMAKADKVRYDREMKDYGPAKGGKKKKDPNAPKRPPSGFFLFCSEFRPKI KSTNPGISIGDVAKKLGEMWNNLNDSEKQPYITKAAKLKEKYEKDVADYKSKGKFD GAKGPAKVARKKVEEEDEEEEEEEEEEEEEEDE (SEQ ID NO: 83). As it would be understood by one of skill in the art that mHMGB3 (C104S) is an equivalent of mHMGB1 (C106S) or mHMGB2 (C106S).

Non-limiting exemplary sequences a modified high mobility group-box 3 domains include but are not limited to: mHMGB3 (C23S) which is SEQ ID NO: 83 further comprising one mutation of C23S, i.e., SEQ ID NO: 84; mHMGB3 (C45S) which is SEQ ID NO: 83 further comprising one mutation of C45S, i.e., SEQ ID NO: 85; mHMGB3 (C104S) which is SEQ ID NO: 83 further comprising one mutation of C104S, i.e., SEQ ID NO: 86; mHMGB3 (C23S, C45S—double mutant) which is SEQ ID NO: 83 further comprising two mutations of C23S and C45S, i.e., SEQ ID NO: 87; mHMGB3 (C23S, C104S—double mutant) which is SEQ ID NO: 83 further comprising two mutations of C23S and C104S, i.e., SEQ ID NO: 88; mHMGB3 (C45S, C104S—double mutant) which is SEQ ID NO: 83 further comprising two mutations of C45S and C104S, i.e., SEQ ID NO: 89; and mHMGB3 (C23S, C45S, C104S—triple mutant) which is SEQ ID NO: 83 further comprising three mutations of C23S, C45S and C104S, i.e., SEQ ID NO: 90.

An exemplified nucleic acid sequence encoding a human HMGB3 is provided below:

(SEQ ID NO: 91)
ATGGCTAAAGGTGACCCCAAGAAACCAAAGGGCAAGATGTCCGCTTATGC
CTTCTTTGTGCAGACATGCCAGAGAAGAACATAAGAAGAAAAACCCAGAG
GTCCCTGTCAATTTTGCGGAATTTTCCAAGAAGTGCTCTGAGAGGTGGAA
GACGATGTCCGGGAAAGAGAAATCTAAATTTGATGAAATGGCAAAGGCAG
ATAAAGTGCGCTATGATCGGGAAATGAAGGATTATGGACCAGCTAAGGGA
GGCAAGAAGAAGAAGGATCCTAATGCTCCCAAAAGGCCACCGTCTGGATT
CTTCCTGTTCTGTTCAGAATTCCGCCCCAAGATCAAATCCACAAACCCCG
GCATCTCTATTGGAGACGTGGCAAAAAAGCTGGGTGAGATGTGGAATAAT
TTAAATGACAGTGAAAAGCAGCCTTACATCACTAAGGCGGCAAAGCTGAA
GGAGAAGTATGAGAAGGATGTTGCTGACTATAAGTCGAAAGGAAAGTTTG
ATGGTGCAAAGGGTCCTGCTAAAGTTGCCCGGAAAAAGGTGGAAGAGGAA
GATGAAGAAGAGGAGGAGGAAGAAGAGGAGGAGGAGGAGGAGGAGGATGA
ATAA.

Nucleic acid sequences encoding these modified high mobility group-box 3 domains are also provided herein below: mHMGB3 (C23S) which is SEQ ID NO: 91 further comprising an AGT encoding the C23S, i.e., SEQ ID NO: 91 further comprising one mutation from T to A at nucleotide residue 67 of SEQ ID NO: 91 and one mutation from C to T at nucleotide residue 69 of SEQ ID NO: 91, see, SEQ ID NO: 92; mHMGB3 (C45S) which is SEQ ID NO: 91 further comprising an AGT encoding the C45S, i.e., SEQ ID NO: 91 further comprising one mutation from T to A at nucleotide residue 133 of SEQ ID NO: 91 and one mutation from C to T at nucleotide residue 135 of SEQ ID NO: 91, see, SEQ ID NO: 93; mHMGB3 (C104S) which is SEQ ID NO: 91 further comprising an AGC encoding the C104S, i.e., SEQ ID NO: 91 further comprising one mutation from T to A at nucleotide residue 310 of SEQ ID NO: 91 and one mutation from T to C at nucleotide residue 312 of SEQ ID NO: 91, see, SEQ ID NO: 94; mHMGB3 (C23S, C45S—double mutant) which is SEQ ID NO: 91 further comprising an AGT encoding the C23S and an AGT encoding the C45S, i.e., SEQ ID NO: 91 further comprising one mutation from T to A at nucleotide residue 67 of SEQ ID NO: 91, one mutation from C to T at nucleotide residue 69 of SEQ ID NO: 91, one mutation from T to A at nucleotide residue 133 of SEQ ID NO: 91 and one mutation from C to T at nucleotide residue 135 of SEQ ID NO: 91, see, SEQ ID NO: 95; mHMGB3 (C23S, C104S—double mutant) which is SEQ ID NO: 91 further comprising an AGT encoding the C23S and an AGC encoding the C104S, i.e., SEQ ID NO: 91 further comprising one mutation from T to A at nucleotide residue 67 of SEQ ID NO: 91, one mutation from C to T at nucleotide residue 69 of SEQ ID NO: 91, one mutation from T to A at nucleotide residue 310 of SEQ ID NO: 91 and one mutation from T to C at nucleotide residue 312 of SEQ ID NO: 91, see, SEQ ID NO: 96; mHMGB3 (C45S, C104S—double mutant), which is SEQ ID NO: 91 further comprising an AGT encoding the C45S and an AGC encoding the C104S, i.e., SEQ ID NO: 91 further comprising one mutation from T to A at nucleotide residue 133 of SEQ ID NO: 91, one mutation from C to T at nucleotide residue 135 of SEQ ID NO: 91, one mutation from T to A at nucleotide residue 310 of SEQ ID NO: 91 and one mutation from T to C at nucleotide residue 312 of SEQ ID NO: 91, see, SEQ ID NO: 97; mHMGB3 (C23S, C45S, C104S—triple mutant) which is SEQ ID NO: 91 further comprising an AGT encoding the C23S, an AGT encoding the C45S and an AGC encoding the C104S, i.e., SEQ ID NO: 91 further comprising one mutation from T to A at nucleotide residue 67 of SEQ ID NO: 91, one mutation from C to T at nucleotide residue 69 of SEQ ID NO: 91, one mutation from T to A at nucleotide residue 133 of SEQ ID NO: 91, one mutation from C to T at nucleotide residue 135 of SEQ ID NO: 91, one mutation from T to A at nucleotide residue 310 of SEQ ID NO: 91 and one mutation from T to C at nucleotide residue 312 of SEQ ID NO: 91, see, SEQ ID NO: 98.

Corresponding amino acid substitutions to yield a modified high mobility group-box 4 (mHMGB4) can be made at the cysteine residues at positions 45, 104, 164, and/or 178 based on the consensus sequence polypeptide of HMGB4 derived from a human:

(SEQ ID NO: 99)
MGKEIQLKPKANVSSYVHFLLNYRNKFKEQQPNTYVGFKEFSRKCSEKWR
SISKHEKAKYEALAKLDKARYQEEMMNYVGKRKKRRKRDPQEPRRPPSSF
LLFCQDHYAQLKRENPNWSVVQVAKATGKMWSTATDLEKHPYEQRVALLR
AKYFEELELYRKQCNARKKYRMSARNRCRGKRVRQS.

Non-limiting exemplary sequences a modified high mobility group-box 4 domains include but are not limited to: mHMGB4 (C45S) which is SEQ ID NO: 99 further comprising one mutation of C45S, i.e., SEQ ID NO: 100; mHMGB4 (C104S) which is SEQ ID NO: 99 further comprising one mutation of C104S, i.e., SEQ ID NO: 101; mHMGB4 (C164S) which is SEQ ID NO: 99 further comprising one mutation of C164S, i.e., SEQ ID NO: 102; mHMGB4 (C178S) which is SEQ ID NO: 99 further comprising one mutation of C178S, i.e., SEQ ID NO: 103; mHMGB4 (C45S, C104S—double mutant) which is SEQ ID NO: 99 further comprising two mutations of C45S and C104S, i.e., SEQ ID NO: 104; mHMGB4 (C45S, C164S—double mutant) which is SEQ ID NO: 99 further comprising two mutations of C45S and C164S, i.e., SEQ ID NO: 105; mHMGB4 (C45S, C178S—double mutant) which is SEQ ID NO: 99 further comprising two mutations of C45S and C178S, i.e., SEQ ID NO: 106; mHMGB4 (C104S, C164S—double mutant) which is SEQ ID NO: 99 further comprising two mutations of C104S and C164S, i.e., SEQ ID NO: 107; mHMGB4 (C104S, C178S—double mutant) which is SEQ ID NO: 99 further comprising two mutations of C104S and C178S, i.e., SEQ ID NO: 108; mHMGB4 (C164S, C178S—double mutant) which is SEQ ID NO: 99 further comprising two mutations of C164S and C178S, i.e., SEQ ID NO: 109; mHMGB4 (C45S, C104S, C164S—triple mutant) which is SEQ ID NO: 99 further comprising three mutations of C45S, C104S, and C164S, i.e., SEQ ID NO: 110; mHMGB4 (C45S, C104S, C178S—triple mutant) which is SEQ ID NO: 99 further comprising three mutations of C45S, C104S, and C178S, i.e., SEQ ID NO: 111; mHMGB4 (C45S, C164S, C178S—triple mutant) which is SEQ ID NO: 99 further comprising three mutations of C45S, C164S, and C178S, i.e., SEQ ID NO: 112; mHMGB4 (C104S, C164S, C178S)—triple mutant) which is SEQ ID NO: 99 further comprising three mutations of C104S, C164S, and C178S, i.e., SEQ ID NO: 113; and mHMGB4 (C45S, C104S, C164S, C178S—quadruple mutant) which is SEQ ID NO: 99 further comprising four mutations of C45S, C104S, C164S, and C178S, i.e., SEQ ID NO: 114.

An exemplified nucleic acid sequence encoding a human HMGB4 is provided below:

(SEQ ID NO: 115)
ATGGGAAAAGAAATCCAGCTAAAGCCTAAGGCAAATGTCTCTTCTTACGT
TCACTTTTTGCTGAATTACAGAAACAAATTCAAGGAGCAGCAGCCAAATA
CCTATGTTGGCTTTAAAGAGTTCTCTAGAAAGTGTTCGGAAAAATGGAGA
TCCATCTCAAAGCATGAAAAGGCCAAATATGAAGCCCTGGCCAAACTCGA
CAAAGCCCGATACCAGGAAGAAATGATGAATTATGTTGGCAAGAGGAAGA
AACGGAGAAAGCGGGATCCCCAGGAACCCAGACGGCCTCCATCATCCTTC
CTACTCTTCTGCCAAGACCACTATGCTCAGCTGAAGAGGGAGAACCCGAA
CTGGTCGGTGGTGCAGGTGGCCAAGGCCACAGGGAAGATGTGGTCAACAG
CGACAGACCTGGAGAAGCACCCTTATGAGCAAAGAGTGGCTCTCCTGAGA
GCTAAGTACTTCGAGGAACTTGAACTCTACCGTAAACATGTAATGCCAGG
AAGAAGTACCGAATGTCAGCTAGAAACCGGTGCAGAGGGAAAAGAGTCAG
GCAGAGCTGA.

Nucleic acid sequences encoding these modified high mobility group-box 4 domains are also provided herein below: mHMGB4 (C45S) which is SEQ ID NO: 115 further comprising an AGT encoding the C45S, i.e., SEQ ID NO: 115 further comprising one mutation from T to A at nucleotide residue 133 of SEQ ID NO: 115, see, SEQ ID NO: 116; mHMGB4 (C104S) which is SEQ ID NO: 115 further comprising an AGT encoding the C104S, i.e., SEQ ID NO: 115 further comprising one mutation from T to A at nucleotide residue 310 of SEQ ID NO: 115 and one mutation from C to T at nucleotide residue 312 of SEQ ID NO: 115, see, SEQ ID NO: 117; mHMGB4 (C164S) which is SEQ ID NO: 115 further comprising an AGT encoding the C164S, see, SEQ ID NO: 118; mHMGB4 (C178S) which is SEQ ID NO: 115 further comprising an AGC encoding the C178S, see, SEQ ID NO: 119; mHMGB4 (C45S, C104S—double mutant) which is SEQ ID NO: 115 further comprising an AGT encoding the C45S and an AGT encoding the C104S, see, SEQ ID NO: 120; mHMGB4 (C45S, C164S—double mutant) which is SEQ ID NO: 115 further comprising an AGT encoding the C45S and an AGT encoding the C164S, see, SEQ ID NO: 121; mHMGB4 (C45S, C178S—double mutant) which is SEQ ID NO: 115 further comprising an AGT encoding the C45S and an AGC encoding the C178S, see, SEQ ID NO: 122; mHMGB4 (C104S, C164S—double mutant) which is SEQ ID NO: 115 further comprising an AGT encoding the C104S and an AGT encoding the C164S, see, SEQ ID NO: 123; mHMGB4 (C104S, C178S—double mutant) which is SEQ ID NO: 115 further comprising an AGT encoding the C104S and an AGC encoding the C178S, see, SEQ ID NO: 124; mHMGB4 (C164S, C178S—double mutant) which is SEQ ID NO: 115 further comprising an AGT encoding the C164S and an AGC encoding the C178S, see, SEQ ID NO: 125; mHMGB4 (C45S, C104S, C164S—triple mutant) which is SEQ ID NO: 115 further comprising an AGT encoding the C45S, an AGT encoding the C104S, and an AGT encoding the C164S, see, SEQ ID NO: 126; mHMGB4 (C45S, C104S, C178S—triple mutant) which is SEQ ID NO: 115 further comprising an AGT encoding the C45S, an AGT encoding the C104S, and an AGC encoding the C178S, see, SEQ ID NO: 127; mHMGB4 (C45S, C164S, C178S—triple mutant) which is SEQ ID NO: 115 further comprising an AGT encoding the C45S, an AGT encoding the C164S, and an AGC encoding the C178S, see, SEQ ID NO: 128; mHMGB4 (C104S, C164S, C178S)—triple mutant) which is SEQ ID NO: 115 further comprising an AGT encoding the C104S, an AGT encoding the C164S, and an AGC encoding the C178S, see, SEQ ID NO: 129; mHMGB4 (C45S, C104S, C164S, C178S—quadruple mutant) which is SEQ ID NO: 115 further comprising an AGT encoding the C45S, an AGT encoding the C104S, an AGT encoding the C164S, and an AGC encoding the C178S, see, SEQ ID NO: 130.

An additional exemplified nucleic acid sequence encoding a human HMGB4 is provided below:

(SEQ ID NO: 135)
ATGGGAAAAGAAATCCAGCTAAAGCCTAAGGCAAATGTCTCTTCTTACGT
TCACTTTTTGCTGAATTACAGAAACAAATTCAAGGAGCAGCAGCCAAATA
CCTATGTTGGCTTTAAAGAGTTCTCTAGAAAGTGTTCGGAAAAATGGAGA
TCCATCTCAAAGCATGAAAAGGCCAAATATGAAGCCCTGGCCAAACTCGA
CAAAGCCCGATACCAGGAAGAAATGATGAATTATGTTGGCAAGAGGAAGA
AACGGAGAAAGCGGGATCCCCAGGAACCCAGACGGCCTCCATCATCCTTC
CTACTCTTCTGCCAAGACCACTATGCTCAGCTGAAGAGGGAGAACCCGAA
CTGGTCGGTGGTGCAGGTGGCCAAGGCCACAGGGAAGATGTGGTCAACAG
CGACAGACCTGGAGAAGCACCCTTATGAGCAAAGAGTGGCTCTCCTGAGA
GCTAAGTACTTCGAGGAACTTGAACTCTACCGTAAACAATGTAATGCCAG
GAAGAAGTACCGAATGTCAGCTAGAAACCGGTGCAGAGGGAAAAGAGTCA
GGCAGAGCTGA.

Nucleic acid sequences encoding these modified high mobility group-box 4 domains are also provided herein below: mHMGB4 (C45S) which is SEQ ID NO: 135 further comprising an AGT encoding the C45S, i.e., SEQ ID NO: 135 further comprising one mutation from T to A at nucleotide residue 133 of SEQ ID NO: 135, see, SEQ ID NO: 136; mHMGB4 (C104S) which is SEQ ID NO: 135 further comprising an AGT encoding the C104S, i.e., SEQ ID NO: 135 further comprising one mutation from T to A at nucleotide residue 310 of SEQ ID NO: 135 and optionally one mutation from C to T at nucleotide residue 312 of SEQ ID NO: 135, see, SEQ ID NO: 137; mHMGB4 (C164S) which is SEQ ID NO: 135 further comprising an AGT encoding the C164S, i.e., SEQ ID NO: 135 further comprising one mutation from T to A at nucleotide residue 490 of SEQ ID NO: 135, see, SEQ ID NO: 138; mHMGB4 (C178S) which is SEQ ID NO: 135 further comprising an AGC encoding the C178S, i.e., SEQ ID NO: 135 further comprising one mutation from T to A at nucleotide residue 532 of SEQ ID NO: 135, see, SEQ ID NO: 139; mHMGB4 (C45S, C104S—double mutant) which is SEQ ID NO: 135 further comprising an AGT encoding the C45S and an AGT encoding the C104S, i.e., SEQ ID NO: 135 further comprising one mutation from T to A at nucleotide residue 133 of SEQ ID NO: 135, one mutation from T to A at nucleotide residue 310 of SEQ ID NO: 135 and optionally one mutation from C to T at nucleotide residue 312 of SEQ ID NO: 135, see, SEQ ID NO: 140; mHMGB4 (C45S, C164S—double mutant) which is SEQ ID NO: 135 further comprising an AGT encoding the C45S and an AGT encoding the C164S, i.e., SEQ ID NO: 135 further comprising one mutation from T to A at nucleotide residue 133 of SEQ ID NO: 135 and one mutation from T to A at nucleotide residue 490 of SEQ ID NO: 135, see, SEQ ID NO: 141; mHMGB4 (C45S, C178S—double mutant) which is SEQ ID NO: 135 further comprising an AGT encoding the C45S and an AGC encoding the C178S, i.e., SEQ ID NO: 135 further comprising one mutation from T to A at nucleotide residue 133 of SEQ ID NO: 135, AND one mutation from T to A at nucleotide residue 532 of SEQ ID NO: 135, see, SEQ ID NO: 142; mHMGB4 (C104S, C164S—double mutant) which is SEQ ID NO: 135 further comprising an AGT encoding the C104S and an AGT encoding the C164S, i.e., SEQ ID NO: 135 further comprising one mutation from T to A at nucleotide residue 310 of SEQ ID NO: 135, optionally one mutation from C to T at nucleotide residue 312 of SEQ ID NO: 135, and one mutation from T to A at nucleotide residue 490 of SEQ ID NO: 135, see, SEQ ID NO: 143; mHMGB4 (C104S, C178S—double mutant) which is SEQ ID NO: 135 further comprising an AGT encoding the C104S and an AGC encoding the C178S, i.e., SEQ ID NO: 135 further comprising one mutation from T to A at nucleotide residue 310 of SEQ ID NO: 135, optionally one mutation from C to T at nucleotide residue 312 of SEQ ID NO: 135, and one mutation from T to A at nucleotide residue 532 of SEQ ID NO: 135, see, SEQ ID NO: 144; mHMGB4 (C164S, C178S—double mutant) which is SEQ ID NO: 135 further comprising an AGT encoding the C164S and an AGC encoding the C178S, i.e., SEQ ID NO: 135 further comprising one mutation from T to A at nucleotide residue 490 of SEQ ID NO: 135 and one mutation from T to A at nucleotide residue 532 of SEQ ID NO: 135, see, SEQ ID NO: 145; mHMGB4 (C45S, C104S, C164S—triple mutant) which is SEQ ID NO: 135 further comprising an AGT encoding the C45S, an AGT encoding the C104S, and an AGT encoding the C164S, i.e., SEQ ID NO: 135 further comprising one mutation from T to A at nucleotide residue 133 of SEQ ID NO: 135, one mutation from T to A at nucleotide residue 310 of SEQ ID NO: 135, optionally one mutation from C to T at nucleotide residue 312 of SEQ ID NO: 135, and one mutation from T to A at nucleotide residue 490 of SEQ ID NO: 135, see, SEQ ID NO: 146; mHMGB4 (C45S, C104S, C178S—triple mutant) which is SEQ ID NO: 135 further comprising an AGT encoding the C45S, an AGT encoding the C104S, and an AGC encoding the C178S, i.e., SEQ ID NO: 135 further comprising one mutation from T to A at nucleotide residue 133 of SEQ ID NO: 135, one mutation from T to A at nucleotide residue 310 of SEQ ID NO: 135, optionally one mutation from C to T at nucleotide residue 312 of SEQ ID NO: 135, and one mutation from T to A at nucleotide residue 532 of SEQ ID NO: 135, see, SEQ ID NO: 147; mHMGB4 (C45S, C164S, C178S—triple mutant) which is SEQ ID NO: 135 further comprising an AGT encoding the C45S, an AGT encoding the C164S, and an AGC encoding the C178S, i.e., SEQ ID NO: 135 further comprising one mutation from T to A at nucleotide residue 133 of SEQ ID NO: 135, one mutation from T to A at nucleotide residue 490 of SEQ ID NO: 135, and one mutation from T to A at nucleotide residue 532 of SEQ ID NO: 135, see, SEQ ID NO: 148; mHMGB4 (C104S, C164S, C178S)—triple mutant) which is SEQ ID NO: 135 further comprising an AGT encoding the C104S, an AGT encoding the C164S, and an AGC encoding the C178S, i.e., SEQ ID NO: 135 further comprising one mutation from T to A at nucleotide residue 310 of SEQ ID NO: 135, optionally one mutation from C to T at nucleotide residue 312 of SEQ ID NO: 135, one mutation from T to A at nucleotide residue 490 of SEQ ID NO: 135, and one mutation from T to A at nucleotide residue 532 of SEQ ID NO: 135, see, SEQ ID NO: 149; mHMGB4 (C45S, C104S, C164S, C178S—quadruple mutant) which is SEQ ID NO: 135 further comprising an AGT encoding the C45S, an AGT encoding the C104S, an AGT encoding the C164S, and an AGC encoding the C178S, e.g., one mutation from T to A at nucleotide residue 133 of SEQ ID NO: 135, SEQ ID NO: 135 further comprising one mutation from T to A at nucleotide residue 310 of SEQ ID NO: 135, optionally one mutation from C to T at nucleotide residue 312 of SEQ ID NO: 135, one mutation from T to A at nucleotide residue 490 of SEQ ID NO: 135, and one mutation from T to A at nucleotide residue 532 of SEQ ID NO: 135, see, SEQ ID NO: 150.

It is appreciated that among the equivalents to a modified high mobility group-box 1 domain are modified high mobility group-box 2, modified high mobility group-box 3, and modified high mobility group-box 4 comprising cysteine to serine substitutions at one or more amino acid positions corresponding to C23S, C45S, and C106S of a modified high mobility group box 1. For modified high mobility group-box 2 the corresponding cysteine residues are found at position 23, 45, and 106; for modified high mobility group-box 3 the corresponding cysteine residues are found at position 23, 45, and 104; and for modified high mobility group-box 4 the corresponding cysteine residues are found at position 45, 104, 164, and 178. Thus, Applicants believe that with respect to a modified high mobility group-box 1 comprising one or more substitutions selected from the group of C23S, C45S, and C106S are equally applicable to the aforementioned high mobility group-box species with one or more cysteine to serine substitutions at the mentioned positions, e.g. a modified high mobility group-box 2 comprising one or more substitutions selected from the group of C23S, C45S, and C106S; a modified high mobility group-box 3 comprising one or more substitutions selected from the group of C23S, C45S, and C104S; and a modified high mobility group-box 4 comprising one or more substitutions selected from the group of C45S, C104S, C164S, and C178S. In one aspect, equivalents of such polypeptides or proteins include those having at least 70% or more (as described herein) percent identify with the proviso that the specific substituted amino acids are maintained, e.g., C23S, C45S, and/or C106S.

An “A Box” polypeptide intends a polypeptide comprising the A box domain of HMGB1 protein. The A Box polypeptide may be mutated or contain additional sequences such as a linker sequence, a signal sequence or a secretion sequence. One or more point mutations in the amino acids K12, C23 and C45 can be introduced. In some embodiments, the A box polypeptide comprises, or consists essentially of, or yet further consists of aa 9 to aa 79 of SEQ ID NO: 51 or an equivalent thereof, such as a fragment of an HMGB1 polypeptide as disclosed herein aligned to aa 9 to aa 79 of SEQ ID NO: 51. In some embodiments, the A box polypeptide comprises, or consists essentially of, or yet further consists of aa 1 to aa 79 of SEQ ID NO: 51 or an equivalent thereof, such as a fragment of an HMGB1 polypeptide as disclosed herein aligned to aa 1 to aa 79 of SEQ ID NO: 51. In some embodiments, the A box polypeptide comprises, or consists essentially of, or yet further consists of aa 1 to aa 70 of SEQ ID NO: 51 or an equivalent thereof, such as a fragment of an HMGB1 polypeptide as disclosed herein aligned to aa 1 to aa 70 of SEQ ID NO: 51. Examples of A Box polypeptides comprise, or consist essentially of, or yet further consist of:

(aa 1 to aa 89 of SEQ ID NO: 132)
MGKGDPKKPRRKMSSYAFFVQTCREEHKKKHPDASVNFSEFSKKCSERWK
TMSAKEKGKFEDMAKADKARYEREMKTYIPPKGETKKKF (murine)
or
(aa 1 to aa 70 of SEQ ID NO: 132)
MGKGDPKKPRRKMSSYAFFVQTCREEHKKKHPDASVNFSEFSKKCSERWK
TMSAKEKGKFEDMAKADKAR;
or
(aa 1 to aa 89 of SEQ ID NO: 51)
MGKGDPKKPRGKMSSYAFFVQTCREEHKKKHPDASVNFSEFSKKCSERWK
TMSAKEKGKFEDMAKADKARYEREMKTYIPPKGETKKKF (human)
or
(aa 1 to aa 70 of SEQ ID NO: 51)
MGKGDPKKPRGKMSSYAFFVQTCREEHKKKHPDASVNFSEFSKKCSERWK
TMSAKEKGKFEDMAKADKAR.

A “B Box” polypeptide intends a polypeptide comprising the B box domain of HMGB1 protein. The B Box polypeptide may be mutated or contain additional sequences such as a linker sequence, a signal sequence or a secretion sequence. A point mutations in the amino acid K114 or C106 can be introduced to effect DNA binding, inflammatory properties, and anti-biofilm activity. In some embodiments, the B box polypeptide comprises, or consists essentially of, or yet further consists of aa 95 to aa 163 of SEQ ID NO: 51 or an equivalent thereof, such as a fragment of an HMGB1 polypeptide as disclosed herein aligned to aa 95 to aa 163 of SEQ ID NO: 51. In some embodiments, the B box polypeptide comprises, or consists essentially of, or yet further consists of aa 88 to aa 164 of SEQ ID NO: 51 or an equivalent thereof, such as a fragment of an HMGB1 polypeptide as disclosed herein aligned to aa 88 to aa 164 of SEQ ID NO: 51. In some embodiments, the B box polypeptide comprises, or consists essentially of, or yet further consists of aa 80 to aa 164 of SEQ ID NO: 51 or an equivalent thereof, such as a fragment of an HMGB1 polypeptide as disclosed herein aligned to aa 80 to aa 164 of SEQ ID NO: 51. In some embodiments, the B box polypeptide comprises, or consists essentially of, or yet further consists of aa 80 to aa 176 of SEQ ID NO: 51 or an equivalent thereof, such as a fragment of an HMGB1 polypeptide as disclosed herein aligned to aa 80 to aa 176 of SEQ ID NO: 51. In some embodiments, the B box polypeptide comprises, or consists essentially of, or yet further consists of aa 90 to aa 176 of SEQ ID NO: 51 or an equivalent thereof, such as a fragment of an HMGB1 polypeptide as disclosed herein aligned to aa 90 to aa 176 of SEQ ID NO: 51. In some embodiments, the B box polypeptide comprises, or consists essentially of, or yet further consists of aa 89 to aa 162 of SEQ ID NO: 51 or an equivalent thereof, such as a fragment of an HMGB1 polypeptide as disclosed herein aligned to aa 89 to aa 162 of SEQ ID NO: 51. Examples of B Box polypeptides comprise, or consists essentially of, or yet further consist of:

(aa 90 to aa 176 of SEQ ID NO: 132)
KDPNAPKRPPSAFFLFCSEYRPKIKGEHPGLSIGDVAKKLGEMWNNTAAD
DKQPYEKKAEKLKEKYEKDIAAYRAKGKPDAAKKGVV; (murine)
or
(aa 90 to aa 176 of SEQ ID NO: 51)
KDPNAPKRPPSAFFLFCSEYRPKIKGEHPGLSIGDVAKKLGEMWNNTAAD
DKQPYEKKAAKLKEKYEKDIAAYRAKGKPDAAKKGVV. (human)

A “C box” polypeptide intends a polypeptide comprising the C tail domain of HMGB1 protein. The C tail polypeptide may be mutated or contain additional sequences such as a linker sequence, a signal sequence or a secretion sequence. In some embodiments, the C box polypeptide comprises, or consists essentially of, or yet further consists of aa 186 to aa 215 of SEQ ID NO: 51 or an equivalent thereof, such as a fragment of an HMGB1 polypeptide as disclosed herein aligned to aa 186 to aa 215 of SEQ ID NO: 51. In some embodiments, the C box polypeptide comprises, or consists essentially of, or yet further consists of EEEEDEEDEEDEEEE EDEEDEDEEE DDDDE (SEQ ID NO: 133).

The “AB Box” polypeptide intends a polypeptide comprising the A and B box domains of HMGB1 protein fused together but absent amino acids that correspond to full length wild-type protein. The AB Box polypeptide may be mutated or contain additional sequences such as a linker sequence, a signal sequence or a secretion sequence. One or more point mutations in the amino acids as described herein (e.g., at amino acids K12, C23, C45, C106, and/or K114) can be introduced to effect DNA binding, inflammatory properties, and anti-biofilm activity. In some embodiments, the AB box polypeptide comprises, or consists essentially of, or yet further consists of aa 1 to aa 176 of SEQ ID NO: 51 or an equivalent thereof, such as a fragment of an HMGB1 polypeptide as disclosed herein aligned to aa 1 to aa 176 of SEQ ID NO: 51. In some embodiments, the AB box polypeptide comprises, or consists essentially of, or yet further consists of aa 1 to aa 162 of SEQ ID NO: 51 or an equivalent thereof, such as a fragment of an HMGB1 polypeptide as disclosed herein aligned to aa 1 to aa 162 of SEQ ID NO: 51. In some embodiments, the AB box polypeptide comprises, or consists essentially of, or yet further consists of aa 1 to aa 164 of SEQ ID NO: 51 or an equivalent thereof, such as a fragment of an HMGB1 polypeptide as disclosed herein aligned to aa 1 to aa 164 of SEQ ID NO: 51.

In some embodiments, an equivalent to an HMGB1 polypeptide as described herein can comprise, or consist essentially of, or yet further consist of an HMG-box domain truncate and/or mutant as described herein, or a protein or a fragment of the protein that contains one or more of the HMG-box domain, truncate, mutant or equivalents of the protein or fragment having the disclosed amino acid substitutions. In some embodiments, a fragment of an HMGB1 polypeptide comprises, or consists essentially of, or yet further consists of one or more of: an A box polypeptide as disclosed herein, a B box polypeptide as disclosed herein, or an AB box polypeptide as disclosed herein.

In some embodiments, an A Box polypeptide can further comprise, or alternatively consist essentially of, or yet consist of one or more amino acid mutations selected from K12, C23 and C45 (e.g., the native K or C modified to an amino acid from the group selected from serine, glycine, alanine, valine, isoleucine or threonine) or an equivalent thereof, the equivalent comprising the one or more amino acid mutations selected from K12, C23 and C45, e.g. the native K or C modified to an amino acid from the group selected from serine, glycine, alanine, valine, isoleucine or threonine. In one aspect, the mutation is a C45S mutation. The A Box polypeptide may further comprise a linker or peptide sequence located at one or both termini. An example of a peptide linker is PPKGETKKKF (SEQ ID NO: 131).

When recombinantly produced, the A Box polypeptides can be partially or fully acetylated, oxidized or phosphorylated, using methods known in the art, e.g., Olia A S, et al. (2015) ACS chemical biology. 10(9):2034-47. doi: 10.1021/acschembio.5b00342, PubMed PMID: 26083674; PubMed Central PMCID: PMC4610810; Ugrinova I, et al. (2102) Molecular Biology Reports, 2012; 39(11):9947-53. Epub 2012/06/29. doi: 10.1007/s11033-012-1863-x. PubMed PMID: 22740141; and Ito T, et al. (2007) JTH, 5(1):109-16. doi: 10.1111/j.1538-7836.2006.02255.x. PubMed PMID: 17239166. In one aspect, the A Box polypeptide comprises, or consists essentially of, or yet further consists of amino acids 1 to 70 of wild-type HMGB1 polypeptide, with the aforementioned mutations.

In some embodiments, a B Box polypeptide can comprise, or alternatively consist essentially of, or yet consist of a mutation at amino acid C106 or K114 or both (e.g. the native cysteine to an amino acid from the group selected from serine, glycine, alanine, valine, isoleucine or threonine), or an equivalent thereof comprising the mutation at amino acid C106 or K114 or both (e.g. the native cysteine to an amino acid from the group selected from serine, glycine, alanine, valine, isoleucine or threonine). In one aspect, the B Box polypeptide comprises, or consists essentially of, or yet further consists of amino acids about 80 to about 176, or about 88 to about 164, or about 89 to about 162, or yet further about 80 to about 164, of the wt HMGB1 polypeptide, with the aforementioned mutations.

The B Box polypeptide may further comprise a linker or peptide sequence located at one or both termini. An examples of a peptide linker is PPKGETKKKF (SEQ ID NO: 131). When recombinantly produced, the disclosed B Box polypeptides can be partially or fully acetylated, oxidized or phosphorylated, using methods known in the art, e.g., Olia A S, et al. (2015) ACS chemical biology. 10(9):2034-47. doi: 10.1021/acschembio.5b00342, PubMed PMID: 26083674; PubMed Central PMCID: PMC4610810; Ugrinova I, et al. (2102) Molecular Biology Reports, 2012; 39(11):9947-53. Epub 2012/06/29. doi: 10.1007/s11033-012-1863-x. PubMed PMID: 22740141; and Ito T, et al. (2007) JTH, 5(1):109-16. doi: 10.1111/j.1538-7836.2006.02255.x. PubMed PMID: 17239166.

In some embodiments, an AB Box polypeptide can comprise, or alternatively consist essentially of, or yet consist of one or more amino acid mutations selected from K12, C23, C45, C106 or K114 (e.g. the native K or C modified to an amino acid from the group selected from serine, glycine, alanine, valine, isoleucine or threonine) or an equivalent thereof comprising the one or more amino acid mutations selected from K12, C23, C45, C106 or K114 (e.g. the native K or C modified to an amino acid from the group selected from serine, glycine, alanine, valine, isoleucine or threonine). In one aspect, the mutation is a C45S mutation. In another aspect, the polypeptide comprises a mutation at amino acid C106 (e.g. the native cysteine to an amino acid from the group selected from serine, glycine, alanine, valine, isoleucine or threonine), or an equivalent thereof comprising the one or more amino acid mutations selected from K12, C23, C45 and a mutation at amino acid C106 (e.g. the native cysteine to an amino acid from the group selected from serine, glycine, alanine, valine, isoleucine or threonine). In one aspect, the AB Box polypeptide comprises C45S and C106S mutations and equivalents retain these mutations. In one aspect, the AB Box polypeptide comprises, or consists essentially of, or yet further consists of amino acids 1 to 176, or 1 to 162, or yet further 1 to 164, of the wild type HMGB1 polypeptide, with the aforementioned mutations.

In some embodiments, the AB Box polypeptide further comprises a linker polypeptide located between the A Box polypeptide and the B Box polypeptide and in one aspect, a second linker linking the B Box and a C Box polypeptide. When recombinantly produced, the AB or A, B and C Box polypeptides can be partially or fully acetylated, oxidized or phosphorylated. An examples of a peptide linker is PPKGETKKKF (SEQ ID NO: 131). In one aspect, an isolated mutated HMGB1 polypeptide is provided with 1 or more amino acid substitutions as described herein, in the A and/or B box domains that can optionally be partially or fully acetylated, oxidized or phosphorylated, using methods known in the art, e.g., Olia A S, et al. (2015) ACS chemical biology. 10(9):2034-47. doi: 10.1021/acschembio.5b00342, PubMed PMID: 26083674; PubMed Central PMCID: PMC4610810; Ugrinova I, et al. (2102) Molecular Biology Reports, 2012; 39(11):9947-53. Epub 2012/06/29. doi: 10.1007/s11033-012-1863-x. PubMed PMID: 22740141; and Ito T, et al. (2007) JTH, 5(1):109-16. doi: 10.1111/j.1538-7836.2006.02255.x. PubMed PMID: 17239166.

Example of an AB Box polypeptides comprise, or consists essentially of, or yet further consist of with the aforementioned mutations:

(SEQ ID NO: 132)
MGKGDPKKPRRKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAEK
LKEKYEKDIAAYRAKGKPDAAKKGVV; (murine)
or
(aa 1 to aa 176 of SEQ ID NO: 51)
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVV (human).

In a further aspect, the AB Box polypeptide further comprises a linker polypeptide located between the A Box polypeptide and the B Box polypeptide.

In some embodiments, an HMGB1 polypeptide or a fragment thereof comprises, or consists essentially of, or yet further consists of the A, B and C domains, wherein the polypeptide comprises, or consists essentially of, or yet further consists of, one or more amino acid mutations selected from K12, C23, C45, C106, or K114, or an equivalent thereof, the equivalent thereof comprising the one or more amino acid mutations selected from K12, C23, C45, C106 or K114.

As used herein, an equivalent of a polypeptide refers to a sequence that is at least about 70%, or alternatively at least about 75%, or at least about 80%, or at least about 85%, or at least about 90%, or at least about 95%, or at least about 98% or at least about 99% identical to the reference polypeptide that in one aspect, retain the mutated amino acid(s). In some aspects, the equivalent of a polypeptide retains the intended function and/or structural characteristics of the polypeptide, e.g., containing an HMG-box domain, and optionally does not induce a pro-inflammatory response. In one aspect, the equivalent polypeptide includes a domain that is at least about 70%, or alternatively at least about 80%, or at least about 85%, or at least about 90%, or at least about 95%, or at least about 98% or at least about 99% identical to the HMG-box domain that in one aspect, retain the mutated amino acid(s). In some aspects, such an equivalent domain retains the function and/or structural characteristics of the H1 MB-box domain, e.g., binding to a HMB-box binding target optionally but does not induce a pro-inflammatory response. In one aspect, the equivalent polypeptide is encoded by a polynucleotide that can hybridize with a polynucleotide encoding the HMB-box domain polypeptide under stringent conditions.

In some embodiments, the HMGB1 Box polypeptide further comprises linker polypeptides located between the A Box polypeptide and the B Box polypeptide and a second linker polypeptide linking the B Box polypeptide and the C Box polypeptide. An example of a peptide linker is PPKGETKKKF (SEQ ID NO: 131).

An immunodominant antigen intends a region of the protein that is recognized and binds with high affinity to an antibody.

An immunoprotective antigen intends a region of the protein that is recognized and binds with high affinity to an antibody to interfere with protein function; the antibodies generated against an immunoprotective antigen are characterized by enhanced or optimal effect against a target indication as a result to the interference with protein function—in this case, an improve capability to clear biofilms.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

The term “isolated” as used herein refers to molecules, biologicals, cellular materials, cells or biological samples being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source.

In some embodiments, the term “engineered” refers to comprising at least one modification not normally found in a naturally occurring counterpart, wild-type or a parent. In some embodiments, the term “engineered” is used interchangeably with “recombinant” refers to being synthetized by human.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen-binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen-binding fragment. In another aspect, the equivalent thereof competes with the binding of the antibody or antigen-binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Examples of biologically equivalent polypeptides are provided in Table 9 of WO 2011/123396 which identifies conservative amino acid substitutions to the disclosed amino acid sequences.

In some aspects, an equivalent to an amino acid sequence comprises a polypeptide having at least 80% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In a further aspect, an equivalent to an amino acid sequence comprises a polypeptide having at least 90% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In yet a further aspect, an equivalent to an amino acid sequence comprises a polypeptide having at least 95% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In one aspect, an equivalent to an amino acid sequence comprises a polypeptide having at least 96% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In a further aspect, an equivalent to an amino acid sequence comprises a polypeptide having at least 97% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In yet a further aspect, an equivalent to an amino acid sequence comprises a polypeptide having at least 98% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence. In one aspect, an equivalent to an amino acid sequence comprises a polypeptide having at least 99% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address:align.genome.jp, last accessed on Mar. 7, 2011). In some embodiments, Clustal Omega, accessible at www.ebi.ac.uk/Tools/msa/clustalo/, is used in a sequence alignment or determining an identity percentage. In further embodiments, default setting is applied.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder. As used herein, “treating” or “treatment” of a disease in a subject can also refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

To prevent intends to prevent a disorder or effect in vitro or in vivo in a system or subject that is predisposed to the disorder or effect. An example of such is preventing the formation of a biofilm in a system that is infected with a microorganism known to produce one.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination, and can be separately packaged with or without specific dosages. The active ingredients of the combination can be administered concurrently or sequentially.

A “biologically active agent” or an active agent disclosed herein intends one or more of an isolated or recombinant polypeptide, an isolated or recombinant polynucleotide, a vector, an isolated host cell, or an antibody, as well as compositions comprising one or more of same.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “conjugated moiety” refers to a moiety that can be added to an isolated chimeric polypeptide by forming a covalent bond with a residue of chimeric polypeptide. The moiety may bond directly to a residue of the chimeric polypeptide or may form a covalent bond with a linker which in turn forms a covalent bond with a residue of the chimeric polypeptide.

A “peptide conjugate” refers to the association by covalent or non-covalent bonding of one or more polypeptides and another chemical or biological compound. In a non-limiting example, the “conjugation” of a polypeptide with a chemical compound results in improved stability or efficacy of the polypeptide for its intended purpose. In one embodiment, a peptide is conjugated to a carrier, wherein the carrier is a liposome, a micelle, or a pharmaceutically acceptable polymer.

“Liposomes” are microscopic vesicles consisting of concentric lipid bilayers. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloteoylphosphatidylcholine (POPC), palmitoyloleoylphosphatidylethanolamine (POPE) and dioleoylphosphatidylethanolamine 4-(N-maleimido-triethyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stearate, amphoteric acrylic polymers, polyethyoxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioteoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs). The biological active agents can be encapsulated in such for administration in accordance with the methods described herein.

A “micelle” is an aggregate of surfactant molecules dispersed in a liquid colloid. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the head groups at the center with the tails extending out (water-in-oil micelle). Micelles can be used to attach a polynucleotide, polypeptide, antibody or composition described herein to facilitate efficient delivery to the target cell or tissue.

The phrase “pharmaceutically acceptable polymer” refers to the group of compounds which can be conjugated to one or more polypeptides described here. It is contemplated that the conjugation of a polymer to the polypeptide is capable of extending the half-life of the polypeptide in vivo and in vitro. Non-limiting examples include polyethylene glycols, polyvinylpyrrolidones, polyvinylalcohols, cellulose derivatives, polyacrylates, polymethacrylates, sugars, polyols and mixtures thereof. The biological active agents can be conjugated to a pharmaceutically acceptable polymer for administration in accordance with the methods described herein.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue or a subject using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

The term “a regulatory sequence” “a regulatory element” “an expression control element” or “promoter” as used herein, intends a polynucleotide that is operatively linked to a polynucleotide to be transcribed and/or replicated, and facilitates the expression and/or replication of the polynucleotide. Non-limiting examples of a regulatory sequence include a promoter, an enhancer, or a polyadenylation sequence.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include a cytomegalovirus CMV promoter or retroviral long terminal repeat (LTR) promoter. See, for example, Weber et al. Hum Gene Ther. 2007 September; 18(9):849-60.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein the term “eDNA” refers to extracellular DNA found as a component to pathogenic biofilms.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., PCT International Application Publication No. WO 95/27071. Ads do not require integration into the host cell genome. Recombinant Ad derived vectors, particularly those that reduce the potential for recombination and generation of wild-type virus, have also been constructed. See, PCT International Application Publication Nos. WO 95/00655 and WO 95/11984, Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or an antigen-binding fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the terms “antibody,” “antibodies” and “immunoglobulin” includes whole antibodies and any antigen-binding fragment or a single chain thereof. Thus, the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region (which is also referred to herein as a variable domain), a heavy chain or light chain constant region (which is also referred to herein as a constant domain), a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-DNABII, anti-IHF, anti-HU, anti-OMP P5, for example, refers to a monoclonal or polyclonal antibody that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

Complementarity determining regions (CDRs) are part of the variable region of an antibody or a T cell receptor generated by B-cell s and T-cells respectively, wherein these molecules bind to their specific antigen (also called epitope). In certain embodiments, the terms “variable region” and “variable domain” are used interchangeably, referring to the polypeptide of a light or heavy chain of an antibody that varies greatly in its sequence of amino acid residues from one antibody to another, and that determines the conformation of the combining site which confers the specificity of the antibody for a particular antigen. In a further embodiment, the variable region is about 90 amino acids long to about 200 amino acids long, including but not limited to about 100 amino acids long, or alternatively about 110 amino acids long, or alternatively about 120 amino acids long, or alternatively about 130 amino acids long, or alternatively about 140 amino acids long, or alternatively about 150 amino acids long, or alternatively about 160 amino acids long, or alternatively about 170 amino acids long, or alternatively about 180 amino acids long, or alternatively about 190 amino acids long. In certain embodiments, a variable region of an amino acid sequence, as used herein, refers to that the first about 100 amino acids, or alternatively about 110 amino acids, or alternatively about 120 amino acids, or alternatively about 130 amino acids, or alternatively about 140 amino acids, or alternatively about 150 amino acids of the amino acid sequence (including or excluding a signal peptide if applicable) is the variable region.

A set of CDRs constitutes a paratope also called an antigen-binding site, which is a part of an antibody that recognizes and binds to an antigen. There are three CDRs (CDR1, CDR2 and CDR3), arranged non-consecutively, optionally from the amino terminus to the carboxyl terminus, on the amino acid sequence of a variable region of an antigen receptor, such as a heavy chain or a light chain. As used herein, CDRn refers to a CDRn in an immunoglobulin chain or derived from an immunoglobulin chain, wherein the number n is selected from 1-3. In one embodiment, CDRLn refers to a CDRn in a light chain or derived from a light chain, wherein the number n is selected from 1-3; while CDRHn refers to a CDRn in a heavy chain or derived from a heavy chain, wherein the number n is selected from 1-3. In certain embodiments, framework region (FR) refers to the part of a variable region which is not a CDR. In certain embodiments, FRn refers to a FR in a heavy chain or a light chain or derived from a heavy chain or a light chain, and wherein the number n is selected from 1-4. In certain embodiments, a variable region comprises or consists essentially of, or yet further consists of the following (optionally following the order as provided, and further optionally from the amino terminus to the carboxyl terminus): FR1, CDR1, FR2, CDR2, FR3, CDR3 and FR4.

Variable regions and/or CDRs of an antibody or a fragment thereof can be determined by one of skill in the art, for example, using publically or commercially available tools. Non-limiting examples of such tools include, IgBlast (accessible at www.ncbi.nlm.nih.gov/igblast/), Scaligner (available from drugdesigntech at www.scaligner.com/), IMGT rules and/or tools (see, for example, www.imgt.org/IMGTScientificChart/Nomenclature/IMGT-FRCDRdefinition.html, also accessible at www.imgt.org/), Chothia Canonical Assignment (accessible at www.bioinforg.uk/abs/chothia.html), Antigen receptor Numbering And Receptor CalssificatiIon (ANARCI, accessible at opig.stats.ox.ac.uk/webapps/newsabdab/sabpred/anarci/), the Kabat numbering method/scheme (e.g., Kabat, E. A., et al., (1991) Sequences of Proteins of Immunological Interest, Fifth Edition, U.S. Department of Health and Human Services, NIH Publication No. 91-3242) or the Paratome web server (accessible at www.ofranlab.org/paratome/, see Vered Kunik, et al, Nucleic Acids Research, Volume 40, Issue W1, 1 Jul. 2012, Pages W521-W524).

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region or a fragment thereof (for example, 1, 2, 3, 4, 5, or all 6 CDRs) of the recipient are replaced by residues from a variable region or a fragment thereof (for example, 1, 2, 3, 4, 5, or all 6 CDRs) of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. Without wishing to be bound by the theory, humanized antibodies produce a reduced immune response in a human host, as compared to a non-humanized version of the same antibody. The humanized antibodies may have conservative amino acid substitutions which have substantially no effect on antigen-binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Specifically, the humanized antibodies as disclosed herein specifically binds to a DNABII polypeptide or a fragment thereof (such as the tip chimeric peptide or the tail chimeric peptide) with certain range(s) of one or more of the following: EC₅₀, Kₒₙ, K_{off}, K_{A} and/or K_{D}, and inhibits or releases certain cytokine(s) upon treating a subject. In a further embodiment, the humanized antibody specifically binding to the tip region of a DNABII polypeptide (such as the tip chimeric peptide) disrupts biofilm both in vivo and in vitro. In addition, the process of humanization, while a rational design process, may produce unexpected changes (positive or negative) in e.g. binding affinity, antigen specificity, or physical properties such as solubility or aggregatability; hence, properties of humanized antibodies are not inherently predictable from the properties of the starting non-human antibody.

In one embodiment, an antibody as used herein may be a recombinant antibody. The term “recombinant antibody”, as used herein, includes all antibodies that are prepared, expressed, created or isolated by recombinant means, such as antibodies isolated from an animal (e.g., a mouse) that is transgenic or transchromosomal for immunoglobulin genes or a hybridoma prepared therefrom, antibodies isolated from a host cell transformed to express the antibody, e.g., from a transfectoma, antibodies isolated from a recombinant, combinatorial antibody library, and antibodies prepared, expressed, created or isolated by any other means that involve splicing of immunoglobulin (Ig) gene sequences to other DNA sequences. In certain embodiments, however, such recombinant antibodies can be subjected to in vitro mutagenesis (or, when an animal transgenic for Ig sequences is used, in vivo somatic mutagenesis) and thus the amino acid sequences of the VH and VL regions of the recombinant antibodies are sequences that may not naturally exist within the antibody germline repertoire in vivo. Methods to making these antibodies are described herein.

In one embodiment, an antibody as used herein may be a chimeric antibody. As used herein, chimeric antibodies are antibodies whose light and heavy chain genes have been constructed, typically by genetic engineering, from antibody variable and constant region genes belonging to different species.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

The terms “antigen” and “antigenic” refer to molecules with the capacity to be recognized by an antibody or otherwise act as a member of an antibody-ligand pair. “Specific binding” or “binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

An “altered antigen” is one having a primary sequence that is different from that of the corresponding wild-type antigen. Altered antigens can be made by synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand. (Ferguson et al. (1988) Ann. Rev. Biochem. 57:285-320). A synthetic or altered antigen disclosed herein is intended to bind to the same TCR as the natural epitope.

A “self-antigen” also referred to herein as a native or wild-type antigen is an antigenic peptide that induces little or no immune response in the subject due to self-tolerance to the antigen. An example of a self-antigen is the melanoma specific antigen gp100.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

The term “passive immunity” refers to the transfer of immunity from one subject to another through the transfer of antibodies. Passive immunity may occur naturally, as when maternal antibodies are transferred to a fetus. Passive immunity may also occur artificially as when antibody compositions are administered to non-immune subjects. Antibody donors and recipients may be human or non-human subjects. Antibodies may be polyclonal or monoclonal, may be generated in vitro or in vivo, and may be purified, partially purified, or unpurified depending on the embodiment. In some embodiments described herein, passive immunity is conferred on a subject in need thereof through the administration of antibodies or antigen-binding fragments that specifically recognize or bind to a particular antigen. In some embodiments, passive immunity is conferred through the administration of an isolated or recombinant polynucleotide encoding an antibody or antigen-binding fragment that specifically recognizes or binds to a particular antigen.

In the context of this disclosure, a “ligand” is a polypeptide. In one aspect, the term “ligand” as used herein refers to any molecule that binds to a specific site on another molecule. In other words, the ligand confers the specificity of the protein in a reaction with an immune effector cell or an antibody to a protein or DNA to a protein. In one aspect it is the ligand site within the protein that combines directly with the complementary binding site on the immune effector cell.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TENTAGEL®, Rapp Polymere, Tubingen, Germany) or polydimethylacrylamide resin (obtained from Milligen/Biosearch, Calif.).

An example of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In one embodiment, the biological sample is suspect of having a biofilm. In another embodiment, the biological sample comprise a biofilm.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

As used herein, a cleavable peptide, which is also referred to as a cleavable linker, means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving (SEQ ID NO: 134).

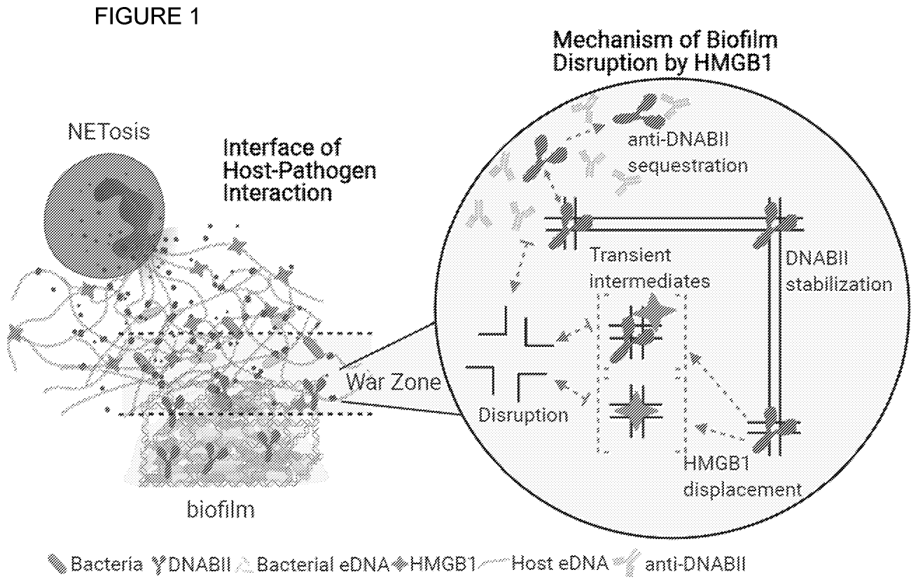

As used herein, the term “chimer” or “chimeric peptide” refers to a recombinant polypeptide comprising or alternatively consisting essentially of, or yet further consisting of, two or more fragments or domains of a DNABII polypeptide conjugated directly or indirectly (such as via a linker) with each other. In one embodiment, the domains are conformational tip domains and/or conformational tail domains. Additionally or alternatively, the two or more fragments or domains is derived from the same or different DNABII polypeptide(s). In one embodiment, the chimeric peptide comprises or alternatively consists essentially of, or yet further consists of, a tip domain of IhfA and a tip domain of IhfB conjugated directly or indirectly (such as via a linker) with each other. In another embodiment, the chimeric peptide comprises or alternatively consists essentially of, or yet further consists of, a tail domain of IhfA and a tail domain of IhfB conjugated directly or indirectly (such as via a linker) with each other. “A conformational tip domain” of a polypeptide refers to a polypeptide that comprises a primary amino acid sequence wherein the structure has an anti-parallel beta ribbon with a sharp turn that is typically mediated by a proline residue. The “tip” of an IHF polypeptide is shown in FIG. 1 of WO2018/129078.

As used herein, the phrase “derived from” means isolated from, purified from, or engineered from, or any combination thereof.

In certain embodiments, the antibody specifically binds the tip-chimeric peptide IhfA5-mIhfB4_{NTHI} that comprises, or consists essentially of, or yet further consists of: the polypeptide sequence:

(SEQ ID NO: 38)
RPGRNPX₁TGDVVPVSARRVV-X-FSLHHRQPRLGRNPX₁TGDSV,

wherein “X” is an optional amino acid linker sequence, optionally comprising, or consisting essentially of, or yet further consisting of between 1 to 20 amino acids; and wherein “X₁” is any amino acid or alternatively “X₁” is selected from the amino acids Q, R, K, S, or T. In a further aspect, “X₁” is a K or Q. In a further embodiment, the tip-chimeric peptide IhfA5-mIhfB4_{NTHI} comprises, or consists essentially of, or yet further consists of: the polypeptide sequence of RPGRNPKTGDVVPVSARRVV-X-FSLHHRQPRLGRNPKTGDSV (SEQ ID NO: 39), wherein “X” is an optional amino acid linker sequence optionally comprising, or consisting essentially of, or yet further consisting of between 1 to 20 amino acids. In yet a further embodiment, the tip-chimeric peptide IhfA5-mIhfB4_{NTHI} comprises or consists essentially of, or yet further consists of: a polypeptide sequence of

(SEQ ID NO: 40)
RPGRNPKTGDVVPVSARRVVGPSLFSLHHRQPRLGRNPKTGDSV.

In certain embodiments, an antibody specifically binds the tail-chimeric peptide IhfA3-IhfB2_{NTHI} that comprises, or consists essentially of, or yet further consists of: the polypeptide sequence of FLEEIRLSLESGQDVKLSGF-X-TLSAKEIENMVKDILEFISQ (SEQ ID NO: 41), wherein “X” is an optional amino acid linker sequence optionally comprising, or consisting essentially of, or yet further consisting of between 1 to 20 amino acids. In certain embodiments, the linker is selected from any one or more of SEQ ID NOs: 42-49. In one embodiment, the tail-chimeric peptide IhfA3-IhfB2_{NTHI} comprises, or consists essentially of, or yet further consists of

(SEQ ID NO: 50)
FLEEIRLSLESGQDVKLSGFGPSLTLSAKEIENMVKDILEFISQ.

As used herein, the term “EC₅₀” refers to the concentration of an antibody or an antigen-binding fragment thereof which induces a response (for example, binding between the antibody or antigen-binding fragment thereof and its target) halfway between the baseline and maximum after a specified exposure time.

Several parameters are used herein to described the binding and unbinding reaction of receptor (R, such as an antibody or an antigen-binding fragment thereof) and ligand (L, such as the target of the antibody or antigen-binding fragment thereof) molecules, which is formalized as: R+L⇄RL. The reaction is characterized by the on-rate constant kₒₙ and the off-rate constant k_{off}, which have units of M⁻¹ s⁻¹ and s⁻¹, respectively. In equilibrium, the forward binding transition R+L→RL should be balanced by the backward unbinding transition RL→R+L. That is kₒₙ[R] [L]=k_{off} [RL] where [R], [L] and [RL] represent the concentration of unbound free receptors, the concentration of unbound free ligand and the concentration of receptor-ligand complexes. Further, the equilibrium dissociation constant “K_{D}” can be calculated as k_{off}/kₒₙ which is [R]×[L]/[RL], while the equilibrium association constant “K_{A}” can be calculated as kₒₙ/k_{off} which is [RL]/([R]×[L]).

As used herein, the term “cytokine” refers to small proteins (about 5-20 kDa) important in cell signaling, including but not limited to chemokines, interferons, interleukins (ILs), lymphokines, and tumour necrosis factors, but generally not hormones. Cytokines are peptides and cannot cross the lipid bilayer of cells to enter the cytoplasm. An inflammatory cytokine or pro-inflammatory cytokine is a type of signaling molecule (a cytokine) that is secreted from immune cells like helper T cells (Th) and macrophages, and certain other cell types that promote inflammation. They include (but are not limited to) interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions. The term “anti-inflammatory cytokines” includes immunoregulatory molecules that control the pro-inflammatory cytokine response. Cytokines act together with specific cytokine inhibitors and soluble cytokine receptors to regulate the human immune response. Major anti-inflammatory cytokines include interleukin (IL)-1 receptor antagonist, IL-4, IL-6, IL-10, IL-11, and IL-13. Specific cytokine receptors for IL-1, tumor necrosis factor-alpha, and IL-18 also function as pro-inflammatory cytokine inhibitors. Methods of measuring cytokine, including anti-inflammatory cytokine and pro-inflammatory cytokine, levels thereof are well known in the art. For example, serum cytokine levels can be measured using commercially available enzyme-linked immuno-sorbent assay (ELISA) kits.

As used herein, the terms “pro-inflammatory response” and “inflammatory response” are used interchangeably, referring to the biological response of a subject to a pathogen, such as a bacterium, a virus or other microorganism that can cause disease. In some embodiments, a pro-inflammatory response or an inflammatory response refers to immune responses involving specific and non-specific defense systems. A specific defense system reaction is a specific immune system reaction to an antigen. Examples of specific defense system reactions include antibody responses. A non-specific defense system reaction is an inflammatory response mediated by leukocytes generally incapable of immunological memory, e.g., macrophages, eosinophils and neutrophils. In some embodiments, an immune response comprises, or consists essentially of, or yet further consists of the secretion of pro-inflammatory cytokines, resulting in elevated pro-inflammatory cytokine levels. Additionally or alternatively, an immune response comprises, or consists essentially of, or yet further consists of reducing the level of an anti-inflammatory cytokine. Accordingly, a pro-inflammatory response can comprise, or consist essentially of, or yet further consist of secretion of a pro-inflammatory cytokine, or reduction of an anti-inflammatory cytokine, or both.

As used herein, the term “anti-infective” refers to a medicine that is capable of inhibiting the spread of an infectious organism or by killing the infectious organism outright. This term encompasses, but is not limited to, antibiotics, antifungals, anthelmintics, antimalarials, antiprotozoals, antituberculosis agents, and antivirals. Antifungal agents are also called antimycotic agents. They kill or inactivate fungi and are used to treat fungal infections (including yeast infections). One non-limiting example, polyene antifungals are not absorbed when given orally, so are used to treat fungal infections of the gastrointestinal tract, such as oral thrush. Another non-limiting examples are azole antifungals which are synthetic, fungistatic agents with broad-spectrum activity, Echinocandins which are lipopeptide molecules that noncompetitively inhibit (1,3) beta-d-glucan synthase enzyme and target the fungal cell wall, Fulvicin U/F (i.e., griseofulvin), Grifulvin V (Pro) (i.e., griseofulvin), Lamisil (Pro) (i.e., terbinafine), Gris-PEG (Pro) (i.e., griseofulvin), Ancobon (Pro) (i.e., flucytosine), Fulvicin P/G (i.e., griseofulvin) and Terbinex (Pro) (i.e., terbinafine). More anti-infective agents can be found, for example, at www.drugbank.ca/categories/DBCAT000065. The term “anti-viral” or “antiviral” refers to a class of medication used for treating viral infections. Most antivirals target specific viruses, while a broad-spectrum antiviral is effective against a wide range of viruses. Unlike most antibiotics, antiviral drugs do not destroy their target pathogen; instead they inhibit their development. Some of the ways they may act include preventing viral replication by inhibiting viral DNA polymerase; binding to specific cell-surface receptors and inhibiting viral penetration or uncoating; inhibiting viral protein synthesis; or blocking late stages of virus assembly. Non-limiting examples of anti-viral agents can be found, for example, at www.drugbank.ca/categories/DBCAT000066.

As used herein, the term “anti-parasitic” refers to a class of medications which are indicated for the treatment of parasitic diseases, such as those caused by helminths, amoeba, ectoparasites, parasitic fungi, and protozoa, among others. Non-limiting examples of anti-parastics can be found, for example, at www.drugbank.ca/categories/DBCAT000522.

### Modes for Carrying Out the Disclosure

Although the constituent molecules of the extracellular polymeric substances (EPS) vary among bacterial species, extracellular DNA (eDNA) serves as a common underlying structural component throughout diverse bacterial biofilms (Flemming et al., Nat Rev Microbiol, 2010. 8(9): p. 623-33). The eDNA structure was further characterized and it was determined that the eDNA lattice is comprised of Holliday junction (HJ)-like structures that are integral to the stability of the eDNA-dependent bacterial biofilm EPS (Devaraj et al., Proc Natl Acad Sci USA, 2019 Dec. 10; 116(50):25068-25077). The bacterial DNABII family of DNA-binding proteins, that includes integration host factor (IHF) and histone-like protein (HU), bind to these HJ-like structures within the eDNA lattice and serve as linchpin proteins that maintain the structural integrity of the eDNA-dependent EPS (Devaraj et al.). Sequestration of free DNABII proteins (via exposure to specific antibodies (α-DNABII) directed against the DNA-binding domain of the DNABII proteins) shifts the equilibrium from the eDNA-bound to the unbound state which subsequently causes bacterial biofilms to collapse (Devaraj et al.; Goodman et al., Mucosal Immunol, 2011. 4(6): p. 625-37; Gustave et al., J Cyst Fibros, 2013. 12(4): p. 384-9; Novotny et al., PLoS One, 2013. 8(6): p. e67629; Brockson et al., Mol Microbiol, 2014. 93(6): p. 1246-58; Brandstetter et al., The Laryngoscope, 2013. 123(11): p. 2626-2632; Rocco et al., Mol Oral Microbiol, 2016; Novotny et al., EBioMedicine, 2016. 10: p. 33-44; Devaraj et al., Microbiologyopen, 2017; Freire et al., Mol Oral Microbiol, 2017. 32(1): p. 74-88; and Novotny et al., NPJ Vaccines, 2019. 4: p. 43).

While DNABII proteins are absent in vertebrates, eukaryotes possess a partial functional orthologue, HMGB1, that binds to similar bent DNA structures such as HJ DNA (Bianchi et al., Science, 1989. 243(4894 Pt 1): p. 1056-9). HMGB1 is a ubiquitous protein in eukaryotes and a native part of the chromatin (Bianchi and Beltrame, Am J Hum Genet, 1998. 63(6): p. 1573-7; and Agresti et al., Mol Cell, 2005. 18(1): p. 109-21). It functions as a monomer and consists of two tandem DNA-binding domains and an acidic C-terminus tail (Bianchi et al., EMBO J, 1992. 11(3): p. 1055-63) and often has post translational modifications (PTMs) that dictate its location (nucleus, cytoplasm or extracellular) and activity (reviewed in Kang et al., Mol Aspects Med, 2014. 40: p. 1-116). HMGB1 serves as an accessory protein in multiple DNA-protein transactions that include recombination, DNA repair and transcription via its ability to bind to and bend DNA in a sequence independent manner (Little et al., Nucleic Acids Res, 2013. 41(5): p. 3289-301; Sutrias-Grau et al., J Biol Chem, 1999. 274(3): p. 1628-34; and Yuan et al., J Biol Chem, 2004. 279(20): p. 20935-40). HMGB1 also functions as a damage associated molecular pattern (DAMP) molecule that induces a pro-inflammatory cascade upon its release from eukaryotic cells into the extracellular milieu via the NF-κB pathway by binding to TLR2, TLR4, TLR9 and RAGE (Klune et al., Mol Med, 2008. 14(7-8): p. 476-84; Park et al., Am J Physiol Cell Physiol, 2003. 284(4): p. C870-9; Silva et al., Intensive Care Med, 2007. 33(10): p. 1829-39; and He et al., Asian Pac J Cancer Prev, 2012. 13(4): p. 1365-70), and thus serves as an alarmin with the potential to cause sepsis, which has devastating consequences for the host (Qin et al., J Exp Med, 2006. 203(7): p. 1637-42; and Diener et al., Immunol Cell Biol, 2013. 91(7): p. 443-50). Extracellularly, HMGB1 also has a wide array of functions that include tissue regeneration and wound healing, senescence and at very high concentrations (1.75 μM-12 μM), bacterial killing (Ranzato et al., Mol Cell Biochem, 2009. 332(1-2): p. 199-205; Gong et al., J Biomed Sci, 2009. 16: p. 83; Davalos et al., J Cell Biol, 2013. 201(4): p. 613-29; and Zetterstrom et al., Pediatr Res, 2002. 52(2): p. 148-54). Perhaps most importantly, extracellular HMGB1 is an integral part of the eDNA of neutrophil extracellular traps (NETs), the host's primary means to sequester bacteria for further elimination (Tadie et al., Am J Physiol Lung Cell Mol Physiol, 2013. 304(5): p. L342-9; Remijsen, et al., Cell Death Differ, 2011. 18(4): p. 581-8; Brinkmann et al., Science, 2004. 303(5663): p. 1532-5; and Peng et al., Sci Rep, 2017. 7(1): p. 16628) and, as proposed herein, can additionally serve as a tactic to prevent proliferation of bacterial biofilms.

The eukaryotic host innate immune effector HMGB1, and the bacterial DNABII proteins serve similar roles as accessory proteins in nucleoprotein transactions in the host and bacteria respectively (reviewed in Kang et al. and Browning et al., Curr Opin Microbiol, 2010. 13(6): p. 773-80). While HMGB1 and DNABII proteins share no discernible sequence identity or secondary structure, they can nonetheless functionally replace each other in in vitro transactions. As such, HMGB1 was originally thought to be a functional orthologue of DNABII proteins (Paull et al., Genes Dev, 1993. 7(8): p. 1521-34; and Segall et al., EMBO J, 1994. 13(19): p. 4536-48). Although both proteins bind to and bend DNA, they do so via distinct mechanisms. Both HMGB1 (as a monomer) and DNABII proteins (as dimers) bind to DNA via its minor groove, however whereas HMGB1 stabilizes the DNA bend from the convex surface, DNABII proteins stabilize the DNA bend from its concave surface (Sanchez-Giraldo et al., Acta Crystallogr D Biol Crystallogr, 2015. 71(Pt 7): p. 1423-32; and Rice et al., Cell, 1996. 87(7): p. 1295-306). Given their extraordinary functional similarities intracellularly, and because HMGB1 and DNABII proteins are also found extracellularly, further disclosed herein is the interaction of HMGB1 within the eDNA-dependent EPS of bacterial biofilms.

Described and exemplified herein is the heretofore unknown extracellular function of the vertebrate High Mobility Group Box 1 protein (HMGB1) in the proliferation of bacterial biofilms. Within host cells, HMGB1 functions as a DNA architectural protein, similar to the ubiquitous DNABII family of bacterial proteins, despite that these proteins share no amino acid sequence identity. Extracellularly, HMGB1 induces a pro-inflammatory immune response, whereas the DNABII proteins stabilize the extracellular DNA-dependent matrix that maintains bacterial biofilms. Without wishing to be bound by the theory, when both proteins converge on eDNA within bacterial biofilms, HMGB1, unlike the DNABII proteins, disrupted biofilms both in vitro (including the high priority ESKAPEE pathogens) and in vivo in two distinct animal models, albeit with induction of a strong inflammatory response that was attenuated by a single engineered amino acid change. A model was proposed herein where extracellular HMGB1 balances the degree of induced inflammation and biofilm containment without excessive release of biofilm-resident bacteria.

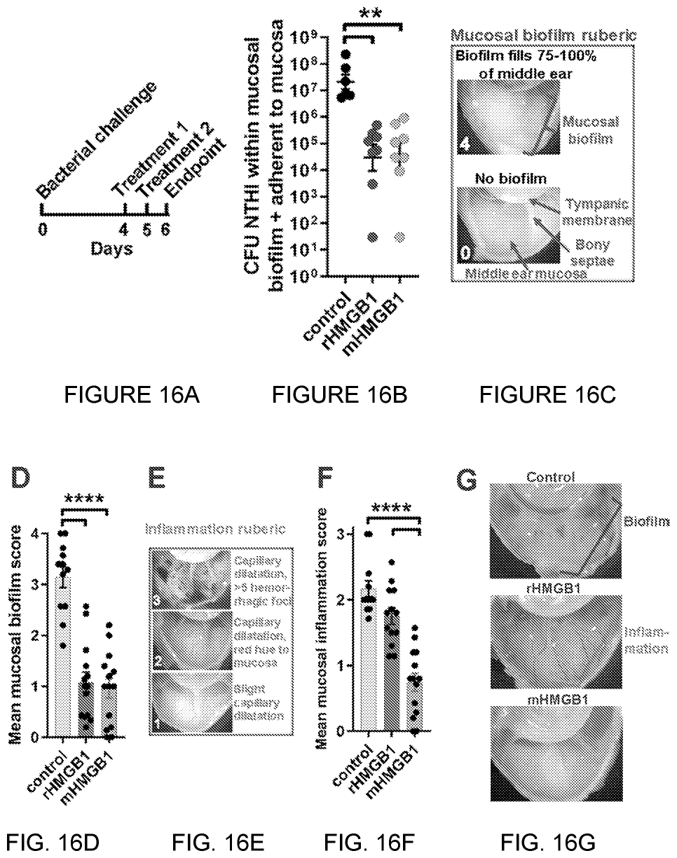

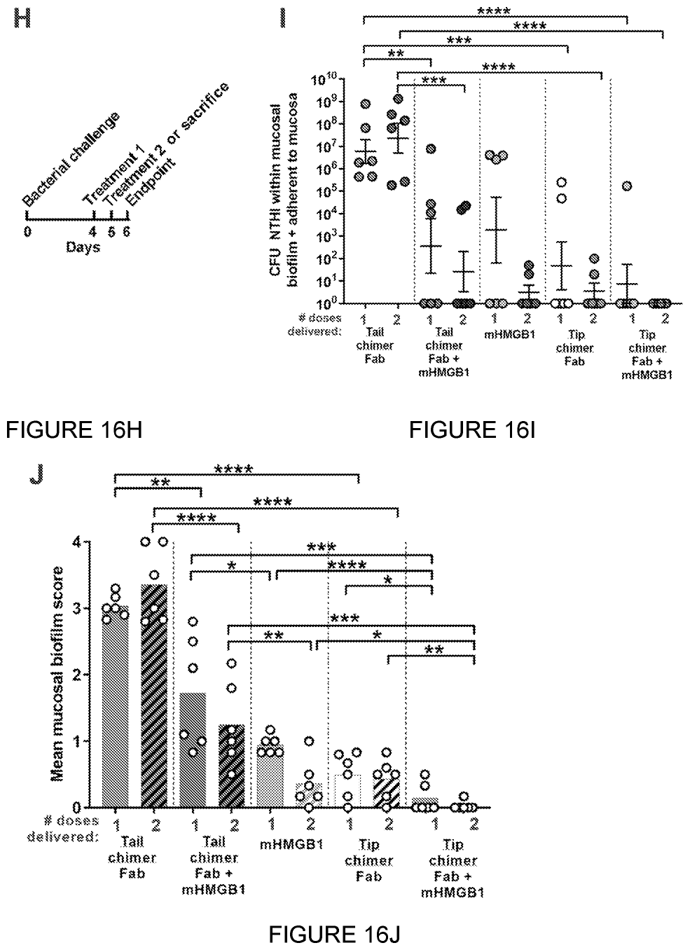

A surprising beneficial effect, i.e., a full biofilm eradication, was exemplified and observed while interfering with the binding of a DNABII protein and an extracellular DNA (for example by administering an anti-DNABII antibody) and providing an HMGB1 polypeptide. See, Example 1 and FIGS. 16I & 16J. It is further noted that repeating the administration of the HMGB1 polypeptide alone or the anti-DNABII antibody alone does not reach the biofilm reduction level achieved by a single administration of the combination of the HMGB1 polypeptide and the anti-DNABII antibody, suggesting a more than additive (i.e., a synergistic) effect. Such observation aligns with the mechanism proposed herein. Without being bound by theory, such synergy is expected for the combination of other HMGB polypeptides and ant-DNA antibody, e.g., an antibody or fragment thereof that binds a tip polypeptide or tip chimer polypeptide. ***

In one aspect, provided is a composition or combination comprising, or alternatively consisting essentially of, or yet further consisting of (a) a high mobility group box 1 protein (HMGB1) polypeptide, or a fragment thereof comprising, or consisting essentially of, or yet further consisting of a B box or an A box or an AB box thereof, and (b) an anti-DNABII antibody or an antigen-binding fragment thereof as disclosed herein.

In another aspect, the composition or combination comprises, or consists essentially of, or yet further consists of (a) the HMGB1 polypeptide further comprising the mutation of C45S; and (b) the anti-DNABII antibody or antigen-binding fragment thereof as disclosed herein.

In one aspect, provided is a composition or combination comprising, or consisting essentially of, or yet further consisting of: (a) a high mobility group box 1 protein (HMGB1) polypeptide, or a fragment thereof, and (b) an antibody or an antigen-binding fragment thereof specifically recognizing and binding a tip domain of a DNABII protein, with the proviso that (i) the composition or combination does not comprise one or more of SEQ ID NOs: 51-58, such as SEQ ID NO: 52, or (ii) the antigen-binding fragment that does not comprise an Fab optionally an Fab of polyclonal antibodies or the antibody that does not comprise polyclonal antibodies, or both (i) and (ii).

In one aspect, provided is a polypeptide comprising, or consisting essentially of, or yet further consists of (a) a high mobility group box 1 protein (HMGB1) polypeptide, or a fragment thereof, and (b) an anti-DNABII antibody or an antigen-binding fragment thereof.

In one aspect, provided is a polypeptide comprising, or consisting essentially of, or yet further consisting of (a) a high mobility group box 1 protein (HMGB1) polypeptide, or a fragment thereof, and (b) an antibody or an antigen-binding fragment thereof specifically recognizing and binding a tip domain of a DNABII protein, with the proviso that (i) the polypeptide does not comprise one or more of SEQ ID NOs: 51-58, such as SEQ ID NO: 52, or (ii) the antigen-binding fragment that does not comprise an Fab optionally an Fab of polyclonal antibodies or the antibody that does not comprise polyclonal antibodies, or both (i) and (ii).

In some embodiments, the polypeptide further comprises a cleavable peptide located between (a) and (b).

In a further aspect, provided is a polynucleotide encoding a polypeptide as disclosed herein, or a polynucleotide complementary thereto.

In one aspect, provided is a polynucleotide encoding (a) a high mobility group box 1 protein (HMGB1) polypeptide, or a fragment thereof, and (b) an anti-DNABII antibody or an antigen-binding fragment thereof, or a polynucleotide complementary thereto.

In one aspect, provided is a polynucleotide encoding: (a) a high mobility group box 1 protein (HMGB1) polypeptide, or a fragment thereof, and (b) an antibody or an antigen-binding fragment thereof specifically recognizing and binding a tip domain of a DNABII protein, or a polynucleotide complementary thereto, with the proviso that (i) the polynucleotide does not encode one or more of SEQ ID NOs: 51-58, such as SEQ ID NO: 52, or (ii) the antigen-binding fragment that does not comprise an Fab optionally an Fab of polyclonal antibodies or the antibody that does not comprise polyclonal antibodies, or both (i) and (ii).

In some embodiments, (a) and (b) are encoded by one contiguous polynucleotide, for example, under the direction of the same regulatory sequence(s). In further embodiments, (a) and (b) are encoded by one contiguous polynucleotide under the direction of different regulatory sequences, i.e., the polynucleotide is bicistronic. In other embodiments, (a) and (b) are encoded by two polynucleotides.

In one aspect, provided is a vector comprising, or consisting essentially of, or yet further consisting of a polynucleotide as disclosed herein.

In one aspect, provided is a host cell comprising one or more of: a composition or combination as disclosed herein, a polypeptide as disclosed herein, a polynucleotide as disclosed herein, or a vector as disclosed herein. In some embodiments, the host cell secrets the HMGB1 polypeptide or the fragment thereof, and the anti-DNABII antibody or antigen-binding fragment thereof. In some embodiments, the host cells are used in producing a composition or combination as disclosed herein, a polypeptide as disclosed herein, a polynucleotide as disclosed herein, or a vector as disclosed herein, for example by culturing the host cells and collecting the composition or combination, polypeptide, polynucleotide, or vector.

In one aspect, provided is a method of producing a composition or combination as disclosed herein. The method comprises, or consists essentially of, or further consists of culturing a host cell comprising a polynucleotide as disclosed herein and isolating the HMBG1 polypeptide or fragment thereof and the anti-DNABII antibody or antigen-binding fragment thereof from the cell culture. In another aspect, provided is a method of producing a polypeptide as disclosed herein. The method comprises, or consists essentially of, or further consists of culturing a host cell comprising a polynucleotide as disclosed herein and isolating the polypeptide from the cell culture. In some embodiments, the method further comprises introducing the polynucleotide, or a vector comprising the polynucleotide to the host cell.

In one aspect, provided is a method for one or more of the following: (A) preventing the formation of or disrupting a biofilm in vitro or ex vivo, (B) preventing the formation of or disrupting a biofilm in a subject, (C) inhibiting, preventing or treating a microbial infection that produces a biofilm in a subject, or (D) treating a condition characterized by the formation of a biofilm in a subject. The method comprises, or alternatively consists essentially of, or yet further consists of administering to the subject:
(a) a high mobility group box 1 protein (HMGB1) polypeptide, or a fragment thereof comprising, or consisting essentially of, or yet further consisting of a B box or an A box or an AB box thereof, and
(b) an anti-DNABII antibody or an antigen-binding fragment thereof as disclosed herein.

In one aspect, provided is a method for inducing or increasing the formation of a neutrophil extracellular trap (NET) immediately adjacent to a biofilm in a subject, and disrupting the biofilm optionally without inducing a pro-inflammatory response. The method comprises, or alternatively consists essentially of, or yet further consists of administering to the subject:
(a) a high mobility group box 1 protein (HMGB1) polypeptide comprising the amino acid sequence of any one or more of SEQ ID NOs: 51-58, or a fragment thereof comprising, or consisting essentially of, or yet further consisting of a B box or an A box or an AB box thereof, and
(b) an anti-DNABII antibody or an antigen-binding fragment thereof as disclosed herein.

In one aspect, provided is a method for one or more of the following: (A) preventing the formation of or disrupting a biofilm in vitro or ex vivo, (B) preventing the formation of or disrupting a biofilm in a subject, (C) inhibiting, preventing or treating a microbial infection that produces a biofilm in a subject, or (D) treating a condition characterized by the formation of a biofilm in a subject. The method comprises, or consists essentially of, or yet further consists of administering to the subject one or more of: a composition or combination as disclosed herein, a polypeptide as disclosed herein, a polynucleotide as disclosed herein, a vector as disclosed herein, or a host cell as disclosed herein.

In one aspect, provided is a method for inducing or increasing the formation of a neutrophil extracellular trap (NET) immediately adjacent to a biofilm in a subject, and disrupting the biofilm optionally without inducing a pro-inflammatory response. The method comprises, or consists essentially of, or yet further consists of administering to the subject one or more of: a composition or combination as disclosed herein, a polypeptide as disclosed herein, a polynucleotide as disclosed herein, a vector as disclosed herein, or a host cell as disclosed herein, with the proviso that the HMGB1 polypeptide comprises, or consists essentially of, or yet further consists of any one or more of SEQ ID NOs: 51-58.

In some embodiments, the method further comprises administering to the subject one or more of: a DNase enzyme, an antibiotic agent, an antimicrobial agent, an anti-infective agent, an anti-fungal agent, an anti-parasitic agent, an anti-viral agent, or an antibody or an antigen-binding fragment thereof specifically recognizing or binding to OMP P5, rsPilA, OMP 26, OMP P2, or Type IV Pilin.

In some embodiments, the condition characterized by the formation of a biofilm comprises, or consists essentially of, or yet further consists of one or more of: a chronic non-healing wound, a Burkholderia infection, a lung infection due to Burkholderia sp., a venous ulcer, a diabetic foot ulcer, an ear infection, a sinus infection, a urinary tract infection, a gastrointestinal tract ailment, a hospital acquired pneumonia, a ventilator-associated pneumonia, a surgical implant-associated infection, a pulmonary infection, a respiratory tract infection, cystic fibrosis, chronic obstructive pulmonary disease, a catheter-associated infection, an indwelling devices associated infection, an infection associated with implanted prostheses, osteomyelitis, cellulitis, abscesses, or periodontal disease.

In some embodiments, the administration of (a) and the administration of (b) are performed concurrently or sequentially. Additionally or alternatively, the administration of (a) and (b) is repeated for at least once, at least twice, at least three times, or more times.

In some aspects relating to any method(s) as disclosed herein, optionally comprising an administration step, the antibody or antigen-binding fragment thereof reduce one or more of pro-inflammatory cytokines and increase one or more of anti-inflammatory cytokines in the subject. For the methods as disclosed herein, Applicant has unexpectedly found that the HMGB1 fragments as disclosed herein are not post-translationally modified but nonetheless retain DNA binding activity. Without being bound by theory, Applicant believes that the modified HMGB1 fragments will perform similarly.

In some aspects relating to any aspect(s), any embodiment(s) and/or method(s) as disclosed herein, the HMGB1 polypeptide or a fragment thereof as disclosed herein and the anti-DNABII antibody or an antigen-binding fragment thereof as disclosed herein are combined and contained in the same composition (such as in the same pharmaceutical composition, which optionally further comprises a pharmaceutical acceptable carrier, and/or contacting or administering in a method as disclosed herein in one composition at the same time).

In another aspects relating to any aspect(s), any embodiment(s) and/or method(s) as disclosed herein, the HMGB1 polypeptide or a fragment thereof as disclosed herein is a first composition (referred to herein as the HMGB1 composition, optionally a pharmaceutical composition, which optionally further comprising a pharmaceutical acceptable carrier) and an anti-DNABII antibody or an antigen-binding fragment thereof is provided as a second or separate discreet composition (referred to herein as the anti-DNABII antibody composition, that may be another pharmaceutical composition). In a further aspect, an admixture of the HMGB1 and the anti-DNABII antibody compositions comprise, or consist essentially of, or consist of both of the HMGB1 polypeptide or a fragment thereof as disclosed herein and the anti-DNABII antibody or an antigen-binding fragment thereof, to be used in a method as disclosed herein. In one aspect, the HMGB1 composition is contacted or administered in a method as disclosed herein separately (i.e., in the absence of the other) with the anti-DNABII antibody composition. Additionally or alternatively, the HMGB1 composition is contacted or administered in a method as disclosed herein concurrently (such as within 0.5 hours) with the anti-DNABII antibody composition. In yet another aspect, the HMGB1 composition is contacted or administered in a method as disclosed herein sequentially with the anti-DNABII antibody composition, with or without time gaps in between. Such gaps may be minutes to weeks, optionally selected from, but not limited to, 0.5 hour, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 1.5 weeks, 2 weeks or longer. Additionally or alternatively, the HMGB1 composition and the anti-DNABII antibody composition may be administered in a method as disclosed herein in the same administration route, such as intravenously, or in different administration routes. In a further aspect, the composition is suitable for the intended administration route(s), such as having a suitable pH. Without wishing to be bound by the theory, in one embodiment, such composition(s), pharmaceutical composition(s), and/or pharmaceutical acceptable carrier(s) stabilize the HMGB1 polypeptide or a fragment thereof and/or the anti-DNABII antibody or an antigen-binding fragment thereof and/or prevent the HMGB1 polypeptide or a fragment thereof and/or the anti-DNABII antibody or an antigen-binding fragment thereof from degradation.

In some aspects relating to a method as disclosed herein, the method further comprises detecting a biofilm by contacting an antibody that binds a DNABII polypeptide or an antigen-binding fragment of the antibody with a sample suspected of containing a biofilm, and detecting the binding of the biofilm and the antibody or fragment thereof. In one aspect, the detecting antibody or antigen-binding fragment thereof binds to a tip region of a DNABII polypeptide. In another aspect, the detecting antibody or antigen-binding fragment thereof binds to a tail region of a DNABII polypeptide.

Also provided is a method for screening subjects for use of a composition or method as described herein. The screening method comprises, or alternatively consists essentially of, or yet further consists of contacting an anti-DNABII antibody, anti-DNABII polypeptide, or an antigen-binding fragment of the antibody as disclosed herein with a biological sample comprising the biofilm and isolated from the subject, and detecting the binding of the antibody or antigen-binding fragment thereof to any biofilm in the sample. In one aspect, the antigen-binding fragment of the antibody is selected from the group of Fab, F(ab′)2, Fab′, scFv, or Fv. Additionally or alternatively, the antibody or antigen-binding fragment thereof specifically binds the tip region of a DNABII peptide. In one aspect, the DNABII peptide is an IHF peptide. In one aspect, the screening antibody or antigen-binding fragment binds to a tip region of a DNABII polypeptide. In another aspect, the screening antibody or antigen-binding fragment binds to a tail region of a DNABII polypeptide.

In one aspect, provided is a kit for use in a method as disclosed herein. The kit comprises, or consists essentially of, or yet further consists of instructions for use and one or more of: a composition or combination as disclosed herein, a polypeptide as disclosed herein, a polynucleotide as disclosed herein (such as the polynucleotide as disclosed herein), a vector as disclosed herein, or a host cell as disclosed herein.

### HMGB Compositions

In some embodiments, the HMGB is a human HMGB1 or a murine HMGB1. In some embodiments, a HMGB polypeptide as used herein comprises, or consists essentially of, or yet further consists of an HMG-box domain as disclosed herein (such as HMGB1, HMGB2, HMGB3 or HMGB4), or a mutated modified high mobility group-box domain (such as mHMGB1, mHMGB2, mHMGB3 or mHMGB4). In some embodiments, the HMGB polypeptide comprises, or consists essentially of, or yet further consists of any one or more of SEQ ID NOs: 51-58, 68-74, 84-90 or 100-114.

In some embodiments, HMGB1 is substituted by HMGB2, or HMGB3, or HMGB4, or another equivalent as disclosed herein.

In some embodiments, the HMGB1 polypeptide further comprises one or more mutations selected from the group of mutations at K12, C23, C45, C106, or K114. In some embodiments, the A box of the HMGB1 polypeptide further comprises one or more mutations selected from the group of mutations at K12, C23, or C45. In some embodiment, the B box of the HMGB1 polypeptide further comprises one or both mutations at C106 or K114. In some embodiments, wherein the one or more mutations are to serine, glycine, alanine, valine, isoleucine or threonine. In some embodiments, the HMGB1 polypeptide further comprises one or more mutations selected from C23S, C45S, and C106S. In some embodiments, the HMGB1 polypeptide further comprises the mutation of C45S. In further embodiments wherein the HMGB polypeptide is HMGB2, HMGB3 or HMGB4 or a modified version having mutations at corresponding positions to the modified HMGB1 at K12, C23, or C45 to serine, glycine, alanine, valine, isoleucine or threonine. Additionally or alternatively, the mutant HMGB (mHMGB) comprises one or more mutations at positions corresponding to C23S, C45S, and C106S. In a further aspect the polypeptide further comprising the mutation at the corresponding C45S.

In some embodiments, the one or more of the HMGB1 mutations are selected from the group of mutations at K12, C23, C45, C106, or K114. In a further aspect, the one or more of the HMGB1 mutations are selected from the group of mutations at K12, C23, C45, C106, or K114 to serine, glycine, alanine, valine, isoleucine or threonine. Additionally or alternatively, the mutant HMGB1 (mHMGB1) comprises one or more mutations selected from C23S, C45S, and C106S. In further embodiments wherein the HMGB polypeptide is HMGB2, HMGB3 or HMGB4 or a modified version having mutations at corresponding positions to the modified HMGB1 at K12, C23, C45, C106 or K114 to serine, glycine, alanine, valine, isoleucine or threonine. Additionally or alternatively, the mutant HMGB (mHMGB) comprises one or more mutations at positions corresponding to C23S, C45S, and C106S.

In some embodiments, the fragment of the HMGB polypeptide, e.g., HMGB1 polypeptide, comprises, or consists essentially of, or yet further consists of a B box or an A box or an AB box thereof. In some embodiments, the fragment of the HMGB1 polypeptide or the HMGB1 polypeptide comprises, or consists essentially of, or yet further consists of a B box polypeptide as disclosed herein or an A box polypeptide as disclosed herein or an AB box polypeptide as disclosed herein.

### Antibody Compositions

In some embodiments, an antibody or an antigen-binding fragment thereof as used herein comprises, or consists essentially of, or yet further consists of a heavy chain (HC) variable domain sequence and a light chain (LC) variable domain sequence, wherein the heavy chain and light chain immunoglobulin variable domain sequences form an antigen-binding site that binds to an epitope of a DNABII protein. In certain embodiments, the antibody or antigen-binding fragment thereof binds to a DNABII peptide (such as the tip region of the DNABII peptide including but not limited to: a tip region or tip chimer of IHF or HU, a tip region of IHFA or IHFB, and/or the tip-chimeric peptide IhfA5-mIhfB4_{NTHI}; and/or the tail region of the DNABII peptide, including but not limited to: a tail region of IHF or HU, a tail region of IHFA or IHFB, and/or the tail-chimeric peptide IhfA3-IhfB2_{NTHI}). In one embodiment, the antibody or antigen-binding fragment thereof binds to the tip-chimeric peptide IhfA5-mIhfB4_{NTHI}. In another embodiment, the antibody or antigen-binding fragment thereof binds to the tail-chimeric peptide IhfA3-IhfB2_{NTHI}.

In some embodiments, the antibody or antigen-binding fragment thereof binds to a tip region (i.e., a tip domain) or a tip chimer of a DNABII peptide. In one aspect, the DNABII peptide is an IHF peptide. In some embodiments, the tip domain comprises, or consists essentially of, or yet further consists of one or more amino acid sequence(s) selected from:

(SEQ ID NO: 31)
NFELRDKSSRPGRNPKTGDVV;
(SEQ ID NO: 32)
SLHHRQPRLGRNPKTGDSVNL;
wherein “X” is an optional amino acid linker
sequence and wherein “X₁” is any amino acid;
(SEQ ID NO: 38)
RPGRNPX₁TGDVVPVSARRVV-X-FSLHHRQPRLGRNPX₁TGDSV,
wherein “X₁” is any amino acid
(SEQ ID NO: 39)
RPGRNPKTGDVVPVSARRVV-X-FSLHHRQPRLGRNPKTGDSV;
or
(SEQ ID NO: 40)
RPGRNPKTGDVVPVSARRVVGPSLFSLHHRQPRLGRNPKTGDSV.

In some embodiments, the antibody or antigen-binding fragment thereof comprises, or consists essentially of, or yet further consists of (i) a heavy chain complementarity-determining region 1 (CDRH1) comprising, or consisting essentially of, or yet further consisting of GFTFRTY (aa 50 to aa 56 of SEQ ID NO: 1 or 2 or 3 or 24); (ii) a heavy chain complementarity-determining region 2 (CDRH2) comprising, or consisting essentially of, or yet further consisting of GSDRRH (aa 76 to aa 81 of SEQ ID NO: 1 or 2 or 3 or 24); (iii) a heavy chain complementarity-determining region 3 (CDRH3) comprising, or consisting essentially of, or yet further consisting of VGPYDGYYGEFDY (aa 121 to aa 133 of SEQ ID NO: 1 or 2 or 3 or 24); (iv) a light chain complementarity-determining region 1 (CDRL1) comprising, or consisting essentially of, or yet further consisting of QSLLDSDGKTF (aa 47 to aa 57 of SEQ ID NO: 7 or 8 or 9 or 25); (v) a light chain complementarity-determining region 2 (CDRL2) comprising, or consisting essentially of, or yet further consisting of LVS (aa 75 to aa 77 of SEQ ID NO: 7 or 8 or 9 or 25); and (vi) a light chain complementarity-determining region 3 (CDRL3) comprising, or consisting essentially of, or yet further consisting of WQGTHFPYT (aa 114 to aa 122 of SEQ ID NO: 7 or 8 or 9 or 25).

In some embodiments, the antibody or antigen-binding fragment thereof comprises, or consists essentially of, or yet further consists of a heavy chain (HC) immunoglobulin variable domain. In further embodiments, the HC immunoglobulin variable domain comprises, or consists essentially of, or yet further consists of amino acids 25 to 144 of any one of SEQ ID NO: 1, SEQ ID NO: 2, or SEQ ID NO: 3. In some embodiments, the antibody or antigen-binding fragment thereof comprises, or consists essentially of, or yet further consists of a light chain (LC) immunoglobulin variable domain. In further embodiments, the LC immunoglobulin variable domain comprises, or consists essentially of, or yet further consist of amino acids 21 to 132 of any one of SEQ ID NO: 7, SEQ ID NO: 8, or SEQ ID NO: 9. In some embodiments, the antibody or antigen-binding fragment thereof comprises, or consist essentially of, or yet further consists of a heavy chain (HC) immunoglobulin variable domain and a LC immunoglobulin variable domain. In further embodiments, the HC immunoglobulin variable domain comprises, or consists essentially of, or yet further consists of amino acids 25 to 144 of any one of SEQ ID NO: 1, SEQ ID NO: 2, or SEQ ID NO: 3. In yet further embodiments, the LC immunoglobulin variable domain comprises, or consists essentially of, or yet further consist of amino acids 21 to 132 of any one of SEQ ID NO: 7, SEQ ID NO: 8, or SEQ ID NO: 9. In some embodiments, the HC immunoglobulin variable domain comprises, or consists essentially of, or yet further consists of amino acids 25 to 144 of any one of SEQ ID NO: 1, and the LC immunoglobulin variable domain comprises, or consists essentially of, or yet further consists of amino acids 21 to 132 of SEQ ID NO: 7. In some embodiments, the HC immunoglobulin variable domain comprises, or consists essentially of, or yet further consists of amino acids 25 to 144 of any one of SEQ ID NO: 1, and the LC immunoglobulin variable domain comprises, or consist essentially of, or yet further consists of amino acids 21 to 132 of SEQ ID NO: 8. In some embodiments, the HC immunoglobulin variable domain comprises, or consists essentially of, or yet further consists of amino acids 25 to 144 of any one of SEQ ID NO: 1, and the LC immunoglobulin variable domain comprises, or consists essentially of, or yet further consists of amino acids 21 to 132 of SEQ ID NO: 9.

In some embodiments, the antibody comprises a constant region optionally selected from an IgA constant region, an IgD constant region, an IgE constant region, an IgG constant region, or an IgM constant region.

In some embodiments, the antigen-binding fragment thereof comprises a Fab, F(ab′)₂, Fab′, scFv, or Fv.

In some embodiments, the antibody or antigen-binding fragment thereof is modified. In further embodiments, the antibody or antigen-binding fragment thereof is modified by a process selected from PEGylation, polysialyation, HESylation or glycosylation.

In some embodiments, the antibody or antigen-binding fragment thereof is a monoclonal antibody or an antigen-binding fragment of the monoclonal antibody.

In some embodiments, the antibody or antigen-binding fragment thereof comprises a humanized or human framework.

In some embodiments, the antibody or antigen-binding fragment is derived from a mammal. In further embodiments, the antibody or antigen-binding fragment is derived from a non-human mammal, such as a mouse, a rat, a pig, a cow, a rabbit, a goat, a chicken. A house, a dog, a cat or a llama. In other embodiments, the antibody or antigen-binding fragment is derived from human. In yet other embodiments, the antibody or antigen-binding fragment is humanized. Non-limiting examples of antibody or antigen-binding fragment can be found in U.S. Pat. Nos. 8,999,291, 9,745,366, 10,940,204; US Patent Application Publication Nos. 2016-0175440, 2018-0303900, 2019-0338018, 2019-0337996, 2020-0190170, and 2021-0139551; and PCT Publication No. WO2021/007260.

In some embodiments, the antibody or antigen-binding fragment thereof comprises, or consists essentially of, or yet consists of a heavy chain (HC) immunoglobulin variable domain sequence comprising, consisting essentially of, or consisting of a sequence selected from the group of amino acid (aa) 25 to aa 144 of SEQ ID NOs: 1-6, 13, 24 or 26 or an equivalent of each thereof, and/or a light chain (LC) immunoglobulin variable domain sequence comprising, consisting essentially of, or consisting of, a sequence selected from the group of aa 21 to aa 132 of SEQ ID NOs: 7-9, 14 or 25, aa 21 to aa 126 of SEQ ID NOs: 10-12 or 27, or an equivalent of each thereof.

In some embodiments, the antibody or antigen-binding fragment thereof comprises, or consists essentially of, or yet consists of a heavy chain (HC) comprising, consisting essentially of, or consisting of, a sequence selected from the group of aa 25 to aa 473 of SEQ ID NOs: 1-6, 13, 24 or 26 or an equivalent of each thereof; and/or a light chain (LC) comprising, consisting essentially of, or consisting of, a sequence selected from the group of aa 21 to aa 239 of SEQ ID NOs: 7-9, 14, or 25, aa 21 to aa 233 of SEQ ID NOs: 10-12 or 27, or an equivalent of each thereof. In some embodiments, the antibody or antigen-binding fragment thereof comprises, or alternatively consists essentially of, or yet further consists of: a heavy chain (HC) comprising, or alternatively consisting essentially of, or yet further consisting of a sequence selected from the group of: amino acid (aa) 25 to aa 473 of SEQ ID NOs: 13, 24 or 26 or an equivalent of each thereof, and/or a light chain (LC) comprising, or alternatively consisting essentially of, or yet further consisting of a sequence selected from the group of: aa 21 to aa 239 of SEQ ID NOs: 14 or 25, aa 21 to aa 233 of SEQ ID NO: 27 or an equivalent of each thereof.

In some embodiments, the antibody or antigen-binding fragment thereof comprises, or consists essentially of, or yet consists of a heavy chain (HC) comprising, consisting essentially of, or consisting of, a sequence selected from the group of SEQ ID NOs: 1-6, 13, 24 or 26 or an equivalent of each thereof; and/or a light chain (LC) comprising, consisting essentially of, or consisting of, a sequence selected from the group of SEQ ID NOs: 7-12, 14, 25, or 27, or an equivalent of each thereof.

In some embodiments, the antibody or antigen-binding fragment thereof comprises, or consists essentially of, or yet consists of any one or any two or all three CDRs of a sequence selected from the group of: SEQ ID NOs: 1-6, 13, 24 or 26, or an equivalent of each thereof; and/or any one or any two or all three CDRs of a sequence selected from the group of: SEQ ID NOs: 7-12, 14, 25 or 27, or an equivalent of each thereof.

In some embodiments, the antibody or antigen-binding fragment thereof comprises, or consists essentially of, or yet consists of a heavy chain (HC) immunoglobulin variable domain sequence comprising, consisting essentially of, or consisting of a sequence selected from the group of aa 25 to aa 144 of SEQ ID NO: 13, 24 or 26, or an equivalent thereof, and a light chain (LC) immunoglobulin variable domain sequence comprising, consisting essentially of, or consisting of, a sequence selected from the group of aa 21 to aa 132 of SEQ ID NOs: 14 or 25, aa 21 to aa 126 of SEQ ID NO: 27, or an equivalent thereof. In some embodiments, the antibody or antigen-binding fragment thereof comprises, or consists essentially of, or yet consists of a heavy chain (HC) immunoglobulin variable domain sequence comprising, consisting essentially of, or consisting of a sequence selected from the group of aa 25 to aa 144 of SEQ ID NOs: 1-6, 13, 24 or 26, or an equivalent of each thereof, and/or a light chain (LC) immunoglobulin variable domain sequence comprising, consisting essentially of, or consisting of a sequence selected from the group of aa 21 to aa 132 of SEQ ID NOs: 7-9, 14 or 25, aa 21 to aa 126 of SEQ ID NOs: 10-12 or 27, or an equivalent of each thereof.

In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of SEQ ID NO: 1 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of SEQ ID NO: 2 or an equivalent thereof and a light chain (LC) immunoglobulin variable domain sequence that comprises, consists essentially of, or alternatively consists of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In some embodiments, the antibody or antigen-binding fragment thereof comprises, or consists essentially of, or yet consists of a heavy chain (HC) immunoglobulin variable domain sequence comprises, consisting essentially of, or consisting of, an amino acid sequence of aa 25 to aa 144 of SEQ ID NO: 3 or an equivalent thereof and a light chain (LC) immunoglobulin variable domain sequence comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof.

In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of SEQ ID NO: 4 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of SEQ ID NO: 5 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of SEQ ID NO: 6 or an equivalent thereof, and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof.

In some embodiments, the antibody or antigen-binding fragment thereof comprises, or consists essentially of, or yet consists of a heavy chain (HC) immunoglobulin variable domain sequence that comprises, consisting essentially of, or consisting of an amino acid sequence of aa 25 to aa 144 of any one of SEQ ID NOs: 1-6, 13, 24 or 26, or an equivalent of each thereof, and a light chain (LC) immunoglobulin variable domain sequence comprises, consisting essentially of, or consisting of, an amino acid sequence of aa 21 to aa 132 of SEQ ID NO 7, or an equivalent thereof. In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of any one of SEQ ID NOs: 1-6, 13, 24 or 26 or an equivalent of each thereof, and a light chain (LC) immunoglobulin variable domain sequence that comprises, consisting essentially of, or consisting of, an amino acid sequence of aa 21 to aa 132 of SEQ ID NO: 8, or an equivalent thereof. In some embodiments, the antibody or antigen-binding fragment thereof comprises, or consists essentially of, or yet consists of a heavy chain (HC) immunoglobulin variable domain sequence that comprises, consisting essentially of, or consisting of, an amino acid sequence of aa 25 to aa 144 of any one of SEQ ID NOs: 1-6, 13, 24 or 26, or an equivalent of each thereof, and a light chain (LC) immunoglobulin variable domain sequence that comprises, consisting essentially of, or consisting of, an amino acid sequence of aa 21 to aa 132 of SEQ ID NO: 9, or an equivalent thereof.

In some embodiments, the antibody or antigen-binding fragment thereof comprises, or consists essentially of, or yet consists of a heavy chain (HC) immunoglobulin variable domain sequence that comprises, or alternatively consists essentially of, or yet further consists of, an amino acid sequence of aa 25 to aa 144 of any one of SEQ ID NOs: 1-6, 13, 24 or 26, or an equivalent of each thereof, and a light chain (LC) immunoglobulin variable domain sequence that comprises, or consists essentially of, or yet further consists of, an amino acid sequence of aa 21 to aa 126 of SEQ ID NO: 10, or an equivalent thereof. In some embodiments, the antibody or antigen-binding fragment thereof comprises, or consists essentially of, or yet consists of a heavy chain (HC) immunoglobulin variable domain sequence that comprises, or consist essentially of, or yet further consists of, an amino acid sequence of aa 25 to aa 144 of any one of f SEQ ID NOs: 1-6, 13, 24 or 26, or an equivalent of each thereof, and a light chain (LC) immunoglobulin variable domain that comprises, or consists essentially thereof, or consists of, an amino acid sequence of aa 21 to aa 126 of SEQ ID NO: 11, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consisting of, an amino acid sequence of aa 25 to aa 144 of any one of SEQ ID NOs: 1-6, 13, 24 or 26, or an equivalent of each thereof, and the light chain (LC) immunoglobulin variable domain sequence comprises an amino acid sequence of aa 21 to aa 126 of SEQ ID NO: 12, or an equivalent thereof.

In some embodiments, the antibody or antigen-binding fragment thereof comprises, or consists essentially of, or yet consists of a heavy chain (HC) immunoglobulin variable domain sequence that comprises, or alternatively consists essentially of, or yet further consists of, an amino acid sequence of aa 25 to aa 144 of SEQ ID NO: 1 or an equivalent thereof and a light chain (LC) immunoglobulin variable domain sequence that comprises, or alternatively consists essentially thereof, or consists of, an amino acid sequence of aa 21 to aa 132 of SEQ ID NO: 7, or an equivalent thereof. In some embodiments, the antibody or antigen-binding fragment thereof comprises, or consists essentially of, or yet consists of a heavy chain (HC) immunoglobulin variable domain sequence that comprises, or alternatively consists essentially of, or yet further consists of, an amino acid sequence of aa 25 to aa 144 of SEQ ID NO: 1 or an equivalent thereof and a light chain (LC) immunoglobulin variable domain sequence that comprises, or alternatively consists essentially thereof, or consists of, an amino acid sequence of aa 21 to aa 132 of SEQ ID NO: 8, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 1 or an equivalent thereof and a light chain (LC) immunoglobulin variable domain sequence that comprises, or consists essentially thereof, or consists of, an amino acid sequence of aa 21 to aa 132 of SEQ ID NO: 9, or an equivalent thereof.

In some embodiments, the antibody or antigen-binding fragment thereof comprises, or consists essentially of, or yet consists of a heavy chain (HC) immunoglobulin variable domain sequence that comprises, or alternatively consists essentially thereof, or consists of, an amino acid sequence of aa 25 to aa 144 of SEQ ID NO: 2 or an equivalent thereof and a light chain (LC) immunoglobulin variable domain sequence that comprises, or consists essentially thereof, of consists of, an amino acid sequence of aa 21 to aa 132 of SEQ ID NO: 7, or an equivalent thereof. In some embodiments, the antibody or antigen-binding fragment thereof comprises, or consists essentially of, or yet consists of a heavy chain (HC) immunoglobulin variable domain sequence comprises an amino acid sequence of aa 25 to aa 144 of SEQ ID NO: 2 or an equivalent thereof, and a light chain (LC) immunoglobulin variable domain sequence that comprises, or consists essentially thereof, or yet further consists of, an amino acid sequence of aa 21 to aa 132 of SEQ ID NO: 8, or an equivalent thereof. In some embodiments, the antibody or antigen-binding fragment thereof comprises, or consists essentially of, or yet consists of a heavy chain (HC) immunoglobulin variable domain sequence that comprises, or consisting essentially thereof, or consisting of, an amino acid sequence of aa 25 to aa 144 of SEQ ID NO: 2 or an equivalent thereof, and a light chain (LC) immunoglobulin variable domain sequence that comprises, consisting essentially thereof, or consists of, an amino acid sequence of aa 21 to aa 132 of SEQ ID NO: 9, or an equivalent thereof.

In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 3 or an equivalent thereof and a light chain (LC) immunoglobulin variable domain sequence comprises, or consists of an amino acid sequence of aa 21 to aa 132 of SEQ ID NO: 7, or an equivalent thereof. In some embodiments, the antibody or antigen-binding fragment thereof comprises, or consists essentially of, or yet consists of a heavy chain (HC) immunoglobulin variable domain sequence that comprises, or consists essentially of, or yet further consists of, an amino acid sequence of aa 25 to aa 144 of SEQ ID NO: 3 or an equivalent thereof, and the light chain (LC) immunoglobulin variable domain sequence comprises, or consists essentially thereof, or consists of, an amino acid sequence of aa 21 to aa 132 of SEQ ID NO: 8, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 3 or an equivalent thereof and the light chain (LC) immunoglobulin variable domain sequence comprises an amino acid sequence of aa 21 to aa 132 of SEQ ID NO: 9, or an equivalent thereof.

In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 4 or an equivalent thereof and a light chain (LC) immunoglobulin variable domain sequence comprises, or consists essentially thereof, or consists of, an amino acid sequence of aa 21 to aa 126 of SEQ ID NO: 10, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 4 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 4 or an equivalent thereof, and a light chain (LC) immunoglobulin variable domain sequence that comprises, or consists essentially thereof, or consists thereof, an amino acid sequence of aa 21 to aa 126 of SEQ ID NO: 12, or an equivalent thereof.

In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, or consists of, an amino acid sequence of aa 21 to aa 126 of SEQ ID NO: 10, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, an amino acid sequence of aa 21 to aa 126 of SEQ ID NO: 12, or an equivalent thereof.

In some embodiments, the antibody or antigen-binding fragment thereof comprises, or consists essentially of, or yet consists of a heavy chain (HC) immunoglobulin variable domain sequence that comprises, or consists essentially thereof, or consists of, an amino acid sequence of aa 25 to aa 144 of SEQ ID NO: 6 or an equivalent thereof, and a light chain (LC) immunoglobulin variable domain sequence that comprises, or consists essentially thereof, or consists of, an amino acid sequence of aa 21 to aa 126 of SEQ ID NO: 10, or an equivalent thereof. In some embodiments, the antibody or antigen-binding fragment thereof comprises, or consists essentially of, or yet consists of a heavy chain (HC) immunoglobulin variable domain sequence that comprises, or consisting essentially thereof, or consists thereof, an amino acid sequence of aa 25 to aa 144 of SEQ ID NO: 6 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In some embodiments, the antibody or antigen-binding fragment thereof comprises, or consists essentially of, or yet consists of a heavy chain (HC) immunoglobulin variable domain sequence that comprises, consisting essentially thereof, or consisting of, an amino acid sequence of aa 25 to aa 144 of SEQ ID NO: 6 or an equivalent thereof, and a light chain (LC) immunoglobulin variable domain sequence that comprises, consists essentially thereof, or consists of, an amino acid sequence of aa 21 to aa 126 of SEQ ID NO: 12, or an equivalent thereof.

In one aspect, the antibody or antigen-binding fragment thereof comprises, or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 24 or an equivalent thereof, and/or a light chain (LC) immunoglobulin variable domain sequence comprising, or consisting essentially of, or yet further consisting of an amino acid sequence of aa 21 to aa 132 of SEQ ID NO: 25 or an equivalent thereof. In a further embodiment, the antibody or antigen-binding fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to the tip-chimeric peptide IhfA5-mIhfB4_{NTHI}. In yet a further embodiment, the fragment is an antigen-binding fragment.

In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 7 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof. In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID NO: 7 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or antigen-binding fragment thereof comprises or consists essentially of, or yet further consists of: a the heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof. In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 7 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof.

In some embodiments, the antibody or antigen-binding fragment thereof comprises, or consists essentially of, or yet consists of a heavy chain (HC) immunoglobulin variable domain sequence comprising, or consisting essentially of, or yet further consisting of an amino acid sequence of aa 25 to aa 144 of SEQ ID NO: 26 or an equivalent thereof, and a light chain (LC) immunoglobulin variable domain sequence comprising, or consisting essentially of, or yet further consisting of an amino acid sequence of aa 21 to aa 126 of SEQ ID NO: 27 or an equivalent thereof. In a further embodiment, the antibody or antigen-binding fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody or antigen-binding fragment thereof binds to the tail-chimeric peptide IhfA3-IhfB2_{NTHI}. In yet a further embodiment, the fragment is an antigen-binding fragment. In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof. In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof. In one embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof.

In some embodiments, the antibody or antigen-binding fragment thereof comprises, or consists essentially of, or yet consists of a heavy chain (HC) comprising, or consisting essentially of, or yet further consisting of an amino acid sequence of SEQ ID NO: 24 or an equivalent thereof, and/or a light chain (LC) comprising, or consisting essentially of, or yet further consisting of an amino acid sequence of SEQ ID NO: 25 or an equivalent thereof. In a further embodiment, the antibody or antigen-binding fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to the tip-chimeric peptide IhfA5-mIhfB4_{NTHI}. In yet a further embodiment, the fragment is an antigen-binding fragment. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 8 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 9 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID of NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID NO: 8 or an equivalent thereof. In yet another embodiment, the antibody or antigen-binding fragment thereof comprises or consists essentially of, or yet further consists of: a the heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID NO: 9 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 8 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 9 or an equivalent thereof.

In some embodiments, the antibody or antigen-binding fragment thereof comprises, or consists essentially of, or yet consists of a heavy chain (HC) comprising, or consisting essentially of, or yet further consisting of an amino acid sequence of SEQ ID NO: 26 or an equivalent thereof, and a light chain (LC) comprising, or consisting essentially of, or yet further consisting of an amino acid sequence of SEQ ID NO: 27 or an equivalent thereof. In a further embodiment, the antibody or antigen-binding fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody or an antigen-binding fragment thereof binds to the tail-chimeric peptide IhfA3-IhfB2_{NTHI}. In yet a further embodiment, the fragment is an antigen-binding fragment. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 12 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 12 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 12 or an equivalent thereof.

In some embodiments, the antibody or antigen-binding fragment thereof comprises, or consists essentially of, or yet consists of a heavy chain (HC) comprising, or consisting essentially of, or yet further consisting of an amino acid sequence of aa 25 to aa 473 of SEQ ID NO: 24 or an equivalent thereof, and/or a light chain (LC) comprising, or consisting essentially of, or yet further consisting of an amino acid sequence of aa 21 to aa 239 of SEQ ID NO: 25 or an equivalent thereof. In a further embodiment, the antibody or antigen-binding fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to the tip-chimeric peptide IhfA5-mIhfB4_{NTHI}. In yet a further embodiment, the fragment is an antigen-binding fragment. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 9 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID of NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or antigen-binding fragment thereof comprises or consists essentially of, or yet further consists of: a the heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID NO: 9 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 7 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 9 or an equivalent thereof.

In some embodiments, the antibody or antigen-binding fragment thereof comprises, or consists essentially of, or yet consists of a heavy chain (HC) comprising, or consisting essentially of, or yet further consisting of an amino acid sequence of aa 25 to aa 473 of SEQ ID NO: 26 or an equivalent thereof, and a light chain (LC) comprising, or consisting essentially of, or yet further consisting of an amino acid sequence of aa 21 to aa 233 of SEQ ID NO: 27 or an equivalent thereof. In a further embodiment, the antibody or antigen-binding fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody or antigen-binding fragment thereof binds to the tail-chimeric peptide IhfA3-IhfB2_{NTHI}. In yet a further embodiment, the fragment is an antigen-binding fragment. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 12 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 12 or an equivalent thereof. In one embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or antigen-binding fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 12 or an equivalent thereof.

In some embodiments, the antibody or antigen-binding fragment thereof comprises, or consists essentially of, or yet consists of any one or any two or all three CDRs of a sequence selected from the group of: SEQ ID NOs: 1-3 or 24, or an equivalent of each thereof; and/or any one or any two or all three CDRs of a sequence selected from the group of: SEQ ID NOs: 7-9 or 25, or an equivalent of each thereof. In a further embodiment, the antibody or antigen-binding fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to the tip-chimeric peptide IhfA5-mIhfB4_{NTHI}. In yet a further embodiment, the fragment is an antigen-binding fragment. In some embodiments, the antibody or antigen-binding fragment thereof comprises, or consists essentially of, or yet consists of all three CDRs of a sequence selected from the group of: SEQ ID NOs: 1-3 or 24, or an equivalent of each thereof; and/or all three CDRs of a sequence selected from the group of: SEQ ID NOs: 7-9 or 25, or an equivalent of each thereof.

In some embodiments, the antibody or antigen-binding fragment thereof comprises, or consists essentially of, or yet consists of any one or any two or all three CDRs of a sequence selected from the group of: SEQ ID NOs: 4-6 or 26, or an equivalent of each thereof; and/or any one or any two or all three CDRs of a sequence selected from the group of: SEQ ID NOs: 10-12 or 27, or an equivalent of each thereof. In a further embodiment, the antibody or antigen-binding fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody of an antigen-binding fragment thereof binds to the tail-chimeric peptide IhfA3-IhfB2_{NTHI}. In yet a further embodiment, the fragment is an antigen-binding fragment. In some embodiments, the antibody or antigen-binding fragment thereof comprises, or consists essentially of, or yet consists of all three CDRs of a sequence selected from the group of: SEQ ID NOs: 4-6 or 26, or an equivalent of each thereof; and/or all three CDRs of a sequence selected from the group of: SEQ ID NOs: 10-12 or 27, or an equivalent of each thereof.

In certain embodiments, the antibody or antigen-binding fragment thereof as provided herein further comprises one or more signal peptide(s). In one embodiment, the signal peptide comprises or consists essentially of, or yet further consists of amino acid (aa) 1 to aa 24 of any one of SEQ ID NOs: 1-6, 13, 24 or 26. In another embodiment, the signal peptide comprises or consists essentially of, or yet further consists of aa 1 to aa 20 of any one of SEQ ID NOs: 7-12, 14, 25 and 27. In a further embodiment, the signal peptide is located at the amino terminus of the light chain variable region. Additionally or alternatively, the same signal peptide or a different signal peptide is located at the amino terminus of the heavy chain variable region.

The antibody or antigen-binding fragment thereof as provided herein may be monospecific or bispecific. In one embodiment, the antibody or antigen-binding fragment thereof is trispecific, or tetraspecific, or pentaspecific. Additionally or alternatively, the antibody is selected from the group of an IgA (such as an IgA1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgA1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region.

In certain embodiments, an equivalent to an amino acid sequence comprises or consists essentially of, or yet further consists of a polypeptide having at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence. Additionally or alternatively, an equivalent to the amino acid sequence comprises or consists essentially of, or yet further consists of a polypeptide that is encoded by a polynucleotide that hybridizes under conditions of high stringency to the complement of the polynucleotide encoding the amino acid sequence. In a further embodiment, an equivalent to an amino acid sequence comprises or consists essentially of, or yet further consists of a polypeptide at least 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) identity to the amino acid sequence. In certain embodiments, an equivalent to an amino acid sequence (such as an antibody or an antigen-binding fragment thereof, or any one or more of SEQ ID NOs: 1-14 and 24-26 or a fragment thereof as disclosed herein, including but not limited to: 25 to aa 144 of SEQ ID NOs: 13, 24 or 26, aa 21 to aa 132 of SEQ ID NOs: 14 or 25, aa 21 to aa 126 of SEQ ID NO: 27, aa 25 to aa 473 of SEQ ID NOs: 13, 24 or 26, aa 21 to aa 239 of SEQ ID NOs: 14 or 25, aa 21 to aa 233 of SEQ ID NO: 27) comprises, or consists essentially of, or yet further consists of a polypeptide comprises one or more or all CDRs of the amino acid sequence. Additionally or alternatively, the polypeptide is at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence.

In certain embodiments, the equivalent to an amino acid sequence, such as an antibody, an antigen-binding fragment thereof, a complementarity-determining region (CDR) thereof, or a CDR-containing polypeptide, lacks an amino acid difference to the amino acid sequence in the CDR(s). However, the equivalent to an amino acid sequence, such as an antibody, an antigen-binding fragment thereof, a CDR thereof, or a CDR-containing polypeptide, may comprises one or more of (for example but not limited to 1, 2, 3, 4, 5, 6, 7, 8, 9, 10, 11, 12, 13, 14, 15, 16, 17, 18, 19, 20, 21, 22, 23, 24 or 25) amino acid differences compared to the amino acid sequence in the non-CDR region(s) with the proviso that the three-dimensional arrangement of the CDR(s) is and/or the CDRs is/are retained. In certain embodiments, the equivalent polypeptide to an amino acid sequence, such as an antibody, an antigen-binding fragment thereof, a CDR thereof, or a CDR-containing polypeptide, is at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence with the proviso that the three-dimensional arrangement of the CDR(s) and/or the CDR(s) is/are retained.

Non-limiting examples of such non-CDR regions includes a framework region (FR), a constant region, an Fc region, a pFc′ region, a constant heavy chain (CH) domain (such as CH1, CH2, CH3 or CH4), a constant light chain (CL) domain, or a hinge region. In one embodiment, such amino acid differences may be a conservative amino acid substitution and/or does not change the three-dimensional arrangement of the antibody, antigen-binding fragment thereof, CDR thereof, or the CDR-containing polypeptide. In another embodiment, the equivalent may comprises a conservative amino acid substitution in the boundaries of a CDR, such as one or two amino acid(s) at the amino termini, the carboxyl termini or both of the CDR.

In one aspect, provided is one or more of CDRs (such as any 1, or 2, or 3, or 4, or 5, or 6 CDR(s)) of an antibody or an antigen-binding fragment thereof as disclosed herein. In one embodiment, provided is a set of CDRs comprising or alternatively consisting essentially of, or yet further consisting of one or more of CDRs (such as any 1, or 2, or 3, or 4, or 5, or 6 CDR(s)) of an antibody or an antigen-binding fragment thereof as disclosed herein. In one embodiment, provided is a set of CDRs comprising, or alternatively consisting essentially of, or yet further consisting of CDRL1, CDRL2, and CDRL3 of a variable region as disclosed herein. In a further embodiment, provided is a set of CDRs comprising, or alternatively consisting essentially of, or yet further consisting of CDRH1, CDRH2, and CDRH3 of a variable region as disclosed herein. In yet a further embodiment, provided is a set of CDRs comprising, or alternatively consisting essentially of, or yet further consisting of CDRL1, CDRL2, and CDRL3 of a variable region as disclosed herein and CDRH1, CDRH2, CDRH3 of another variable region as disclosed herein. In certain embodiments, the CDR set constitutes a paratope. Additionally or alternatively, the CDR set specifically binds to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In a further embodiment, provided is an antibody, an antigen-binding fragment thereof, or an equivalent of each thereof, comprising, or alternatively consisting essentially of, or yet further consisting of any one or more CDRs as disclosed herein. In yet a further embodiment, provided is an antibody, an antigen-binding fragment thereof, or an equivalent of each thereof, comprising, or alternatively consisting essentially of, or yet further consisting of a CDR set as disclosed herein.

In certain embodiments, CDRs of SEQ ID NOs: 1-13 are illustrated in the following table. In certain embodiments, CDRH1 of any one of SEQ ID NOs: 1-6, 13, 24 or 26 comprises or consists essentially of, or yet further consists of amino acid (aa) 50 to aa 57 of SEQ ID NOs: 1-6, 13, 24 or 26, respectively. In certain embodiments, CDRH2 of any one of SEQ ID NOs: 1-6, 13, 24 or 26 comprises or consists essentially of, or yet further consists of amino acid (aa) 75 to aa 82 of SEQ ID NO: 1-6, 13, 24 or 26, respectively. In certain embodiments, CDRH3 of any one of SEQ ID NOs: 1-6, 13, 24 or 26 comprises or consists essentially of, or yet further consists of amino acid (aa) 121 to aa 133 of SEQ ID NO: 1-6, 13, 24 or 26, respectively. In certain embodiments, CDRL1 of any one of SEQ ID NOs: 7-9, 14 or 25 comprises or consists essentially of, or yet further consists of amino acid (aa) 47 to aa 57 of SEQ ID NO: 7-9, 14 or 25, respectively. In certain embodiments, CDRL2 of any one of SEQ ID NOs: 7-9, 14 or 25 comprises or consists essentially of, or yet further consists of amino acid (aa) 75 to aa 77 of SEQ ID NO: 7-9, 14 or 25, respectively. In certain embodiments, CDRL3 of any one of SEQ ID NOs: 7-9, 14 or 25 comprises or consists essentially of, or yet further consists of amino acid (aa) 114 to aa 122 of SEQ ID NO: 7-9, 14 or 25, respectively. In certain embodiments, CDRL1 of any one of SEQ ID NOs: 10-12 or 27 comprises or consists essentially of, or yet further consists of amino acid (aa) 47 to aa 52 of SEQ ID NOs: 10-12 or 27, respectively. In certain embodiments, CDRL2 of any one of SEQ ID NOs: 10-12 or 27 comprises or consists essentially of, or yet further consists of amino acid (aa) 70 to aa 72 of SEQ ID NOs: 10-12 or 27, respectively. In certain embodiments, CDRL3 of any one of SEQ TD NOs: 10-12 or 27 comprises or consists essentially of, or yet further consists of amino acid (aa) 109 to aa 116 of SEQ ID NO: 10-12 or 27, respectively.

| SEQ | | | | |
|---|---|---|---|---|
| ID | | | | |
| NO: | CDR1 | CDR2 | CDR3 | Variable Region |
| 4 | GFTFSRYG | ISSGGSYT | ERHGGDGYWYFDV | EVQLVESGGGLVKPGGSLRLSCAASGFTFSRYGM |
| | (residues | (residues | (residues 121- | SWVRQAPGKGLEWVATISSGGSYTYYTDSVKGRF |
| | 50-57 of | 75-82 of | 133 of SEQ | TISRDNAKNSLYLQMNSLRAEDTAVYYCERHGG |
| | SEQ ID | SEQ ID | ID NO: 4) | DGYWYFDVWGQGTMVTVSS (residues 25-144 of |
| | NO: 4) | NO: 4) | | SEQ ID NO: 4) |
| 5 | GFTFSRYG | ISSGGSYT | ERHGGDGYWYFDV | EVQLVESGGGLVKPGGSLRLSCAASGFTFSRYGM |
| | (residues | (residues | (residues 121- | SWVRQAPGKGLEWVSTISSGGSYTYYTDSVKGRF |
| | 50-57 of | 75-82 of | 133 of SEQ | TISRDNAKNSLYLQMNSLRAEDTAVYYCERHGG |
| | SEQ ID | SEQ ID | ID NO: 5) | DGYWYFDVWGQGTMVTVSS (residues 25-144 of |
| | NO: 5) | NO: 5) | | SEQ ID NO: 5) |
| 6 | GFTFSRYG | ISSGGSYT | ERHGGDGYWYFDV | EVQLVESGGGLVQPGRSLRLSCTASGFTFSRYGM |
| | (residues | (residues | (residues 121- | SWVRQAPGKGLEWVATISSGGSYTYYTDSVKGRF |
| | 50-57 of | 75-82 of | 133 of SEQ | TISRDNAKNILYLQMNSLKTEDTAVYYCERHGGD |
| | SEQ ID | SEQ ID | ID NO: 6) | GYWYFDVWGQGTMVTVSS (residues 25-144 of |
| | NO: 6) | NO: 6) | | SEQ ID NO: 6) |
| 10 | QDISNY | YTS | QQGNPLRT | DIQMTQSPSSLSASVGDRVTITCRASQDISNYLNW |
| | (residues | (residues | (residues 109- | YQQKPGKAVKLLIYYTSRLHSGVPSRFSGSGSGTD |
| | 47-52 of | 70-72 of | 116 of SEQ | YTLTISSLQPEDFATYFCQQGNPLRTFGGGTKVEI |
| | SEQ ID | SEQ ID | ID NO: 10) | K (residues 21-126 of SEQ ID NO: 10) |
| | NO: 10) | NO: 10) | | |
| 11 | QDISNY | YTS | QQGNPLRT | DIQMTQSPSSLSASVGDRVTITCRASQDISNYLNW |
| | (residues | (residues | (residues 109- | YQQKPGKAVKLLIYYTSRLHSGVPSRFSGSGSGTD |
| | 47-52 of | 70-72 of | 116 of SEQ | YTLTISSLQPEDFATYYCQQGNPLRTFGGGTKVEI |
| | SEQ ID | SEQ ID | ID NO: 11) | K (residues 21-126 of SEQ ID NO: 11) |
| | NO: 11) | NO: 11) | | |
| 12 | QDISNY | YTS | QQGNPLRT | DIVMTQSPATLSLSPGERATLSCRASQDISNYLNW |
| | (residues | (residues | (residues 109- | YQQKPGQAVRLLIYYTSRLHSGIPARFSGSGSGTD |
| | 47-52 of | 70-72 of | 116 of SEQ | YTLTISSLEPEDFAVYFCQQGNPLRTFGGGTKVEI |
| | SEQ ID | SEQ ID | ID NO: 12) | K (residues 21-126 of SEQ ID NO: 12) |
| | NO: 12) | NO: 12) | | |
| 1 | GFTFRTYA | IGSDRRHT | VGPYDGYYGEFDY | EVKLVESGGGLVQPGGSLRLSCAASGFTFRTYAM |
| | (residues | (residues | (residues 121- | SWVRQAPGKGLEWVATIGSDRRHTYYPDSVKGR |
| | 50-57 of | 75-82 of | 133 of SEQ | FTISRDNAKNTLYLQMNSLRAEDTAVYYCVGPYD |
| | SEQ ID | SEQ ID | ID NO: 1) | GYYGEFDYWGQGTLVTVSS (residues 25-144 of |
| | NO: 1) | NO: 1) | | SEQ ID NO: 1) |
| 2 | GFTFRTYA | IGSDRRHT | VGPYDGYYGEFDY | EVQLVESGGGLVQPGGSLRLSCAASGFTFRTYAM |
| | (residues | (residues | (residues 121- | SWVRQAPGKGLEWVATIGSDRRHTYYPDSVKGR |
| | 50-57 of | 75-82 of | 133 of SEQ | FTISRDNSKNTLYLQMNSLRAEDTAVYYCVGPYD |
| | SEQ ID | SEQ ID | ID NO: 2) | GYYGEFDYWGQGTLVTVSS (residues 25-144 of |
| | NO: 2) | NO: 2) | | SEQ ID NO: 2) |
| 3 | GFTFRTYA | IGSDRRHT | VGPYDGYYGEFDY | EVKLVQSGAEVKKPGASVKVSCKASGFTFRTYA |
| | (residues | (residues | (residues 121- | MSWVRQAPGQRLEWVATIGSDRRHTYYPDKFQG |
| | 50-57 of | 75-82 of | 133 of SEQ | RVTITRDNAKNTLYMELSSLRSEDTAVYYCVGPY |
| | SEQ ID | SEQ ID | ID NO: 3) | DGYYGEFDYWGQGTLVTVSS (residues 25-144 of |
| | NO: 3) | NO: 3) | | SEQ ID NO: 3) |
| 7 | QSLLDSDGKTF | LVS | WQGTHFPYT | DVVMTQSPLSLPVTLGQPASISCRSSQSLLDSDGK |
| | (residues | (residues | (residues | TFLNWLQQRPGQSPRRLIYLVSKLDSGVPDRFSGS |
| | 47-57 of | 75-77 of | 114-122 of | GSGTDFTLKISRVEAEDVGVYYCWQGTHFPYTFG |
| | SEQ ID | SEQ ID | SEQ ID NO: 7) | QGTKLEIK (residues 21-132 of SEQ ID NO: 7) |
| | NO: 7) | NO: 7) | | |
| 8 | QSLLDSDGKTF | LVS | WQGTHFPYT | DVVMTQSPLSLPVTLGQPASISCRSSQSLLDSDGK |
| | (residues | (residues | (residues | TFLNWLQQRPGQSPRRLIYLVSKRDSGVPDRFSGS |
| | 47-57 of | 75-77 of | 114-122 of | GSGTDFTLKISRVEAEDVGVYYCWQGTHFPYTFG |
| | SEQ ID | SEQ ID | SEQ ID NO: 8) | QGTKLEIK (residues 21-132 of SEQ ID NO: 8) |
| | NO: 8) | NO: 8) | | |
| 9 | QSLLDSD | LVS | WQGTHFPYT | DVVMTQSPDSLAVSLGERATINCKSSQSLLDSDG |
| | GKTF | (residues | (residues | KTFLNWLQQKPGQPPKRLIYLVSKLDSGVPDRFS |
| | (residues | 75-77 of | 114-122 of | GSGSGTDFTLTISSLQAEDVAVYYCWQGTHFPYT |
| | 47-57 of | SEQ ID | SEQ ID NO: 9) | FGQGTKLEIK (residues 21-132 of SEQ ID NO: 9) |
| | SEQ ID | NO: 9) | | |
| | NO: 9) | | | |

In certain embodiments, provided are CDRs as identified in the following two Tables below.

| SEQ | | | 1 | 2 | 3 | 4 | 5 | 6 | 7 | 8 |
|---|---|---|---|---|---|---|---|---|---|---|
| 1 | VH1 | CDR1 | AASGFTFR | GFTFRTYA | GFTFRTY | AASGFTFR | GFTFRTYA | GFTFRTY | GFTFRTYA | GFTFRTY |
| | | | TYAMS | (residues | (residues | TYAMS | (residues | (residues | (residues | (residues |
| | | | (residues | 50-57 of | 50-56 of | (residues | 50-57 of | 50-56 of | 50-57 of | 50-56 of |
| | | | 47-59 of | SEQ ID | SEQ ID | 47-59 of | SEQ ID | SEQ ID | SEQ ID | SEQ ID |
| | | | SEQ ID | NO: 1) | NO: 1) | SEQ ID | NO: 1) | NO: 1) | NO: 1) | NO: 1) |
| | | | NO: 1) | | | NO: 1) | | | | |
| | | CDR2 | TIGSDRRH | IGSDRRHT | GSDRRH | TIGSDRRH | IGSDRRHT | GSDRRH | IGSDRRHT | GSDRRH |
| | | | TY | (residues | (residues | TY | (residues | (residues | (residues | (residues |
| | | | (residues | 75-82 of | 76-81 of | (residues | 75-82 of | 76-81 of | 75-82 of | 76-81 of |
| | | | 74-83 of | SEQ ID | SEQ ID | 74-83 of | SEQ ID | SEQ ID | SEQ ID | SEQ ID |
| | | | SEQ ID | NO: 1) | NO: 1) | SEQ ID | NO: 1) | NO: 1) | NO: 1) | NO: 1) |
| | | | NO: 1) | | | NO: 1) | | | | |
| | | CDR3 | Not | Not | Not | Not | Not | Not | VGPYDGYY | VGPYDGYY |
| | | | deter- | deter- | deter- | deter- | deter- | deter- | GEFDY | GEFDY |
| | | | mined | mined | mined | mined | mined | mined | (residues | (residues |
| | | | | | | | | | 121-133 | 121-133 |
| | | | | | | | | | of SEQ | of SEQ |
| | | | | | | | | | ID NO: 1) | ID NO: 1) |
| 2 | VH2 | CDR1 | AASGFTFR | GFTFRTYA | GFTFRTY | AASGFTFR | GFTFRTYA | GFTFRTY | GFTFRTYA | GFTFRTY |
| | | | TYAMS | (residues | (residues | TYAMS | (residues | (residues | (residues | (residues |
| | | | (residues | 50-57 of | 50-56 of | (residues | 50-57 of | 50-56 of | 50-57 of | 50-56 of |
| | | | 47-59 of | SEQ ID | SEQ ID | 47-59 of | SEQ ID | SEQ ID | SEQ ID | SEQ ID |
| | | | SEQ ID | NO: 2) | NO: 2) | SEQ ID | NO: 2) | NO: 2) | NO: 2) | NO: 2) |
| | | | NO: 2) | | | NO: 2) | | | | |
| | | CDR2 | TIGSDRRH | IGSDRRHT | GSDRRH | TIGSDRRH | IGSDRRHT | GSDRRH | IGSDRRHT | GSDRRH |
| | | | TY | (residues | (residues | TY | (residues | (residues | (residues | (residues |
| | | | (residues | 75-82 of | 76-81 of | (residues | 75-82 of | 76-81 of | 75-82 of | 76-81 of |
| | | | 74-83 of | SEQ ID | SEQ ID | 74-83 of | SEQ ID | SEQ ID | SEQ ID | SEQ ID |
| | | | SEQ ID | NO: 2) | NO: 2) | SEQ ID | NO: 2) | NO: 2) | NO: 2) | NO: 2) |
| | | | NO: 2) | | | NO: 2) | | | | |
| | | CDR3 | Not | Not | Not | Not | Not | Not | VGPYDGYY | VGPYDGYY |
| | | | deter- | deter- | deter- | deter- | deter- | deter- | GEFDY | GEFDY |
| | | | mined | mined | mined | mined | mined | mined | (residues | (residues |
| | | | | | | | | | 121-133 | 121-133 |
| | | | | | | | | | of SEQ | of SEQ |
| | | | | | | | | | ID NO: | ID NO: |
| | | | | | | | | | 2) | 2) |
| 3 | VH3 | CDR1 | KASGFTFR | GFTFRTYA | GFTFRTY | KASGFTFR | GFTFRTYA | GFTFRTY | GFTFRTYA | GFTFRTY |
| | | | TYAMS | (residues | (residues | TYAMS | (residues | (residues | (residues | (residues |
| | | | (residues | 50-57 of | 50-56 of | (residues | 50-57 of | 50-56 of | 50-57 of | 50-56 of |
| | | | 47-59 of | SEQ ID | SEQ ID | 47-59 of | SEQ ID | SEQ ID | SEQ ID | SEQ ID |
| | | | SEQ ID | NO: 3) | NO: 3) | SEQ ID | NO: 3) | NO: 3) | NO: 3) | NO: 3) |
| | | | NO: 3) | | | NO: 3) | | | | |
| | | CDR2 | TIGSDRRH | IGSDRRHT | GSDRRH | TIGSDRRH | IGSDRRHT | GSDRRH | IGSDRRHT | GSDRRH |
| | | | TY | (residues | (residues | TY | (residues | (residues | Y | (residues |
| | | | (residues | 75-82 of | 76-81 of | (residues | 75-82 of | 76-81 of | (residues | 76-81 |
| | | | 74-83 of | SEQ ID | SEQ ID | 74-83 of | SEQ ID | SEQ ID | 75-83 of | of SEQ |
| | | | SEQ ID | NO: 3) | NO: 3) | SEQ ID | NO: 3) | NO: 3) | SEQ ID | ID NO: |
| | | | NO: 3) | | | NO: 3) | | | NO: 3) | 3) |
| | | CDR3 | Not | Not | Not | Not | Not | Not | VGPYDGYY | VGPYDGYY |
| | | | deter- | deter- | deter- | deter- | deter- | deter- | GEFDY | GEFDY |
| | | | mined | mined | mined | mined | mined | mined | (residues | (residues |
| | | | | | | | | | 121-133 | 121-133 |
| | | | | | | | | | of SEQ | of SEQ |
| | | | | | | | | | ID NO: | ID NO: |
| | | | | | | | | | 3) | 3) |
| 7 | VL1 | CDR1 | RSSQSLLD | QSLLDSDG | RSSQSLLD | RSSQSLLD | QSLLDSDG | RSSQSLLD | QSLLDSDG | QSLLDSDG |
| | | | SDGKTFLN | KTF | SDGKTFLN | SDGKTFLN | KTF | SDGKTFLN | KTF | KTF |
| | | | (residues | (residues | (residues | (residues | (residues | (residues | (residues | (residues |
| | | | 44-59 of | 47-57 of | 44-59 of | 44-59 of | 47-57 of | 44-59 of | 47-57 of | 47-57 of |
| | | | SEQ ID | SEQ ID | SEQ ID | SEQ ID | SEQ ID | SEQ ID | SEQ ID | SEQ ID |
| | | | NO: 7) | NO: 7) | NO: 7) | NO: 7) | NO: 7) | NO: 7) | NO: 7) | NO: 7) |
| | | CDR2 | YLVSKLDS | LVS | LVSKLDS | YLVSKLDS | LVS | LVSKLDS | LVS | LVS |
| | | | (residues | (residues | (residues | (residues | (residues | (residues | (residues | (residues |
| | | | 74-81 of | 75-77 of | 75-81 of | 74-81 of | 75-77 of | 75-81 of | 75-77 of | 75-77 of |
| | | | SEQ ID | SEQ ID | SEQ ID | SEQ ID | SEQ ID | SEQ ID | SEQ ID | SEQ ID |
| | | | NO: 7) | NO: 7) | NO: 7) | NO: 7) | NO: 7) | NO: 7) | NO: 7) | NO: 7) |
| | | CDR3 | WQGTHFPY | WQGTHFPY | WOGTHFPY | WQGTHFPY | WQGTHFPY | WQGTHFPY | WQGTHFPY | WQGTHFPY |
| | | | T | T | T | T | T | T | T | T |
| | | | (residues | (residues | (residues | (residues | (residues | (residues | (residues | (residues |
| | | | 114-122 of | 114-122 | 114-122 | 114-122 | 114-122 | 114-122 | 114-122 | 114-122 |
| | | | SEQ ID | of SEQ | of SEQ | of SEQ ID | of SEQ ID | of SEQ ID | of SEQ | of SEQ ID |
| | | | NO: 7) | ID NO: 7) | ID NO: 7) | NO: 7) | NO: 7) | NO: 7) | ID NO: | NO: 7) |
| 8 | VL2 | CDR1 | RSSQSLLD | QSLLDSDG | RSSQSLLD | RSSQSLLD | QSLLDSDG | RSSQSLLD | QSLLDSDG | QSLLDSDG |
| | | | SDGKTFLN | KTF | SDGKTFLN | SDGKTFLN | KTF | SDGKTFLN | KTF | KTF |
| | | | (residues | (residues | (residues | (residues | (residues | (residues | (residues | (residues |
| | | | 44-59 of | 47-57 of | 44-59 of | 44-59 of | 47-57 of | 44-59 of | 47-57 of | 47-57 of |
| | | | SEQ ID | SEQ ID | SEQ ID | SEQ ID | SEQ ID | SEQ ID | SEQ ID | SEQ ID |
| | | | NO: 8) | NO: 8) | NO: 8) | NO: 8) | NO: 8) | NO: 8) | NO: 8) | NO: 8) |
| | | CDR2 | YLVSKRDS | LVS | LVSKRDS | YLVSKRDS | LVS | LVSKRDS | LVS | LVS |
| | | | (residues | (residues | (residues | (residues | (residues | (residues | (residues | (residues |
| | | | 74-81 of | 75-77 of | 75-81 of | 74-81 of | 75-77 of | 74-81 of | 75-77 of | 75-77 of |
| | | | SEQ ID | SEQ ID | SEQ ID | SEQ ID | SEQ ID | SEQ ID | SEQ ID | SEQ ID |
| | | | NO: 8) | NO: 8) | NO: 8) | NO: 8) | NO: 8) | NO: 8) | NO: 8) | NO: 8) |
| | | CDR3 | WQGTHFPY | WQGTHFPY | WQGTHFPY | WQGTHFPY | WQGTHFPY | WQGTHFPY | WQGTHFPY | WQGTHFPY |
| | | | T | T | T | T | T | T | T | T |
| | | | (residues | (residues | (residues | (residues | (residues | (residues | (residues | (residues |
| | | | 114-122 | 114-122 | 114-122 | 114-122 | 114-122 | 114-122 | 114-122 | 114-122 |
| | | | of SEQ ID | of SEQ ID | of SEQ ID | of SEQ ID | of SEQ ID | of SEQ ID | of SEQ ID | of SEQ ID |
| | | | NO: 8) | NO: 8) | NO: 8) | NO: 8) | NO: 8) | NO: 8) | NO: 8) | NO: 8) |
| 9 | VL3 | CDR1 | KSSQSLLD | QSLLDSDG | KSSQSLLD | KSSQSLLD | QSLLDSDG | KSSQSLLD | QSLLDSDG | QSLLDSDG |
| | | | SDGKTFLN | KTF | SDGKTFLN | SDGKTFLN | KTF | SDGKTFLN | KTF | KTF |
| | | | (residues | (residues | (residues | (residues | (residues | (residues | (residues | (residues |
| | | | 44-59 of | 47-57 of | 44-59 of | 44-59 of | 47-57 of | 44-59 of | 47-57 of | 47-57 of |
| | | | SEQ ID | SEQ ID | SEQ ID | SEQ ID | SEQ ID | SEQ ID | SEQ ID | SEQ ID |
| | | | NO: 9) | NO: 9) | NO: 9) | NO: 9) | NO: 9) | NO: 9) | NO: 9) | NO: 9) |
| | | CDR2 | YLVSKLDS | LVS | LVSKLDS | YLVSKLDS | LVS | LVSKLDS | LVS | LVS |
| | | | (residues | (residues | (residues | (residues | (residues | (residues | (residues | (residues |
| | | | 74-81 of | 75-77 of | 75-81 of | 74-81 of | 75-77 of | 75-81 of | 75-77 of | 75-77 of |
| | | | SEQ ID | SEQ ID | SEQ ID | SEQ ID | SEQ ID | SEQ ID | SEQ ID | SEQ ID |
| | | | NO: 9) | NO: 9) | NO: 9) | NO: 9) | NO: 9) | NO: 9) | NO: 9) | NO: 9) |
| | | CDR3 | WQGTHFPY | WQGTHFPY | WQGTHFPY | WQGTHFPY | WQGTHFPY | WQGTHFPY | WQGTHFPY | WQGTHFPY |
| | | | T | T | T | T | T | T | T | T |
| | | | (residues | (residues | (residues | (residues | (residues | (residues | (residues | (residues |
| | | | 114-122 | 114-122 | 114-122 | 114-122 | 114-122 | 114-122 | 114-122 | 114-122 |
| | | | of SEQ ID | of SEQ ID | of SEQ ID | of SEQ ID | of SEQ ID | of SEQ ID | of SEQ ID | of SEQ ID |
| | | | NO: 9) | NO: 9) | NO: 9) | NO: 9) | NO: 9) | NO: 9) | NO: 9) | NO: 9) |

SEQ: SEQ ID NO

| | | | | | | | | | | |
|---|---|---|---|---|---|---|---|---|---|---|
| 4 | VH1 | CDR1 | AASGFTFS | GFTFSRYG | GFTFSRY | AASGFTFS | GFTFSRYG | GFTFSRY | GFTFSRYG | GFTFSRY |
| | | | RYGMS | (residues | (residues | RYGMS | (residues | (residues | (residues | (residues |
| | | | (residues | 50-57 | 50-56 | (residues | 50-57 | 50-56 | 50-57 | 50-56 |
| | | | 47-59 of | of SEQ | of SEQ | 47-59 of | of SEQ | of SEQ | of SEQ | of SEQ |
| | | | SEQ ID | ID NO: 4) | ID NO: 4) | SEQ ID | ID NO: 4) | ID NO: 4) | ID NO: 4) | ID NO: 4) |
| | | | NO: 4) | | | NO: 4) | | | | |
| | | CDR2 | TISSGGSY | ISSGGSYT | SSGGSY | TISSGGSY | ISSGGSYT | SSGGSY | ISSGGSYT | SSGGSY |
| | | | TY | (residues | (residues | TY | (residues | (residues | (residues | (residues |
| | | | (residues | 75-82 of | 76-81 of | (residues | 75-82 of | 76-81 of | 75-82 of | 76-81 of |
| | | | 74-83 of | SEQ ID | SEQ ID | 74-83 of | SEQ ID | SEQ ID | SEQ ID | SEQ ID |
| | | | SEQ ID | NO: 4) | NO: 4) | SEQ ID | NO: 4) | NO: 4) | NO: 4) | NO: 4) |
| | | | NO: 4) | | | NO: 4) | | | | |
| | | CDR3 | Not | Not | Not | Not | Not | Not | ERHGGDGY | ERHGGDGY |
| | | | deter- | deter- | deter- | deter- | deter- | deter- | WYFDV | WYFDV |
| | | | mined | mined | mined | mined | mined | mined | (residues | (residues |
| | | | | | | | | | 121-133 | 121-133 |
| | | | | | | | | | of SEQ ID | of SEQ ID |
| | | | | | | | | | NO: 4) | NO: 4) |
| 5 | VH2 | CDR1 | AASGFTFS | GFTFSRYG | GFTFSRY | AASGFTFS | GFTFSRYG | GFTFSRY | GFTFSRY | GFTFSRY |
| | | | RYGMS | (residues | (residues | RYGMS | (residues | (residues | (residues | (residues |
| | | | (residues | 50-57 of | 50-56 of | (residues | 50-57 of | 50-56 of | 50-57 of | 50-56 of |
| | | | 47-59 of | SEQ ID | SEQ ID | 47-59 of | SEQ ID | SEQ ID | SEQ ID | SEQ ID |
| | | | SEQ ID | NO: 5) | NO: 5) | SEQ ID | NO: 5) | NO: 5) | NO: 5) | NO: 5) |
| | | | NO: 5) | | | NO: 5) | | | | |
| | | CDR2 | TISSGGSY | ISSGGSYT | SSGGSY | TISSGGSY | ISSGGSYT | SSGGSY | ISSGGSYT | SSGGSY |
| | | | TY | (residues | (residues | TY | (residues | (residues | (residues | (residues |
| | | | (residues | 75-82 of | 76-81 of | (residues | 75-82 of | 76-81 of | 75-82 of | 76-81 of |
| | | | 74-83 of | SEQ ID | SEQ ID | 74-83 of | SEQ ID | SEQ ID | SEQ ID | SEQ ID |
| | | | SEQ ID | NO: 5) | NO: 5) | SEQ ID | NO: 5) | NO: 5) | NO: 5) | NO: 5) |
| | | | NO: 5) | | | NO: 5) | | | | |
| | | CDR3 | Not | Not | Not | Not | Not | Not | ERHGGDGY | ERHGGDGY |
| | | | deter- | deter- | deter- | deter- | deter- | deter- | WYFDV | WYFDV |
| | | | mined | mined | mined | mined | mined | mined | (residues | (residues |
| | | | | | | | | | 121-133 | 121-133 |
| | | | | | | | | | of SEQ ID | of SEQ ID |
| | | | | | | | | | NO: 5) | NO: 5) |
| 6 | VH3 | CDR1 | TASGFTFS | GFTFSRYG | GFTFSRY | TASGFTFS | GFTFSRYG | GFTFSRY | GFTFSRYG | GFTFSRY |
| | | | RYGMS | (residues | (residues | RYGMS | (residues | (residues | (residues | (residues |
| | | | (residues | 50-57 of | 50-56 of | (residues | 50-57 of | 50-56 of | 50-57 of | 50-56 of |
| | | | 47-59 of | SEQ ID | SEQ ID | 47-59 of | SEQ ID | SEQ ID | SEQ ID | SEQ ID |
| | | | SEQ ID | NO: 6) | NO: 6) | SEQ ID | NO: 6) | NO: 6) | NO: 6) | NO: 6) |
| | | | NO: 6) | | | NO: 6) | | | | |
| | | CDR2 | TISSGGSY | ISSGGSYT | SSGGSY | TISSGGSY | ISSGGSYT | SSGGSY | ISSGGSYT | SSGGSY |
| | | | TY | (residues | (residues | TY | (residues | (residues | (residues | (residues |
| | | | (residues | 75-82 of | 76-81 of | (residues | 75-82 of | 76-81 of | 75-82 of | 76-81 of |
| | | | 74-83 of | SEQ ID | SEQ ID | 74-83 of | SEQ ID | SEQ ID | SEQ ID | SEQ ID |
| | | | SEQ ID | NO: 6) | NO: 6) | SEQ ID | NO: 6) | NO: 6) | NO: 6) | NO: 6) |
| | | | NO: 6) | | | NO: 6) | | | | |
| | | CDR3 | Not | Not | Not | Not | Not | Not | ERHGGDGY | ERHGGDGY |
| | | | deter- | deter- | deter- | deter- | deter- | deter- | WYFDV | WYFDV |
| | | | mined | mined | mined | mined | mined | mined | (residues | (residues |
| | | | | | | | | | 121-133 | 121-133 |
| | | | | | | | | | of SEQ ID | of SEQ ID |
| | | | | | | | | | NO: 6) | NO: 6) |
| 10 | VL1 | CDR1 | RASQDISN | QDISNY | RASQDISN | RASQDISN | QDISNY | RASQDISN | QDISNY | QDISNY |
| | | | YLN | (residues | YL | YLN | (residues | YL | (residues | (residues |
| | | | (residues | 47-52 of | (residues | (residues | 47-52 of | (residues | 47-52 of | 47-52 of |
| | | | 44-54 of | SEQ ID | 44-54 of | 44-54 of | SEQ ID | 44-54 of | SEQ ID | SEQ ID |
| | | | SEQ ID | NO: 10) | SEQ ID | SEQ ID | NO: 10) | SEQ ID | NO: 10) | NO: 10) |
| | | | NO: 10) | | NO: 10) | NO: 10) | | NO: 10) | | |
| | | CDR2 | YYTSRLHS | YTS | YTSRLHS | YYTSRLHS | YTS | YTSRLHS | YTS | YTS |
| | | | (residues | (residues | (residues | (residues | (residues | (residues | (residues | (residues |
| | | | 69-76 of | 70-72 of | 70-76 of | 69-76 of | 70-72 of | 70-76 of | 70-72 of | 70-72 of |
| | | | SEQ ID | SEQ ID | SEQ ID | SEQ ID | SEQ ID | SEQ ID | SEQ ID | SEQ ID |
| | | | NO: 10) | NO: 10) | NO: 10) | NO: 10) | NO: 10) | NO: 10) | NO: 10) | NO: 10) |
| | | CDR3 | QQGNPLRT | QQGNPLRT | QQGNPLRT | QQGNPLRT | QQGNPLRT | QQGNPLRT | QQGNPLRT | QQGNPLRT |
| | | | (residues | (residues | (residues | (residues | (residues | (residues | (residues | (residues |
| | | | 109-116 | 109-116 | 109-116 | 109-116 | 109-116 | 109-116 | 109-116 | 109-116 |
| | | | of SEQ ID | of SEQ ID | of SEQ ID | of SEQ ID | of SEQ ID | of SEQ ID | of SEQ ID | of SEQ ID |
| | | | NO: 10) | NO: 10) | NO: 10) | NO: 10) | NO: 10) | NO: 10) | NO: 10) | NO: 10) |
| 11 | VL2 | CDR1 | RASQDISN | QDISNY | RASQDIS | RASQDISN | QDISNY | RASQDISN | QDISNY | QDISNY |
| | | | YLN | (residues | NYLN | YLN | (residues | YLN | (residues | (residues |
| | | | (residues | 47-52 of | (residues | (residues | 44-54 of | (residues | 47-52 of | 47-52 of |
| | | | 44-54 of | SEQ ID | 44-54 of | 47-52 | SEQ ID | 44-54 of | SEQ ID | SEQ ID |
| | | | SEQ ID | NO: 11) | SEQ ID | SEQ ID | NO: 11) | SEQ ID | NO: 11) | NO: 11) |
| | | | NO: 11) | | NO: 11) | NO: 11) | | NO: 11) | | |
| | | CDR2 | YYTSRLHS | YTS | YTSRLHS | YYTSRLHS | YTS | YTSRLHS | YTS | YTS |
| | | | (residues | (residues | (residues | (residues | (residues | (residues | (residues | (residues |
| | | | 69-76 of | 70-72 of | 70-76 of | 69-76 of | 70-72 of | 70-76 of | 70-72 of | 70-72 of |
| | | | SEQ ID | SEQ ID | SEQ ID | SEQ ID | SEQ ID | SEQ ID | SEQ ID | SEQ ID |
| | | | NO: 11) | NO: 11) | NO: 11) | NO: 11) | NO: 11) | NO: 11) | NO: 11) | NO: 11) |
| | | CDR3 | QQGNPLRT | QQGNPLRT | QQGNPLRT | QQGNPLRT | QQGNPLRT | QQGNPLRT | QQGNPLRT | QQGNPLRT |
| | | | (residues | (residues | (residues | (residues | (residues | (residues | (residues | (residues |
| | | | 109-116 | 109-116 | 109-116 | 109-116 | 109-116 | 109-116 | 109-116 | 109-116 |
| | | | of SEQ ID | of SEQ | of SEQ | of SEQ | of SEQ | of SEQ | of SEQ | of SEQ |
| | | | NO: 11) | ID NO: | ID NO: | ID NO: | ID NO: | ID NO: | ID NO: | ID NO: |
| | | | | 11) | 11) | 11) | 11) | 11) | 11) | 11) |
| 12 | VL3 | CDR1 | RASQDISN | QDISNY | RASQDISN | RASQDISN | QDISNY | RASQDISN | QDISNY | QDISNY |
| | | | YLN | (residues | YLN | YLN | (residues | YLN | (residues | (residues |
| | | | (residues | 47-52 of | (residues | (residues | 47-52 of | (residues | 47-52 of | 47-52 of |
| | | | 44-54 of | SEQ ID | 44-54 of | 44-54 of | SEQ ID | 44-54 of | SEQ ID | SEQ ID |
| | | | SEQ ID | NO: 12) | SEQ ID | SEQ ID | NO: 12) | SEQ ID | NO: 12) | NO: 12) |
| | | | NO: 12) | | NO: 12) | NO: 12) | | NO: 12) | | |
| | | CDR2 | YYTSRLHS | YTS | YTSRLHS | YYTSRLHS | YTS | YTSRLHS | YTS | YTS |
| | | | (residues | (residues | (residues | (residues | (residues | (residues | (residues | (residues |
| | | | 69-76 of | 70-72 of | 70-76 of | 69-76 of | 70-72 of | 70-76 of | 70-72 of | 70-72 of |
| | | | SEQ ID | SEQ ID | SEQ ID | SEQ ID | SEQ ID | SEQ ID | SEQ ID | SEQ ID |
| | | | NO: 12) | NO: 12) | NO: 12) | NO: 12) | NO: 12) | NO: 12) | NO: 12) | NO: 12) |
| | | CDR3 | QQGNPLRT | QQGNPLRT | QQGNPLRT | QQGNPLRT | QQGNPLRT | QQGNPLRT | QQGNPLRT | QQGNPLRT |
| | | | (residues | (residues | (residues | (residues | (residues | (residues | (residues | (residues |
| | | | 109-116 | 109-116 | 109-116 | 109-116 | 109-116 | 109-116 | 109-116 | 109-116 |
| | | | of SEQ | of SEQ | of SEQ | of SEQ | of SEQ | of SEQ | of SEQ | of SEQ |
| | | | ID NO: | ID NO: | ID NO: | ID NO: | ID NO: | ID NO: | ID NO: | ID NO: |
| | | | 12) | 12) | 12) | 12) | 12) | 12) | 12) | 12) |

In certain embodiments, provided is an alternative CDR that is a CDR as identified herein further comprising an additional 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids at its amino terminus, or carboxyl terminus or both in the corresponding variable region sequence. Additionally or alternatively, provided is an alternative CDR that is a CDR as identified herein having 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids truncated at its amino terminus, or carboxyl terminus or both in the corresponding variable region sequence. For example, CDR1 of SEQ ID NO: 1 may be amino acid 50 to amino acid 57 of SEQ ID NO: 1. However, the CDR1 of SEQ ID NO: 1 can also start from amino acid 42, or 43, or 44, or 45, or 46, or 47, or 48, or 49, or 50, or 51, or 52, or 53, or 54, or 55, or 56, or 57, or 58 of SED ID NO: 1. Further, CDR1 of SEQ ID NO: 1 can end at amino acid 49, or 50, or 51, or 52, or 53, or 54, or 55, or 56, or 57, or 58, or 59, or 60, or 61, or 62, or 63, or 64, or 65 of SEQ ID NO: 1 with proviso that the CDR1 ends after its start. Additionally or alternatively, the CDR is about 1, or alternatively about 2, or alternatively about 3, or alternatively about 4, or alternatively about 5, or alternatively about 6, or alternatively about 7, or alternatively about 8, or alternatively about 9, or alternatively about 10, or alternatively about 11, or alternatively about 12, or alternatively about 13, or alternatively about 14, or alternatively about 15 amino acids long.

In certain embodiments, CDR2 of any one of SEQ ID NOs: 1-6 comprises or consists essentially of, or yet further consists of amino acid 71 to amino acid 85 of each of SEQ ID NOs: 1-6, respectively. In certain embodiments, CDR3 of any one of SEQ ID NOs: 1-6 comprises or consists essentially of, or yet further consists of amino acid 121 to amino acid 133 of each of SEQ ID NOs: 1-6, respectively. In certain embodiments, CDR2 of any one of SEQ ID NOs: 7-9 comprises or consists essentially of, or yet further consists of amino acid 71 to amino acid 81 of each of SEQ ID NOs: 7-9, respectively. In certain embodiments, CDR3 of any one of SEQ ID NOs: 7-9 comprises or consists essentially of, or yet further consists of amino acid 114 to amino acid 121 of each of SEQ ID NOs: 7-9, respectively. In certain embodiments, CDR2 of any one of SEQ ID NOs: 10-12 comprises or consists essentially of, or yet further consists of amino acid 66 to amino acid 76 of each of SEQ ID NOs: 10-12, respectively. In certain embodiments, CDR3 of any one of SEQ ID NOs: 10-12 comprises or consists essentially of, or yet further consists of amino acid 109 to amino acid 115 of each of SEQ ID NOs: 10-12, respectively.

In certain embodiments, CDR1 of any one of SEQ ID NOs: 1-6 comprises or consists essentially of, or yet further consists of amino acid 50 to amino acid 57 of each of SEQ ID NOs: 1-6, respectively. In certain embodiments, CDR2 of any one of SEQ ID NOs: 1-6 comprises or consists essentially of, or yet further consists of amino acid 75 to amino acid 82 of each of SEQ ID NOs: 1-6, respectively. In certain embodiments, CDR3 of any one of SEQ ID NOs: 1-6 comprises or consists essentially of, or yet further consists of amino acids 121 and 122 of each of SEQ ID NOs: 1-6, respectively. In certain embodiments, CDR1 of any one of SEQ ID NOs: 7-9 comprises or consists essentially of, or yet further consists of amino acid 47 to amino acid 57 of each of SEQ ID NOs: 7-9, respectively. In certain embodiments, CDR2 of any one of SEQ ID NOs: 7-9 comprises or consists essentially of, or yet further consists of amino acid 75 to amino acid 77 of each of SEQ ID NOs: 7-9, respectively. In certain embodiments, CDR3 of any one of SEQ ID NOs: 7-9 comprises or consists essentially of, or yet further consists of amino acids 114 and 120 of each of SEQ ID NOs: 7-9, respectively. In certain embodiments, CDR1 of any one of SEQ ID NOs: 10-12 comprises or consists essentially of, or yet further consists of amino acid 47 to amino acid 52 of each of SEQ ID NOs: 10-12, respectively. In certain embodiments, CDR2 of any one of SEQ ID NOs: 10-12 comprises or consists essentially of, or yet further consists of amino acid 70 to amino acid 72 of each of SEQ ID NOs: 10-12, respectively. In certain embodiments, CDR3 of any one of SEQ ID NOs: 10-12 comprises or consists essentially of, or yet further consists of amino acids 109 and 110 of each of SEQ ID NOs: 10-12, respectively.

In certain embodiments, CDR1 of any one of SEQ ID NOs: 1-6 comprises or consists essentially of, or yet further consists of amino acid 47 to amino acid 59 of each of SEQ ID NOs: 1-6, respectively. In certain embodiments, CDR2 of any one of SEQ ID NOs: 1-6 comprises or consists essentially of, or yet further consists of amino acid 74 to amino acid 83 of each of SEQ ID NOs: 1-6, respectively. In certain embodiments, CDR1 of any one of SEQ ID NOs: 7-9 comprises or consists essentially of, or yet further consists of amino acid 44 to amino acid 59 of each of SEQ ID NOs: 7-9, respectively. In certain embodiments, CDR2 of any one of SEQ ID NOs: 7-9 comprises or consists essentially of, or yet further consists of amino acid 74 to amino acid 81 of each of SEQ ID NOs: 7-9, respectively. In certain embodiments, CDR3 of any one of SEQ ID NOs: 7-9 comprises or consists essentially of, or yet further consists of amino acids 114 and 122 of each of SEQ ID NOs: 7-9, respectively. In certain embodiments, CDR1 of any one of SEQ ID NOs: 10-12 comprises or consists essentially of, or yet further consists of amino acid 44 to amino acid 54 of each of SEQ ID NOs: 10-12, respectively. In certain embodiments, CDR2 of any one of SEQ ID NOs: 10-12 comprises or consists essentially of, or yet further consists of amino acid 79 to amino acid 76 of each of SEQ ID NOs: 10-12, respectively. In certain embodiments, CDR3 of any one of SEQ ID NOs: 10-12 comprises or consists essentially of, or yet further consists of amino acids 109 and 116 of each of SEQ ID NOs: 10-12, respectively.

In one aspect, provided is one or more of variable region(s) of an antibody or an antigen-binding fragment thereof as disclosed herein, and/or one or more of equivalent(s) of the variable regions(s). In a further embodiment, provided is an antibody, an antigen-binding fragment thereof, or an equivalent of each thereof, comprising, or alternatively consisting essentially of, or yet further consisting of any one or any two or more of: the variable regions as disclosed herein and/or one or more of equivalent(s) of the variable regions(s). Additionally or alternatively, the one or more of variable region(s) specifically binds to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In certain embodiments, the variable region is selected from the following: amino acid (aa) 25 to aa 144 of SEQ ID NOs: 1-6, 13, 24 and 26; aa 21 to aa 132 of SEQ ID NOs: 7-9, 14, and 25; aa 21 to aa 126 of SEQ ID NOs: 10-12 or 27; amino acid 24 to amino acid 144 of SEQ ID NOs: 1-6, 13, 24 and 26; amino acid 20 to amino acid 132 of SEQ ID NOs: 7-12, 14, 25 and 27; amino acid 20 to amino acid 126 of SEQ ID NOs: 7-12, 14, 25 and 27.

In a further embodiment, the variable region or an equivalent thereof is a variable region as disclosed herein further comprising an additional 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids at its amino terminus, or carboxyl terminus or both in the corresponding sequence provided herein with a SEQ ID NO. Additionally or alternatively, the variable region or an equivalent thereof is a variable region as disclosed herein having 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids truncated at its amino terminus, or carboxyl terminus or both in the corresponding sequence provided herein with a SEQ ID NO. For example, of SEQ ID NO: 1 may be amino acid 50 to amino acid 57 of SEQ ID NO: 1. However, the variable region or an equivalent thereof relating to the variable region consisting of amino acid 24 to amino acid 144 of SEQ ID NO: 1 can also start from amino acid 16, or 17, or 18, or 19, or 20, or 21, or 22, or 23, or 24, or 25, or 26, or 27, or 28, or 29, or 30, or 31, or 32 of SED ID NO: 1. Further, the variable region or an equivalent thereof relating to the variable region consisting of amino acid 24 to amino acid 144 of SEQ ID NO: 1 can end at amino acid 136, or 137, or 138, or 139, or 140, or 141, or 142, or 143, or 144, or 145, or 146, or 147, or 148, or 149, or 150, or 151, or 152 of SEQ ID NO: 1 with proviso that the variable region ends after its start. Additionally or alternatively, the variable region is about 90 amino acids long to about 200 amino acids long, for example, about 100 amino acid long, or alternatively about 110 amino acid long, or alternatively about 120 amino acid long, or alternatively about 130 amino acid long, or alternatively about 140 amino acid long, or alternatively about 150 amino acid long, or alternatively about 160 amino acid long, or alternatively about 170 amino acid long, or alternatively about 180 amino acid long, or alternatively about 190 amino acid long, or alternatively about 200 amino acid long.

Additionally or alternatively, the equivalent to an antibody or an antigen-binding fragment thereof comprises one or more of (for example but not limited to 1, 2, 3, 4, 5, 6, 7, 8, 9, 10, 11, 12, 13, 14, 15, 16, 17, 18, 19, 20, 21, 22, 23, 24 or 25) amino acid differences compared to the antibody or antigen-binding fragment thereof in the regions other than the variable domain(s) (referred to herein as non-VH regions). Such non-VH regions include, but are not limited to, a constant region, an Fc region, a pFc′ region, a constant heavy chain (CH) domain (such as CH1, CH2, CH3 or CH4), a constant light chain (CL) domain, or a hinge region. It would be understood by one of skill in the art that an antibody, an antigen-binding fragment thereof, or an equivalent of each thereof as disclosed herein, may be further modified in the non-VH regions (such as for increasing the assembly of a heavy chain with a light chain, conjugating directly or indirectly to a detectable or purification marker or a drug, increasing or decreasing activation of complement, enhancing or reducing antibody-dependent cellular cytotoxicity (ADCC), or increasing or decreasing activation and recruitment of an immune cell), providing a further equivalent.

In certain embodiments, the equivalent further comprises up to 50, or alternatively up to 30, or alternatively up to 25, or alternatively up to 20, or alternatively up to 15, or alternatively up to 10, or alternatively up to 5 random amino acids on either the amino or carboxyl termini or on both. In certain embodiments, the equivalent to an amino acid sequence comprises or consists essentially of, or yet further consists of the amino acid sequence truncated at the amino or carboxyl termini or both, for example, by 1, 2, 3, 4, 5, 6, 7, 8, 9, 10, 11, 12, 13, 14, 15, 20 or 25 amino acids. Such addition or truncation may not change the three-dimensional arrangement of the CDR(s) and/or the three-dimensional arrangement of the antibody, antigen-binding fragment thereof, CDR thereof, or the CDR-containing polypeptide.

In some aspects, the antibody or antigen-binding fragment thereof comprises, or alternatively consists essentially of, or yet further consists of a light chain and a heavy chain, further optionally wherein the light chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 and comprises one or two or three CDR(s) of the antibody or antigen-binding fragment thereof, and further optionally wherein the heavy chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 1-6, 13, 24 or 26 and comprises one or two or three CDR(s) of the antibody or antigen-binding fragment thereof.

In one aspect, the CDRH1 comprises, or consists essentially of, or yet further consists of GFTFRTYA (aa 50 to aa 57 of SEQ ID NO: 1 or 2 or 3 or 24). In another aspect, the CDRH1 comprises, or consists essentially of, or yet further consists of aASGFTFRTYAMS (aa 47 to aa 59 of SEQ ID NO: 24), wherein the small letter a is A (aa 47 to aa 59 of SEQ ID NO: 1 or 2) or wherein the small letter a is K (aa 47 to aa 59 of SEQ ID NO: 3).

Additionally or alternatively, in one aspect, the CDRH2 comprises, or consists essentially of, or yet further consists of IGSDRRHT (aa 75 to aa 82 of SEQ ID NO: 1 or 2 or 3 or 24). In another aspect, the CDRH2 comprises, or consists essentially of, or yet further consists of IGSDRRHTY (aa 75 to aa 83 of SEQ ID NO: 1 or 2 or 3 or 24). In yet another aspect, the CDRH2 comprises, or consists essentially of, or yet further consists of TIGSDRRHTY (aa 74 to aa 83 of SEQ ID NO: 1 or 2 or 3 or 24). In a further aspect, the CDRH2 comprises, or consists essentially of, or yet further consists of WVATIGSDRRHTYYP (aa 71 to aa 85 of SEQ ID NO: 1 or 2 or 3 or 24).

Additionally or alternatively, the CDRL1 comprises, or consists essentially of, or yet further consists of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9).

Additionally or alternatively, in one aspect, the CDRL2 comprises, or consists essentially of, or yet further consists of LVSKlDS (aa 75 to aa 81 of SEQ ID NO: 25), wherein the smaller letter l is L (aa 75 to aa 81 of SEQ ID NO: 7 or 9) or wherein the smaller letter l is R (aa 75 to aa 81 of SEQ ID NO: 8). In another aspect, the CDRL2 comprises, or consists essentially of, or yet further consists of YLVSKlDS (aa 74 to aa 81 of SEQ ID NO: 25), wherein the smaller letter l is L (aa 74 to aa 81 of SEQ ID NO: 7 or 9) or wherein the smaller letter l is R (aa 74 to aa 81 of SEQ ID NO: 8). In yet another aspect, the CDRL2 comprises, or consists essentially of, or yet further consists of LVSKlDSG (aa 75 to aa 82 of SEQ ID NO: 25), wherein the smaller letter l is L (aa 75 to aa 82 of SEQ ID NO: 7 or 9) or wherein the smaller letter l is R (aa 75 to aa 82 of SEQ ID NO: 8). In a further aspect, the CDRL2 comprises, or consists essentially of, or yet further consists of YLVSKlDSGV (aa 74 to aa 83 of SEQ ID NO: 25), wherein the smaller letter l is L (aa 74 to aa 83 of SEQ ID NO: 7 or 9) or wherein the smaller letter l is R (aa 74 to aa 83 of SEQ ID NO: 8). In yet a further aspect, the CDRL2 comprises, or consists essentially of, or yet further consists of RLIYLVSKlDSGVPD (aa 71 to aa 85 of SEQ ID NO: 25), wherein the smaller letter l is L (aa 71 to aa 85 of SEQ ID NO: 7 or 9) or wherein the smaller letter l is R (aa 71 to aa 85 of SEQ ID NO: 8).

Additionally or alternatively, in one aspect, the CDRL3 comprises, or consists essentially of, or yet further consists of WQGTHFPY (aa 114 to aa 121 of SEQ ID NO: 7 or 8 or 9 or 25). In another aspect, the CDRL3 comprises, or consists essentially of, or yet further consists of WQGTHFPYT (aa 114 to aa 122 of SEQ ID NO: 7 or 8 or 9 or 25).

In some aspects, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In some aspects, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTYA (aa 50 to aa 57 of SEQ ID NO: 1 or 2 or 3 or 24);
(ii) a heavy chain complementarity-determining region 2 (CDRH2) comprising, or alternatively consisting essentially of, or yet further consisting of IGSDRRHT (aa 75 to aa 82 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In one aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of aASGFTFRTYAMS (aa 47 to aa 59 of SEQ ID NO: 24), wherein the small letter a is A (aa 47 to aa 59 of SEQ ID NO: 1 or 2) or wherein the small letter a is K (aa 47 to aa 59 of SEQ ID NO: 3);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9);
(v) a light chain complementarity-determining region 2 (CDRL2) comprising, or alternatively consisting essentially of, or yet further consisting of YLVSKlDS (aa 74 to aa 81 of SEQ ID NO: 25), wherein the smaller letter l is L (aa 74 to aa 81 of SEQ ID NO: 7 or 9) or wherein the smaller letter l is R (aa 74 to aa 81 of SEQ ID NO: 8); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of YLVSKLDS (aa 74 to aa 81 of SEQ ID NO: 7); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In yet another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of YLVSKRDS (aa 74 to aa 81 of SEQ ID NO: 8); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In one aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of YLVSKLDS (aa 74 to aa 81 of SEQ ID NO: 9); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of YLVSKLDS (aa 74 to aa 81 of SEQ ID NO: 7); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In yet another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of YLVSKRDS (aa 74 to aa 81 of SEQ ID NO: 8); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In one aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of YLVSKLDS (aa 74 to aa 81 of SEQ ID NO: 9); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9);
(v) a light chain complementarity-determining region 2 (CDRL2) comprising, or alternatively consisting essentially of, or yet further consisting of LVSKlDS (aa 75 to aa 81 of SEQ ID NO: 25), wherein the smaller letter l is L (aa 75 to aa 81 of SEQ ID NO: 7 or 9) or wherein the smaller letter l is R (aa 75 to aa 81 of SEQ ID NO: 8); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In yet another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of LVSKLDS (aa 75 to aa 81 of SEQ ID NO: 7); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In one aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of LVSKRDS (aa 75 to aa 81 of SEQ ID NO: 8); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of LVSKLDS (aa 75 to aa 81 of SEQ ID NO: 9); and
(vi) a light chain complementarity-determining region 3 (CDRL3) comprising, or alternatively consisting essentially of, or yet further consisting of WQGTHFPYT (aa 114 to aa 122 of SEQ ID NO: 7 or 8 or 9 or 25).

In yet another aspect, the antibody or antigen-binding fragment thereof comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFRTYA (aa 50 to aa 57 of SEQ ID NO: 1 or 2 or 3 or 24);
(ii) a heavy chain complementarity-determining region 2 (CDRH2) comprising, or alternatively consisting essentially of, or yet further consisting of IGSDRRHT (aa 75 to aa 82 of SEQ ID NO: 1 or 2 or 3 or 24);
(iii) a heavy chain complementarity-determining region 3 (CDRH3) comprising, or alternatively consisting essentially of, or yet further consisting of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of WQGTHFP (aa 114 to aa 120 of SEQ ID NO: 7 or 8 or 9 or 25).

Additionally provided is a composition or combination comprising or alternatively consisting essentially of, or yet further consisting of:
(a) a high mobility group box 1 protein (HMGB1) polypeptide optionally comprising one or more mutations (i.e., a mutant HMGB1 (mHMGB1) polypeptide), or a fragment thereof optionally comprising, or consisting essentially of, or consisting of a B box or an A box or both (e.g., an AB box) of the HMGB1 polypeptide, optionally wherein the HMGB1 polypeptide or a fragment thereof is isolated or engineered or both; and
(b) an anti-DNABII antibody or an antigen-binding fragment thereof comprising, or alternatively consisting essentially of, or yet further consisting of one or two or three or four or five or all six of the following (i) to (vi):
   (i) a heavy chain complementarity-determining region 1 (CDRH1) comprising, or alternatively consisting essentially of, or yet further consisting of GFTFSRY (aa 50 to aa 56 of SEQ ID NO: 4 or 5 or 6 or 26);
   (ii) a heavy chain complementarity-determining region 2 (CDRH2) comprising, or alternatively consisting essentially of, or yet further consisting of SSGGSY (aa 76 to aa 81 of SEQ ID NO: 4 or 5 or 6 or 26);
   (iii) a heavy chain complementarity-determining region 3 (CDRH3) comprising, or alternatively consisting essentially of, or yet further consisting of ER (aa 121 to aa 122 of SEQ ID NO: 4 or 5 or 6 or 26);
   (iv) a light chain complementarity-determining region 1 (CDRL1) comprising, or alternatively consisting essentially of, or yet further consisting of QDISNY (aa 47 to aa 52 of SEQ ID NO: 10 or 11 or 12 or 27);
   (v) a light chain complementarity-determining region 2 (CDRL2) comprising, or alternatively consisting essentially of, or yet further consisting of YTS (aa 70 to aa 72 of SEQ ID NO: 10 or 11 or 12 or 27); and
   (vi) a light chain complementarity-determining region 3 (CDRL3) comprising, or alternatively consisting essentially of, or yet further consisting of QQ (aa 109 to aa 110 of SEQ ID NO: 10 or 11 or 12 or 27),

In one aspect, the CDRH1 comprises, or consists essentially of, or yet further consists of GFTFSRYG (aa 50 to aa 57 of SEQ ID NO: 4 or 5 or 6 or 26). In another aspect, the CDRH1 comprises, or consists essentially of, or yet further consists of aASGFTFSRYGMS (aa 47 to aa 59 of SEQ ID NO:26), wherein the small letter a is A (aa 47 to aa 59 of SEQ ID NO: 4 or 5) or wherein the small letter a is T (aa 47 to aa 59 of SEQ ID NO: 6).

Additionally or alternatively, in one aspect, the CDRH2 comprises, or consists essentially of, or yet further consists of ISSGGSYT (aa 75 to aa 82 of SEQ ID NO: 4 or 5 or 6 or 26). In another aspect, the CDRH2 comprises, or consists essentially of, or yet further consists of TISSGGSYTY (aa 74 to aa 83 of SEQ ID NO: 4 or 5 or 6 or 26).

Additionally or alternatively, the CDRH3 comprises, or consists essentially of, or yet further consists of ERHGGDGYWYFDV (aa 121 to aa 133 of SEQ ID NO: 4 or 5 or 6 or 26).

Additionally or alternatively, the CDRL1 comprises, or consists essentially of, or yet further consists of RASQDISNYLN (aa 44 to aa 54 of SEQ ID NO: 10 or 11 or 12 or 27).

Additionally or alternatively, in one aspect, the CDRL2 comprises, or consists essentially of, or yet further consists of YTSRLHS (aa 70 to aa 76 of SEQ ID NO: 10 or 11 or 12 or 27). In another aspect, the CDRL2 comprises, or consists essentially of, or yet further consists of YYTSRLHS (aa 69 to aa 76 of SEQ ID NO: 10 or 11 or 12 or 27).

Additionally or alternatively, the CDRL3 comprises, or consists essentially of, or yet further consists of QQGNPLRT (aa 109 to aa 116 of SEQ ID NO: 10 or 11 or 12 or 27).

In one aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFSRY (aa 50 to aa 56 of SEQ ID NO: 4 or 5 or 6 or 26);
(ii) a heavy chain complementarity-determining region 2 (CDRH2) comprising, or alternatively consisting essentially of, or yet further consisting of SSGGSY (aa 76 to aa 81 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In another aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFSRYG (aa 50 to aa 57 of SEQ ID NO: 4 or 5 or 6 or 26);
(ii) a heavy chain complementarity-determining region 2 (CDRH2) comprising, or alternatively consisting essentially of, or yet further consisting of ISSGGSYT (aa 75 to aa 82 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In yet another aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of aASGFTFSRYGMS (aa 47 to aa 59 of SEQ ID NO:26), wherein the small letter a is A (aa 47 to aa 59 of SEQ ID NO: 4 or 5) or wherein the small letter a is T (aa 47 to aa 59 of SEQ ID NO: 6);
(ii) a heavy chain complementarity-determining region 2 (CDRH2) comprising, or alternatively consisting essentially of, or yet further consisting of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In one aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of AASGFTFSRYGMS (aa 47 to aa 59 of SEQ ID NO: 4 or 5);
(ii) a heavy chain complementarity-determining region 2 (CDRH2) comprising, or alternatively consisting essentially of, or yet further consisting of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In another aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of TASGFTFSRYGMS (aa 47 to aa 59 of SEQ ID NO: 6);
(ii) a heavy chain complementarity-determining region 2 (CDRH2) comprising, or alternatively consisting essentially of, or yet further consisting of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In yet another aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFSRY (aa 50 to aa 56 of SEQ ID NO: 4 or 5 or 6 or 26);
(ii) a heavy chain complementarity-determining region 2 (CDRH2) comprising, or alternatively consisting essentially of, or yet further consisting of SSGGSY (aa 76 to aa 81 of SEQ ID NO: 4 or 5 or 6 or 26);
(iii) a heavy chain complementarity-determining region 3 (CDRH3) comprising, or alternatively consisting essentially of, or yet further consisting of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of YTSRLHS (aa 70 to aa 76 of SEQ ID NO: 10 or 11 or 12 or 27); and
(vi) a light chain complementarity-determining region 3 (CDRL3) comprising, or alternatively consisting essentially of, or yet further consisting of QQGNPLRT (aa 109 to aa 116 of SEQ ID NO: 10 or 11 or 12 or 27).

In one aspect, the composition or combination comprises, or alternatively consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of GFTFSRYG (aa 50 to aa 57 of SEQ ID NO: 4 or 5 or 6 or 26);
(ii) a heavy chain complementarity-determining region 2 (CDRH2) comprising, or alternatively consisting essentially of, or yet further consisting of ISSGGSYT (aa 75 to aa 82 of SEQ ID NO: 4 or 5 or 6 or 26);
(iii) a heavy chain complementarity-determining region 3 (CDRH3) comprising, or alternatively consisting essentially of, or yet further consisting of a sequence ER (aa 121 to aa 122 of SEQ ID NO: 4 or 5 or 6 or 26);
(iv) a light chain complementarity-determining region 1 (CDRL1) comprising, or alternatively consisting essentially of, or yet further consisting of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of QQ (aa 109 to aa 110 of SEQ ID NO: 10 or 11 or 12 or 27).

In certain aspects, the antibody or antigen-binding fragment thereof comprises, or alternatively consists essentially of, or yet further consists of:
(i) CDRs 1-3 of a sequence selected from the group of: SEQ ID NOs: 1-6, 13, 24 or 26; and/or
(ii) CDRs 1-3 of a sequence selected from the group of: SEQ ID NOs: 7-12, 14, 25 or 27.

In some aspects, the antibody is selected from the group of: a bispecific antibody, a trispecific antibody, a tetraspecific antibody, or a pentaspecific antibody.

In some aspects, the antibody is selected from the group of an IgA, an IgD, an IgE, an IgG, or an IgM antibody.

In some aspects, the antibody or antigen-binding fragment thereof further comprises a constant region selected from the group of: an IgA constant region, an IgD constant region, an IgE constant region, an IgG constant region or an IgM constant region. In a further aspect, the constant region is an IgG1 constant region. In yet a further aspect, the antibody or antigen-binding fragment thereof further comprises a heavy chain (HC) constant region of SEQ ID NOs: 1-6, 13, 24 or 26, and/or a light chain (LC) constant region of SEQ ID NOs: 7-12, 14, 25 or 27. In one aspect, the HC constant region comprises, or alternatively consists essentially of, or yet further consists of a constant region of SEQ ID NOs: 1-6, 13, 24 or 26 (optionally a sequence selected from aa 145 to aa 473 of SEQ ID NOs: 1-6, 13, 24 or 26). In another aspect, the HC constant regions comprises, or alternatively consists essentially of, or yet further consists of a constant region of any one of SEQ ID NOs: 15-22. In one aspect, the LC constant region comprises, or alternatively consists essentially of, or yet further consists of a constant region of SEQ ID NOs: 7-12 or 27 (optionally a sequence selected from aa 133 to aa 239 of SEQ ID NOs: 7-9, 14 or 25, and/or aa 127 to aa 233 of SEQ ID NOs: 10-12 or 27). In another aspect, the LC constant regions comprises, or alternatively consists essentially of, or yet further consists of a constant region of SEQ ID NOs: 23.

In some aspects, the antibody or antigen-binding fragment thereof competes for binding to a tip chimeric peptide IhfA5-mIhfB4_{NTHI}. In one aspect, the tip chimeric peptide IhfA5-mIhfB4_{NTHI} comprises, or alternatively consists essentially of, or yet further consists of RPGRNPX1TGDVVPVSARRVV-X-FSLHHRQPRLGRNPX1TGDSV (SEQ ID NO: 38), wherein “X” is an optional amino acid linker sequence, optionally comprising, or consisting essentially of, or yet further consisting of between 1 to 20 amino acids, and wherein “X1” is any amino acid or alternatively “X1” is selected from the amino acids Q, R, K, S, or T. In a further aspect, the tip chimeric peptide IhfA5-mIhfB4_{NTHI} comprises, or alternatively consists essentially of, or yet further consists of RPGRNPKTGDVVPVSARRVV-X-FSLHHRQPRLGRNPKTGDSV (SEQ ID NO: 39), wherein “X” is an optional amino acid linker sequence, optionally comprising, or consisting essentially of, or yet further consisting of between 1 to 20 amino acids. In yet a further aspect, the tip chimeric peptide IhfA5-mIhfB4_{NTHI} comprises, or alternatively consists essentially of, or yet further consists of

(SEQ ID NO: 40)
RPGRNPKTGDVVPVSARRVVGPSLFSLHHRQPRLGRNPKTGDSV.

In some aspects, the antibody or antigen-binding fragment thereof competes for binding to a tail chimeric peptide IhfA3-IhfB2_{NTHI}. In one aspect, the tail chimeric peptide IhfA3-IhfB2_{NTHI} comprises, or alternatively consists essentially of, or yet further consists of FLEEIRLSLESGQDVKLSGF-X-TLSAKEIENMVKDILEFISQ (SEQ ID NO: 41), wherein “X” is an optional amino acid linker sequence, optionally comprising, or consisting essentially of, or yet further consisting of between 1 to 20 amino acids. In a further aspect, the tail-chimeric peptide IhfA3-IhfB2_{NTHI} comprises, or consists essentially of, or yet further consists of FLEEIRLSLESGQDVKLSGFGPSLTLSAKEIENMVKDILEFISQ (SEQ ID NO: 50).

In some aspects, the amino acid linker is selected from the group of: GGSGGS (SEQ ID NO: 42), GPSLKL (SEQ ID NO: 43), GGG (SEQ ID NO: 44), GPSL (SEQ ID NO: 45), GPS (SEQ ID NO: 46), PSLK (SEQ ID NO: 47), GPSLK (SEQ ID NO: 48), or SLKL (SEQ ID NO: 49).

In some aspects, the antibody is selected from the group of a polyclonal, a monoclonal or a humanized antibody.

In some aspects, the fragment is an antigen-binding fragment. In a further aspect, the antigen-binding fragment is selected from the group of Fab, F(ab′)2, Fab′, scFv, or Fv.

In some aspects, either or both of the HMGB polypeptide or a fragment thereof, or the anti-DNABII antibody or an antigen-binding fragment thereof further comprises a detectable marker or a purification marker.

In certain embodiments, an antibody or an antigen-binding fragment thereof comprises a signal peptide at the amino terminus of VH and/or the amino terminus of VL. In one embodiment, the VH signal peptide is different to the VL signal peptide. In another embodiment, the VH signal peptide is the same compared to the VL signal peptide. In a further embodiment, the signal peptide comprises or consists essentially of, or yet further consists of an amino acid sequence of amino acids 1-24 of SEQ ID NO: 1. In yet a further embodiment, the signal peptide comprises or consists essentially of, or yet further consists of an amino acid sequence of amino acids 1-20 of SEQ ID NO: 7. In certain embodiments, the equivalent to an antibody or an antigen-binding fragment thereof comprises a signal peptide which is different from the signal peptide(s) of the antibody with the proviso that the signal peptide of the equivalent directs VH and/or VL to the same cellular location as the signal peptide(s) of the antibody.

In certain embodiments, the equivalent to an antibody or an antigen-binding fragment thereof retains at least 50% (such as at least 60%, at least 70%, at least 80%, at least 90%, at least 95%, or at least 99%) of or improves one or more of functional activities of the antibody or antigen-binding fragment. Such functional activities include but are not limited to binding specificity, binding avidity and/or affinity to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}), preventing the formation of a biofilm in vivo or in vitro, or disrupting a biofilm in vivo or in vitro. Methods of quantifying such functional activities are illustrated in the examples.

In a further aspect, the antibody or antigen-binding fragment thereof competes for binding to an epitope with an antibody or an antigen-binding fragment thereof as disclosed herein. The antibody or antigen-binding fragment thereof may be a polyclonal, a monoclonal and/or a humanized antibody or antigen-binding fragment thereof.

In one aspect, the antibody is a bispecific antibody or a trispecific, tetraspecific or pentaspecific antibody. In a further aspect, the antibody is an IgA, an IgD, an IgE, an IgG or an IgM antibody. In another aspect, the antibodies further comprise a constant region selected from an IgA, an IgD, an IgE, an IgG or an IgM constant region. In a specific aspect, the constant region is an IgG1 constant region. In another aspect, the antibodies compete for binding to an epitope with an antibody as disclosed herein. These can be identified using conventional techniques, e.g. a competitive ELISA.

The antibodies as disclosed herein can be polyclonal, monoclonal or humanized. In one aspect, the antibodies bind the “tip” region of a DNABII polypeptide, e.g., HU or IHF (such as IhfA and IhfB). In a further aspect, the antibodies bind the “tail” region of a DNABII polypeptide, e.g., HU or IHF (such as IhfA and IhfB). As noted above, this disclosure provides antigen-binding fragments. The antigen-binding fragments are any one of Fab, F(ab′)2, Fab′, scFv, or Fv, that can be prepared using conventional techniques known to those of skill in the art. In some of the aspects of the antibodies provided herein, the antibody or antigen-binding fragment is soluble Fab. In another aspect, this disclosure provides a Fab fragment of the antibody as disclosed herein, wherein the antibody or antigen-binding fragment thereof specifically binds the tip region of a DNABII peptide (including but not limited to: a tip region of IHF or HU, a tip region of IHFA or IHFB, and/or the tip-chimeric peptide IhfA5-mIhfB4_{NTHI}). In one aspect of the disclosure, the DNABII is an IHF or an HU peptide. In a specific aspect, the DNABII is an IHF peptide.

As noted above, this disclosure provides equivalents to antibodies and antigen-binding fragments. An equivalent can comprise a polypeptide having at least 80% amino acid identity to polypeptide, or a polypeptide that is encoded by a polynucleotide that hybridizes under conditions of high stringency to the complement of a polynucleotide encoding the polypeptide.

In some of the aspects of the antibodies provided herein, the antibody, antigen-binding fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) at a half maximal effective concentration (EC₅₀) of less than 500 ng/mL, or alternatively less than 250 ng/mL, or alternatively less than 200 ng/mL, or alternatively less than 150 ng/mL, or alternatively less than 100 ng/mL, or alternatively less than 90 ng/mL, or alternatively less than 80 ng/mL, or alternatively less than 70 ng/mL, or alternatively less than 65 ng/mL, or alternatively less than 60 ng/mL, or alternatively less than 55 ng/mL, or alternatively less than 50 ng/mL, or alternatively less than 45 ng/mL, or alternatively less than 40 ng/mL, or alternatively less than 35 ng/mL, or alternatively less than 30 ng/mL. In a further embodiment, such EC₅₀ is determined using the ELISA methods shown in the Examples.

In some of the aspects of the antibodies provided herein, the antibody, antigen-binding fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with an equilibrium constant K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In one embodiment, the antibody, antigen-binding fragment thereof, polypeptide or CDR binds a DNABII protein with a K_{D} of less than 1000 nM, or alternatively less than 900 nM, or alternatively less than 800 nM, or alternatively less than 700 nM, or alternatively less than 600 nM, or alternatively less than 500 nM, or alternatively less than 400 nM, or alternatively less than 300 nM, or alternatively less than 200 nM, or alternatively less than 100 nM, or alternatively less than 90 nM, or alternatively less than 80 nM, or alternatively less than 70 nM, or alternatively less than 60 nM, or alternatively less than 50 nM, or alternatively less than 40 nM, or alternatively less than 30 nM, or alternatively less than 20 nM, or alternatively less than 15 nM, or alternatively less than 10 nM, or alternatively less than 9 nM, or alternatively less than 8 nM, or alternatively less than 7 nM, or alternatively less than 6 nM, or alternatively less than 5 nM, or alternatively less than 4 nM, or alternatively less than 3 nM, or alternatively less than 2 nM, or alternatively less than 1 nM. In one embodiment, such K_{D} is determined using the surface plasmon resonance (SPR) methods shown in the Examples. In some of the aspects of the antibodies provided herein, the antigen-binding site specifically binds to a DNABII protein.

In some of the aspects of the antibodies provided herein, the antibody, antigen-binding fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with a K_{off} of less than 1.0E-02 s⁻¹, or alternatively less than 9.0E-03 s⁻¹, or alternatively less than 8.0E-03 s⁻¹, or alternatively less than 7.0E-03 s⁻¹, or alternatively less than 6.0E-03 s⁻¹, or alternatively less than 5.0E-03 s⁻¹, or alternatively less than 4.0E-03 s⁻¹, or alternatively less than 3.0E-03 s⁻¹, or alternatively less than 2.0E-03 s⁻¹ or alternatively less than 1.0E-03 s⁻¹, or alternatively less than 9.0E-04 s⁻¹, or alternatively less than 8.0E-04 s⁻¹, or alternatively less than 7.0E-04 s⁻¹, or alternatively less than 6.0E-04 s⁻¹, or alternatively less than 5.0E-04 s⁻¹, or alternatively less than 4.0E-04 s⁻¹, or alternatively less than 3.0E-04 s⁻¹, or alternatively less than 2.0E-04 s⁻¹ or alternatively less than 1.0E-04 s⁻¹, or alternatively less than 9.0E-05 s⁻¹, or alternatively less than 8.0E-05 s⁻¹, or alternatively less than 7.0E-05 s⁻¹, or alternatively less than 6.0E-05 s⁻¹, or alternatively less than 5.0E-05 s⁻¹, or alternatively less than 4.0E-05 s⁻¹, or alternatively less than 3.0E-05 s⁻¹, or alternatively less than 2.0E-05 s⁻¹ or alternatively less than 1.0E-05 s⁻¹. In one embodiment, such K_{off} is determined using the surface plasmon resonance (SPR) methods shown in the Examples.

In some of the aspects of the antibodies provided herein, the antibody, antigen-binding fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with a Kₒₙ of less than 9.0E-02 M⁻¹ s⁻¹, or alternatively less than 8.0E-02 M⁻¹ s⁻¹, or alternatively less than 7.0E-02 M⁻¹ s⁻¹, or alternatively less than 6.0E-02 M⁻¹ s⁻¹, or alternatively less than 5.0E-02 M⁻¹ s⁻¹, or alternatively less than 4.0E-02 M⁻¹ s⁻¹, or alternatively less than 3.0E-02 M⁻¹ s⁻¹, or alternatively less than 2.0E-02 M⁻¹ s⁻¹ or alternatively less than 1.0E-02 M⁻¹ s⁻¹, or alternatively less than 9.0E-03 M⁻¹ s⁻¹, or alternatively less than 8.0E-03 M⁻¹ s⁻¹, or alternatively less than 7.0E-03 M⁻¹ s⁻¹, or alternatively less than 6.0E-03 M⁻¹ s⁻¹, or alternatively less than 5.0E-03 M⁻¹ s⁻¹, or alternatively less than 4.0E-03 M⁻¹ s⁻¹, or alternatively less than 3.0E-03 M⁻¹ s⁻¹, or alternatively less than 2.0E-03 M⁻¹ s⁻¹ or alternatively less than 1.0E-03 M⁻¹ s⁻¹, or alternatively less than 9.0E-04 M⁻¹ s⁻¹, or alternatively less than 8.0E-04 M⁻¹ s⁻¹, or alternatively less than 7.0E-04 M⁻¹ s⁻¹, or alternatively less than 6.0E-04 M⁻¹ s⁻¹, or alternatively less than 5.0E-04 M⁻¹ s⁻¹, or alternatively less than 4.0E-04 M⁻¹ s⁻¹, or alternatively less than 3.0E-04 M⁻¹ s⁻¹, or alternatively less than 2.0E-04 M⁻¹ s⁻¹ or alternatively less than 1.0E-04 M⁻¹ s⁻¹, or alternatively less than 9.0E-05 M⁻¹ s⁻¹, or alternatively less than 8.0E-05 M⁻¹ s⁻¹, or alternatively less than 7.0E-05 M⁻¹ s⁻¹, or alternatively less than 6.0E-05 M⁻¹ s⁻¹, or alternatively less than 5.0E-05 M⁻¹ s⁻¹, or alternatively less than 4.0E-05 M⁻¹ s⁻¹, or alternatively less than 3.0E-05 M⁻¹ s⁻¹, or alternatively less than 2.0E-05 M⁻¹ s⁻¹ or alternatively less than 1.0E-05 M⁻¹ s⁻¹. In one embodiment, such Kₒₙ is determined using the surface plasmon resonance (SPR) methods shown in the Examples.

In some aspects of this invention, the association constant K_{A} for the IhfA5-mIhfB4_{NTHI} Tip chimeric peptide (in 1/M) is about 3E+05 to about 2E+08. In another aspect, the K_{A} is about 3E+05 to about 1E+08, or alternatively about 2E+05 to about 1E+08, or alternatively about 1E+05 to about 1E+08, or alternatively about 1E+06 to about 1E+08, or alternatively about 1E+07 to about 1E+08, or alternatively about 1E+04 to about 1E+09, alternatively about 1E+05 to about 1E+09, alternatively about 1E+06 to about 1E+09, alternatively about 1E+07 to about 1E+09, alternatively about 1E+08 to about 1E+09, alternatively about 1E+04 to about 1E+09, or alternatively about 1E+03 to about 1E+10.

In another aspect, the dissociation constant K_{D} for the IhfA5-mIhfB4_{NTHI} Tip chimeric peptide (in M) is about 5E-09 to about 3E-06, or alternatively about 1E-09 to about 1E-06, or alternatively about 1E-08 to about 1E-05, or alternatively about 1E-07 to about 1E-05, or alternatively about 1E-06 to about 1E-05, or alternatively about 1E-09 to about 1E-08, or alternatively about 1E-08 to about 1E-07, or alternatively about 1E-9 to about 1E-08, or alternatively about 1E-10 to about 1E-09, or alternatively about 1E-11 to about 1E-10.

In one aspect, the K_{A} for the IhfA3-IhfB2_{NTHI} Tail chimeric peptide (in 1/M) is from about 7E+06 to about 2E+09, or alternatively about 1E+05 to about 1E+08, or alternatively about 1E+06 to about 1E+08, or alternatively about 1E+07 to about 1E+08, or alternatively about 1E+04 to about 1E+09, alternatively about 1E+05 to about 1E+09, alternatively about 1E+06 to about 1E+09, alternatively about 1E+07 to about 1E+09, alternatively about 1E+08 to about 1E+09, alternatively about 1E+04 to about 1E+09, or alternatively about 1E+03 to about 1E+10, or alternatively about 1E+03 to about 1E+11, or alternatively about 1E+03 to about 1E+12, or alternatively about 1E+09 to about 1E+10, or alternatively about 1E+10 to about 1E+11, or alternatively about 1E+11 to about 1E+12.

In another aspect, the K_{D} for the IhfA3-IhfB2_{NTHI} Tail chimeric peptide (in M) is about 6E-10 to about 2E-07, or alternatively about 1E-09 to about 1E-06, or alternatively about 1E-08 to about 1E-05, or alternatively about 1E-07 to about 1E-05, or alternatively about 1E-06 to about 1E-05, or alternatively about 1E-09 to about 1E-08, or alternatively about 1E-08 to about 1E-07, or alternatively about 1E-9 to about 1E-08, or alternatively about 1E-10 to about 1E-09, or alternatively about 1E-11 to about 1E-10, or alternatively about 1E-11 to about 1E-12.

In some of the aspects of the antibodies provided herein, the antibody, antigen-binding fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces biomass of a biofilm in vitro by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 95%. In some of the aspects of the antibodies provided herein, the antibody, antigen-binding fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces biomass of a biofilm in vitro by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such biomass change is determined using the methods shown in Example 3 or 4.

In some of the aspects of the antibodies provided herein, the antibody, antigen-binding fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces bacterial load in a subject by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In some of the aspects of the antibodies provided herein, the antibody, antigen-binding fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces bacterial load in a subject by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such change in the bacterial load is determined using the methods shown in the Examples.

In some of the aspects of the antibodies provided herein, the antibody, antigen-binding fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces middle ear occlusion in a subject having otitis media (OM) by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In some of the aspects of the antibodies provided herein, the antibody, antigen-binding fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces middle ear occlusion in a subject having otitis media (OM) by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such change in middle ear occlusion is determined using the methods shown in the Examples in an experimental OM model.

In some of the aspects of the antibodies provided herein, the antibody, antigen-binding fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces the relative mucosal biofilm score and/or biomass score in a subject having a mucosal biofilm (such as having OM) by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In one embodiment, the antibody, antigen-binding fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces the relative mucosal biofilm score and/or biomass score in a subject having a mucosal biofilm (such as having OM) by at least about 0.5, or alternatively at least about 1, or alternatively at least about 1.5, or alternatively at least about 2, or alternatively at least about 2.5, or alternatively at least about 3, or alternatively at least about 3.5, or alternatively at least about 4. In some of the aspects of the antibodies provided herein, the antibody, antigen-binding fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces the relative mucosal biofilm score and/or biomass score in a subject having a mucosal biofilm (such as having OM) by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In some of the aspects of the antibodies provided herein, the antibody, antigen-binding fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces the relative mucosal biofilm score and/or biomass score in a subject having a mucosal biofilm (such as having OM) by less than about 0.1, or alternatively less than about 0.2, or alternatively less than about 0.3, or alternatively less than about 0.4, or alternatively less than about 0.5, or alternatively less than about 0.6, or alternatively less than about 0.7, or alternatively less than about 0.8, or alternatively less than about 0.9, or alternatively less than about 1, or alternatively less than about 1.5, or alternatively less than about 2, or alternatively less than about 2.5. In one embodiment, such score is determined using the methods shown in the Examples.

In certain embodiments, the DNABII protein is an HU or an THF. In a further embodiment, the DNABII protein is an IhfA, an IhfB or both. In yet a further embodiment, the antibody, antigen-binding fragment thereof, polypeptide or CDR binds the tip region or the tail region of the DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In one embodiment, the antibody, antigen-binding fragment thereof, polypeptide or CDR binds the IhfA5-mIhfB4_{NTHI} Tip chimeric peptide. In another embodiment, the antibody, antigen-binding fragment thereof, polypeptide or CDR binds the IhfA3-IhfB2_{NTHI} Tail chimeric peptide.

In some of the aspects of the antibodies provided herein, the antibody or antigen-binding fragment thereof is soluble Fab.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In some of the aspects of the antibodies provided herein, the antibody is a full-length antibody.

In some of the aspects of the antibodies provided herein, the antibody or antigen-binding fragment thereof is chimeric or humanized.

In some of the aspects of the antibodies provided herein, the antibody or antigen-binding fragment thereof comprises an Fe domain. In some of the aspects of the antibodies provided herein, the antibody is a non-human animal such as a rat, sheep, bovine, canine, feline or rabbit antibody. In some of the aspects of the antibodies provided herein, the antibody is a human or humanized antibody or is non-immunogenic in a human.

In some of the aspects of the antibodies provided herein, the antibody or antigen-binding fragment thereof comprises a human antibody framework region. Examples of framework regions that can be fused to the LC and HC sequences are known in the art, examples of such are provided in SEQ ID NOs: 15-23, or equivalents of each thereof.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.
1) Amino acids with basic side chains: lysine, arginine, histidine.
2) Amino acids with acidic side chains: aspartic acid, glutamic acid
3) Amino acids with uncharged polar side chains: asparagine, glutamine, serine, threonine, tyrosine.
4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind a DNABII protein with similar specificity and sensitivity profiles as the disclosed antibodies. This may be tested by way of the binding assays, such as ELISA or SPR.

In a further aspect, the antibodies are characterized by being both immunodominant and immunoprotective, as determined using appropriate assays and screens.

In some aspects, the antibody or antigen-binding fragment thereof is modified. In another aspect, the antibodies can be modified by conventional techniques, that may in one aspect increase the half-life of the antibody, e.g., PEGylation, a PEG mimetic, polysialyation, HESylation or glycosylation.

The antibodies and antigen-binding fragments can further comprise a detectable marker or a purification marker.

### Antibodies and Derivatives Thereof

In some embodiments, the antibody or an antigen-binding fragment thereof binds and/or specifically recognizes and binds an isolated polypeptide for use in the methods disclosed herein. The antibody can be any of the various antibodies described herein, non-limiting, examples of such include a polyclonal antibody, a monoclonal antibody, a chimeric antibody, a human antibody, a veneered antibody, a diabody, a humanized antibody, an antibody derivative, a recombinant humanized antibody, or an equivalent (such as a derivative) or fragment of each thereof. In one aspect, the fragment comprises, or alternatively consists essentially of, or yet further consists of the CDR of the antibody. In one aspect, the antibody is detectably labeled or further comprises a detectable label conjugated to it.

Also provided is a hybridoma cell line that produces a monoclonal antibody disclosed herein. Compositions comprising, or alternatively consisting essentially of, or yet further, consisting of one or more of the above embodiments are further provided herein. Further provided are polynucleotides that encode the amino acid sequence of the antibodies and fragments as well as methods to produce recombinantly or chemically synthesize the antibody polypeptides and fragments thereof. The antibody polypeptides can be produced in a eukaryotic or prokaryotic cell, or by other methods known in the art and described herein.

Examples of CDR sequences include without limitation comprise, consist essentially of, or yet further consist of, the following: the heavy chain variable region of the antibody or a fragment thereof comprises, or alternatively consists essentially of, or yet further consists of, the polypeptide encoded by the below polynucleotide sequence:

Antibodies can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, induces the B-lymphocytes to produce immunoglobulins specific for the antigen. Immunoglobulins may be purified from the mammal's serum.

Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a stow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods known in the art some of which are described in U.S. Pat. Nos. 7,279,559; 7,119,179; 7,060,800; 6,709,659; 6,656,746; 6,322,788; 5,686,073; and 5,670,153.

Monoclonal antibodies can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, those at the following web addresses, e.g., atcc.org, lifetech.com, last accessed on Nov. 26, 2007), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, in particular embodiments, the spleen or lymph nodes, of humans or other suitable animals that have been immunized with the antigen of interest and then screened for the activity of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, cDNA, or the like, display library; e.g., as available from various commercial vendors such as MorphoSys (Martinsreid/Planegg, Del.), BioInvent (Lund, Sweden), Affitech (Oslo, Norway) using methods known in the art. Art known methods are described in the patent literature some of which include U.S. Pat. Nos. 4,704,692; 5,723,323; 5,763,192; 5,814,476; 5,817,483; 5,824,514; and 5,976,862. Alternative methods rely upon immunization of transgenic animals (e.g., SCID mice, Nguyen et al. (1977) Microbiol. Immunol. 41:901-907 (1997); Sandhu et al. (1996) Crit, Rev. Biotechnol. 16:95-118; Eren et al. (1998) Mumma 93:154-161 that are capable of producing a repertoire of human antibodies, as known in the art and/or as described herein. Such techniques, include, but are not limited to, ribosome display Wanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052; Wen et al. (1987) J. Immunol 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134).

In some embodiments, the antibody or antigen-binding fragment thereof comprises, or consists essentially of, or yet further consists of an antibody derivative. Antibody derivatives of the present disclosure can also be prepared by delivering a polynucleotide encoding an antibody disclosed herein to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fe-mediated cellular toxicity, and glycoproteins so generated.

The antibodies disclosed herein also include derivatives that are modified by the covalent attachment of any type of molecule to the antibody such that covalent attachment does not prevent the antibody from generating an anti-idiotypic response. Antibody derivatives include, but are not limited to, antibodies that have been modified by glycosylation, acetylation, pegylation, phosphorylation, amidation, derivatization by known protecting/blocking groups, proteolytic cleavage, linkage to a cellular ligand or other protein, etc. Additionally, the derivatives may contain one or more non-classical amino acids.

Antibody derivatives also can be prepared by delivering a polynucleotide disclosed herein to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize has been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and references cited therein. Thus, antibodies can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids or variable or constant regions from other isotypes.

In general, the CDR residues are directly and most substantially involved in influencing antigen-binding. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Chimeric, humanized or primatized antibodies of the present disclosure can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762; and 6,180,370). Similarly, to create a primatized antibody the murine CDR regions can be inserted into a primate framework using methods known in the art (WO 93/02108 and WO 99/55369).

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies disclosed herein also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies disclosed herein can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al., which discloses antibody variants that have one or more amino acids inserted into a hypervariable region of the parent antibody and a binding affinity for a target antigen which is at least about two fold stronger than the binding affinity of the parent antibody for the antigen).

The term “antibody derivative” further includes engineered antibody molecules, fragments and single domains such as scFv, dAbs, nanobodies, minibodies, Unibodies, and Affibodies & Hudson (2005) Nature Biotech 23(9):1126-36; U.S. Pat. Application Publication No. 2006/0211088; PCT International Application Publication No. WO 2007/059782; U.S. Pat. No. 5,831,012).

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Ed segments (V_{H}-C_{H}1-V_{H}-C_{H}1) which form a pair of antigen-binding regions. Linear antibodies can be bispecific or monospecific.

The antibodies disclosed herein can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from a prokaryotic host as described above. A number of antibody production systems are described in Birch & Radner (2006) Adv. Drug Delivery Rev. 58: 671-685.

If an antibody being tested binds with protein or polypeptide, then the antibody being tested and the antibodies provided by this disclosure are equivalent. It also is possible to determine without undue experimentation, whether an antibody has the same specificity as the antibody disclosed herein by determining whether the antibody being tested prevents an antibody disclosed herein from binding the protein or polypeptide with which the antibody is normally reactive. If the antibody being tested competes with the antibody disclosed herein as shown by a decrease in binding by the monoclonal antibody disclosed herein, then it is likely that the two antibodies bind to the same or a closely related epitope. Alternatively, one can pre-incubate the antibody disclosed herein with a protein with which it is normally reactive, and determine if the antibody being tested is inhibited in its ability to bind the antigen. If the antibody being tested is inhibited then, in all likelihood, it has the same, or a closely related, epitopic specificity as the antibody disclosed herein.

The term “antibody” also is intended to include antibodies of all immunoglobulin isotypes and subclasses. Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307. Alternatively, recombinant DNA techniques may be used.

The isolation of other monoclonal antibodies with the specificity of the monoclonal antibodies described herein can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody of interest.

In some aspects disclosed herein, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The variable region of the antibodies of the present disclosure can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or antigen-binding fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²⁵I, ¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabinc, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrietocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (Yu et al. 1994 Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies. These antibodies are particularly useful in applications where it is desired to deplete an NK cell.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

In certain aspects, the disclosure relates to an antibody or antigen-binding fragment that specifically recognizes or binds a tip or tail domain of a DNABII protein or fragment thereof, the tail fragment or tip fragment. The DNABII protein or fragment thereof can be an IHF or an HU polypeptide.

Functional Analysis with Antibodies

Antibodies disclosed herein can be used to purify the polypeptides disclosed herein and to identify biological equivalent polypeptide and/or polynucleotides. They also can be used to identify agents that modify the function of the polypeptides disclosed herein. These antibodies include polyclonal antisera, monoclonal antibodies, and various reagents derived from these preparations that are familiar to those practiced in the art and described above.

Antibodies that neutralize the activities of proteins encoded by identified genes can also be used in vivo and in vitro to demonstrate function by adding such neutralizing antibodies into in vivo and in vitro test systems. They also are useful as pharmaceutical agents to modulate the activity of polypeptides disclosed herein.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

The antibodies disclosed herein may be used for vaccination or to boost vaccination, alone or in combination with peptides or protein-based vaccines or dendritic-cell based vaccines.

### Diagnostic and Therapeutic Methods

A method is provided for preventing, or inhibiting, or competing with the binding of a DNABII polypeptide or protein to a microbial DNA, by contacting the DNABII polypeptide or protein or the microbial DNA with an effective amount of one or more of agents as described above, e.g., an antibody or antigen-binding fragment thereof, a polypeptide or CDR as disclosed herein, and an HMGB1 polypeptide or a fragment thereof as disclosed herein, thereby preventing or inhibiting or competing with the binding of the DNABII protein or polypeptide to the microbial DNA. The DNABII polypeptide can be an IHF or HU peptide. In one aspect, the antibody or antigen-binding fragment thereof selectively binds to a tip region of the DNABII polypeptide (including but not limited to: a tip region of IHF or HU, a tip region of IHFA or IHFB, and/or the tip-chimeric peptide IhfA5-mIhfB4_{NTHI}). In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 24 or an equivalent thereof, and/or a light chain (LC) immunoglobulin variable domain sequence comprising, or consisting essentially of, or yet further consisting of an amino acid sequence of SEQ ID NO: 25 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 1, 2 or 3, or an equivalent of each thereof, and/or a light chain (LC) immunoglobulin variable domain sequence comprising, or consisting essentially of, or yet further consisting of an amino acid sequence selected from the group of SEQ ID NOs: 7, 8 or 9, or an equivalent of each thereof. In yet a further embodiment, the contacting is in vivo or in vitro.

In another aspect, the antibody or antigen-binding fragment thereof for use in a method as disclosed herein, comprises or consists essentially of, or yet further consists of: a heavy chain (HC) immunoglobulin variable domain sequence comprising a sequence selected from the group of SEQ ID NO: 13, 24, or 26, or an equivalent of each thereof, and/or a light chain (LC) immunoglobulin variable domain sequence comprising a sequence selected from the group of SEQ ID NO: 14, 25, or 27, or an equivalent of each thereof.

In a further aspect, one or more of the DNABII polypeptide and/or the microbial DNA and/or the antibody or antigen-binding fragment thereof and/or the polypeptide or CDRs as disclosed herein are detectably labeled, for example with a radioisotope or luminescent molecules that will emit a signal when brought into close contact with each other. The contacting can be performed in vitro or in vivo. These methods can be combined with diagnostic methods to detect and/or monitor biofilm formation and/or disruption, for example, using one or more of the DNABII polypeptide and/or the microbial DNA and/or the antibody or antigen-binding fragment thereof and/or the polypeptide or CDRs as disclosed herein. In one aspect, the diagnostic methods comprise the use of an antibody or antigen-binding fragment as disclosed herein that in one aspect, specifically binds to a tail region or fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment are detectably labeled. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof.

In another aspect, a method for disrupting a microbial biofilm is provided by contacting the biofilm with an effective amount of one or more of agent as described above, e.g., an antibody or antigen-binding fragment thereof, a polypeptide, or a CDR, thereby disrupting the microbial biofilm, and an HMGB1 polypeptide or a fragment thereof as disclosed herein. In one aspect, the antibody or antigen-binding fragment thereof selectively binds to a tip region of the DNABII polypeptide (including but not limited to: a tip region of IHF or HU, a tip region of IHFA or IHFB, and/or the tip-chimeric peptide IhfA5-mIhfB4_{NTHI}). The DNABII polypeptide can be an IHF or HU peptide. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 24 or an equivalent thereof, and/or a light chain (LC) immunoglobulin variable domain sequence comprising, or consisting essentially of, or yet further consisting of an amino acid sequence of SEQ ID NO: 25 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 1, 2 or 3, or an equivalent of each thereof, and/or a light chain (LC) immunoglobulin variable domain sequence comprising, or consisting essentially of, or yet further consisting of an amino acid sequence selected from the group of SEQ ID NOs: 7, 8 or 9, or an equivalent of each thereof. In yet a further embodiment, the contacting is in vivo or in vitro.

In one aspect, the microbial biofilm is produced by a microorganism that exports a DNABII polypeptide. The DNABII polypeptide can be an IHF or HU peptide. In a further aspect, one or more of the antibody, antigen-binding fragment, DNABII polypeptide and the microbial DNA are detectably labeled, for example with a radioisotope or luminescent molecules that will emit a signal when brought into close contact with each other. The contacting can be performed in vitro or in vivo. In one aspect, the agent is one or more antibodies and/or antigen-binding fragments that are the same or different from each other. In some embodiments, such antibodies or antigen-binding fragments are administered alone or in combination with each other, or an agent other than the antibody, or yet a further pharmaceutically effective agent, alone or in combination with a pharmaceutically acceptable carrier. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or tail fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment are detectably labeled.

Also provided are methods to prevent formation of or to disrupt a biofilm on a surface comprising, or consisting essentially of, or yet further consisting of, treating the surface susceptible to or containing a biofilm with an effective amount of one or more of an antibody or antigen-binding fragment thereof, polypeptide, or CDR as described herein, and an HMGB1 polypeptide or a fragment thereof as disclosed herein, wherein the antibody or the antigen-binding fragment thereof, binds a tip region of a DNABII peptide (including but not limited to: a tip region of IHF or HU, a tip region of IHFA or IHFB, and/or the tip-chimeric peptide IhfA5-mIhfB4_{NTHI}). The DNABII polypeptide can be an IHF or HU peptide. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof.

In one aspect, the antibody or antigen-binding fragment comprises a detectable label. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment are detectably labeled. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof.

Further provided herein are methods to detect a biofilm in a subject, comprising, or consisting essentially of, or consisting of, administering to the subject an effective amount of one or more of an antibody or an antigen-binding fragment thereof, a polypeptide, or a CDR as disclosed herein to the subject. In one aspect, the diagnostic methods comprise the use of an antibody or antigen-binding fragment as disclosed herein that in one aspect, specifically binds to a tail region or fragment of a DNABII polypeptide (including but not limited to: a tail region of IHF or HU, a tail region of IHFA or IHFB, and/or the tail-chimeric peptide IhfA3-IhfB2_{NTHI}). The DNABII polypeptide can be an IHF or HU peptide. In a further aspect, the antibody or antigen-binding fragment are detectably labeled. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof.

Methods to prevent or disrupt a biofilm in a subject are provided. The methods, comprise, or consist essentially of, or consist of, administering to the subject an antibody or an antigen-binding fragment thereof as disclosed herein that binds to a tip region of a DNABII peptide (including but not limited to: a tip region of IHF or HU, a tip region of IHFA or IHFB, and/or the tip-chimeric peptide IhfA5-mIhfB4_{NTHI}), and an HMGB1 polypeptide or a fragment thereof as disclosed herein. In one aspect, methods to prevent or disrupt a biofilm in a subject are provided, comprising, or alternatively consisting of, or yet further consisting of, administering to the subject an effective amount of one or more of the antibody, antigen-binding fragment thereof, polypeptide, or CDR as disclosed herein, and an HMGB1 polypeptide or a fragment thereof as disclosed herein, and/or an effective amount of one or more of a polynucleotide or a vector encoding the antibody, antigen-binding fragment thereof, polypeptide or CDR. The DNABII peptide can be an IHF or HU peptide. In one aspect, the antibody or antigen-binding fragment thereof selectively binds to a tip region of the DNABII polypeptide (including but not limited to: a tip region of IHF or HU, a tip region of IHFA or IHFB, and/or the tip-chimeric peptide IhfA5-mIhfB4_{NTHI}). In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 24 or an equivalent thereof, and/or a light chain (LC) immunoglobulin variable domain sequence comprising, or consisting essentially of, or yet further consisting of an amino acid sequence of SEQ ID NO: 25 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 1, 2 or 3, or an equivalent of each thereof, and/or a light chain (LC) immunoglobulin variable domain sequence comprising, or consisting essentially of, or yet further consisting of an amino acid sequence selected from the group of SEQ ID NOs: 7, 8 or 9, or an equivalent of each thereof. In yet a further embodiment, the contacting is in vivo or in vitro. The antibody or antigen-binding fragment thereof can be detectably labeled. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment are detectably labeled.

Methods to treat a condition characterized by the formation of biofilm in a subject are provided, the methods, comprising, or consisting essentially of, or consisting of, administering to the subject an antibody or an antigen-binding fragment thereof as disclosed herein that binds to a tip region of a DNABII polypeptide (including but not limited to: a tip region of IHF or HU, a tip region of IHFA or IHFB, and/or the tip-chimeric peptide IhfA5-mIhfB4_{NTHI}), and an HMGB1 polypeptide or a fragment thereof as disclosed herein. In one aspect, methods to prevent or treat a condition characterized by the formation of biofilm in a subject are provided by administering to the subject an effective amount of one or more of the antibody, the antigen-binding fragment thereof, polypeptide, or CDR as disclosed herein, and an HMGB1 polypeptide or a fragment thereof as disclosed herein, and/or an effective amount of one or more of a polynucleotide or a vector encoding the antibody, antigen-binding fragment thereof, polypeptide or CDR. In another aspect, methods for inhibiting, preventing or treating a microbial infection that produces a biofilm in a subject are provided, comprising, or alternatively consisting of, or yet further consisting of, administering to the subject an effective amount of one or more of the antibody, antigen-binding fragment thereof, polypeptide, or CDR as disclosed herein, and an HMGB1 polypeptide or a fragment thereof as disclosed herein, and/or an effective amount of one or more of a polynucleotide or a vector encoding the antibody, antigen-binding fragment thereof, polypeptide or CDR. The DNABII polypeptide can be an IHF or HU peptide. In one aspect, the antibody or antigen-binding fragment thereof selectively binds to a tip region of the DNABII polypeptide (including but not limited to: a tip region of IHF or HU, a tip region of IHFA or IHFB, and/or the tip-chimeric peptide IhfA5-mIhfB4_{NTHI}). In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 24 or an equivalent thereof, and/or a light chain (LC) immunoglobulin variable domain sequence comprising, or consisting essentially of, or yet further consisting of an amino acid sequence of SEQ ID NO: 25 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 1, 2 or 3, or an equivalent of each thereof, and/or a light chain (LC) immunoglobulin variable domain sequence comprising, or consisting essentially of, or yet further consisting of an amino acid sequence selected from the group of SEQ ID NOs: 7, 8 or 9, or an equivalent of each thereof. In yet a further embodiment, the contacting is in vivo or in vitro. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or tail fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment are detectably labeled.

In the above methods, the biofilm is derived from a gram negative or a gram positive biofilm producing bacteria. Non-limiting examples of conditions are selected from the group of: chronic non-healing wounds, including venous ulcers and diabetic foot ulcers, ear infections, sinus infections, urinary tract infections, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease.

In certain embodiments of a method disclosed herein, administration of one or more of the antibody, antigen-binding fragment thereof, polypeptide, or CDR, and an HMGB1 polypeptide or a fragment thereof as disclosed herein reduces one or more of pro-inflammatory cytokines in the subject. Non-limiting examples of the pro-inflammatory cytokines includes: IL-1β, IL6, IL8, IL12p70, IL17A, Interferon (IFN) and tumor necrosis factor (TNF). Additionally or alternatively, administration of an effective amount of one or more of the antibody, antigen-binding fragment thereof, polypeptide, or CDR, and an HMGB1 polypeptide or a fragment thereof as disclosed herein increases one or more of anti-inflammatory cytokines in the subject. In one embodiment, the anti-inflammatory cytokines include, but are not limited to, IL10, IL13, IL-1ra, IL-4, IL-11, and transforming growth factor-β (TGF-β).

Also provided are methods for one or more of: inducing a pro-inflammatory response or treating a condition mediated by a reduced inflammatory response in a subject in need thereof, the method comprising, or alternatively consisting essentially of, or yet further consisting of, administering to the subject an effective amount of an antibody or an antigen-binding fragment thereof that binds to a tail region of a DNABII polypeptide (including but not limited to: a tail region of IHF or HU, a tail region of IHFA or IHFB, and/or the tail-chimeric peptide IhfA3-IhfB2_{NTHI}), and an HMGB1 polypeptide or a fragment thereof as disclosed herein. The DNABII polypeptide can be an IHF or HU peptide. These methods can be combined with diagnostic methods to detect and/or monitor cytokine release or levels in a tissue or the subject. In one aspect, the diagnostic methods comprise the use of an antibody or antigen-binding fragment as disclosed herein that in one aspect, specifically binds to a tail region or tail fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment is detectably labeled. Non-limiting cytokines include, e.g., IL1beta, IL6, IL8, IL12p70, IL10, IL13 and IFN.

Also provided are methods for one of more of inhibiting a pro-inflammatory response or treating a condition mediated by an enhanced inflammatory response in a subject in need thereof, the method comprising, or alternatively consisting essentially of, or yet further consisting of, administering to the subject an effective amount of an antibody or antigen-binding fragment thereof binds to a tip region of a DNABII polypeptide (including but not limited to: a tip region of IHF or HU, a tip region of IHFA or IHFB, and/or the tip-chimeric peptide IhfA5-mIhfB4_{NTHI}), and an HMGB1 polypeptide or a fragment thereof as disclosed herein. The DNABII polypeptide can be an IHF or HU peptide. These methods can be combined with diagnostic methods to detect and/or monitor cytokine release or levels in a tissue or the subject. In one aspect, the diagnostic methods comprise the use of an antibody or antigen-binding fragment as disclosed herein that in one aspect, specifically binds to a tail region or tail fragment of a DNABII polypeptide (including but not limited to: a tail region of IHF or HU, a tail region of IHFA or IHFB, and/or the tail-chimeric peptide IhfA3-IhfB2_{NTHI}). In a further aspect, the antibody or antigen-binding fragment is detectably labeled. Non-limiting cytokines include, e.g., IL1beta, IL6, IL8, IL12p70, IL10, IL13 and IFN.

Additionally or alternatively provided are methods to detect a biofilm on a surface comprising, or alternatively consisting of, or yet further consisting of, contacting the surface (in one aspect susceptible to or containing a biofilm) with an effective amount of one or more of the antibody, antigen-binding fragment, polypeptide or CDR as described herein, wherein the antibody, antigen-binding fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, tail region of IHF or HU, a tail region of IHFA or IHFB, and/or or the tail-chimeric peptide IhfA3-IhfB2_{NTHI}). In one embodiment, the contacting is in vivo or in vitro. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof. In a further aspect, the antibody or antigen-binding fragment are detectably labeled.

Additionally or alternatively provided is a method for detecting a microbial infection that produces a biofilm in a subject. The method comprises, or alternatively consists of, or yet further consists of, contacting an effective amount of one or more of the antibody, antigen-binding fragment thereof, polypeptide, or CDR as disclosed herein with a biological sample suspected of comprising the biofilm and isolated from the subject and detecting the binding of the antibody, antigen-binding fragment thereof, polypeptide, or CDR to any biofilm in the sample. In one embodiment, the antibody, antigen-binding fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, tail region of IHF or HU, a tail region of IHFA or IHFB, and/or or the tail-chimeric peptide IhfA3-IhfB2_{NTHI}). In a further embodiment, the contacting is in vivo or in vitro. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof. In a further aspect, the antibody or antigen-binding fragment are detectably labeled.

Additionally or alternatively provided is a method for screening subjects having a biofilm, comprising, or alternatively consisting of, or yet further consisting of, contacting an effective amount of one or more of an antibody, an antigen-binding fragment thereof, a polypeptide, or a CDR as disclosed herein with a biological sample comprising the biofilm and isolated from the subject, and detecting the binding of the antibody, antigen-binding fragment thereof, polypeptide, or CDR to any biofilm in the sample. In one embodiment, the antibody, antigen-binding fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, tail region of IHF or HU, a tail region of IHFA or IHFB, and/or or the tail-chimeric peptide IhfA3-IhfB2_{NTHI}). In a further embodiment, a subject detected with the binding is selected for administration with an effective amount of one or more of an antibody, an antigen-binding fragment thereof, a polypeptide, or a CDR as disclosed herein, and/or an effective amount of one or more of a polynucleotide or a vector encoding the antibody, antigen-binding fragment thereof, polypeptide or CDR, wherein the antibody, antigen-binding fragment thereof, polypeptide, or CDR binds a tip region of a DNABII peptide (including but not limited to: a tip region of IHF or HU, a tip region of IHFA or IHFB, or the tip-chimeric peptide IhfA5-mIhfB4_{NTHI}). In yet a further embodiment, the contacting is in vivo or in vitro. In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 26 or an equivalent thereof, and/or a light chain (LC) immunoglobulin variable domain sequence comprising, or consisting essentially of, or yet further consisting of an amino acid sequence of SEQ ID NO: 27 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 4, 5 or 6, or an equivalent of each thereof, and/or a light chain (LC) immunoglobulin variable domain sequence comprising, or consisting essentially of, or yet further consisting of an amino acid sequence selected from the group of SEQ ID NOs: 10, 11 or 12, or an equivalent of each thereof. In a further aspect, the antibody or antigen-binding fragment are detectably labeled.

Further provided are method to prepare an interfering nucleic acid comprising preparing a nucleic acid consisting of about 10-20 nucleotides that specifically binds a specific binding partner to the antibody or antigen-binding fragment thereof as disclosed herein and optionally isolating the interfering nucleic acid prepared by the method.

When practiced in vitro, the several of the disclosed methods are useful to screen for or confirm agents (e.g., antibodies, fragments and mimics thereof) having the same, similar or opposite ability as the polypeptides, polynucleotides, antibodies, host cells, small molecules and compositions disclosed herein. Alternatively, they can be used to identify which agent is best suited to treat a microbial infection or if the treatment has been effective. For example, one can screen for new agents or combination therapies by having two samples containing for example, the DNABII polypeptide and microbial DNA and the agent to be tested. The DNABII polypeptide can be an IHF or HU peptide. The second sample contains the DNABII polypeptide and microbial DNA and an agent known to active, e.g., an agent as described above, e.g., an antibody or antigen-binding fragment thereof, to serve as a positive control. In a further aspect, several samples are provided and the agents are added to the system in increasing dilutions to determine the optimal dose that would likely be effective in treating a subject in the clinical setting. As is apparent to those of skill in the art, a negative control containing the DNABII polypeptide and the microbial DNA can be provided. In a further aspect, the DNABII polypeptide and the microbial DNA are detectably labeled, for example with luminescent molecules that will emit a signal when brought into close contact with each other. The samples are contained under similar conditions for an effective amount of time for the agent to inhibit, compete or titrate the interaction between the DNABII polypeptide and microbial DNA and then the sample is assayed for emission of signal from the luminescent molecules. If the sample emits a signal, then the agent is not effective to inhibit binding.

In another aspect, the in vitro method is practiced in a miniaturized chamber slide system wherein the microbial (such as a bacterial) isolate causing an infection could be isolated from the human/animal then cultured to allow it to grow as a biofilm in vitro. The agent (e.g., an antibody or antigen-binding fragment thereof) or potential agent biofilm is added alone or in combination with another agent to the culture with or without increasing dilutions of the potential compound or agent such as an antibody or antigen-binding fragment thereof, to find the optimal dose that would likely be effective at treating that patient when delivered to the subject where the infection existed. As apparent to those of skill in the art, a positive and negative control can be performed simultaneously.

In a further aspect, the method is practiced in a high throughput platform with an agent as described above, e.g., an antibody or antigen-binding fragment thereof, and/or potential agent (alone or in combination with another agent) in a flow cell. The agent as described above, e.g., an antibody or antigen-binding fragment thereof, or potential agent is added alone or in combination with another agent to the culture with or without increasing dilutions of the potential agent or the agent as described above, e.g., an antibody or antigen-binding fragment thereof, (or other antibody, small molecule, agent, etc.) to find the optimal dose that would likely be effective at treating that patient when delivered to the subject where the infection existed. Biofilm isolates are sonicated to separate biofilm bacteria from DNABII polypeptide such as IHF bound to microbial DNA. The DNABII polypeptide-DNA complexes are isolated by virtue of the anti-DNABII or IHF antibody on the platform. The microbial DNA is then released with e.g., a salt wash, and used to identify the biofilm bacteria added. The freed DNA is then identified, e.g., by PCR sequenced. If DNA is not freed, then the agent(s) successfully performed or bound the microbial DNA. If DNA is found in the sample, then the agent did not interfere with DNABII polypeptide-microbial DNA binding. As is apparent to those of skill in the art, a positive and/or negative control can be simultaneously performed.

The above methods also can be used as a diagnostic test since it is possible that a given bacterial species will respond better to reversal of its biofilm by one agent more than another, this rapid high throughput assay system could allow one skilled the art to assay a panel of possible anti-DNABII or IHF-like agents to identify the most efficacious of the group.

The advantage of these methods is that most clinical microbiology labs in hospitals are already equipped to perform these sorts of assays (i.e., determination of MIC, MBC values) using bacteria that are growing in liquid culture (or planktonically). As is apparent to those of skill in the art, bacteria generally do not grow planktonic ally when they are causing diseases. Instead they are growing as a stable biofilm and these biofilms are significantly more resistant to treatment by antibiotics, antibodies or other therapeutics. This resistance is why most MIC/MBC values fail to accurately predict efficacy in vivo. Thus, by determining what “dose” of agent could reverse a bacterial biofilm in vitro (as described above) Applicants' pre-clinical assay would be a more reliable predictor of clinical efficacy, even as an application of personalized medicine.

In addition to the clinical setting, the methods can be used to identify the microbe causing the infection and/or confirm effective treatments and agents in an industrial setting. Thus, the agents can be used to treat, inhibit or disrupt a biofilm in an industrial setting.

In a further aspect of the above methods, an antibiotic or antimicrobial known to inhibit growth of the underlying infection is added sequentially or concurrently, to determine if the infection can be inhibited. It is also possible to add the interfering agent to the microbial DNA or DNABII polypeptide before adding the missing complex to assay for biofilm inhibition. In one aspect, DNase treatment is excluded from the method of use.

When practiced in vivo in non-human animal such as a chinchilla, the method provides a pre-clinical screen to identify agents that can be used alone or in combination with other agents to disrupt biofilms.

In another aspect, provided herein is a method of inhibiting, preventing or disrupting a biofilm in a subject by administering to the subject an effective amount of an agent as described above, e.g., an antibody or antigen-binding fragment thereof, and an HMGB1 polypeptide or a fragment thereof as disclosed herein, thereby inhibiting, preventing or disputing the microbial biofilm. The methods, comprise, or consist essentially of, or consist of, administering to the subject an antibody or an antigen-binding fragment thereof as disclosed herein that binds to a tip region of a DNABII peptide (including but not limited to: a tip region of IHF or HU, a tip region of IHFA or IHFB, and/or the tip-chimeric peptide IhfA5-mIhfB4_{NTHI}), and an HMGB1 polypeptide or a fragment thereof as disclosed herein. The DNABII peptide can be an IHF or HU peptide. The antibody or antigen-binding fragment thereof can be detectably labeled. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or fragment of a DNABII polypeptide (including but not limited to: a tail region of IHF or HU, a tail region of IHFA or IHFB, or the tail-chimeric peptide IhfA3-IhfB2_{NTHI}). In a further aspect, the antibody or antigen-binding fragment are detectably labeled.

In one aspect, the agent is one or more antibodies and/or antigen-binding fragments that are the same or different from each other. In some embodiments, such antibodies or antigen-binding fragments are administered alone or in combination with each other, or an agent other than the antibody, or yet a further pharmaceutically effective agent, alone or in combination with a pharmaceutically acceptable carrier. Non-limiting examples of such subjects include mammals, e.g., pets, and human patients.

Also provided herein is a method for inducing an immune response in or conferring passive immunity in a subject in need thereof, comprising, or alternatively consisting essentially of, or yet further consisting of, administering to the subject an effective amount of an HMGB1 polypeptide or a fragment thereof as disclosed herein and one or more of the antibodies or antigen-binding fragments thereof as described therein that bind to a tip region of a DNABII peptide, e.g., IHF or HU peptide. Such tip region of a DNABII peptide includes, but is not limited to: a tip region of IHF or HU, a tip region of IHFA or IHFB, or the tip-chimeric peptide IhfA5-mIhfB4_{NTHI}. In one aspect, provided is a method for conferring passive immunity in a subject, comprising, or alternatively consisting essentially of, or yet further consisting of administering to the subject an effective amount of an HMGB1 polypeptide or a fragment thereof as disclosed herein and one or more of an antibody, antigen-binding fragment thereof of, polypeptide, or CDR as disclosed herein, and/or an effective amount of one or more of a polynucleotide or a vector encoding the antibody, antigen-binding fragment thereof, polypeptide or CDR, wherein the antibody, antigen-binding fragment, polypeptide or CDR binds to a tip region of a DNABII peptide (including but not limited to: a tip region of IHF or HU, a tip region of IHFA or IHFB, or the tip-chimeric peptide IhfA5-mIhfB4_{NTHI}). In one embodiment, provided is an antibody or an antigen-binding fragment thereof, that comprises or consists essentially of, or yet further consists of: a heavy chain (HC) immunoglobulin variable domain sequence comprising, or consisting essentially of, or yet further consisting of an amino acid sequence of SEQ ID NO: 24 or an equivalent thereof, and/or a light chain (LC) immunoglobulin variable domain sequence comprising, or consisting essentially of, or yet further consisting of an amino acid sequence of SEQ ID NO: 25 or an equivalent thereof. In a further embodiment, the antibody or antigen-binding fragment thereof comprises or consists essentially of, or yet further consists of: a heavy chain (HC) immunoglobulin variable domain sequence comprising, or consisting essentially of, or yet further consisting of an amino acid sequence selected from the group of SEQ ID NOs: 1, 2 or 3, or an equivalent of each thereof, and/or a light chain (LC) immunoglobulin variable domain sequence comprising, or consisting essentially of, or yet further consisting of an amino acid sequence selected from the group of SEQ ID NOs: 7, 8 or 9, or an equivalent of each thereof. The antibody or antigen-binding fragment thereof can be detectably labeled. These methods can be combined with diagnostic methods to detect and/or monitor biofilm formation and/or disruption. In one aspect, the diagnostic methods comprise the use of an antibody or antigen-binding fragment as disclosed herein that in one aspect, specifically binds to a tail region or tail fragment of a DNABII polypeptide (including but not limited to: a tail region of IHF or HU, a tail region of IHFA or IHFB, or the tail-chimeric peptide IhfA3-IhfB2_{NTHI}). In a further aspect, the antibody or antigen-binding fragment are detectably labeled.

In one aspect, the agent is one or more antibodies and/or antigen-binding fragments that are the same or different from each other. In some embodiments, such antibodies or antigen-binding fragments are administered alone or in combination with each other, or an agent other than the antibody, or yet a further pharmaceutically effective agent, alone or in combination with a pharmaceutically acceptable carrier.

In a further aspect, the methods further comprise, or alternatively consist essentially of, or yet further consist of administering to the subject an effective amount of one or more of an antimicrobial, an antigenic peptide or an adjuvant.

A non-limiting example of an antimicrobial agent is another vaccine component such as a surface antigen, e.g., an OMP P5, rsPilA, OMP 26, OMP P2, or Type IV Pilin protein (see Jurcisek and Bakaletz (2007) J. Bacteriology 189(10):3868-3875; Murphy, T. F. et al. (2009) The Pediatric Infectious Disease Journal 28:S121-S126).

The agents and compositions disclosed herein can be concurrently or sequentially administered with other antimicrobial agents and/or surface antigens. In one particular aspect, administration is locally to the site of the infection by direct injection or by inhalation for example. Other non-limiting examples of administration include by one or more method comprising transdermally, urethrally, sublingually, rectally, vaginally, ocularly, subcutaneous, intramuscularly, intraperitoneally, intranasally, by inhalation or orally.

Microbial infections and disease that can be treated by the methods disclosed herein include infection by a gram-positive or gram-negative organism that produces a biofilm, e.g., Streptococcus agalactiae, Neisseria meningitidis, Treponemes, denticola, pallidum, Burkholderia cepacia, or Burkholderia pseudomallei. In one aspect, the microbial infection is one or more of Haemophilus influenzae (nontypeable), Moraxella catarrhalis, Streptococcus pneumoniae, Streptococcus pyogenes, Pseudomonas aeruginosa, Mycobacterium tuberculosis. These microbial infections may be present in the upper, mid and lower airway (otitis, sinusitis, bronchitis but also exacerbations of chronic obstructive pulmonary disease (COPD), chronic cough, complications of and/or primary cause of cystic fibrosis (CF) and community acquired pneumonia (CAP). Thus, by practicing the in vivo methods disclosed herein, these diseases and complications from these infections can also be prevented or treated.

Infections might also occur in the oral cavity (caries, periodontitis) and caused by Streptococcus mutans, Porphyromonas gingivalis, Aggregatibacter actinomvctemcomitans. Infections might also be localized to the skin (abscesses, ‘staph’ infections, impetigo, secondary infection of burns, Lyme disease) and caused by Staphylococcus aureus, Staphylococcus epidermidis, Pseudomonas aeruginosa and Borrelia burdorferi. Infections of the urinary tract (UTI) can also be treated and are typically caused by Escherichia coli. Infections of the gastrointestinal tract (GI) (diarrhea, cholera, gall stones, gastric ulcers) are typically caused by Salmonella enterica serovar, Vibrio cholerae and Helicobacter pylori. Infections of the genital tract include and are typically caused by Neisseria gonorrhoeae. Infections can be of the bladder or of an indwelling device caused by Enterococcus faecalis. Infections associated with implanted prosthetic devices, such as artificial hip or knee replacements, or dental implants, or medical devices such as pumps, catheters, stents, or monitoring systems, typically caused by a variety of bacteria, can be treated by the methods disclosed herein. These devices can be coated or conjugated to an agent as described herein. Thus, by practicing the in vivo methods disclosed herein, these diseases and complications from these infections can also be prevented or treated.

Infections caused by Streptococcus agalactiae can also be treated by the methods disclosed herein and it is the major cause of bacterial septicemia in newborns. Infections caused by Neisseria meningitidis which can cause meningitis can also be treated.

Thus, routes of administration applicable to the methods disclosed herein include intranasal, intramuscular, urethrally, intratracheal, subcutaneous, intradermal, transdermal, topical application, intravenous, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery include systemic or localized routes. In general, routes of administration suitable for the methods disclosed herein include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The agents disclosed herein can also be delivered to the subject by enteral administration. Enteral routes of administration include, but are not limited to, oral and rectal (e.g., using a suppository) delivery.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods disclosed herein, the interfering agent will be administered by inhalation, injection or orally on a continuous, daily basis, at least once per day (QD), and in various embodiments two (BID), three (TID), or even four times a day. Typically, the therapeutically effective daily dose will be at least about 1 mg, or at least about 10 mg, or at least about 100 mg, or about 200 to about 500 mg, and sometimes, depending on the compound, up to as much as about 1 g to about 2.5 g.

Dosing of can be accomplished in accordance with the methods disclosed herein using capsules, tablets, oral suspension, suspension for intra-muscular injection, suspension for intravenous infusion, get or cream for topical application, or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In certain embodiments, compositions exhibit high therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies (in certain embodiments, within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Polypeptides

Also provided herein are isolated polypeptides comprising the heavy and light chains of the antibodies, antigen-binding fragments thereof, CDRs and equivalents of each thereof with the addition of up to 25, or alternatively 20, or alternatively 15, or alternatively up to 10, or alternatively up to 5 random amino acids on either the amino or carboxyl termini (or on both).

In one aspect, provided herein are isolated polypeptides comprising, or consisting essentially of, or yet further consisting of, an amino acid sequence of the group of SEQ ID NOs: 1-14, or 24-27, or an equivalent of each thereof. The polypeptides can further comprise a detectable or purification marker.

This disclosure also provides isolated or recombinant polypeptides comprising or alternatively consisting essentially of, or yet further consisting of, two or more, or three or more, four or more, five or more, six or more, seven or more, eight or more, nine or more, ten or more, eleven or more, twelve or more, thirteen or more of all fourteen of the isolated polypeptides or a fragment or an equivalent of each thereof.

In any of the above embodiments, a peptide linker can be added to the N-terminus or C-terminus of the polypeptide. A “linker” or “peptide linker” refers to a peptide sequence linked to either the N-terminus or the C-terminus of a polypeptide sequence. In one aspect, the linker is from about 1 to about 20 amino acid residues long or alternatively 2 to about 10, about 3 to about 5 amino acid residues long. An example of a peptide linker is Gly-Pro-Ser-Leu-Lys-Leu (SEQ ID NO: 33). Other examples include Gly-Gly-Gly; Gly-Pro-Ser-Leu (SEQ ID NO: 34); Gly-Pro-Ser; Pro-Ser-Leu-Lys (SEQ ID NO: 35); Gly-Pro-Ser-Leu-Lys (SEQ ID NO: 36) and Ser-Leu-Lys-Leu (SEQ ID NO: 37).

In some aspects, either or both of the polynucleotides further comprises a signal peptide coding polynucleotide sequence located upstream of the variable domain, chain, the HMGB1 polypeptide or a fragment thereof, or CDR. Additional regulatory elements for expression of the polynucleotides are, in some embodiments, are operatively linked for expression and/or replication of the polypeptide. These elements are known in the art and include for example, promoters, enhancers or vector element. In one aspect, either or both of the HMGB1 polypeptide or a fragment thereof as disclosed herein or the anti-DNABII antibody or an antigen-binding fragment thereof as disclosed herein further comprise a signal peptide located upstream of the variable domain, chains, CDRs, HMGB1 polypeptide or a fragment thereof.

The isolated polypeptides disclosed herein are intended to include recombinantly produced polypeptides and proteins from prokaryotic and eukaryotic host cells, as well as muteins, analogs and fragments thereof, examples of such cells are described above. In some embodiments, the term also includes antibodies and anti-idiotypic antibodies as described herein. Such polypeptides can be isolated or produced using the methods known in the art and briefly described herein.

It is understood that functional equivalents or variants of the wild type polypeptide or protein also are within the scope of this disclosure, for example, those having conservative amino acid substitutions of the amino acids.

In a further aspect, the polypeptides are conjugated or linked to a detectable label or an agent to increase the half-life of the polypeptide, e.g., PEGylation a PEG mimetic, polysialyation, HESylation or glycosylation. Suitable labels are known in the art and described herein.

In a yet further aspect, the polypeptides with or without a detectable label can be contained or expressed on the surface of a host prokaryotic or eukaryotic host cell, such as a dendritic cell.

The proteins and polypeptides are obtainable by a number of processes known to those of skill in the art, which include purification, chemical synthesis and recombinant methods. Polypeptides can be isolated from preparations such as host cell systems by methods such as immunoprecipitation with antibody, and standard techniques such as gel filtration, ion-exchange, reversed-phase, and affinity chromatography. For such methodology, see for example Deutscher et al. (1999) Guide To Protein Purification: Methods In Enzymology (Vol. 182, Academic Press). Accordingly, this disclosure also provides the processes for obtaining these polypeptides as well as the products obtainable and obtained by these processes.

The polypeptides also can be obtained by chemical synthesis using a commercially available automated peptide synthesizer such as those manufactured by Perkin/Elmer/Applied Biosystems, Inc., Model 430A or 431A, Foster City, Calif., USA. The synthesized polypeptide can be precipitated and further purified, for example by high performance liquid chromatography (HPLC). Accordingly, this disclosure also provides a process for chemically synthesizing the proteins disclosed herein by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989) supra, using a host cell and vector systems described herein.

Also provided by this application are the polypeptides described herein conjugated to a detectable agent for use in the diagnostic methods. For example, detectably labeled polypeptides can be bound to a column and used for the detection and purification of antibodies. They also are useful as immunogens for the production of antibodies. The polypeptides disclosed herein are useful in an in vitro assay system to screen for agents or drugs, which modulate cellular processes. In another aspect, antibodies that are specific for the tail regions of the DNABII polypeptides (including but not limited to: a tail region of IHF or HU, a tail region of IHFA or IHFB, and/or the tail-chimeric peptide IhfA3-IhfB2_{NTHI}) are particularly useful in diagnostic assays for the detection of biofilms and can be used alone or in combination of one or more antibodies as described herein. In one aspect, antibodies specific for the tail regions are used as a companion diagnostic for an antibody or an antigen-binding fragment thereof that is specific for a tip region of the DNABII polypeptide (including but not limited to: a tip region of IHF or HU, a tip region of IHFA or IHFB, and/or the tip-chimeric peptide IhfA5-mIhfB4_{NTHI}). The DNABII polypeptide can be an IFH or an HU polypeptide.

It is well known to those skilled in the art that modifications can be made to the peptides disclosed herein to provide them with altered properties. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

Peptides disclosed herein can be modified to include unnatural amino acids. Thus, the peptides may comprise D-amino acids, a combination of and L-amino acids, and various “designer” amino acids (e.g., beta-methyl amino acids, C-alpha-methyl amino acids, and N-alpha-methyl amino acids, etc.) to convey special properties to peptides. Additionally, by assigning specific amino acids at specific coupling steps, peptides with alpha-helices, beta. turns, beta. sheets, gamma-turns, and cyclic peptides can be generated. Generally, it is believed that alpha-helical secondary structure or random secondary structure may be of particular use.

The polypeptides disclosed herein also can be combined with various solid phase carriers, such as an implant, a stent, a paste, a gel, a dental implant, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils. When used to prepare antibodies or induce an immune response in vivo, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides. Other suitable adjuvants include monophosphoryl lipid A (MPL), mutant derivatives of the heat labile enterotoxin of E. coli, mutant derivatives of cholera toxin, CPG oligonucleotides, and adjuvants derived from squalene.

This disclosure also provides a pharmaceutical composition or combinations comprising or alternatively consisting essentially of, or yet further consisting of, any of a polypeptide, analog, mutein, or fragment disclosed herein, alone or in combination with each other or other agents, such an antibiotic and an acceptable carrier or solid support. These compositions or combinations are useful for various diagnostic and therapeutic methods as described herein.

### Polynucleotides

This disclosure also provides isolated or recombinant polynucleotides encoding one or more of the above-identified antibodies, fragments thereof, CDRs, isolated or recombinant polypeptides and their respective complementary strands. Vectors comprising the isolated or recombinant polynucleotides are further provided examples of which are known in the art and briefly described herein. In one aspect where more than one isolated or recombinant polynucleotide is to be expressed as a single unit, the isolated or recombinant polynucleotides can be contained within a polycistronic vector. The polynucleotides can be DNA, RNA, mRNA or interfering RNA, such as siRNA, miRNA or dsRNA.

In another aspect, this disclosure provides an interfering agent that is a polynucleotide that interferes with the binding of the DNA to a polypeptide or protein in a microbial biofilm, or a four-way junction polynucleotide resembling a Holliday junction, a 3 way junction polynucleotide resembling a replication fork, a polynucleotide that has inherent flexibility or bent polynucleotide which can treat or inhibit DNABII polynucleotide (HU or IHF) from binding to microbial DNA as well treat, prevent or inhibit biofilm formation and associated infections and disorders. One of skill in the art can make such polynucleotides using the information provided herein and knowledge of those of skill in the art. See Goodman and Kay (1999) J. Biological Chem. 274(52):37004-37011 and Kamashev and Rouviere-Yaniv (2000) EMBO J. 19(23):6527-6535.

The disclosure further provides the isolated or recombinant polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)) which contains maps, functional properties, commercial suppliers and a reference to GenEMBL accession numbers for various suitable vectors.

In one embodiment, polynucleotides derived from the polynucleotides disclosed herein encode polypeptides or proteins having diagnostic and therapeutic utilities as described herein as well as probes to identify transcripts of the protein that may or may not be present. These nucleic acid fragments can by prepared, for example, by restriction enzyme digestion of larger polynucleotides and then labeled with a detectable marker. Alternatively, random fragments can be generated using nick translation of the molecule. For methodology for the preparation and labeling of such fragments, see, Sambrook et al. (1989) supra.

Expression vectors containing these nucleic acids are useful to obtain host vector systems to produce proteins and polypeptides. It is implied that these expression vectors must be replicable in the host organisms either as episomes or as an integral part of the chromosomal DNA. Non-limiting examples of suitable expression vectors include plasmids, yeast vectors, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See, Sambrook et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this disclosure also provides a host cell, e.g., a mammalian cell, an animal cell (rat or mouse), a human cell, or a prokaryotic cell such as a bacterial cell, containing a polynucleotide encoding a protein or polypeptide or antibody.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

When the vectors are used in an method as disclosed herein as gene therapy in vivo or ex vivo, a pharmaceutically acceptable vector, such as a replication-incompetent retroviral or adenoviral vector, are exemplary (but non-limiting) and may be of particular use. Pharmaceutically acceptable vectors containing the nucleic acids disclosed herein can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller et al. (1989) BioTechniques 7:980-990). The methodology of using replication-incompetent retroviruses for retroviral-mediated gene transfer of gene markers has been established. (Bordignon (1989) PNAS USA 86:8912-8952; Culver (1991) PNAS USA 88:3155; and Rill (1991) Blood 79(10):2694-2700).

This disclosure also provides genetically modified cells that contain and/or express the polynucleotides disclosed herein. The genetically modified cells can be produced by insertion of upstream regulatory sequences such as promoters or gene activators (see, U.S. Pat. No. 5,733,761). In one embodiment, the modified cells are eukaryotic cells or prokaryotic cells.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide disclosed herein under conditions permitting hybridization (optionally moderately stringent hybridization conditions) of complementary single-stranded polynucleotides, or optionally, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotide embodied in this disclosure can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

The polynucleotides disclosed herein can be isolated or replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds., Birkhauser Press, Boston (199.4)) or MacPherson et al. (1991) and (1995) supra, and references cited therein. Alternatively, one of skill in the art can use the sequences provided herein and a commercial DNA synthesizer to replicate the DNA. Accordingly, this disclosure also provides a process for obtaining the polynucleotides disclosed herein by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the poly-nucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, mRNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide disclosed herein are useful as hybridization probes or as an equivalent of the specific polynucleotides identified herein. Since the full coding sequence of the transcript is known, any portion of this sequence or homologous sequences can be used in the methods disclosed herein.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. In some embodiments, a probe useful for detecting the aforementioned mRNA is at least about 80% identical to the homologous region. In some embodiments, the probe is 85% identical to the corresponding gene sequence after alignment of the homologous region; in some embodiments, it exhibits 90% identity.

These probes can be used in radioassays (e.g., Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide disclosed herein. Accordingly, this disclosure also provides a probe comprising or corresponding to a polynucleotide disclosed herein, or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally well suited, so as to increase stability and selectivity of the hybrid, and thereby improving the specificity of particular hybrid molecules obtained. In certain embodiments, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present disclosure can serve as primers for the detection of genes or gene transcripts that are expressed in cells described herein. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustration purposes only, a primer is the same length as that identified for probes.

One method to amplify polynucleotides is PCR and kits for PCR amplification are commercially available. After amplification, the resulting DNA fragments can be detected by any appropriate method known in the art, e.g., by agarose gel electrophoresis followed by visualization with ethidium bromide staining and ultraviolet illumination.

Methods for administering an effective amount of a gene delivery vector or vehicle to a cell have been developed and are known to those skilled in the art and described herein. Methods for detecting gene expression in a cell are known in the art and include techniques such as in hybridization to DNA microarrays, in situ hybridization, PCR, RNase protection assays and Northern blot analysis. Such methods are useful to detect and quantify expression of the gene in a cell. Alternatively, expression of the encoded polypeptide can be detected by various methods. In particular, it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level of the expressed polynucleotide.

### Production Methods

Also provided are methods to produce the antibodies, fragments, CDRs, or polypeptides comprising, or alternatively consisting of, or yet further consisting of, culturing a host cell comprising a polynucleotide encoding the antibody, antigen-binding fragment, polypeptide, or CDR and an HMGB1 polypeptide or a fragment thereof as disclosed herein under conditions for expression of the polynucleotide, and optionally isolating the antibody, antigen-binding fragment, CDR polypeptide, and/or the HMGB1 polypeptide or fragment thereof as disclosed herein from the cell and/or culture. Additionally provided is a host cell comprising a polynucleotide encoding the antibody, antigen-binding fragment, polypeptide, CDR, and/or an HMGB1 polypeptide or a fragment thereof as disclosed herein under conditions for expression of the polynucleotide. In one embodiment, the host cell is a eukaryotic cell or a prokaryotic cell. In a further embodiment, the host cell is a mammalian cell.

### Compositions

Compositions are further provided. The compositions comprise a carrier and one or more of an isolated polypeptide disclosed herein, an isolated polynucleotide disclosed herein, a vector disclosed herein, an isolated host cell disclosed herein, a small molecule or an antibody, and/or an antigen-binding fragment disclosed herein. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, a small molecule, an isolated host cell disclosed herein, or an antibody or an antigen-binding fragment thereof of the disclosure, formulated with one or more pharmaceutically acceptable substances.

For oral preparations, any one or more of an isolated or recombinant polypeptide as described herein, an isolated or recombinant polynucleotide as described herein, a vector as described herein, an isolated host cell as described herein, a small molecule or an antibody or antigen-binding fragment thereof as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated polypeptide disclosed herein, an isolated polynucleotide disclosed herein, a vector disclosed herein, an isolated host cell disclosed herein, or an antibody or an antigen-binding fragment thereof disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., an OMP P5, OMP 26, OMP P2, or Type IV Pilin protein (see Jurcisek and Bakaletz (2007) J. of Bacteriology 189(10):3868-3875 and Murphy, T F, Bakaletz, L O and Smeesters, P R (2009) The Pediatric Infectious Disease Journal, 28:S121-S126) and antibacterial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of an isolated polypeptide disclosed herein, an isolated polynucleotide disclosed herein, a vector disclosed herein, a small molecule for use in the disclosure, an isolated host cell disclosed herein, or an antibody or antigen-binding fragment thereof as disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of an isolated polypeptide disclosed herein, an isolated polynucleotide disclosed herein, a vector disclosed herein, an isolated host cell disclosed herein, or an antibody or an antigen-binding fragment thereof disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation, polymers such as for example poly(glycolide-co-lactide) (PGLA) that is commercially available from a number of vendors, e.g., BioDegmer and Sigma-Aldrich. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer (e.g., PGLA) and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carbocylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the polypeptide, antibody or antigen-binding fragment thereof (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The present disclosure provides methods and compositions for the administration of a one or more of an interfering agent to a host (e.g., a human) for the treatment of a microbial infection. In various embodiments, these methods disclosed herein span almost any available method and route suitable for drug delivery, including in vivo and ex vivo methods, as well as systemic and localized routes of administration.

### Screening Assays

The present disclosure provides methods for screening for equivalent agents, such as equivalent monoclonal antibodies to a polyclonal antibody as described herein and various agents that modulate the activity of the active agents and pharmaceutical compositions disclosed herein or the function of a polypeptide or peptide product encoded by the polynucleotide disclosed herein. For the purposes of this disclosure, an “agent” is intended to include, but not be limited to a biological or chemical compound such as a simple or complex organic or inorganic molecule (referred to herein as a small molecule, such as a nucleic acid), a peptide, a protein (e.g., antibody), a polynucleotide anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One embodiment is a method for screening agents capable of interacting with, binding to, or inhibiting the DNA-DNABII (e.g., IHF or HU) interaction. Accordingly, the disclosure permits the use of virtual design techniques, also known as computer-aided, in silico design or modeling, to design, select, and synthesize agents capable of interacting with, binding to, or inhibiting the DNA-DNABII (e.g., IHF or HU) interaction. In turn, the candidate agents may be effective in the treatment of biofilms and associated diseases or conditions (medical, industrial or veterinary) as described herein. Thus, the present disclosure also provides agents identified or designed by the in silico methods.

A candidate agent is found to be able to bind to DNA and/or DNABII protein if a desired interaction between the candidate agent and either or both is found. The interaction can be quantitative, e.g., strength of interaction and/or number of interaction sites, or qualitative, e.g., interaction or lack of interaction. The output of the method, accordingly, can be quantitative or qualitative. In one aspect, therefore, the present disclosure also provides a method for identifying an agent that does not inhibit the interaction or alternatively, strengthens the interaction between the DNA and protein.

The potential inhibitory or binding effect (i.e., interaction or association) of an agent such as a small molecule compound may be analyzed prior to its actual synthesis and testing by the use of computer modeling techniques. If the theoretical structure of the given compound suggests insufficient interaction and association between it and microbial DNA in the biofilm and/or DNABII protein, synthesis and testing of the agent can be obviated. However, if computer modeling indicates a strong interaction, the agent can then be synthesized and tested for its ability to bind to or inhibit the interaction using various methods such as in vitro or in vivo experiments. Methods of testing an agent's ability to inhibit or titrate a biofilm, alone or in connection with another agent, are disclosed herein. In this manner, synthesis of inoperative agents and compounds can be avoided.

One skilled in the art may use any of several methods to screen chemical or biological entities or fragments for their ability to associate with DNABII or microbial DNA and more particularly with the specific binding sites. Selected fragments or chemical entities may then be positioned in a variety of orientations, or docked, within an individual binding site of DNA or DNABII polypeptide. Docking may be accomplished using software such as QUANTA, SYBYL, followed by energy minimization and molecular dynamics with standard molecular mechanical force fields, such as CHARMM and AMBER.

Commercial computer programs are also available for in silico design. Examples include, without limitation, GRID (Oxford University, Oxford, UK), MCSS (Molecular Simulations, Burlington, Mass.), AUTODOCK (Scripps Research Institute, La Jolla, Calif.), DOCK (University of California, San Francisco, Calif.), GLIDE (Schrodinger Inc.), FlexX (Tripos Inc.) and GOLD (Cambridge Crystallographic Data Centre).

Once an agent or compound has been designed or selected by the above methods, the efficiency with which that agent or compound may bind to each other can be tested and optimized by computational evaluation. For example, an effective DNABII fragment or may demonstrate a relatively small difference in energy between its bound and free states (i.e., a small deformation energy of binding).

A compound designed or selected can be further computationally optimized so that in its bound state it may optionally lack repulsive electrostatic interaction with the target protein. Such non-complementary (e.g., electrostatic) interactions include repulsive charge-charge, dipole-dipole, and charge-dipole interactions. Specifically, the sum of all electrostatic interactions between the agent and DNABII and/or microbial DNA in the biofilm when the agent or compound is bound to either agent, optionally making a neutral or favorable contribution to the enthalpy of binding.

Computer softwares are also available in the art to evaluate compound deformation energy and electrostatic interaction. Examples include, without limitation, Gaussian 92 [Gaussian, Inc., Pittsburgh, Pa.]; AMBER [University of California at San Francisco]; QUANTA/CHARMM [Molecular Simulations, Inc., Burlington, Mass.]; and Insight II/Discover [Biosysm Technologies Inc., San Diego, Calif.].

Once a binding agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to the DNABII protein and/or microbial DNA in the biofilm by the same computer methods described in detail, above.

Certain embodiments relate to a method for screening small molecules capable of interacting with the protein or polynucleotide disclosed herein. For the purpose of this disclosure, “small molecules” are molecules having low molecular weights (MW) that are, in one embodiment, capable of binding to a protein of interest thereby altering the function of the protein. In some embodiments, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell culture or tissue infected with the microbial to be treated are first provided. The cells are cultured under conditions (temperature, growth or culture medium and gas (C02)) and for an appropriate amount of time to attain exponential proliferation without density dependent constraints. It also is desirable to maintain an additional separate cell culture that is not infected as a control.

As is apparent to one of skill in the art, suitable cells can be cultured in micro-titer plates and several agents can be assayed at the same time by noting genotypic changes, phenotypic changes or a reduction in microbial titer.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined,

When the agent is an antibody or antigen-binding fragment, the agent can be contacted or incubated with the target antigen and polyclonal antibody as described herein under conditions to perform a competitive ELISA. Such methods are known to the skilled artisan.

The assays also can be performed in a subject. When the subject is an animal such as a rat, chinchilla, mouse or simian, the method provides a convenient animal model system that can be used prior to clinical testing of an agent in a human patient. In this system, a candidate agent is a potential drug if symptoms of the disease or microbial infection is reduced or eliminated, each as compared to untreated, animal having the same infection. It also can be useful to have a separate negative control group of cells or animals that are healthy and not treated, which provides a basis for comparison.

The agents and compositions can be used in the manufacture of medicaments and for the treatment of humans and other animals by administration in accordance with conventional procedures, such as an active ingredient in pharmaceutical compositions.

### Combination Therapy

The compositions and related methods of the present disclosure may be used in combination with the administration of other therapies. These include, but are not limited to, the administration of DNase enzymes, antibiotics, antimicrobials, anti-infectives, antifungals, anti-parasitics, anti-virals, or other antibodies and HMGB polypeptides as described herein.

In some embodiments, the methods and compositions include a deoxyribonuclease (DNase) enzyme that acts synergistically with the anti-DNABII antibody or an antigen-binding fragment thereof. A DNase is any enzyme that catalyzes the cleavage of phosphodiester linkages in the DNA backbone. Three non-limiting examples of DNase enzymes that are known to target not only cruciform structures, but also a variety of secondary structure of DNA include DNAse I, T4 EndoVII, T7 Endo I, RuvABC, and RusA. In certain embodiments, the effective amount of anti-DNABII antibody or an antigen-binding fragment thereof needed to destabilize the biofilm is reduced when combined with a DNase. When administered in vitro, the DNase can be added directly to the assay or in a suitable buffer known to stabilize the enzyme. The effective Unit dose of DNase and the assay conditions may vary, and can be optimized according to procedures known in the art.

In other embodiments, the methods and compositions can be combined with antibiotics and/or antimicrobials. Antimicrobials are substances that kill or inhibit the growth of microorganisms such as bacteria, fungi, or protozoans. Although biofilms are generally resistant to the actions of antibiotics, compositions and methods described herein can be used to sensitize the infection involving a biofilm to traditional therapeutic methods for treating infections. In other embodiments, the use of antibiotics or antimicrobials in combination with methods and compositions described herein allow for the reduction of the effective amount of the antimicrobial and/or biofilm reducing agent. Some non-limiting examples of antimicrobials and antibiotics useful in combination with methods of the current disclosure include amoxicillin, amoxicillin-clavulanate, cefdinir, azithromycin, and sulfamethoxazole-trimethoprim. The therapeutically effective dose of the antimicrobial and/or antibiotic in combination with the biofilm reducing agent can be readily determined by traditional methods. In some embodiments, the dose of the antimicrobial agent in combination with the biofilm reducing agent is the average effective dose which has been shown to be effective in other bacterial infections, for example, bacterial infections wherein the etiology of the infection does not include a biofilm. In other embodiments, the dose is 0.1, 0.15, 0.2, 0.25, 0.30, 0.35, 0.40, 0.45, 0.50, 0.55, 0.60, 0.65, 0.70, 0.75, 0.8, 0.85, 0.9, 0.95, 1.1, 1.2, 1.3, 1.4, 1.5, 1.6, 1.7, 1.8, 1.9, 2.0, 2.5, 3.0 or 5 times the average effective dose. The antibiotic or antimicrobial can be added prior to, concurrent with, or subsequent to the addition of the anti-DNABII antibody or antigen-binding fragment thereof.

In other embodiments, the methods and compositions can be combined with antibodies that treat the bacterial infection. One example of an antibody or an antigen-binding fragment thereof useful in combination with the methods and compositions described herein is an antibody or an antigen-binding fragment thereof directed against an unrelated outer membrane protein (i.e., OMP P5). Treatment with this antibody or antigen-binding fragment thereof alone does not debulk a biofilm in vitro. Combined therapy with this antibody or antigen-binding fragment thereof and a biofilm reducing agent results in a greater effect than that which could be achieved by either reagent used alone at the same concentration. Other antibodies that may produce a synergistic effect when combined with a biofilm reducing agent or methods to reduce a biofilm include anti-rsPilA anti-OMP26, anti-OMP P2, and anti-whole OMP preparations.

The compositions and methods described herein can be used to sensitize the bacterial infection involving a biofilm to common therapeutic modalities effective in treating bacterial infections without a biofilm but are otherwise ineffective in treating bacterial infections involving a biofilm. In other embodiments, the compositions and methods described herein can be used in combination with therapeutic modalities that are effective in treating bacterial infections involving a biofilm, but the combination of such additional therapy and biofilm reducing agent or method produces a synergistic effect such that the effective dose of either the biofilm reducing agent or the additional therapeutic agent can be reduced. In other instances, the combination of such additional therapy and biofilm reducing agent or method produces a synergistic effect such that the treatment is enhanced. An enhancement of treatment can be evidenced by a shorter amount of time required to treat the infection.

The additional therapeutic treatment can be added prior to, concurrent with, or subsequent to methods or compositions used to reduce the biofilm, and can be contained within the same formation/composition or as a separate formulation/composition.

### Kits

Kits containing the agents and instructions necessary to perform the in vitro and in vivo methods as described herein also are claimed. Accordingly, the disclosure provides kits for performing these methods which may include an antibody, antigen-binding fragment thereof, polypeptide, polynucleotide, vector or host cell, as well as instructions for carrying out the methods disclosed herein such as collecting tissue and/or performing the screen, and/or analyzing the results, and/or administration of an effective amount of an antibody, antigen-binding fragment, polypeptide, polynucleotide, vector or host cell, as defined herein. These can be used alone or in combination with other suitable antimicrobial agents.

For example, a kit can comprise, or alternatively consist essentially of, or yet further consist of any one or more of agents identified above, e.g., antibody, antigen-binding fragment, polypeptide, polynucleotide, vector or host cell, and instructions for use. The kit can further comprise one or more of an adjuvant, an antigenic peptide or an antimicrobial. Examples of carriers include a liquid carrier, a pharmaceutically acceptable carrier, a solid phase carrier, a pharmaceutically acceptable carrier, a pharmaceutically acceptable polymer, a liposome, a micelle, an implant, a stent, a paste, a gel, a dental implant, or a medical implant.

The following examples and Appendices (incorporated by reference in their entireties) are intended to illustrate, and not limit the embodiments disclosed herein.

### EXPERIMENTS

### Experiment No. 1—The Extracellular Innate Immune Effector, HMGB1, Limits Pathogenic Bacterial Biofilm Proliferation

The effects of HMGB1 were examined on bacterial biofilms. Remarkably, it was found that, despite the similarities in DNA substrate preference, whereas DNABII proteins stabilized biofilm structural integrity, HMGB1 disrupted bacterial biofilm structure. The breadth of anti-biofilm activity of HMGB1 was determined against multiple pathogenic biofilms in vitro via an assay against multiple high priority pathogens followed by assessment of HMGB1's ability to therapeutically resolve biofilm-mediated infections in two distinct animal models of human disease. Moreover, it was assessed whether HMGB1 can be engineered to eradicate its pro-inflammatory activity without loss of its anti-biofilm activity via modification of a single key amino acid. A therapeutic cocktail of host-derived modified HMGB1 plus pathogen-directed antibody against DNABII protein was then tested to determine the ability of this cocktail to eradicate biofilms formed by a predominant bacterial pathogen of the human respiratory tract via use of an experimental model of otitis media (OM). Finally, a model was proposed to describe the native extracellular functions of HMGB1, now inclusive of its newly recognized anti-biofilm activity.

HMGB1 and DNABII Proteins Localized to Distinct Regions on the Lattice Structure of eDNA within an In Vivo Biofilm

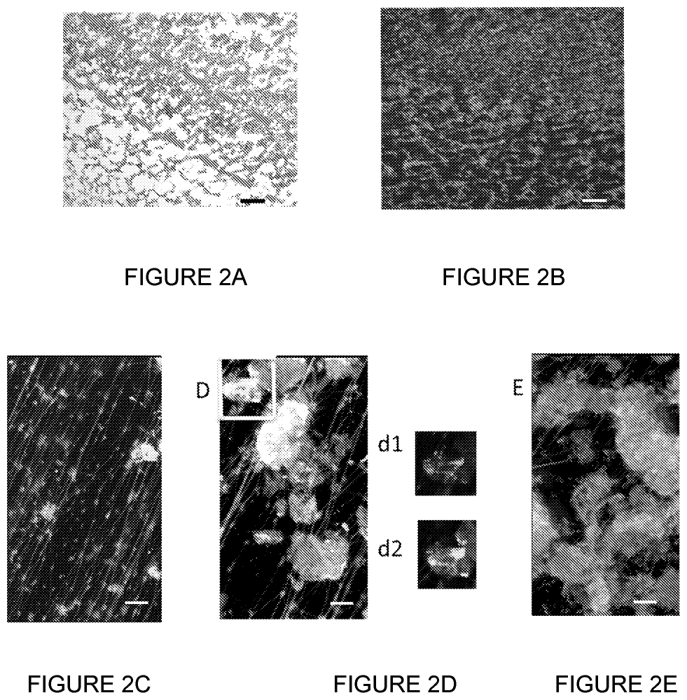

Since HMGB1 is bound to DNA within NETs (Peng et al.), the primary host defense against pathogens and that HMGB1 and DNABII proteins functionally complement each other in several in vitro transactions, by virtue of their shared ability to bind to and bend DNA, it was first attempted to localize host HMGB1 and bacterial DNABII proteins within a biofilm that had formed in vivo to begin to characterize the potential role of HMGB1 in host defense against bacterial biofilms. To localize HMGB1 and DNABII proteins within the eDNA-rich matrix of biofilms recovered from the chinchilla middle ear during otitis media induced by nontypeable Haemophilus influenzae (NTHI), biofilm specimens were probed with antibodies specific for either HMGB1 or DNABII proteins. A region was identified with clear delineation of bacterial biofilm from that of neutrophils/NETs, with an approximately 236 micron region of apparent overlap (FIGS. 2A&2B). Immunofluorescence microscopy was then used to label eDNA, DNABII proteins or HMGB1 within the regions of exclusive bacterial biofilm, the overlapping interface and that region of exclusively neutrophil/NETs (FIGS. 2C, 2D & 2E). eDNA lattice can be seen in all three panels with DNABII proteins bound to the eDNA exclusively at the vertices of the eDNA in biofilm panel (FIG. 2C) and HMGB1 bound to eDNA exclusively in the neutrophil/NET panel (FIG. 2E). In contrast, at the interface there is clear labeling of both DNABII proteins and HMGB1 on the eDNA. HMGB1 was detected in close proximity to DNABII protein at the crossed strands of eDNA, however these proteins did not co-localize at the vertices (FIG. 2) despite what might appear to be a small amount of overlaying within the demarked box in the upper left hand corner of FIG. 2D which is an artifact due to compression of multiple Z-stack images. This is evident within the individual Z-stack images (FIG. 2D, right panels). This result suggested that host HMGB1 was indeed incorporated into the eDNA-dependent extracellular matrix of bacterial biofilms, similar to bacterial DNABII proteins. However, unlike DNABII proteins, HMGB1 was never observed at the vertices of crossed strands of eDNA within the lattice, which suggested that it did not stabilize the HJ-like structures, yet another function that is dissimilar to that of the DNABII proteins.

Recombinant HMGB1 disrupted biofilms formed by diverse critical and high priority human pathogens, in vitro.

Since HMGB1 could be incorporated within the bacterial biofilm EPS, the effect of recombinant HMGB1 (rHMGB1) was determined directly on bacterial biofilm architecture. To do so, examined herein were in vitro multiple human pathogens that in part mediate their virulence through the biofilm state. These include uropathogenic Escherichia coli (UPEC), Burkholderia cenocepacia (Bc), NTHI, Enterobacter spp (E), Staphylococcus aureus (S), Klebsiella pneunomiae (K), Acinetobacter baumanii (A), Pseudomonas aeruginosa (P), and Enterococcus faecium (E) that are defined as critical and high priority by the World Health Organization (WHO, Global Priority List of Antibiotic-resistant Bacteria to Guide Research, discovery, and Development of New Antibiotics, WHO PPL Short Summary (2017)). Twenty-four hour biofilms formed by each of these pathogens were incubated with 200 nM rHMGB1 for 16 hours with the exception of E. faecium, wherein the incubation period was shortened to 1 h to avoid potential degradation of rHMGB1 by its secreted protease (Ch'ng et al., Nat Rev Microbiol, 2019. 17(2): p. 82-94). Antibody directed against E. coli IHF (α-IHF_{Ec}; recognizes both IHF and HU), that disrupts biofilms formed by multiple bacterial species (Devaraj et al.; Goodman et al., 2011; Gustave et al.; Novotny et al., 2013; Brandstetter et al.; Brockson et al., Mol Microbiol. 2014 September; 93(6):1246-58; and Freire et al., Mol Oral Microbiol, 2016. 2017 February; 32(1):74-88) was used as a positive control.

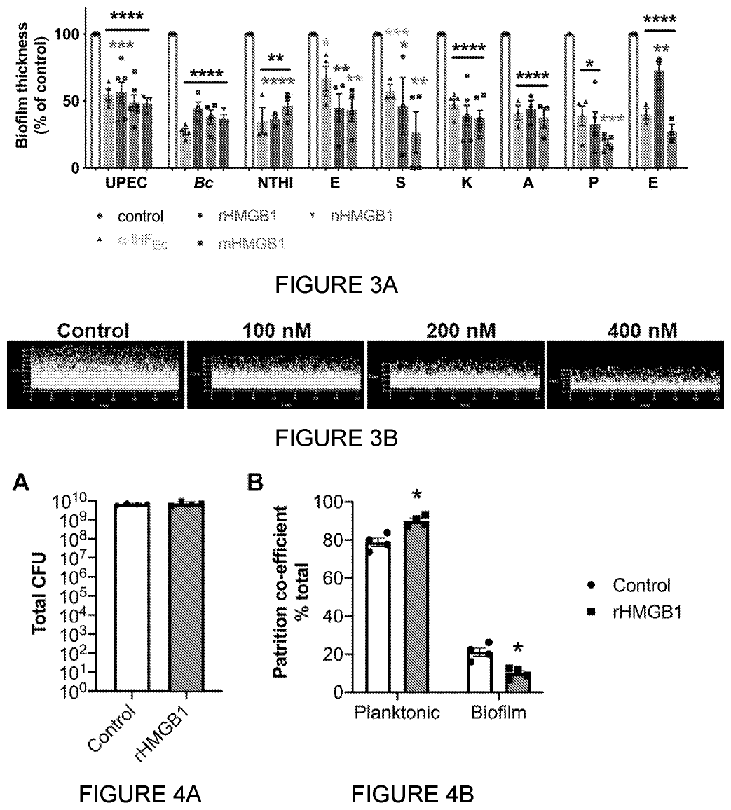

As shown in FIG. 3, it was found that rHMGB1 disrupted biofilms formed by each of these pathogens as evidenced by a significant reduction in biofilm average thickness compared to the control, wherein biofilms were incubated in medium only (FIG. 3A). Only S. aureus and E. faecium required a greater, albeit non-bactericidal, dose (800 nM) of rHMGB1 to achieve a similar reduction in biofilm average thickness (FIG. 3A). Additionally, the anti-biofilm activity of native HMGB1 (nHMGB1), purified from calf thymus, on UPEC and B. cenocepacia biofilms was equivalent to that of rHMGB1 (FIG. 3A), which indicated that any potential differences in PTMs between nHMGB1 and rHMGB1 did not significantly impact the anti-biofilm activity.

Notably, the dose-dependent activity of rHMGB1 to disrupt UPEC biofilms is demonstrated in FIG. 3B. Next enumerated were the relative concentrations of the planktonic versus the biofilm-resident UPEC after incubation with rHMGB1 and found that rHMGB1 did not exhibit any bactericidal effect as there was no statistically significant difference in total CFUs, compared to the control (FIG. 4A). However, and interestingly, rHMGB1 appeared to induce a shift of bacteria from biofilm-residence into the planktonic state as evidenced by the statistically significant increase in the planktonic bacteria and the concomitant statistically significant decrease in biofilm-resident bacteria within this culture system (FIG. 4B).

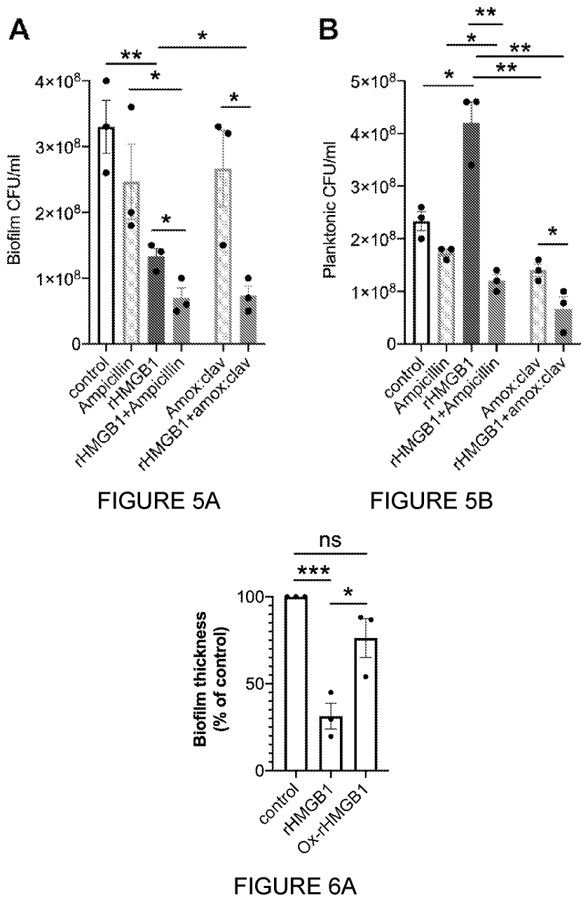

Finally, to directly test the efficacy of antibiotics in the presence of rHMGB1, NTHI biofilms were incubated with either antibiotics (ampicillin (32 μg/ml) or amoxicillin-clavulanate (1 μg/ml)) alone (Brockson et al.) or in combination with rHMGB1 (200 nM) for 16 hours. The relative concentrations of the planktonic versus the biofilm-resident NTHI were enumerated and it was found that while rHMGB1 induced a shift of bacteria from biofilm-residence into the planktonic state, rHMGB1 in combination with either ampicillin or amoxicillin-clavulanate killed a statistically significant amount of the total bacteria, likely by the ability of these antibiotics to kill the planktonic bacteria and rHMGB1 to drive the bacteria into the vulnerable planktonic state (FIG. 5). Without being bound by theory, rHMGB1 synergized with antibiotics in vitro in the clearance of bacterial biofilms.

Oxidation of rHMGB1 Negatively Affected the Antibiofilm Activity of rHMGB1

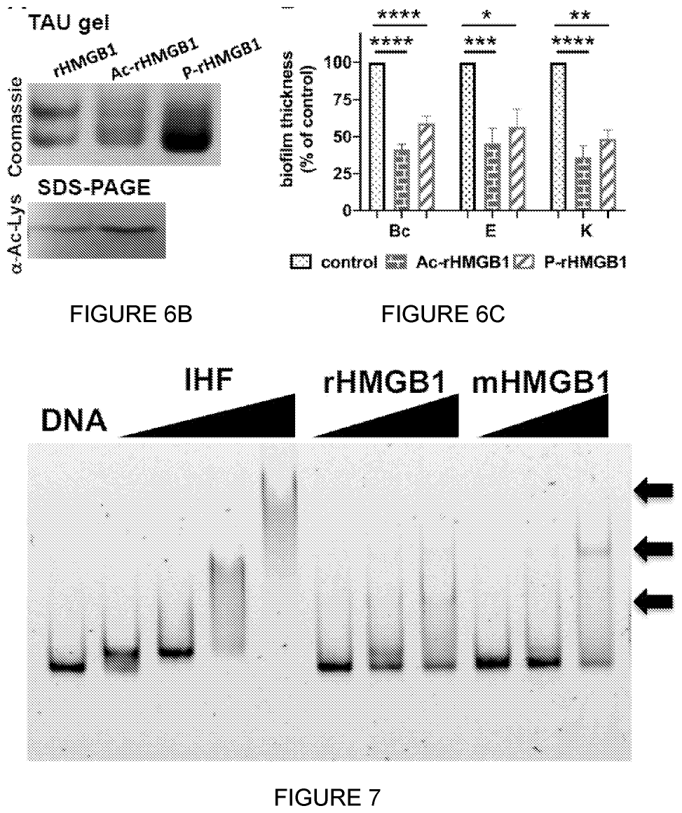

Several PTMs are described for HMGB1 that modulate its location, function, and ability to bind DNA (reviewed in Kang et al.). Although LC-MS/MS analysis of rHMGB1 and nHMGB1 revealed that <20% of the peptides from either exhibited any PTM (data not shown), the effect of oxidation on the anti-biofilm activity of rHMGB1 was nonetheless examined given that the redox state of HMGB1 is known to modulate its function (Venereau et al., J Exp Med, 2012. 209(9): p. 1519-28). The oxidation state of the three cysteine (C) residues at positions 23, 45, and 106 influences the inflammatory properties (Antoine et al., Mol Med, 2014. 20: p. 135-7). HMGB1 with the C106 thiol group and a C23-C45 disulfide bond triggers inflammation, whereas terminally oxidized or reduced cysteines promote resolution of inflammation (Yang et al., Mol Med, 2012. 18: p. 250-9). To this end, HMGB1 was first oxidized with hydrogen peroxide as described (Liu et al., PLoS One, 2012. 7(4): p. e35379). Ox-rHMGB1 was then evaluated to determine its relative ability to disrupt biofilms formed by UPEC. Ox-rHMGB1 lost its anti-biofilm effect as compared to rHMGB1 (FIG. 6A). This result suggested that oxidation of rHMGB1 significantly impaired its biofilm disruption ability.

Neither Acetylation Nor Phosphorylation Affected the Antibiofilm Activity of rHMGB1

The effect of acetylation or phosphorylation on the anti-biofilm activity of rHMGB1 was examined given that only acetylation and phosphorylation are known to enhance the affinity of HMGB1 to HJ DNA (Pasheva et al., 2004, Biochemistry, 43, 2935-40; and Ugrinova et al., 2012, Mol Biol Rep, 39, 9947-53). Since HJ-like structures are critical to bacterial biofilm stability (Devaraj et al., 2019), the anti-biofilm activity of acetylated (Ac-rHMGB1) or phosphorylated (P-rHMGB1) isoforms of HMGB1 was evaluated. Ac-rHMGB1 migrated slower and PrHMGB1 migrated faster than rHMGB1 as seen in FIG. 6B, as expected (Ryan and Annunziato, 2001, Curr Protoc Mol Biol, Chapter 21, Unit 21 2). It was further confirmed the acetylation of rHMGB1 via use of α-acetyllysine antibody (FIG. 6B; bottom). Ac-rHMGB1 and P-rHMGB1 were then evaluated to determine their relative abilities to disrupt biofilms formed by B. cenocepacia, Enterobacter spp, or K. pneumoniae. Ac-rHMGB1 and P-rHMGB1 significantly disrupted biofilms formed by each of these pathogens as evidenced by the significant reduction in biofilm average thickness compared to control (FIG. 6C). Importantly, the anti-biofilm activity of these isoforms was indistinguishable from that of rHMGB1 (FIGS. 3A & 6C).

Recombinant HMGB1 with an Engineered Single Amino Acid Mutation C45S Retained its Anti-Biofilm Function In Vitro Against Multiple Human Pathogens

Thereby, to alleviate any undesired pro-inflammatory effect of rHMGB1, a C45S point mutation was engineered to generate modified HMGB1 (mHMGB1) that abrogates disulfide bond formation with C23. To validate this variant, it was first demonstrated that mHMGB1 retained its ability to bind to HJ DNA, a known binding substrate of HMGB1 (FIG. 7). Next, the anti-biofilm function of mHMGB1 was assayed as described above and it was demonstrated that mHMGB1 fully retained the anti-biofilm activity (FIG. 3A). Only E. faecium required a higher dose of mHMGB1 to achieve a similar reduction in biofilm average thickness. Without being bound by theory, the engineered C45S variant of HMGB1 retained biofilms disruption capability.

Unlike DNABII Proteins, HMGB1 Did not to Stabilize Model HJs and the Lattice-Like eDNA Network within K. pneumoniae Biofilms In Vitro

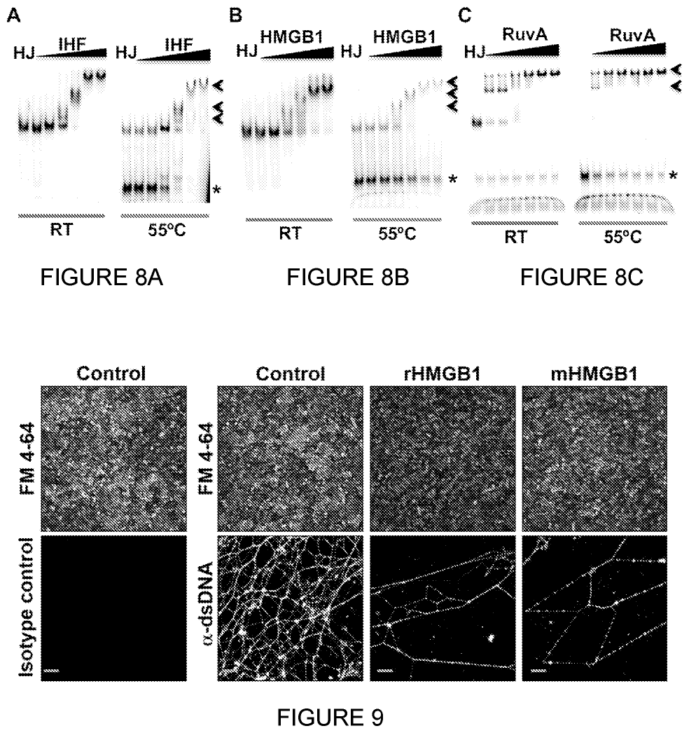

The presence of HJ-like structures within the eDNA lattice of bacterial biofilms was shown (Devaraj et al., 2019), and given its high affinity for such branched DNA structures (Bianchi et al.), it was hypothesized without wishing to be bound by the theory that HMGB1 likely destabilized HJ DNA upon binding, which was why it was never observed at the vertices of crossed strands of eDNA (see FIG. 2). To test the hypothesis, HJ DNA was incubated with either HMGB1, the DNABII protein IHF, or the prototypic HJ DNA binding protein, RuvA at room temperature (RT) or 55° C. (melting temperature of HJ DNA) and resolved the complexes by non-denaturing polyacrylamide gel electrophoresis (PAGE). While all three proteins bound to HJ DNA to form stable complexes at RT, only HMGB1 was unable to efficiently stabilize the HJ DNA at 55° C. (FIG. 8) as indicated by the decrease in abundance of the shifted HJ DNA-HMGB1 complex (arrow) and the concomitant increase in the constituent melted oligos (asterisk). These data suggested that despite a similar HJ DNA binding site preference to both IHF and RuvA, HMGB1 was unable to likewise stabilize the HJ structure.

To directly evaluate the effect of HMGB1 isoforms on the eDNA-dependent extracellular matrix, incubation with 24-hour K. pneumoniae biofilms (used here as a representative model bacterial biofilm) with either rHMGB1 or mHMGB1 was performed for 16 hours. Unfixed biofilms were labeled with a monoclonal antibody against double stranded (ds) DNA to visualize the eDNA within the biofilm matrix. Bacteria were labeled with FilmTracer FM 4-64 (top panels). eDNA within K. pneumoniae biofilms was organized into a complex web-like structure (bottom panels), that was notably disrupted upon incubation with rHMGB1 or mHMGB1. This outcome corresponded with a concurrent substantial reduction in biofilm bacteria in the presence of either isoform of HMGB1 (FIG. 9). Collectively, these data suggested that both rHMGB1 and mHMGB1 directly destabilized the biofilm extracellular matrix by specifically disrupting the eDNA lattice which resulted in biofilm disruption in vitro. This result was consistent with what was observed when labelling a biofilm that had formed in vivo, wherein HMGB1 was not found at the vertices of crossed strands of eDNA within the scaffold. Per the model as disclosed herein, HMGB1 would compete with DNABII proteins, wherein instead of stabilization, HMGB1 would de-stabilize these HJ-like structures and consequently disrupt the biofilm.

HMGB1 Disrupted Biofilms Via its Ability to Bind to HJ-Like Structures within the Biofilm Extracellular Matrix

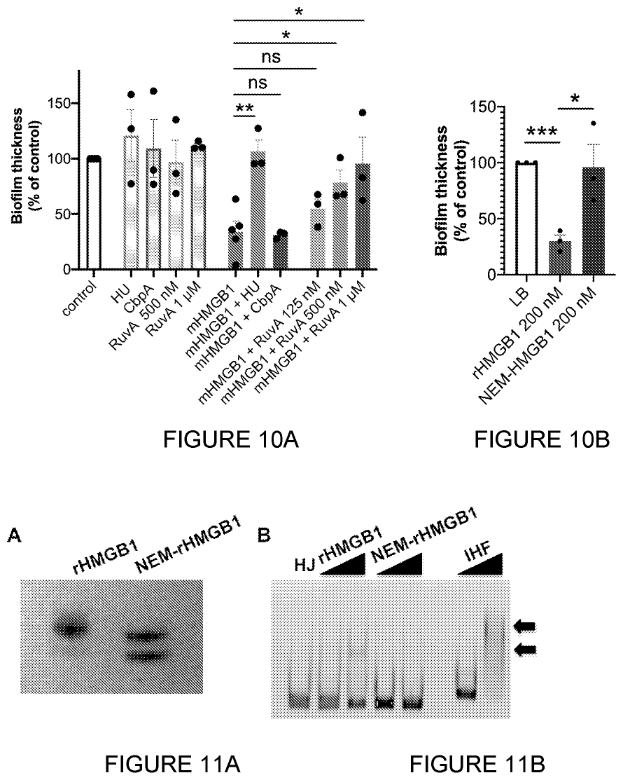

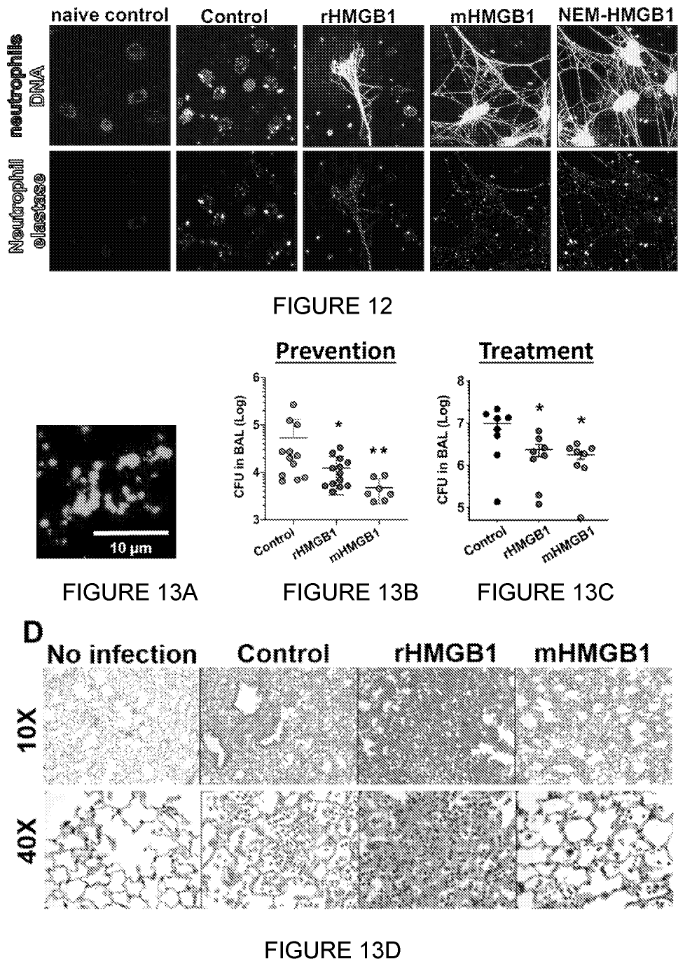

Without wishing to be bound by the theory, it was hypothesized that HMGB1 could mediate its anti-biofilm effect either by binding directly to HJ DNA within the eDNA matrix or by binding to DNABII proteins such that these proteins could no longer stabilize the bacterial extracellular matrix. First, the possibility that HMGB1 mediated its anti-biofilm effect was tested by directly binding to HJ DNA. To this end, the anti-biofilm function of HMGB1 was assayed as described above but only in the presence of exogenously added HU (DNABII protein or RuvA (prototypic HJ DNA-binding protein) that would compete with mHMGB1 for similar binding sites within the eDNA or CbpA, another bacterial nucleoid associated protein as a negative control; it was previously shown that RuvA can replace DNABII proteins for function in biofilm EPS (Devaraj et al. 2019), whereas CbpA is not required for the maintenance of the eDNA lattice structure within the EPS (Devaraj et al., 2017). It was observed that mHMGB1 was unable to disrupt biofilms formed by UPEC in the presence of either added DNABII protein or RuvA in a dose-dependent manner, but could still disrupt UPEC biofilms in the presence of CbpA (FIG. 10A), which suggested that mHMGB1 mediated its anti-biofilm effect via its ability to bind to HJ-like structures within the biofilm extracellular matrix. Second, since alkylation of HMGB1 with N-ethylmaleimide (NEM) has been shown to inhibit HMGB1's ability to bind to DNA (Sheflin et al., Biochemistry, 1993. 32(13): p. 3238-48), NEM treated rHMGB1 was used to directly test the mechanism of HMGB1-mediated biofilm disruption. It was first verified that rHMGB1 was modified by NEM by triton-acetic acid-urea gel that revealed a shift in NEM-rHMGB1 as compared to rHMGB1 (FIG. 11A). Next, it was then demonstrated that NEM-rHMGB1 was unable to bind to HJ DNA (FIG. 11B). To verify that NEM-HMGB1 was still folded properly and otherwise functional, the ability of NEM-HMGB1 to induce neutrophils to form NETs was assayed. Human neutrophils were isolated from fresh blood and were incubated in the absence or presence of NEM-rHMGB1 for 3.5 hours. Neutrophils were fixed and then labeled with a monoclonal antibody against double stranded (ds) DNA to visualize the eDNA (NETs, top panels of FIG. 12) and a polyclonal antibody against neutrophil elastase to demark the NET-derived eDNA (as shown in the bottom panels of FIG. 12). Neutrophils themselves were labeled with wheat germ agglutinin (WGA) conjugated with ALEXAFLUOR® 350 (as shown in the top panels of FIG. 12). As shown in FIG. 12, NEM-HMGB1 induced neutrophils to form NETs, which suggested that NEM modification of rHMGB1 only specifically interfered with its ability to bind to HJ DNA. Finally, the anti-biofilm function of NEM-rHMGB1 was assayed as described above and it was demonstrated that NEM-rHMGB1 lost its anti-biofilm activity (FIG. 10B). These results suggested that HMGB1 disrupted biofilms via its ability to directly bind to HJ-like structures within the biofilm extracellular matrix.

Next, the possibility that HMGB1 mediated its anti-biofilm effect was tested by binding to DNABII proteins. To this end, the equilibrium dissociation constant (K_{d}) was measured as a measure of binding affinity of mHMGB1 to IHF_{NTHI} and HU_{NTHI} by BIACORE™ surface plasmon resonance (SPR) analysis and found that the K_{d} for mHMGB1 binding to IHF_{NTHI} was 579 nM and to HU_{NTHI} was 104 nM (Table 1). Since full-length HMGB1 binds to HJ DNA with at least an order of magnitude higher affinity (K_{d}˜10 nM) (Xin et al., Nucleic Acids Res, 2000. 28(20): p. 4044-50) as compared to either of the two DNABII proteins, collectively these results suggested that HMGB1 anti-biofilm effects were predominated by directly binding to its high affinity HJ DNA target and thus destabilized HJs rather than via protein-protein interactions with DNABII proteins.

HMGB1 Promoted Clearance of B. cenocepacia Aggregates from the Mouse Lung

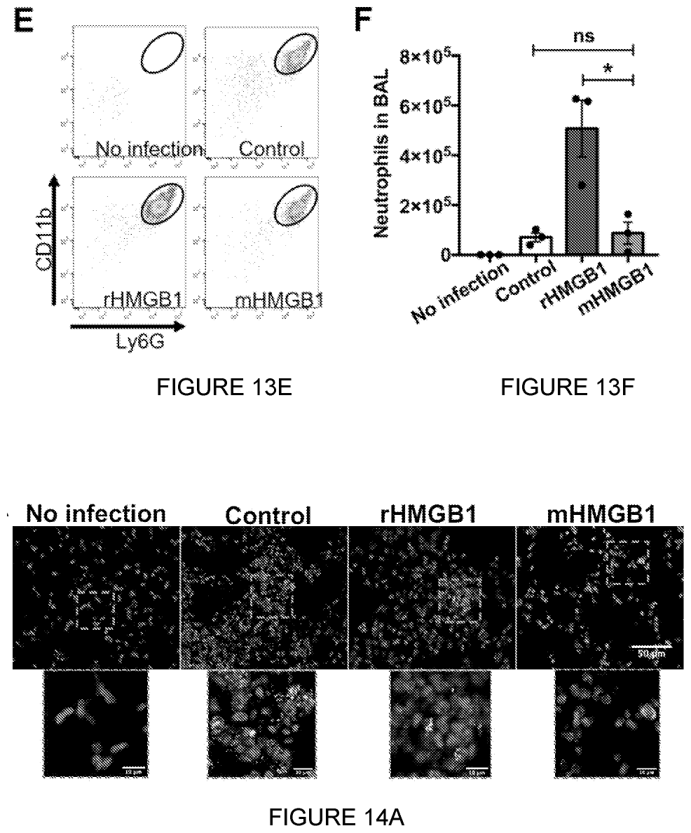

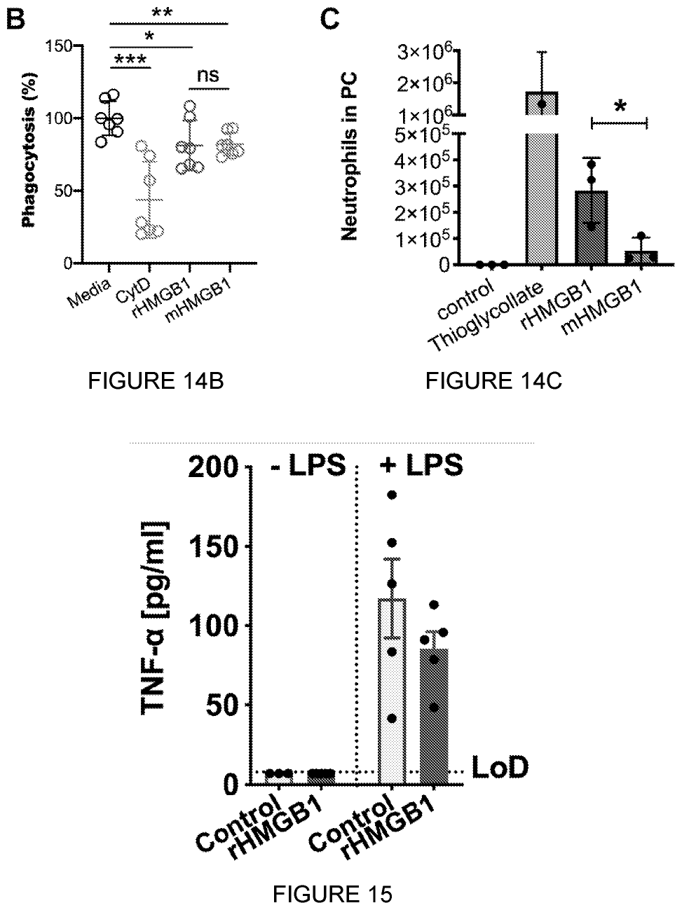

Since rHMGB1 and mHMGB1 disrupted biofilms formed by multiple bacteria in vitro, the potential anti-biofilm activity was evaluated in a murine model of lung infection mediated by B. cenocepacia. C57BL/6 mice were challenged intratracheally (i.t.) with 10⁷ CFU of B. cenocepacia, and at either the time of challenge (prevention cohort) or at 24 hours post infection (hpi) (treatment cohort), mice received 0.2 nmol of either rHMGB1 or mHMGB1 i.t. B. cenocepacia was immunolabeled within the mouse lungs at 72 hpi via use of a monoclonal antibody against E. coli Elongation factor Tu (α-EF-Tu; cross reacts with B. cenocepacia) and it was demonstrated that B. cenocepacia formed aggregates within the lungs (FIG. 13A). Next, the bacterial burden was determined within the lungs either 18 hpi (prevention) or at 72 hpi (treatment) and demonstrated that both rHMGB1 and mHMGB1 significantly facilitated the clearance of B. cenocepacia from the lungs, regardless of preventative (FIG. 13B) versus treatment strategy used (FIG. 13C). B. cenocepacia was stained within the lung tissue at 72 hpi and observed a large number of bacteria within the lungs of the control mice (FIG. 14A). Strikingly, there was a significantly reduced bacterial load in mice treated with either rHMGB1 or mHMGB1 compared to the control (FIG. 14A). These data suggested that both the preventative and treatment approaches with either rHMGB1 or mHMGB1 facilitated clearance of B. cenocepacia from the mouse lung.

Although both rHMGB1 and mHMGB1 facilitated clearance of B. cenocepacia from the mouse lung, since HMGB1 is known to reduce the phagocytic capacity of macrophages (Banerjee et al., J Immunol, 2011. 187(9): p. 4686-94), the effect of mHMGB1 was evaluated on phagocytosis by macrophages. It was found that mHMGB1 moderately reduced the phagocytic capacity of macrophages as compared to control and was indistinguishable from rHMGB1 (FIG. 14B). Cytochalasin D was used as a positive control. These results suggested that a slight reduction in phagocytosis of macrophages upon incubation with rHMGB1 or mHMGB1 did not affect bacterial clearance at 48 hpi, further suggesting that the primary therapeutic function of HMGB1 was not phagocytosis dependent.

Since HMGB1 can induce a potentially detrimental pro-inflammatory response, the ability of rHMGB1 or mHMGB1 to induce the inflammatory recruitment of neutrophils into the murine peritoneum was assessed. Mice were injected intraperitoneally (i.p.) with 0.2 nmol of either rHMGB1 or mHMGB1 and relative concentration of neutrophils within the peritoneal cavity was determined after 4 hours by flow cytometry with α-CD45, α-CD11b and α-Ly6G antibodies. Thioglycollate was used as a positive control to induce peritoneal inflammation. Whereas thioglycolate-injected mice showed significant neutrophil migration (2×10⁶ neutrophils) to the peritoneal cavity, those that received rHMGB1 showed a moderate (3×10⁵ neutrophils) yet significant neutrophil migration to the peritoneal cavity as compared to control (FIG. 14C). Strikingly, mice injected with mHMGB1 demonstrated a significant reduction in neutrophil migration (<1×10⁵ neutrophils) compared to rHMGB1 (FIG. 14), which suggested that mHMGB1 induced an attenuated inflammatory neutrophil response.

Further evaluated was inflammatory cell recruitment into the lungs of mice that had been challenged with B. cenocepacia, then treated with either rHMGB1 or mHMGB1 at 72 hpi. Whereas lungs from mice treated with rHMGB1 displayed a significant infiltration of inflammatory cells compared to control, as evidenced from the Haemotoxylin and Eosin (H&E) stain, lungs from mice treated with mHMGB1 showed no signs of inflammation and instead more closely resembled uninfected lungs (FIG. 13D). To identify the inflammatory cells that had migrated into the lungs after treatment with rHMGB1, flow cytometry was performed (using α-CD45, α-Ly6G and α-CD11b antibodies), on bronchoalveolar lavage (BAL) samples from mice infected with B. cenocepacia and simultaneously treated with either rHMGB1 or mHMGB1 at 18 hpi. While infected mice showed an infiltrate of primarily neutrophils, those similarly infected but also treated with rHMGB1 showed a significantly greater number of neutrophils in BAL compared to the control (FIGS. 13E & 13F). Conversely, mice treated with mHMGB1 showed a significant reduction of neutrophils in BAL compared to those treated with rHMGB1 and were indistinguishable from the control (FIGS. 13E & 13F).

Since excess HMGB1 can mediate dysregulated host response to infection associated with septic shock, the ability of rHMGB1 (0.2 nmol; the concentration used to treat in vivo biofilms) to induce septic shock was evaluated in mice. Mice were injected i.p. with either 0.2 nmol endotoxin-free rHMGB1, lipopolysaccharide (LPS; 5 mg/kg) or both, then monitored for signs of septic shock for 24 hours. Serum TNF-α, a gold standard for induction of septic shock (Stevens et al., Sci Rep, 2017. 7(1): p. 5850) revealed that while LPS alone induced a significant amount of TNF-α, rHMGB1 did not induce detectable TNF-α and was instead comparable to the control (FIG. 15). This result suggested that rHMGB1, which was used at the same concentration as a therapeutic in pre-clinical efficacy studies to resolve biofilms and induce disease resolution, was unlikely to promote systemic inflammation given the lack of evidence for such in these murine studies.

Lastly, since rHMGB1 not only induces neutrophil migration to the site of infection but also induces neutrophils to form neutrophil extracellular traps (NETs), the effect of mHMGB1 was evaluated on induction of NETs by neutrophils. Human neutrophils were isolated from fresh blood and were incubated in the absence or presence of either rHMGB1 or mHMGB1 for 3.5 hours. Neutrophils were fixed, then labeled as described above to visualize the NETs. While rHMGB1 had a modest effect on induction of NETs, mHMGB1 more strongly induced NET formation of neutrophils in vitro (FIG. 12). Collectively, these data suggested that while both rHMGB1 and mHMGB1 facilitated clearance of B. cenocepacia aggregates from the mouse lungs, only mHMGB1 did so without the pro-inflammatory activity of rHMGB1 in a mouse model of lung infection.

rHMGB1 and mHMGB1 Promoted Resolution of NTHI Biofilms within the Middle Ears in a Chinchilla Model of Experimental Otitis Media

An established model of experimental otitis media induced by NTHI was utilized to further evaluate the ability of rHMGB1 or mHMGB1 to disrupt adherent mucosal biofilms. Four days after transbullar challenge with NTHI, biofilms within both middle ears of chinchillas were treated with either rHMGB1 or mHMGB1 by direct instillation into the middle ear on two consecutive days (FIG. 16A). As a negative control, an equivalent volume of sterile saline was delivered. One day after receipt of the second therapeutic dose, all animals were sacrificed and NTHI within the middle ear biofilms was enumerated. The relative amount of mucosal biofilm and mucosal inflammation were also qualitatively assessed.

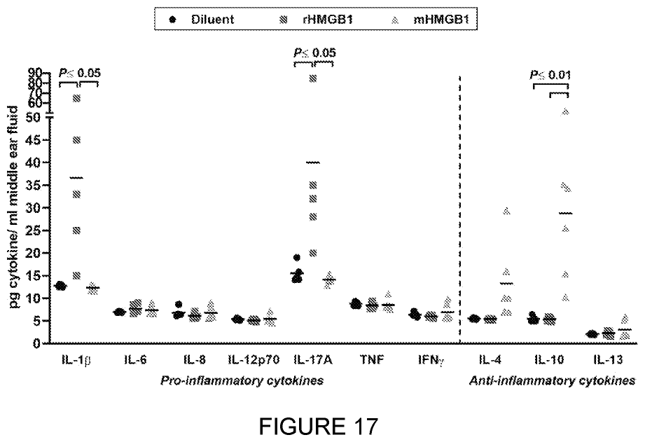

One day after receipt of the second treatment with rHMGB1 or mHMGB1, there were a significant >3-log fewer NTHI within mucosal biofilms and/or adherent to the middle ear mucosa, compared to the control cohort (FIG. 16B) (P<0.01). No difference in clearance was observed between these two treatments. Additionally, six blinded evaluators qualitatively ranked the relative amount of mucosal biofilm that remained within the chinchilla middle ears using an established rubric wherein a score of 0 indicated no biofilm present and 4+ indicated a middle ear filled with biofilm (FIG. 16C) (Novotny et al., Mucosal Immunol, 2011. 4(4): p. 456-67). Middle ears in the negative control cohort scored 3.1, i.e. ≥75% of the middle ear remained filled with mucosal biofilm (FIG. 16D). In contrast, those treated with rHMGB1 or mHMGB1 received a mean score of 1.0, i.e. <25% of the middle ear was occupied by biofilm (P<0.0001). Again, no difference was observed between treatments for relative amount of remaining biofilm. Next, middle ears were blindly qualitatively ranked for relative inflammation. A rubric wherein a score of 0 indicated no inflammation and 3+ indicated extensive capillary dilatation and presence of multiple hemorrhagic foci within the middle ear mucosa was used (FIG. 16E). The rHMGB1 cohort scored just slightly lower than the sterile saline control cohort (FIG. 16F; 1.7 vs. 2.2, respectively). Conversely, minimal inflammation was seen in the mHMGB1 cohort, significantly less than both the control and rHMGB1 cohorts (score, 0.7; P<0.0001). Images of representative middle ears are in FIG. 16G. Importantly, a statistically significant increase in pro-inflammatory cytokines (IL-1β and IL-17A) was also found in the middle ear fluids recovered from chinchillas that had been treated with rHMGB1 as compared to treatment with either mHMGB1 or diluent alone. Notably, there was also a statistically significant increase in anti-inflammatory cytokine (IL-10) in the middle ear fluids recovered from chinchillas that had been treated with mHMGB1 as compared with to treatment with either rHMGB1 or diluent alone (FIG. 17). Therefore, whereas both rHMGB1 and mHMGB1 promoted clearance of mucosal biofilms, only mHMGB1 did so without induction of overt inflammation (FIG. 16F & 16G).

Combinatorial Treatment with Host-Derived rHMGB1 and Pathogen-Directed α-DNABII Antibody Eradicated NTHI Biofilms within the Chinchilla Middle Ears

To determine if there might be added value in treating biofilms that had formed in vivo with a combination of mHMGB1 plus antibody (in the form of an antigen-binding fragment; Fab) against a DNABII protein to thereby develop a host-derived+pathogen-directed therapeutic strategy, the chinchilla model of experimental NTHI-induced otitis media was utilized. Four days after challenge with NTHI biofilms within the middle ears of chinchillas were treated with mHMGB1 (host-derived) and/or antibody against a chimeric peptide designed to mimic the immunoprotective domains of IHF (‘tip-chimer’, pathogen-directed), either alone or in combination (FIG. 16H). First, effectiveness of single treatment was evaluated. To this end, a subset of animals in each cohort was sacrificed one day after receipt of a single therapeutic dose (FIG. 16H) whereas the remaining animals in each cohort were sacrificed 24 h after receipt of a second treatment. Treatments included 200 nM mHMGB1 alone or admixed with 342 nM Fab fragments derived from rabbit anti-tip chimer IgG (‘tip chimer Fabs’), or tip chimer Fabs alone. As a negative control, Fab fragments derived from antibody directed against non-protective domains of IHF were used (‘tail-chimer Fabs’) (Novotny et al., 2019), either alone or combined with mHMGB1.

Infected middle ears were then treated with either of the aforementioned HMGB1 proteins alone or in combination with Fab fragments against either the tail or tips of IHF. Tail chimer Fabs failed to alter the extant biofilms regardless of the number of doses, as expected (FIG. 16I, labelled as “Tail chimer Fab”). Receipt of mHMGB1 plus tail chimer Fabs promoted a significant reduction in NTHI (P≤0.01) and 50% (3 of 6) of middle ears cleared NTHI from this niche (labelled as “Tail chimer Fab+mHMGB1”). This latter clearance was however due to the action of mHMGB1 which alone mediated significant clearance of NTHI from 50% (3 of 6) of middle ears after one dose (labelled as “mHMGB1”) which was further reduced after receipt of two doses. A single therapeutic dose of the DNABII-derived tip-chimer Fabs resulted in a significant 5-log reduction in biofilm-resident NTHI and 67% (4 of 6) of middle ears were culture-negative (labelled as “Tip chimer Fab”, P<0.05) which was further reduced by receipt of a second dose, as reported previously (Novotny et al., EBioMedicine, 2020. 59: p. 102867). Combining a single dose of mHMGB1 with tip-chimer Fabs was the most effective therapy tested, as 86% (5 of 6) of middle ears cleared NTHI from the middle ear (labelled as “Tip chimer Fab+mHMGB1”), which demonstrated at least an additive effect. Strikingly, treatment with 2 doses of mHMGB1 plus tip-chimer Fab eradicated NTHI from the middle ears of all animals in this cohort below the level of detection, indicating at least additive effect.

The middle ears of animals in these cohorts were also qualitatively evaluated for relative amount of remaining NTHI biofilm. Receipt of one or two doses of tail chimer Fabs was not effective and mean mucosal biofilm scores were >3.0 at each time point, which again indicated that ≥75% of the middle ear remained filled with NTHI biofilm (FIG. 16J, labelled as “Tail chimer Fab”). Admixing mHMGB1 with tail chimer Fabs significantly reduced the amount of biofilm present by half, however biofilms still filled between 25-50% of the middle ears in this cohort (labelled as “Tail chimer Fab+mHMGB1”; P<0.01 vs. tail chimer Fabs). Receipt of mHMGB1 alone was highly effective (labelled as “mHMGB1”) as was receipt of one or two treatments with tip-chimer Fabs (labelled as “Tip chimer Fab”). Overall, however, the most effective therapeutic treatment was mHMGB1 admixed with tip-chimer Fabs (labelled as “Tip chimer Fab+mHMGB1”). A single dose cleared pre-formed NTHI biofilms from 67% (4 of 6) of middle ears, with a mucosal biofilm score of 0.1. A second dose further enhanced this response, and no NTHI biofilms were seen in 5 of 6 of middle ears (83%). Further, after receipt of one or two doses, significantly less mucosal biofilm was observed compared to tip chimer Fabs alone (P<0.05) or mHMGB1 alone (P<0.0001, single dose). Collectively, these data demonstrated significant reduction in both bacterial load and eradication of mucosal NTHI biofilms after receipt of mHMGB1, an outcome that was further augmented when co-delivered with tip-chimer Fabs. These results suggested that a host-derived+pathogen-directed therapeutic strategy was highly effective, likely due to the fact that α-DNABII tip Fabs and mHMGB1 disrupted biofilms by distinct mechanisms that, when combined, accounted for the observed combined outcome, as evidenced by virtually complete eradication of pre-existing pathogenic biofilms in this animal model.

Host HMGB1 and bacterial DNABII proteins are found in the extracellular milieu wherein HMGB1 is involved in several processes that include inflammation, cell migration, invasion and proliferation, tissue regeneration and antimicrobial defense (reviewed in Kang et al.). In contrast, it was discovered that extracellularly, DNABII proteins serve as part of an underlying structural component of the eDNA-dependent EPS of diverse bacterial biofilms (Devaraj et al., 2019; Goodman et al., 2011; Gustave et al., 2013; Novotny et al., 2013; Brockson et al., 2014; Rocco et al., 2016; Novotny et al., 2016; Devaraj et al., 2017; Freire et al., 2017; Novotny et al., 2019; Brandstetter et al., Laryngoscope, 2013. 123(11): p. 2626-32; Devaraj et al., Mol Microbiol. 2015 June; 96(6):1119-35; Idicula et al., Laryngoscope. 2016 August; 126(8):1946-51; and Jurcisek et al., Proc Natl Acad Sci USA. 2017 Aug. 8; 114(32):E6632-E6641). Without wishing to be bound by the theory, HMGB1 to be present within the bacterial biofilm EPS was in line with the localization of other NET components such as neutrophil elastase and histone H3 during in vivo infection (Alhede et al., Pathog Dis, 2020. 78(2)).

Given that HMGB1 was localized within the bacterial biofilm EPS, it was hypothesized without wishing to be bound by the theory that biofilm disruption by HMGB1 was mediated by its ability to bind to DNA. The vertices of the crossed strands of eDNA that form the lattice structure of the bacterial biofilm is comprised of HJ-like structures wherein DNABII proteins bind and stabilize the biofilm EPS (Devaraj et al., 2019). Although HMGB1 has a high affinity for HJ DNA, heretofore it was never observed at the vertices of the eDNA lattice, in vivo. This result suggested that when HMGB1 bound to these HJ-like structures of the eDNA lattice, it was unable to stabilize them, and as a consequence collapsed the structures, the opposite effect to that of DNABII proteins which stabilize this structure. This would argue that the anti-biofilm activity of HMGB1 was mediated via a competitive inhibition mechanism wherein HMGB1 bound directly to free HJs. Hints to these biological differences between HMGB1 and DNABII proteins can be derived from biochemical analyses. While DNABII and HMGB1 proteins both bind in the minor groove of DNA, their contacts with HJs occur in different manners (Pontiggia et al., Mol Microbiol, 1993. 7(3): p. 343-50). DNABII proteins have a binding preference to HJs that adopt an X-like structure (Bonnefoy et al., J Mol Biol, 1994. 242(2): p. 116-29; Vitoc and Mukerji, Biochemistry, 2011. 50(9): p. 1432-41; and Deng, V. Y., Mukerji, I., Stability of DNA Four-way junctions and characterization of binding to integration host factor. 2016, Wesleyan University. p. 160), while HJs in a square planar structure is the preferred conformation for HMGB1 (JR P. o. et al., EMBO J, 1998. 17(3): p. 817-26). It was previously shown that RuvA, a prototypic HJ DNA-binding protein that specifically binds to HJs in the square planar conformation (Hargreaves et al., Nat Struct Biol, 1998. 5(6): p. 441-6), compensates for the loss of DNABII proteins within the biofilm EPS (Devaraj et al., 2019). This result implied that the eDNA lattice within the bacterial biofilm EPS was comprised of HJs that are sufficiently similar to a conformation that is compatible for RuvA and thus HMGB1 to bind and yet RuvA stabilizes the eDNA lattice and HMGB1 does not. While this is corroborated by the EMSA stabilization studies with HJ substrates, there are clearly other factors in DNA binding as yet to be determined to explain HMGB1's inability to stabilize the eDNA lattice. Finally, either exogenous addition of DNA-binding proteins that compete with HMGB1 for HJs within the eDNA matrix, or modification of HMGB1 with NEM that directly abrogated the ability of HMGB1 to bind to HJ DNA provided direct evidence that supported competitive inhibition of HJ sites for DNABII proteins as the mechanism of action for HMGB1. To verify that this was the sole mechanism of action, also tested was the possibility that HMGB1 could bind directly to free DNABII proteins as a means to mediate the observed biofilm disruption. SPR analysis demonstrated that HMGB1 bound poorly to DNABII proteins and preferentially binds with high affinity directly to HJ DNA. Thus, within the eDNA, HMGB1 likely destabilized HJs rather than via any limited binding to DNABII proteins.

The capacity for HMGB1 to act as an anti-biofilm therapeutic, rests with its modular structure with their individual functions. HMGB1 has two tandem DNA-binding domains; an A box, a B box and a C-terminal tail that consists of 30 consecutive acidic amino acid residues (Bianchi et al., 1992). Extracellularly, while A box alone exhibits an anti-inflammatory activity and acts as an antagonist for HMGB1, B box alone fully retains the pro-inflammatory activity of HMGB1 and the C-terminal tail is involved in bacterial killing. When in excess, HMGB1 also functions as late mediator of sepsis, a form of systemic inflammation in response to microbial infection (Qin et al., 2006; and Diener et al, 2013). Hence, in the development of HMGB1 as a therapeutic targeted against bacterial biofilms, it is essential to attenuate its pro-inflammatory activity. While it was demonstrated herein that use of rHMGB1 at concentrations that effectively eradicated biofilms did not induce a dysregulated host response to infection associated with septic shock, shown herein is the successful separation of the anti-biofilm activity from the pro-inflammatory functions of HMGB1 via generation of an engineered derivative of rHMGB1 with a single C45S amino acid mutation (mHMGB1) that exhibited potent anti-biofilm function as well as highly attenuated pro-inflammatory function. Further characterization of each of the individual domains of mHMGB1 in an effort to generate a minimal mHMGB1 molecule that retains its ability to disrupt biofilms with minimal pro-inflammatory function is currently under investigation.

Given the results with HMGB1 and its newfound anti-biofilm activity, suggested herein is a new augmented paradigm of NET function. Neutrophils, the first line of host defense against pathogens, release their DNA as NETs in response to pro-inflammatory stimuli that includes LPS, IL-8, TNF-α and HMGB1 to entrap pathogens in an effort to prevent dissemination. Additionally, NETs are also decorated with HMGB1, histones, elastase, and myeloperoxidase that facilitate eradication of microbes (reviewed in Delgado-Rizo et al., Front Immunol, 2017. 8: p. 81). Given that HMGB1 exhibits antibacterial activity and also acts as pro-inflammatory cytokine to induce NET formation, the following model was proposed without wishing to be bound by the theory. For bacteria to persist and resist clearance in a host, they adopt either an aggregated or attached biofilm state that protects them from host immune effectors. Further, partial sloughing of these communities permits propagation of the pathogens without risk of clearance of the core resident biofilm bacteria. To prevent release of bacteria and subsequent propagation however, the host releases sufficient concentrations of HMGB1 during NETosis to confine bacterial pathogens, without induction of an uncontrolled inflammatory response. Thus, HMGB1 acts both to cordon off and limit biofilm proliferation and to alert the innate immune system. This model is biologically relevant, as bacteria actively build biofilms to evade the host immune response, whereas the host actively attempts to prevent pathogenic bacterial biofilm propagation via secretion of a well-balanced level of HMGB1 i.e. too little HMGB1 leads to proliferation of biofilms whereas too much HMGB1 leads to excessive pro-inflammatory effects that causes a dysregulated host response to infection associated with septic shock. It was demonstrated herein that a combinatorial host-directed plus pathogen-derived therapeutic approach achieved via delivery of mHMGB1 with antibodies against a DNABII protein facilitated rapid clearance of biofilms without induction of an inflammatory response in a preclinical model and thereby tipped the balance in favor of the host in the eradication of highly recalcitrant bacterial biofilms.

### Experiment No. 2—Methods

Bacterial strains: UPEC strain UTI89 was isolated from a patient with cystitis (Mulvey et al., Infect Immun, 2001. 69(7): p. 4572-9). B. cenocepacia strain K56-2 was isolated from a cystic fibrosis (CF) patient (Baldwin et al., J Clin Microbiol, 2005. 43(9): p. 4665-73). NTHI strain 86-028NP was isolated from the nasopharynx of a child with chronic otitis media at Nationwide Children's Hospital (Harrison et al., J Bacteriol, 2005. 187(13): p. 4627-36). Enterobacter spp. and K. pneumoniae were isolated from a patient with urinary tract infection. S. aureus strain ATCC® 29213™ was obtained from ATCC. A. baumanii strain ATCC® 17978™ was obtained from ATCC. P. aeruginosa strain ATCC® 27853™ was obtained from ATCC. E. faecium Com12 strain was isolated from feces of healthy human volunteers (Palmer et al., mBio, 2012. 3(1): p. e00318-11). Each of these strains have been maintained at low passage number in liquid nitrogen.

Protein purification: Tagless recombinant HMGB1 was generated with the IMPACT kit (New England Biolabs) as previously described (Devaraj et al., 2017). Human HMGB1 was PCR amplified from a plasmid that contained human HMGB1 tagged with haemagglutinin with the oligonucleotides 5′-ggtggttgctcttccaacatgggcaaaggag-3′ (SEQ ID NO:) and 5′-ggtggtccatggtcattattcatcatcatc-3′ (SEQ ID NO:). The PCR products were cloned into the pTXB1 vector as described (Devaraj et al., 2017) to generate tagless human rHMGB1. The constructs were transformed into the E. coli expression strain ER2566 (New England Biolabs) and selected on lysogeny broth (LB) agar that contained 100 μg/ml ampicillin. rHMGB1 with a single amino acid mutation C45S (mHMGB1) was generated by following the Agilent QuickChange II Site Directed Mutagenesis Method. HMGB1 was PCR amplified from pSG899 with PAGE purified oligos 5′-ggtcttccacctctctgaactcttcttagaaaactctgag-3′ (SEQ ID NO:) and 5′-ctcagagttttctaagaagagttcagagaggtggaagacc-3′ (SEQ ID NO:) (Integrated DNA Technologies) and Phusion DNA polymerase (New England BioLabs) according to the manufacturer's instructions for a total of 16 cycles. The PCR product was then digested with DpnI, followed by transformation into NEB® Stable Competent E. coli following the manufacture's protocol and selection on LB agar that contained 100 μg/ml ampicillin. Random transformants were selected, plasmids were purified and the C45S mutation was confirmed via sequencing. Constructs that contained the C45S mutation were further transformed into the expression strain E. coli ER2566. rHMGB1 and mHMGB1 were overexpressed and purified on a chitin resin column as described (Devaraj et al., 2017). The eluted proteins were dialyzed in heparin binding buffer (10 mM Na-Phosphate buffer (pH 7.0), 200 mM NaCl) overnight at 4° C. The dialyzed protein was loaded onto an AKTA PURE FPLC using a 1 ml HI-Trap Heparin-Sepharose column (GE Healthcare) equilibrated in binding buffer. The column was washed with 20 column volumes of binding buffer, and the bound protein was eluted with 30 column volumes of elution buffer (10 mM phosphate buffer (pH 7), 2M NaCl) with a linear gradient and were fractionated by an AKTA F9-C fraction collector. Each protein was further purified (if <95% purity by SDS-PAGE analysis) by FPLC using a 1 ml Hi-Trap DEAE-Sepharose resin column (GE Healthcare) equilibrated in 40 mM Tris pH 8.5, 2 mM EDTA. The bound protein was eluted with 30 column volumes of a linear gradient of elution buffer that contained 40 mM Tris pH 8.5, 2 mM EDTA and 1M NaCl. The fractions were analyzed by SDS-PAGE, pooled, and dialyzed in storage buffer (50 mM Tris (pH 7.4), 600 mM KCl, 1 mM EDTA). The proteins were concentrated in a centrifugal filter (3000 MWCO), quantified using Pierce BCA Protein assay kit (Thermo Scientific) with bovine serum albumin as the standard, as recommended by the manufacturer. The protein was purified to approximately 95% homogeneity. Lastly, proteins were supplemented with 10% glycerol and stored at −80° C.

Oxidation and NEM modification of rHMGB1: rHMGB1 was purified as described above in ‘Protein Purification’. Purified rHMGB1 was oxidized with hydrogen peroxide as described (Liu et al., 2012). rHMGB1 was purified as described previously. Pooled fractions of rHMGB1 were incubated with 10 mM DTT (Dithiothreitol) for 15 minutes at 4° C. and immediately followed by the addition of NEM (N-Ethylmaleimide) at a final concentration of 50 mM for 5 minutes as described (Kwak et al., Redox Biol, 2019. 24: p. 101203). NEM-HMGB1 was dialyzed overnight in 50 mM Tris-HCl pH 7.4 with 600 mM KCl. The protein was quantified by BCA assay (Pierce).

Acetylation and Phosphorylation: Purified rHMGB1 was resuspended and equilibrated in 20 mM Tris-HCl pH 8.0 200 mM NaCl at RT for 1 h. Acetylation of rHMGB1 was performed as described (Olia et al., 2015, ACS Chem Biol, 10, 2034-47). Briefly, rHMGB1 was incubated with 100 mM Acetyl-Coenzyme A (Sigma Aldrich catalog number 10101893001) at a final concentration of 300 μM for 2 h at 30° C. Unbound acetyl-CoA was removed by buffer exchange. Phosphorylation of rHMGB1 was performed as described (Ugrinova et al., 2012, Mol Biol Rep, 39, 9947-53). Briefly, recombinant Protein Kinase C (cPKCα) was incubated with rHMGB1 at a ratio of 20 ng per 2 μg respectively in 20 mM MOPS pH 7.2 that contained 10 mM MgCl2, 1 mM CaCl2), 140 μM phosphatidylserine, 50 μM ATP for 30 minutes at 30° C. Non-protein bound reagents were removed by buffer exchange. Ac-HMGB1 and P-HMGB1 were concentrated and quantified by BCA assay. Acetylated and phosphorylated forms of rHMGB1 were confirmed by triton-urea gel (Ryan and Annunziato, 2001) and stained with Coomassie blue. Ac-HMGB1 was further confirmed by Western blot with α-Ac-Lys antibody.

Visualization of HMGB1 and DNABII proteins within biofilms formed in vivo: Bullae from chinchillas which had been transbullarly challenged with NTHI were used. Upon sacrifice, bullae were removed, aseptically opened, any fluid collected, washed with sterile saline and filled with OCT compound then snap frozen over liquid nitrogen and stored at −80° C. until use. Prior to sectioning, OCT filled bullae were placed on bed of dry ice while the external bone of the middle ear was carefully chipped away. Bulla was then split in the plane through the tympanic membrane and re-embedded in OCT for orientation. Ten micron serial sections were cut and placed on slides (Mercedes Medical, Lakewood Ranch, Fla.). Slides were either stained with H&E for morphology or by immunofluorescence using standard laboratory techniques. The following commercial antibodies were used: murine monoclonal antibody against human neutrophil elastase (NE-elastase) monoclonal antibody (NOVUS), α-HU and α-HMGB1, AlexaFluor 488 Goat anti-guinea Pig, AlexaFluor 546 Goat anti-Mouse and AlexaFluor 594 Goat anti-Rabbit secondary antibodies (Invitrogen). Slides were allowed to air dry at room temp, fixed in ice cold acetone and blocked with ITX-image enhancer (Invitrogen, Carlsbad, Calif.) and Super Block (Scytek, West Logan, Utah) per manufacturer's instructions. Sections were incubated with a cocktail of primary antibodies ((mouse anti-Elastase and rabbit anti-NTHI OMP P5) or (mouse-anti-HMGB1 and guinea pig anti-HU)) for 1 hour at room temp, washed, incubated with fluorescently tagged secondary antibodies (Invitrogen, Carlsbad, Calif.) for 30 minutes, and washed prior to coverslipping using Prolong Gold plus DAPI (Invitrogen, Carlsbad, Calif.). Low magnification images were capture using a Zeiss Axioskop microscope and Zeiss Zen software. High magnification 3D images were captured on a Zeiss LSM 8000 confocal microscope with airyscan.

Disruption of bacterial biofilms by various isoforms of HMGB1: UPEC strain UTI89, B. cenocepacia, K. pneumoniae, Enterobacter spp, and A. baumanii were cultured on LB agar for 18-20 h at 37° C., in a humidified atmosphere that contained 5% C02, and then suspended in LB broth to an OD of 0.65 at 490 nm. Cultures were then diluted 1:12 in LB broth, and then incubated statically at 37° C., 5% C02 until an OD of 0.6 was reached at 490 nm. The cultures were then diluted in LB broth to contain 2×10⁵ CFU/ml, and 200 μl of this suspension was inoculated into each well of an eight-well chambered cover glass slide (Fisher Scientific). NTHI and S. aureus were cultured on chocolate agar for 18-20 h at 37° C., in a humidified atmosphere that contained 5% C02, and then NTHI was resuspended in sBHI (brain heart infusion broth supplemented with heme (2 μg/ml) and b-NAD (2 μg/ml)) broth to an OD of 0.65 at 490 nm. S. aureus was resuspended in tryptic soy broth (TSB) to an OD of 0.65 at 490 nm. NTHI and S. aureus were diluted 1:6 in sBHI and TSB respectively, and then incubated statically at 37° C., 5% C02 for 3 hours. The cultures were then diluted in their respective media to contain 2×10⁵ CFU/ml, and 200 μl of this suspension was inoculated into each well of an eight-well chambered coverglass slide. P. aeruginosa and E. faecium were cultured on TSB agar and BHI agar respectively for 18-20 h at 37° C., in a humidified atmosphere that contained 5% CO₂, and then suspended in TSB broth or BHI broth respectively to an OD of 0.65 at 490 nm. Cultures were then diluted 1:12 in the respective broth, and then incubated statically at 37° C., 5% CO₂ until an OD of 0.6 was reached at 490 nm. The cultures were then diluted in their respective media to contain 2×10⁵ CFU/ml, and 200 μl of this suspension was inoculated into each well of an eight-well chambered coverglass slide. After 16 hours of incubation of each of the bacterial species at 37° C., 5% CO₂, the medium was replaced with the respective fresh medium and incubated at 37° C., 5% C02 for another 8 h. At 24 hours, the medium was replaced with the respective fresh medium (control) or fresh medium that contained one of the HMGB1 (200 nM) isoforms (rHMGB1, mHMGB1, nHMGB1, Ac-rHMGB1 or P-rHMGB1) or α-IHF_{Ec} (1000 nM) or rHMGB1 in combination with ampicillin (32 μg/ml) or amoxicillin-clavulanate (1 μg/ml) and incubated at 37° C., 5% CO₂ for 16 hours. S. aureus was incubated with 800 nM rHMGB1 for 16 hours and E. faecium was incubated with 800 nM rHMGB1 or 800 nM mHMGB1 for 1 hour at 37° C., 5% C02. nHMGB1 was purchased from Chondrex Inc (Redmond, Wash.). All biofilms except UPEC and K. pneumoniae were washed twice with sterile saline (0.9%) and stained with LIVE/DEAD® stain (Molecular probes, Eugene, Oreg.) in accordance with the manufacturer's instructions. UPEC and K. pneumoniae were washed once with sterile saline and then stained with LIVE/DEAD® stain. Biofilms were then washed once with sterile saline and were fixed with 1.6% paraformaldehyde, 0.025% glutaraldehyde and 4% acetic acid in 0.1M phosphate buffer at pH 7.4. The biofilms were then imaged and analyzed as described (Devaraj et al., 2019). All in vitro biofilm assays were repeated a minimum of three times on separate days. Data are presented as mean±SEM. Planktonic and biofilm-resident bacteria were enumerated as described (Devaraj et al., 2015).

Visualization of eDNA lattice structure within biofilms formed by K. pneumonia: K. pneumoniae biofilms were established for 24 hours as described above in ‘Disruption of bacterial biofilms by various isoforms of HMGB1’ and then incubated with one of the following: LB broth (control), rHMGB1 (200 nM), or mHMGB1 (200 nM) for 16 hours at 37° C., 5% CO₂. eDNA lattice structure was visualized by immunofluorescence as described (Devaraj et al., 2019).

Visualization of NETs structure upon incubation of neutrophils with various isoforms of HMGB1: Human neutrophils were isolated from blood using the EASYSEP™ isolation kit (Stemcell Technologies Inc.). Neutrophils (1×10⁵) were allowed to attach to chambered coverglass followed by incubation with 200 nM rHMGB1, mHMGB1 or NEM-rHMGB1 at 37° C. for 3.5 hours. NETs were fixed in formalin, blocked with 10% normal goat serum (Life Technologies), and labeled with α-dsDNA monoclonal antibody (1 μg), and α-neutrophil elastase polyclonal antibody (1 μg) or naive rabbit and mouse isotype controls (1 μg) in 200 μl PBS for 16 h hours at 4° C. NETs were washed with PBS and incubated for 1 h with 1:200 dilution goat α-rabbit IgG conjugated to ALEXAFLUOR® 594, goat α-mouse IgG conjugated to ALEXAFLUOR® 488, and 1:500 dilution wheat germ agglutinin conjugated to ALEXAFLUOR® 350 (PMN membrane stain). NETs were imaged with a Zeiss LSM 800 confocal microscope (Carl Zeiss Inc.) and rendered with Zeiss Zen software.

Intratracheal challenge of the murine lung with B. cenocepacia and treatment with HMGB1 isoforms: B. cenocepacia strain K56-2, was cultured in LB broth (Difco, Md.) at 37° C. overnight at 200 rpm. Bacterial concentration was adjusted before each experiment based on OD at 600 nm. C57BL/6 mice were infected intratracheally (i.t.) with 10⁷ colony forming units (CFU) of B. cenocepacia. Mice received 0.2 nmol of rHMGB1 or mHMGB1 (i.t.) at the same time of the infection (prevention) or 24 hpi (treatment). Animals were euthanized 24 or 48 hpi, and bronchoalveolar lavage (BAL) was collected with 1 ml of PBS plus 1 mM EDTA. Cells from BAL were stained with α-CD45 brilliant violet 650 (Biolegend), α-CD11b Alexa Fluor 700 (Biolegend) and α-Ly6G PerCP-Cy5.5 (Biolegend) antibodies and LIVE/DEAD blue discriminator (Invitrogen, Eugene Oreg.). Cells were acquired with a LSR II flow cytometer (BD, Franklin Lakes, N.J.). The number of cells were calculated based on the number of neutrophils in BAL (Live, CD45⁺, CD11b^{high}, Ly6G^{high}) and the total count of cells performed by hemocytometer. Two lungs per cohort was fixed and embedded in paraffin; slides were stained with H&E stain. To visualize B. cenocepacia, paraffin section from lungs infected with 10⁷ CFU B. cenocepacia and treated with 0.2 nmol of rHMGB1 or mHMGB1 at 72 hpi were deparaffinated and antigen retrieval was performed in sodium citrate buffer (10 mM sodium citrate, 0.05% Tween 20, pH 6.0) for 15 min at 121° C. The slides were permeabilized with 0.5% Triton X-100 for 10 minutes, blocked with 10% normal goat serum (Abcam) for 30 minutes and stained with a monoclonal antibody against E. coli Elongation Factor-Tu (α-EF-Tu; cross reacts with B. cenocepacia) (LSBio) overnight to label B. cenocepacia. The samples were then stained with goat α-mouse IgG Alexa Fluor 488 (Abcam) for 1 h at RT, mounted with fluoroshield media with DAPI (Abcam), visualized via the Nikon Eclipse Ti at 40× magnification (Nikon Instrument Inc. Americas) and analyzed by ImageJ software (NIH). To enumerate bacterial burden, the animals were euthanized 18 or 72 hpi, bronchoalveolar lavage was collected with 1 ml of PBS plus 1 mM EDTA, an aliquot of BAL was serially diluted and plated on LB agar. Mice work was performed in accordance with the NIH Guide for the Care and Use of Laboratory Animals and under protocol #AR1300020 approved by Abigail Wexner Research Institute at Nationwide Children's Hospital Animal Care and Use Committee. Neither animals nor samples were excluded from evaluation in any study.

Phagocytosis assay: Human monocytes were purified from peripheral blood by using α-CD14 antibodies and magnetic beads (Miltenyi Biotec). In order to differentiate the monocytes to macrophages (MD), the cells were cultured for 5 days in plates coated with collagen (Corning) and RPMI media supplemented with 10% FBS. MΦ were detached by using collagenase IV (Worthington) and seeded in 96 well plates at density of 80,000 cells per well and cultured for 48 h in RPMI supplemented with 10% FBS. MΦ were treated with 5 μg/ml rHMGB1, 5 μg/ml mHMGB1 or 10 μM cytochalasin D for 2 h, then pHrodo red E. coli bioparticles (Thermofisher) were added according to the manufacturer's instructions and incubated for 2 h. Any excess bioparticles were removed by washing the cells with HBSS, the phagocyted bioparticles were measured by plate reader (560/585 nm) and by fluorescent microscopy (Nikon Eclipse Ti).

Assay of neutrophil influx into the peritoneal cavity of mice: C57BL/6 mice were injected i.p. with one of the following: 2 ml of 4% thioglycollate (Fluka Analytical), or 0.2 nmol of rHMGB1 or mHMGB1. Four hours later, peritoneal cells were harvested in PBS plus 1 mM EDTA and stained with α-CD45 brilliant violet 421 (Biolegend), α-CD11b Alexa Fluor 700 (Biolegend) and α-Ly6G PerCP-Cy5.5 (Biolegend) antibodies and LIVE/DEAD blue discriminator (Invitrogen, Eugene Oreg.). Cells were acquired with a LSR II flow cytometer (BD, Franklin Lakes, N.J.). The number of neutrophils were calculated based on the number of neutrophils in peritoneal cavity (Live, CD45+, CD11b^{high}, Ly6G^{high}) and the total counts performed by hemocytometer.

Mouse model of endotoxemia: C57BL/6 mice were injected i.p. with 5 mg/kg of E. coli lipopolysaccharide (Sigma), or 0.2 nmol rHMGB1 alone or in combination with LPS. Animals were euthanized 24 h later, serum was collected, and TNF-α was quantified by ELISA (ThermoFisher).

Generation of polyclonal rabbit anti-tip or tail chimer Fabs: Polyclonal rabbit anti-tip chimer peptide and anti-tail chimer peptide were generated at Rockland Immunochemical, Inc. In some embodiments, the tip chimer comprises, or consists essentially of, or yet further consists of the polypeptide of any one of SEQ ID NOs: 38-40. In further embodiments, the tip chimer comprises, or consists essentially of, or yet further consists of the polypeptide of SEQ ID NO: 40. In some embodiments, the tail chimer comprises, or consists essentially of, or yet further consists of the polypeptide of SEQ ID NO: 41 or 50. In further embodiments, the tail chimer comprises, or consists essentially of, or yet further consists of the polypeptide of SEQ ID NO: 50. IgG was enriched from each rabbit serum by passage through rProtein A and Protein G GraviTrap columns (GE Healthcare) according to manufacturer's instructions. Fabs were then generated via Pierce Fab Preparation kit. Digestion of rabbit IgG to Fabs was confirmed by SDS-PAGE with COOMASSIE FLUOR™ Orange Protein Gel stain (ThermoFisher). Bacterial endotoxin test via ToxinSensor Chromogenic LAL endotoxin kit (Genscript) was performed prior to use.

Chinchilla model of experimental otitis media (OM): Adult chinchillas (Chinchilla lanigera) were obtained from Rauscher's Chinchilla Ranch, LLC. These outbred, non-specific pathogen-free animals were randomly divided into cohorts based on body weight and both male and female animals were enrolled. To test biofilm disruption induced by 200 nM rHMHB1 compared to 200 nM mHMGB1 or equivalent volume of sterile saline, 4-6 animals were enrolled in each cohort. Efficacy of 200 nM mHMGB1 versus 342 nM tip chimer Fabs+200 nM mHMGB1, 342 nM tip chimer Fabs alone, 342 nM tail chimer Fabs+200 nM mHMGB1 or 342 nM tail chimer Fabs alone was evaluated with three animals per cohort. A concentration of 342 nM was based on prior studies wherein 5 μg intact IgG per 0.1 ml volume was injected into the middle ears of chinchillas (Goodman et al., 2011; Novotny et al., 2016; and Novotny et al., 2019). Chinchilla work was performed in accordance with the NIH Guide for the Care and Use of Laboratory Animals and under protocol #01304AR approved by Abigail Wexner Research Institute at Nationwide Children's Hospital Animal Care and Use Committee. Neither animals nor samples were excluded from evaluation in any study.

Disruption of NTHI biofilms resident within the middle ears of chinchillas with experimental OM: Both middle ears of each animal were challenged with 1000 CFU NTHI strain 86-028NP by direct injection to induce experimental OM. Four days later, NTHI mucosal biofilms fill >50% of the middle ear (Novotny et al., 2011). At this time, treatments were injected into each middle ear (0.1 ml delivered per bulla). Animals were either sacrificed 24 h later or received an identical treatment as described. Animals that received a second treatment were sacrificed after an additional 24 h. To determine the outcome of treatment, animals were sacrificed one day after completion of antibody therapy, images of mucosal biofilms captured with a Zeiss SV6 dissecting microscope, then mucosal biofilms and middle ear mucosa collected, homogenized and plated on to chocolate agar to semi-quantitate the non-planktonic bacterial load within the middle ear (Goodman et al., 2011). Mucosal biofilms were collected and processed as described (Goodman et al., 2011). As an additional assessment, the amount of biofilm in each middle ear was qualitatively determined. Images of each middle ear were captured, randomized and ranked by six blinded reviewers using an established rubric wherein 0=no mucosal biofilm visible; 1=<25% of middle ear occluded by mucosal biofilm; 2=≥25-<50% occluded; 3=≥50-<75% occluded; 4=≥75-100% occluded (Novotny et al., 2011). To evaluate relative mucosal inflammation in each middle ear, a second rubric was developed: 0=no inflammation, mucosa has white hue; 1=thin capillary dilatation, mucosa has white hue; 2=think and thick capillary dilatation, mucosa has red hue, ≤5 small hemorrhagic foci; 3=thick capillary dilatation, mucosa has red hue, >5 small and large hemorrhagic foci. For both quantitation of bacterial load and qualitative assessment of mucosal biofilm, each middle ear was considered independent.

Quantitation of cytokines in middle ear fluids: To quantitate cytokines in middle ear fluids (MEF), a BD™ Cytometric Bead Array was performed with fluids collected at the time of animal sacrifice. With BD™ human-specific Flex Sets, each MEF was individually examined for relative quantity of IL-1β, IL-4, IL-6, IL-8, IL-10, IL12-p70, IL-13, IL-17A, TNF or IFNγ according to manufacturer's instructions. MEFs from each animal were assayed individually. Data were captured on a BD ACCURI™ C6 cytometer (BD Biosciences) and analyzed with FlowJo software (FlowJo, LLC). The concentration of cytokines in each MEF was determined using a standard curve and calculated using GraphPad Prism software.

Surface plasmon resonance: To determine the affinity of HMGB1 and the DNABII proteins IHF_{NTHI} and HU_{NTHI}, surface plasmon resonance using a Biacore 3000 instrument (GE Healthcare Life Sciences) was performed. All experiments were conducted at 25° C. and 10 mM HEPES (pH 7.4)-150 mM NaCl-3 mM EDTA supplemented with 5 mg/ml BSA served as the running buffer. Via amine couple chemistry and at a flow rate of 5 μl/min, mHMGB1 were immobilized to a flow cell of a CM5 sensor chip (GE Healthcare) to 250 resonance units. Next, native IHF_{NTHI} or HU_{NTHI} was suspended in running buffer plus NSB reducer (GE Healthcare) and serial two-fold dilutions from 50 nM to 3.1 nM, including a buffer-only sample, were injected across the antibody-bound and reference surface at a flow rate of 30 μl/min, 3 min injection time, 3 min dissociation time using KINJECT. All data was reference cell and buffer control subtracted. BiaEvaluation software (GE Healthcare) was used to align sensorgram curves and determine K_{D} values.

Electrophoretic mobility shift assay (EMSA): EMSA was performed as previously described (Devaraj et al., 2019).

Statistical analyses: Graphical results and statistical tests were performed with GraphPad Prism 8 for all in vitro and in vivo assays. Statistical significance for in vitro assays was assessed by unpaired or paired t tests. Statistical significance for the mouse model was assessed by Mann-Whitney test. Differences in bacterial load, mean mucosal biomass score and relative mucosal inflammation in the chinchilla model were determined by one-way ANOVA with multiple comparisons. Statistical significance was denoted as *P<0.05, **P<0.01, ***P<0.001 and ****P<0.0001.

TABLE 1
Summary of binding constants of HMGB1
binding to DNABII proteins and HJ DNA.

| Binding of mHMGB1 to | K_{D} (nM) | Source |
|---|---|---|
| HJ DNA | 10 | Xin et al., 2000 |
| IHF_{NTHI} | 579 | This study |
| HU_{NTHI} | 103 | This study |

### Experiment No. 3

This experiment describes an in vitro model for reversal of an established biofilm in 8-well chamber slide. The materials used in this experiment are: Chocolate Agar; sBHI (BHI with 2 mg heme/mL and 2 mg b-NAD/mL); 8-well Chamber slides (Nunc* Lab-Tek* Fisher catalog #12-565-18); Sterile 0.9% saline; LIVE/DEAD BacLight Bacterial Viability Kit (Fisher catalog #NC9439023) and Formalin.

NTHI 86-028NP colonies were collected from overnight culture on chocolate agar and suspended in brain heart infusion broth supplemented with 2 μg j-NAD and heme per ml medium (sBHI). The optical density at 490 nm was then adjusted to 0.65 and the culture diluted 1:6 in sBHI prior to incubation at 37° C. with 5% C02 for 3 hr, static. Next, the culture was diluted 1:2500 in fresh sBHI and 200 μl of the suspension aliquoted into each well of an 8-well chamber slide. The slide was then incubated at 37° C. with 5% C02 for 3 hr, static. After 16 hr, 200 μl fresh sBHI was added to each well, and the slide incubated an additional 8 hr. At this time point, medium was aspirated from each well and a composition comprising the antibody or antigen-binding fragment thereof and the HMGB1 polypeptide or a fragment thereof was added. The biofilms were incubated an additional 16 hr. Biofilms were then washed and stained with FM1-43FX bacterial cell membrane stain (Invitrogen) and fixed overnight at 4° C. in 16% paraformaldehyde, 2.5% glutaraldehyde, 4.0% acetic acid in 0.1 M phosphate buffer (pH 7.4). Fixative was aspirated an 200 μl 0.9% Hank's Balanced Salt Solution was added to each well prior to viewing of biofilms on a Zeiss 800 Meta-laser scanning confocal microscope. Images were compiled with Zeiss Zen Blue software and biofilm biomass calculated with COMSTAT2 software.

### Experiment No. 4

Efficacy of a composition or combination comprising the antibody or antigen-binding fragment thereof and the HMGB1 polypeptide or a fragment thereof can be established in two well-established pre-clinical models of otitis media induced by the prevalent respiratory tract pathogen nontypeable Haemophilus influenzae, a common biofilm disease.

In vitro biofilm disruption assays are repeated three times on separate days. For in vivo experiments, chinchillas are randomly divided into cohorts based on body weight; both male and female animals are enrolled. 3 or 4 animals are enrolled into each cohort. Neither animals nor samples are excluded from the study. Evaluation of relative mucosal biofilm that remained in the middle ear after treatment is performed by 6-8 individuals not involved in the study and who are blinded to therapy delivered. For each study assessment, each middle ear is considered independent.

### In Vitro Biofilm Disruption

Biofilms formed by NTHI strain 86-028NP (a minimally passaged clinical isolate from the nasopharynx of a child with chronic OM, see for example, Bakaletz L O et al. (1988) Infect Immun 1988; 56: 331-5), M. catarrhalis strain 7169 (a minimally passaged clinical isolate from the middle ear of a child with chronic OM, see for example, Luke N R et al (1999) Infect Immun 1999; 67: 681-7), P. aeruginosa strain 27853, B. cenocepacia strain K56 (isolated from the sputum of a patient with cystic fibrosis, see, for example, Mahenthiralingam E et al (2000) J Clin Microbiol 2000; 38: 910-3) and S. aureus strain 29213 were first established in 8-well chambered coverglass (CellVis) for 24 h prior to incubation with 170 nM intact IgG or Fab fragments for an additional 16 h (S. D. Goodman et al. (2011) Mucosal Immunol 4:625-637; J. A. Jurcisek et al. (2011) J Vis Exp). In one embodiment, NTHI strain 86-028NP is kept no more than passage #4 since it came out of a child in 1986. Additionally or alternatively, the Mcat strain has similarly been kept at a very low passage number since its isolation. In a further embodiment, the Burkholderia is isolated from the sputum of a cystic fibrosis (CF) patient. In yet a further embodiment, the Staphylococcus aureus isolate is from the ATCC. A concentration of 170 nM is based on prior studies wherein 5 μg intact IgG per 0.2 ml volume was applied to in vitro biofilms (Goodman S D et al (2011) Mucosal Immunol 2011; 4: 625-37; Brockson M E et al (2014) Mol Microbiol 2014; 93: 1246-58; Novotny L A et al (2019) NPJ Vaccines 2019; 4: 43; Novotny L A et al (2013) PLoS One 2013; 8: e67629; and Novotny L A et al (2016) EBioMedicine 2016; 10: 33-44) to permit direct comparison between IgG- versus Fabs-mediated disruption. Bacteria within the biofilms are then stained with FM1-43FX (ThermoFisher), fixed overnight in a solution of 16% paraformaldehyde-4% acetic acid-2.5% glutaraldehyde in 0.1 M phosphate buffer (pH 7.4) then washed with 10 mM phosphate buffered saline (pH 7.4). Biofilms are viewed with a Zeiss 800 scanning confocal laser microscope, images rendered in Zeiss Zen Pro software and biomass determined with COMSTAT2 software (A. Heydorn et al. (2000) Microbiology 146 (pt 10): 2395-2407). In vitro biofilm disruption assays are repeated three times on separate days.

### Animals

Juvenile or adult chinchillas (Chinchilla lanigera; juvenile animals were 250-499 g in body mass; adult animals were 500-850 g in body mass) are obtained from Rauscher's Chinchilla Ranch, LLC. These outbred, non-specific pathogen-free animals are housed in individual cages with autoclaved corncob bedding and sterile water provided ad libitum. Animals are randomly divided into cohorts based on body weight (as an indication of juvenile or adult status) and both male and female animals are used. In some embodiments, experimental groups are as follows: to examine disruption of NTHI biofilms from the middle ear as induced by (a) a composition comprising the antibody or antigen-binding fragment thereof and the HMGB1 polypeptide or a fragment thereof, (b) a composition comprising the antibody or antigen-binding fragment thereof only, (c) a composition comprising the HMGB1 polypeptide or a fragment thereof only, and (d) control(s), three or four animals were enrolled into each cohort. Efficacy is evaluated.

### Disruption of NTHI Biofilms Formed in the Middle Ears of Chinchillas in Experimental OM

Both middle ears of each animal are challenged with 1000 CFU NTHI strain 86-028NP by direct injection to induce experimental OM. Four days later, NTHI mucosal biofilms fill >50% of the middle ear (L. A. Novonty et al. (2011) Mucosal Immunol 4: 456-467). At this time, the compositions are administered, with identical treatment delivered 24 h later. To determine the immediate outcome of treatment, animals are sacrificed one day after completion of the therapy, images of mucosal biofilms captured with a Zeiss SV6 dissecting microscope, then mucosal biofilms and middle ear mucosa collected, homogenized and plated on to chocolate agar to quantitate the bacterial load within the middle ear (S. D. Goodman et al. (2011) Mucosal Immunol 4: 625-637). A subset of animals in each cohort is monitored an extra seven days without additional treatment to examine whether NTHI biofilms would re-form upon cessation of antibody therapy as well as determine how the cytokine profile might change over time. Mucosal biofilms are collected and processed as described (S. D. Goodman et al. (2011) Mucosal Immunol 4: 625-637).

### Quantitative Assessment of Amount of Mucosal Biofilm

As an additional assessment, the amount of biofilm in each middle ear is qualitatively determined. Images of each middle ear are captured, randomized and ranked by six reviewers blinded to treatment delivered using an established rubric wherein 0=no mucosal biofilm visible, 1=<25% of middle ear occluded by mucosal biofilm, 2=≥25-50% occluded, 3=≥50-75% occluded, 4=≥75-100% occluded (L. A. Novotny et al. (2011) Mucosal Immunol 4: 456-467). For both quantitation of bacterial load and qualitative assessment of mucosal biofilm, each middle ear is considered independent.

### Quantitation of Cytokines in Middle Ear Fluids

To quantitate cytokines in middle ear fluids (MEF) a BD™ Cytometric Bead Array is performed with fluids collected at the time of animal sacrifice. With BD™ human-specific Flex Sets each MEF is individually examined for quantity of IL-1β (cat #558279), IL-6 (cat #558276), IL-8 (cat #558277), IL-10 (cat #558274), IL12-p70 (cat #558283), IL-17A (cat #560383) and TNF (cat #560112) using according to manufacturer's instructions. The cytokine IL-13 (cat #558450) is added to the panel used to assay MEF collected. MEF from each animal are assayed individually. Data are captured on a BD ACCURI™ C6 cytometer (BD Biosciences) and analyzed with FlowJo software (FlowJo, LLC). The concentration of cytokines in each MEF is determined using a standard curve and calculated using GraphPad Prism software.

### Viral-Bacterial Co-Infection Model of Experimental NTHI-Induced OM

### Video Otoscopy

Video otoscopy is performed on all animals using a Welch Allyn MACROVIEW™ otoscope and Welch Allyn Viewer software. Each tympanic membrane is blindly ranked on a on an established scale of 0 to 4, and middle ears with a score of ≥2.0 are considered positive for OM as inflammation (erythema) and MEF are visible (L. A. Novotny et al. (2006) Vaccine 24: 4804-4811). If one middle ear is ranked ≥2.0, but the contralateral ear is ranked <2.0, the animal is considered positive for OM. To calculate vaccine efficacy, the number of observations of OM during the 20-day study period is first determined and converted to a percentage relative to the total number of observations for each cohort. This value is then subtracted from the percentage computed for the cohort given dmLT only. Video otoscopy is performed by an individual blinded to formulation delivered.

### Statistical Analyses

Graphical results and statistical tests are performed with GraphPad Prism 8. Differences in biomass for in vitro biofilm disruption assays are determined by one-way ANOVA with multiple comparisons. Differences in quantity of cytokines in middle ear fluids among cohorts are determined by determined by one-way ANOVA with multiple comparisons. Differences in bacterial load and mean mucosal biomass score are determined by one-way ANOVA with multiple comparisons. Delay to onset of OM and time to resolution of disease are determined by Mantel-Cox test.

### Experiment No. 5

A number of oral bacteria (e.g., Aggregatibacter actinomycetemcomitans, Porphyromonas gingivalis) have been implicated in the pathogenesis of inflammatory diseases such as periodontitis and peri-implantitis, which destroy alveolar bone and gingiva. Investigations of the pathogenesis of these bacteria are hampered by lack of effective animal models. One of the challenges of investigating the pathogenicity of specific bacteria is the difficulty of establishing a biofilm when exogenous bacteria are introduced into the oral cavity of animals. Though animal models of periodontitis have been developed, cultivable bacteria are rarely recovered from the oral cavity of inoculated animals. Developing an effective animal model which can assess the pathogenicity of specific bacteria wilt greatly aid in elucidating their pathogenic mechanisms.

A rat model of peri-implantitis where bacterial biofilms (e.g. A. actinomycetemcomitans, P. gingivalis) are grown on the heads of titanium screw implants (Freire, M. O., (2011) J. Periodontology 82(5):778-89 and Freire, M. O., (2017) Molecular Oral Microbiology 32(1):74-88) can be used to establish efficacy for these diseases. The biofilm laden screws are then surgically implanted into the rat maxilla aveolar bone and monitored over time. Micro computed tomography can be used to measure bone loss surrounding the screws, and DNA extracted from the screws as well as the tissue and bone surrounding the screws can be utilized for qPCR or microbiome analyses to reveal the species present. This established animal model revealed the presence of bone loss surrounding the screw implant and the detection of the inoculated bacterial pathogens at least 2 weeks post implantation.

Efficacy of a composition or combination comprising the antibody or antigen-binding fragment thereof and the HMGB polypeptide or a fragment thereof, for example, as compared with a composition or combination comprising the antibody or antigen-binding fragment thereof alone or a composition or combination comprising the HMGB polypeptide or a fragment thereof alone, is tested in this model.

### Experiment No. 6

This experiment provides a mouse model for pre-clinical testing of interfering agents to treat lyme disease. See Dresser et al. Pathogens 5(12)e1000680, Epub 2009 Dec. 4. Lyme disease is the most common tick-borne disease in the United States. Reported cases have more than doubled between 1992 and 2006, with approximately 29,000 new cases confirmed in 2008. Estimates are that the actual number of cases of Lyme disease may exceed that reported by a factor of 6-12 in endemic areas. By definition, these endemic areas are expanding as populations continue to move from cities to suburban and rural areas and whitetail deer (which carry the tick species Ixodes) increasingly roam these areas. Lyme disease is caused by the microorganism Borrelia burgdorferi, a spirochete. B. burgdorferi is transmitted via the bite of the Ixodes tick and subsequently disseminates, via the bloodstream, to other tissues and organs.

In this animal model, C3H/HeN mice are injected with spirochetes via dorsal subcutaneous and intraperitoneal injection, or via intravenous injection. Blood and biopsy specimens are recovered at approximately 7 days post infection for evaluation of microbial burden and assessment of pathology in tissues and organs. The methods and compositions disclosed herein are contemplated to develop both therapeutic as well as preventative strategies for reduction and/or elimination of the resulting B. burgdorferi biofilms which form subsequent to challenge and are believed to contribute to both the pathogenesis and chronic nature of the disease.

Efficacy of a composition or combination comprising the antibody or antigen-binding fragment thereof and the HMGB polypeptide or a fragment thereof, for example, as compared with a composition or combination comprising the antibody or antigen-binding fragment thereof alone or a composition or combination comprising the HMGB polypeptide or a fragment thereof alone, is tested in this model.

### Experiment No. 7

This experiment provides a porcine model for pre-clinical testing of interfering agents to treat cystic fibrosis. See Stoltz et al. (2010) Science Translational Medicine 2(29):29-31. Cystic fibrosis is an autosomal recessive disease due to mutations in a gene that encodes the CF transmembrane conductance regulator (called CFTR) anion channel. In this model, pigs which have been specifically bred to carry a defect in the genes called “CFTR” and called CF pigs spontaneously develop hallmark features of CF lung disease that includes infection of the lower airway by multiple bacterial species. The pigs can be immunized with the interfering agents to either 1) immunize these CF pigs with a polypeptide or other immunogenic agent thereby inducing the formation of antibodies which will eradicate bacterial biofilms in the lungs to deliver antibodies, or fragments or derivatives thereof to the lungs of these animals by nebulization to assess the amelioration of the signs of disease and associated pathologies.

Efficacy of a composition or combination comprising the antibody or antigen-binding fragment thereof and the HMGB polypeptide or a fragment thereof, for example, as compared with a composition or combination comprising the antibody or antigen-binding fragment thereof alone or a composition or combination comprising the HMGB polypeptide or a fragment thereof alone, is tested in this model.

### Experiment No. 8

Applicants also provide a pre-clinical model for tuberculosis (TB). See Ordway et al. (2010) Anti. Agents and Chemotherapy 54:1820. The microorganism Mycobacterium tuberculosis is responsible for a growing global epidemic. Current figures suggest that there are approximately 8 million new cases of TB and about 2.7 million deaths due to TB annually. In addition to the role of this microbe as a co-infection of individuals with HIV (of the ˜45 million infected with HIV, estimates are that ˜3 are also co-infected with M. tuberculosis), its particularly troublesome that isolates have become highly resistant to multiple drugs and no new drug for TB has been introduced in over a quarter of a century. In this animal model, SPF guinea pigs are maintained in a barrier colony and infected via aerosolized spray to deliver ˜20 cfu of M. tuberculosis strain Erdman K01 bacilli into their lungs. Animals are sacrificed with determination of bacterial load and recovery of tissues for histopathological assessment on days 25, 50, 75, 100, 125 and 150 days post-challenge. Unlike mice which do not develop classic signs of TB, guinea pigs challenged in this manner develop well-organized granulomas with central necrosis, a hallmark of human disease. Further, like humans, guinea pigs develop severe pyogranulomatous and necrotizing lymphadenitis of the draining lymph nodes as part of the primary lesion complex. Use of this model will provide a pre-clinical screen to confirm and identify therapeutic as well as preventative strategies for reduction and/or elimination of the resulting M. tuberculosis biofilms which have been observed to form in the lungs of these animals subsequent to challenge and are believed to contribute to both the pathogenesis and chronicity of the disease.

Efficacy of a composition or combination comprising the antibody or antigen-binding fragment thereof and the HMGB polypeptide or a fragment thereof, for example, as compared with a composition or combination comprising the antibody or antigen-binding fragment thereof alone or a composition or combination comprising the HMGB polypeptide or a fragment thereof alone, is tested in this model.

### Experiment No. 9

Multiple animal models of catheter/indwelling device biofilm infections are known. See Otto (2009) Nature Reviews Microbiology 7:555. While typically considered normal skin flora, the microbe Staphylococcus epidermidis has become what many regard as a key opportunistic pathogen, ranking first among causative agents of nosocomial infections. Primarily, this bacterium is responsible for the majority of infections that develop on indwelling medical devices which are contaminated by this common skin colonizer during device insertion. While not typically life-threatening, the difficulty associated with treatment of these biofilm infections, combined with their frequency, makes them a serious public health burden. Current costs associated with treatment of vascular catheter associated bloodstream infections alone that are due to S. epidermidis amount to $2 billion annually in the United States. In addition to S. epidermidis, E. faecalis and S. aureus are also contaminations found on indwelling medical devices. There are several animal models of catheter-associated S. epidermidis infections including rabbits, mice, guinea pigs and rats all of which are used to study the molecular mechanisms of pathogenesis and which lend themselves to studies of prevention and/or therapeutics. Rat jugular vein catheters have been used to evaluate therapies that interfere with E. faecalis, S. aureus and S. epidermidis biofilm formation. Biofilm reduction is often measured three ways-(i) sonicate catheter and calculate CFUs, (ii) cut slices of catheter or simply lay on a plate and score, or (iii) the biofilm can be stained with crystal violet or another dye, eluted, and OD measured as a proxy for CFUs.

Efficacy of a composition or combination comprising the antibody or antigen-binding fragment thereof and the HMGB polypeptide, e.g., HMGB1 polypeptide or a fragment thereof, for example, as compared with a composition or combination comprising the antibody or antigen-binding fragment thereof alone or a composition or combination comprising the HMGB polypeptide or a fragment thereof alone, is tested in this model.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### SEQUENCES

SEQ ID NO: 1 (H10210 (1F8.F1 Humanized HC1))

Bold font indicates an exemplified variable region while the bold, italic and underlined font indicates exemplified CDRs.

MDPKGSLSWRILLFLSLAFELSYGEVKLVESGGGLVQPGGSLRLSCAAS [symbol] [symbol]
MSWVRQAPGKGLEWVAT [symbol] YYPDSVKGRFTI
SRDNAKNTLYLQMNSLRAEDTAVYYC [symbol] WGQGTLVTV
SSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSK
LTVDKSRWQQGNVFSCSVMHEALHNHYTQKSLSLSPG**

SEQ ID NO: 2 (H10211 (1F8.F1 Humanized HC2))

Bold font indicates an exemplified variable region while the bold, italic and underlined font indicates exemplified CDRs.

MDPKGSLSWRILLFLSLAFELSYGEVQLVESGGGLVQPGGSLRLSCAAS [symbol] [symbol]
MSWVRQAPGKGLEWVAT [symbol] YYPDSVKGRFTI
SRDNSKNTLYLQMNSLRAEDTAVYYC [symbol] WGQGTLVT
VSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYS
KLTVDKSRWQQGNVFSCSVMHEALHNHYTQKSLSLSPG**

SEQ ID NO: 3 (H10212 (1F8.F1 Humanized HC3))

Bold font indicates an exemplified variable region while the bold, italic and underlined font indicates exemplified CDRs.

MDPKGSLSWRILLFLSLAFELSYGEVKLVQSGAEVKKPGASVKVSCKAS [symbol] [symbol]
MSWVRQAPGQRLEWVAT [symbol] YYPDKFQGRVTI
TRDNAKNTLYMELSSLRSEDTAVYYC [symbol] WGQGTLVT
VSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLY
SKLTVDKSRWQQGNVFSCSVMHEALHNHYTQKSLSLSPG**

SEQ ID NO: 4 (H10213 (11E7.C7 Humanized HC1)) Bold font indicates an exemplified variable region while the bold, italic and underlined font indicates exemplified CDRs.

MDPKGSLSWRILLFLSLAFELSYGEVQLVESGGGLVKPGGSLRLSCAAS [symbol] [symbol]
MSWVRQAPGKGLEWVAT [symbol] YYTDSVKGRFT
ISRDNAKNSLYLQMNSLRAEDTAVYYC [symbol] WGQGTMV
TVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLY
SKLTVDKSRWQQGNVFSCSVMHEALHNHYTQKSLSLSPG**

SEQ ID NO: 5 (H10214 (11E7.C7 Humanized HC2))

Bold font indicates an exemplified variable region while the bold, italic and underlined font indicates exemplified CDRs.

MDPKGSLSWRILLFLSLAFELSYGEVQLVESGGGLVKPGGSLRLSCAAS [symbol] [symbol]
MSWVRQAPGKGLEWVST [symbol] YYTDSVKGRFTI
SRDNAKNSLYLQMNSLRAEDTAVYYC [symbol] WGQGTMVT
VSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYS
KLTVDKSRWQQGNVFSCSVMHEALHNHYTQKSLSLSPG**

SEQ ID NO: 6 (H10215 (11E7.C7 Humanized HC3))

Bold font indicates an exemplified variable region while the bold, italic and underlined font indicates exemplified CDRs.

MDPKGSLSWRILLFLSLAFELSYGEVQLVESGGGLVQPGRSLRLSCTAS [symbol] [symbol]
MSWVRQAPGKGLEWVAT [symbol] YYTDSVKGRFT
ISRDNAKNILYLQMNSLKTEDTAVYYC [symbol] WGQGT
MVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPG**

SEQ ID NO: 7 (L10210 (1F8.F1 Humanized LC1))

Bold font indicates an exemplified variable region while the bold, italic and underlined font indicates exemplified CDRs.

METDTLLLWVLLLWVPGSTGDVVMTQSPLSLPVTLGQPASISCRSS [symbol] [symbol]
LNWLQQRPGQSPRRLIY [symbol] KLDSGVPDRFS
GSGSGTDFTLKISRVEAEDVGVYYC [symbol] FGQGTKLEIK
RTVAAPSVFIFPPSDEQLKSGTASVVCLLNNFYPREAKVQWKVDNA
LQSGNSQESVTEQDSKDSTYSLSSTLTLSKADYEKHKVYACEVTHQ
GLSSPVTKSFNRGEC*

SEQ ID NO: 8 (L10211 (1F8.F1 Humanized LC2))

Bold font indicates an exemplified variable region while the bold, italic and underlined font indicates exemplified CDRs.

METDTLLLWVLLLWVPGSTGDVVMTQSPLSLPVTLGQPASISCRSS [symbol] [symbol]
LNWLQQRPGQSPRRLIY [symbol] KRDSGVPDRFS
GSGSGTDFTLKISRVEAEDVGVYYC [symbol] FGQGTKLEIK
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC*

SEQ ID NO: 9 (L10212 (1F8.F1 Humanized LC3))

Bold font indicates an exemplified variable region while the bold, italic and underlined font indicates exemplified CDRs.

METDTLLLWVLLLWVPGSTGDVVMTQSPDSLAVSLGERATINCKSS [symbol] [symbol]
LNWLQQKPGQPPKRLIY [symbol] KLDSGVPDRF
SGSGSGTDFTLTISSLQAEDVAVYYC [symbol] FGQGTKLEI
KRTVAAPSVFIFPPSDEQLKSGTASVVCLLNNFYPREAKVQWKVDN
ALQSGNSQESVTEQDSKDSTYSLSSTLTLSKADYEKHKVYACEVTH
QGLSSPVTKSFNRGEC*

SEQ ID NO: 10 (L10213 (11E7.C7 Humanized LC1))

Bold font indicates an exemplified variable region while the bold, italic and underlined font indicates exemplified CDRs.

METDTLLLWVLLLWVPGSTGDIQMTQSPSSLSASVGDRVTITCRA
S [symbol] LNWYQQKPGKAVKLLIY [symbol] RLHSGVPSRFSGSGS
GTDYTLTISSLQPEDFATYFC [symbol] [symbol] FGGGTKVEIKRTV
AAPSVFIFPPSDEQLKSGTASVVCLLNNFYPREAKVQWKVDNALQ
SGNSQESVTEQDSKDSTYSLSSTLTLSKADYEKHKVYACEVTHQG
LSSPVTKSFNRGEC*

SEQ ID NO: 11 (L10214 (11E7.C7 Humanized LC2))

Bold font indicates an exemplified variable region while the bold, italic and underlined font indicates exemplified CDRs.

METDTLLLWVLLLWVPGSTGDIQMTQSPSSLSASVGDRVTITCR
AS [symbol] LNWYQQKPGKAVKLLIY [symbol] RLHSGVPSRFSGS
GSGTDYTLTISSLQPEDFATYYC [symbol] [symbol] FGGGTKVEIKR
TVAAPSVFIFPPSDEQLKSGTASVVCLLNNFYPREAKVQWKVDN
ALQSGNSQESVTEQDSKDSTYSLSSTLTLSKADYEKHKVYACEV
THQGLSSPVTKSFNRGEC*

SEQ ID NO: 12 (L10215 (11E7.C7 Humanized LC3))

Bold font indicates an exemplified variable region while the bold, italic and underlined font indicates exemplified CDRs.

METDTLLLWVLLLWVPGSTGDIVMTQSPATLSLSPGERATLSCRAS [symbol]
LNWYQQKPGQAVRLLIY [symbol] RLHSGIPARFSGSGSGTDYTLTI
SSLEPEDFAVYFC [symbol] [symbol] FGGGTKVEIKRTVAAPSVFIFPPSDEQL
KSGTASVVCLLNNFYPREAKVQWKVDNALQSGNSQESVTEQDSKDSTYSLS
STLTLSKADYEKHKVYACEVTHQGLSSPVTKSFNRGEC*

SEQ ID NO: 13 (Heavy Chain Consensus Sequence)

| | |
|---|---|
| MDPKGSLSWR ILLFLSLAFE LSYGEVqLVe SGgglvXPGg SlrlSCaASG | 50 |
| FTFXXYXMSW VRQAPGkgLE WVaTIXSXXX XTYYXDsvkG RfTIsRDNaK | 100 |
| NtLYlqmnSL raEDTAVYYC XXXXXXXYXX FDXWGQGTXV TVSSASTKGP | 150 |
| SVFPLAPSSK STSGGTAALG CLVKDYFPEP VTVSWNSGAL TSGVHTFPAV | 200 |
| LQSSGLYSLS SVVTVPSSSL GTQTYICNVN HKPSNTKVDK KVEPKSCDKT | 250 |
| HTCPPCPAPE LLGGPSVFLF PPKPKDTLMI SRTPEVTCVV VDVSHEDPEV | 300 |
| KFNWYVDGVE VHNAKTKPRE EQYNSTYRVV SVLTVLHQDW LNGKEYKCKV | 350 |
| SNKALPAPIE KTISKAKGOP REPQVYTLPP SREEMTKNQV SLTCLVKGFY | 400 |
| PSDIAVEWES NGQPENNYKT TPPVLDSDGS FFLYSKLTVD KSRWQQGNVF | 450 |
| SCSVMHEALH NHYTQKSLSL SPG** | 475 |

Wherein X and a small letter can be substituted with any amino acid or alternatively, with an amino acid selected from SEQ ID NOs: 1-6, in the corresponding position. In one embodiment, X may also indicate absence of an amino acid residue.

SEQ ID NO: 14 (Light Chain Consensus Sequence)

| | |
|---|---|
| METDTLLLWV LLLWVPGSTG DXvMTQSPXs LsvslGXrat isCrXSQXXX | 50 |
| XXXXXXXLNW XQQkPGqaXX XLIYXXSXIX SGvPdRFSGS GSGTDXTLtI | 100 |
| SslXXEDXav YyCXQGXXXX XTFGXGTKXE IKRTVAAPSV FIFPPSDEQL | 150 |
| KSGTASVVCL LNNFYPREAK VQWKVDNALQ SGNSQESVTE QDSKDSTYSL | 200 |
| SSTLTLSKAD YEKHKVYACE VTHQGLSSPV TKSFNRGEC* | 240 |

Wherein X and a small letter can be substituted with any amino acid or alternatively, with an amino acid selected from SEQ ID NOs: 7-12, in the corresponding position. In one embodiment, X may also indicate absence of an amino acid residue.

SEQ ID NO: 15 Human IgD Constant Region, Uniprot: P01880

APTKAPDVFPIISGCRHPKDNSPVVLACLITGYHPTSVTVTWYMGTQSQPQ
RTFPEIQRRDSYYMTSSQLSTPLQQWRQGEYKCVVQHTASKSKKEIFRWPE
SPKAQASSVPTAQPQAEGSLAKATTAPATTRNTGRGGEEKKKEKEKEEQEE
RETKTPECPSHTQPLGVYLLTPAVQDLWLRDKATFTCFVVGSDLKDAHLTW
EVAGKVPTGGVEEGLLERHSNGSQSQHSRLTLPRSLWNAGTSVTCTLNHPS
LPPQRLMALREPAAQAPVKLSLNLLASSDPPEAASWLLCEVSGFSPPNILL
MWLEDQREVNTSGFAPARPPPQPGSTTFWAWSVLRVPAPPSPQPATYTCVV
SHEDSRTLLNASRSLEVSYVTDHGPMK

SEQ ID NO: 16 Human IgG1 Constant Region, Uniprot: P01857

ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVF
SCSVMHEALHNHYTQKSLSLSPGK

SEQ ID NO: 17 Human IgG2 Constant Region, Uniprot: P01859

ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSHEDPEVQ
FNWYVDGVEVHNAKTKPREEQFNSTFRVVSVLTVVHQDWLNGKEYKCKVSN
KGLPAPIEKTISKTKGQPREPQVYTLPPSREEMTKNQVSLTCLVKGFYPSD
ISVEWESNGQPENNYKTTPPMLDSDGSFFLYSKLTVDKSRWQQGNVFSCSV
MHEALHNHYTQKSLSLSPGK

SEQ ID NO: 18 Human IgG3 Constant Region, Uniprot: P01860

ASTKGPSVFPLAPCSRSTSGGTAALGCLVKDYFPEPVTVSWNSGALTSGVH
TFPAVLQSSGLYSLSSVVTVPSSSLGTQTYTCNVNHKPSNTKVDKRVELKT
PLGDTTHTCPRCPEPKSCDTPPPCPRCPEPKSCDTPPPCPRCPEPKSCDTP
PPCPRCPAPELLGGPSVFLFPPKPKDTLMISRTPEVTCVVVDVSHEDPEVQ
FKWYVDGVEVHNAKTKPREEQYNSTFRVVSVLTVLHQDWLNGKEYKCKVSN
KALPAPIEKTISKTKGQPREPQVYTLPPSREEMTKNQVSLTCLVKGFYPSD
IAVEWESSGQPENNYNTTPPMLDSDGSFFLYSKLTVDKSRWQQGNIFSCSV
MHEALHNRFTQKSLSLSPGK

SEQ ID NO: 19 Human IgM Constant Region, Uniprot: P01871

GSASAPTLFPLVSCENSPSDTSSVAVGCLAQDFLPDSITLSWKYKNNSDIS
STRGFPSVLRGGKYAATSQVLLPSKDVMQGTDEHVVCKVQHPNGNKEKNVP
LPVIAELPPKVSVFVPPRDGFFGNPRKSKLICQATGFSPRQIQVSWLREGK
QVGSGVTTDQVQAEAKESGPTTYKVTSTLTIKESDWLGQSMFTCRVDHRGL
TFQQNASSMCVPDQDTAIRVFAIPPSFASIFLTKSTKLTCLVTDLTTYDSV
TISWTRQNGEAVKTHTNISESHPNATFSAVGEASICEDDWNSGERFTCTVT
HTDLPSPLKQTISRPKGVALHRPDVYLLPPAREQLNLRESATITCLVTGFS
PADVFVQWMQRGQPLSPEKYVTSAPMPEPQAPGRYFAHSILTVSEEEWNTG
ETYTCVAHEALPNRVTERTVDKSTGKPTLYNVSLVMSDTAGTCY

SEQ ID NO: 20 Human IgG4 Constant Region, Uniprot: P01861

ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK

SEQ ID NO: 21 Human IgA1 Constant Region, Uniprot: P01876

ASPTSPKVFPLSLCSTQPDGNVVIACLVQGFFPQEPLSVTWSESGQGVTAR
NFPPSQDASGDLYTTSSQLTLPATQCLAGKSVTCHVKHYTNPSQDVTVPCP
VPSTPPTPSPSTPPTPSPSCCHPRLSLHRPALEDLLLGSEANLTCTLTGLR
DASGVTFTWTPSSGKSAVQGPPERDLCGCYSVSSVLPGCAEPWNHGKTFTC
TAAYPESKTPLTATLSKSGNTFRPEVHLLPPPSEELALNELVTLTCLARGF
SPKDVLVRWLQGSQELPREKYLTWASRQEPSQGTTTFAVTSILRVAAEDWK
KGDTFSCMVGHEALPLAFTQKTIDRLAGKPTHVNVSVVMAEVDGTCY

SEQ ID NO: 22 Human IgA2 Constant Region, Uniprot: P01877

ASPTSPKVFPLSLDSTPQDGNVVVACLVQGFFPQEPLSVTWSESGQNVTAR
NFPPSQDASGDLYTTSSQLTLPATQCPDGKSVTCHVKHYTNPSQDVTVPCP
VPPPPPCCHPRLSLHRPALEDLLLGSEANLTCTLTGLRDASGATFTWTPSS
GKSAVQGPPERDLCGCYSVSSVLPGCAQPWNHGETFTCTAAHPELKTPLTA
NITKSGNTFRPEVHLLPPPSEELALNELVTLTCLARGFSPKDVLVRWLQGS
QELPREKYLTWASRQEPSQGTTTFAVTSILRVAAEDWKKGDTFSCMVGHEA
LPLAFTQKTIDRMAGKPTHVNVSVVMAEVDGTCY

SEQ ID NO: 23 Human Ig Kappa Constant Region, Uniprot: P01834

TVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFN
RGEC

SEQ ID NO: 24 (Tip Heavy Chain Consensus Sequence)

| | |
|---|---|
| MDPKGSLSWR ILLFLSLAFE LSYGEVkLVe SGgglvqPGg SlrlSCaASG | 50 |
| FTFRTYAMSW VRQAPGkgLE WVATIGSDRR HTYYPDsvkG RfTIsRDNaK | 100 |
| NTLYlqmnSL RaEDTAVYYC VGPYDGYYGE FDYWGQGTLV TVSSASTKGP | 150 |
| SVFPLAPSSK STSGGTAALG CLVKDYFPEP VTVSWNSGAL TSGVHTFPAV | 200 |
| LQSSGLYSLS SVVTVPSSSL GTQTYICNVN HKPSNTKVDK KVEPKSCDKT | 250 |
| HTCPPCPAPE LLGGPSVFLF PPKPKDTLMI SRTPEVTCVV VDVSHEDPEV | 300 |
| KFNWYVDGVE VHNAKTKPRE EQYNSTYRVV SVLTVLHQDW LNGKEYKCKV | 350 |
| SNKALPAPIE KTISKAKGQP REPQVYTLPP SREEMTKNQV SLTCLVKGFY | 400 |
| PSDIAVEWES NGQPENNYKT TPPVLDSDGS FFLYSKLTVD KSRWQQGNVF | 450 |
| SCSVMHEALH NHYTQKSLSL SPG** | 475 |

Wherein a small letter can be substituted with an amino acid selected from SEQ ID NOs: 1-3 in the corresponding position.

SEQ ID NO: 25 (Tip Light Chain Consensus Sequence)

| | |
|---|---|
| METDTLLLWV LLLWVPGSTG DVVMTQSPIS LpVtLGqpAs IsCrSSQSLL | 50 |
| DSDGKTFLNW LQQrPGQsPr RLIYLVSKID SGVPDRFSGS GSGTDFTLkI | 100 |
| SrveAEDVgV YYCWOGTHFP YTFGQGTKLE IKRTVAAPSV FIFPPSDEQL | 150 |
| KSGTASVVCL LNNFYPREAK VQWKVDNALQ SGNSQESVTE QDSKDSTYSL | 200 |
| SSTLTLSKAD YEKHKVYACE VTHQGLSSPV TKSENRGEC** | 240 |

Wherein a small letter can be substituted with an amino acid selected from SEQ ID NOs: 7-9 in the corresponding position.

SEQ ID NO: 26 (Tail Heavy Chain Consensus Sequence)

| | |
|---|---|
| MDPKGSLSWR ILLFLSLAFE LSYGEVQLVE SGGGLVkPGg SLRLSCaASG | 50 |
| FTFSRYGMSW VRQAPGKGLE WVaTISSGGS YTYYTDSVKG RFTISRDNAK | 100 |
| NsLYLQMNSL raEDTAVYYC ERHGGDGYWY FDVWGQGTMV TVSSASTKGP | 150 |
| SVFPLAPSSK STSGGTAALG CLVKDYFPEP VTVSWNSGAL TSGVHTFPAV | 200 |
| LQSSGLYSLS SVVTVPSSSL GTQTYICNVN HKPSNTKVDK KVEPKSCDKT | 250 |
| HTCPPCPAPE LLGGPSVFLF PPKPKDTLMI SRTPEVTCVV VDVSHEDPEV | 300 |
| KFNWYVDGVE VHNAKTKPRE EQYNSTYRVV SVLTVLHQDW LNGKEYKCKV | 350 |
| SNKALPAPIE KTISKAKGQP REPQVYTLPP SREEMTKNQV SLTCLVKGFY | 400 |
| PSDIAVEWES NGQPENNYKT TPPVLDSDGS FFLYSKLTVD KSRWQQGNVF | 450 |
| SCSVMHEALH NHYTQKSLSL SPG** | 475 |

Wherein a small letter can be substituted with an amino acid selected from SEQ ID NOs: 4-6 in the corresponding position.

SEQ ID NO: 27 (Tail Light Chain Consensus Sequence)

METDTLLLWV LLLWVPGSTG DIqMTQSPss LSaSvGdRvT itCRASQDIS 50
NYLNWYQQKP GkAVkLLIYY TSRLHSGvPs RFSGSGSGTD YTLTISSLqP 100
EDFAtYfCQQ GNPLRTFGGG TKVEIKRTVA APSVFIFPPS DEQLKSGTAS 150
VVCLLNNFYP REAKVQWKVD NALQSGNSQE SVTEQDSKDS TYSLSSTLTL 200
SKADYEKHKV YACEVTHQGL SSPVTKSFNR GEC* 234

Wherein a small letter can be substituted with an amino acid selected from SEQ ID NOs: 10-12 in the corresponding position.

SEQ ID NO: 28 (The Consensus Sequence that IHF Binds)

### WATCAANNNNTTR

Wherein W is A or T and R is a purine.

SEQ ID NO. 29 (E. coli hupA, Genbank Accession No.: AP_003818, Last Accessed Mar. 21, 2011)

MNKTQLIDVIAEKAELSKTQAKAALESTLAAITESLKEGDAVQLVGFGT
FKVNHRAERTGRNPQTGKEIKIAAANVPAFVSGKALKDAVK

SEQ ID NO. 30 (E. coli hupB, Genbank Accession No.: AP_001090.1, Last Accessed Mar. 21, 2011)

MNKSQLIDKIAAGADISKAAAGRALDAIIASVTESLKEGDDVALVGFGT
FAVKERAARTGRNPQTGKEITIAAAKVPSFRAGKALKDAVN

SEQ ID NO: 31 (IhfA, a Tip Fragment)

NFELRDKSSRPGRNPKTGDVV

SEQ ID NO: 32 (IhfB, B Tip Fragment)

SLHHRQPRLGRNPKTGDSVNL

SEQ ID NO: 33 (a Peptide Linker)

Gly-Pro-Ser-Leu-Lys-Leu

SEQ ID NO: 34 (a Peptide Linker)

Gly-Pro-Ser-Leu

SEQ ID NO: 35 (a Peptide Linker)

Pro-Ser-Leu-Lys

SEQ ID NO: 36 (a Peptide Linker)

Gly-Pro-Ser-Leu-Lys

SEQ ID NO: 37 (a Peptide Linker)

Ser-Leu-Lys-Leu

SEQ ID NO: 38 (Tip-Chimeric Peptide IhfA5-mIhfB4_{NTHI})

RPGRNPX₁TGDVVPVSARRVV-X-FSLHHRQPRLGRNPX₁TGDSV

wherein “X” is an optional amino acid linker sequence, optionally comprising, or consisting essentially of, or yet further consisting of between 1 to 20 amino acids
wherein “X₁” is any amino acid or alternatively “X₁” is selected from the amino acids Q, R, K, S, or T.

SEQ ID NO: 39 (Tip-Chimeric Peptide IhfA5-mIhfB4_{NTHI})

RPGRNPKTGDVVPVSARRVV-X-FSLHHRQPRLGRNPKTGDSV

wherein “X” is an optional amino acid linker sequence optionally comprising between 1 to 20 amino acids.

SEQ ID NO: 40 (Tip-Chimeric Peptide IhfA5-mIhfB4_{NTHI})

RPGRNPKTGDVVPVSARRVVGPSLFSLHHRQPRLGRNPKTGDSV

SEQ ID NO: 41 (Tail-Chimeric Peptide IhfA3-IhfB2_{NTHI})

FLEEIRLSLESGQDVKLSGF-X-TLSAKEIENMVKDILEFISQ

SEQ ID NO. 42: Non-limiting
exemplary linker: GGSGGS
SEQ ID NO. 43: Non-limiting
exemplary linker: GPSLKL.
SEQ ID NO. 44: Non-limiting
exemplary linker: GGG.
SEQ ID NO. 45: Non-limiting
exemplary linker: GPSL.
SEQ ID NO. 46: Non-limiting
exemplary linker: GPS.
SEQ ID NO. 47: Non-limiting
exemplary linker: PSLK.
SEQ ID NO. 48: Non-limiting
exemplary linker: GPSLK.
SEQ ID NO. 49: Non-limiting
exemplary linker: SLKL.
SEQ ID NO: 50
(tail-chimeric peptide IhfA3-IhfB2_{NTHI})
FLEEIRLSLESGQDVKLSGFGPSLTLSAKEIENMVKDILEFISQ

### EMBODIMENTS

Embodiment 1. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the s of HMGB1 polypeptide, optionally wherein the polypeptide or a fragment thereof is isolated and/or engineered; and
(b) an anti-DNABII antibody or a fragment thereof comprising, or alternatively consisting essentially of, or yet further consisting of:
   (i) a heavy chain (HC) immunoglobulin variable domain sequence comprising, or alternatively consisting essentially of, or yet further consisting of a sequence selected from the group of: amino acid (aa) 25 to aa 144 of SEQ ID NOs: 13, 24 or 26 or an equivalent of each thereof, and/or
   (ii) a light chain (LC) immunoglobulin variable domain sequence comprising, or alternatively consisting essentially of, or yet further consisting of a sequence selected from the group of: aa 21 to aa 132 of SEQ ID NOs: 14 or 25, aa 21 to aa 126 of SEQ ID NO: 27 or an equivalent of each thereof.

Embodiment 2. The composition of embodiment 1, wherein the antibody comprises, or consists essentially of, or yet further consists of:
(i) a heavy chain (HC) immunoglobulin variable domain sequence comprising, or alternatively consisting essentially of, or yet further consisting of a sequence of aa 25 to aa 144 of SEQ ID NO: 24 or an equivalent thereof; and/or
(ii) a light chain (LC) immunoglobulin variable domain sequence comprising, or alternatively consisting essentially of, or yet further consisting of a sequence of aa 21 to aa 132 of SEQ ID NO: 25 or an equivalent thereof.

Embodiment 3. The composition of embodiment 1, wherein the antibody comprises, or consists essentially of, or yet further consists of:
(i) a heavy chain (HC) immunoglobulin variable domain sequence comprising, or alternatively consisting essentially of, or yet further consisting of a sequence selected from the group of: aa 25 to aa 144 of SEQ ID NOs: 1-6, 13, 24 or 26 or an equivalent of each thereof, and/or
(ii) a light chain (LC) immunoglobulin variable domain sequence comprising, or alternatively consisting essentially of, or yet further consisting of a sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 4. The composition of embodiment 1, wherein the heavy chain (HC) immunoglobulin variable domain sequence comprises, or consists essentially of, or yet further consists of an amino acid sequence of aa 25 to aa 144 of SEQ ID NO: 1 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 5. The composition of embodiment 1, wherein the heavy chain (HC) immunoglobulin variable domain sequence comprises, or consists essentially of, or yet further consists of an amino acid sequence of aa 25 to aa 144 of SEQ ID NO: 2 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 6. The composition of embodiment 1, wherein the heavy chain (HC) immunoglobulin variable domain sequence comprises, or consists essentially of, or yet further consists of an amino acid sequence of aa 25 to aa 144 of SEQ ID NO: 3 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 7. The composition of embodiment 1, wherein the heavy chain (HC) immunoglobulin variable domain sequence comprises, or consists essentially of, or yet further consists of an amino acid sequence of aa 25 to aa 144 of SEQ ID NO: 4 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 8. The composition of embodiment 1, wherein the heavy chain (HC) immunoglobulin variable domain sequence comprises, or consists essentially of, or yet further consists of an amino acid sequence of aa 25 to aa 144 of SEQ ID NO: 5 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 9. The composition of embodiment 1, wherein the heavy chain (HC) immunoglobulin variable domain sequence comprises, or consists essentially of, or yet further consists of an amino acid sequence of aa 25 to aa 144 of SEQ ID NO: 6 or an equivalent thereof, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27, or an equivalent of each thereof.

Embodiment 10. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 132 of SEQ ID NO: 7 or an equivalent thereof.

Embodiment 11. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 132 of SEQ ID NO: 8 or an equivalent thereof.

Embodiment 12. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 132 of SEQ ID NO: 9 or an equivalent thereof.

Embodiment 13. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 126 of SEQ ID NO: 10 or an equivalent thereof.

Embodiment 14. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 126 of SEQ ID NO: 11 or an equivalent thereof.

Embodiment 15. The composition of embodiment 1, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26 or an equivalent of each thereof, and/or wherein the light chain (LC) immunoglobulin variable domain sequence comprises, or consists essentially of, or yet further consists of an amino acid sequence of aa 21 to aa 126 of SEQ ID NO: 12 or an equivalent thereof.

Embodiment 16. The composition of embodiment 1, wherein the heavy chain (HC) immunoglobulin variable domain sequence comprises, or consists essentially of, or yet further consists of an amino acid sequence of aa 25 to aa 144 of SEQ ID NO: 1 or an equivalent thereof, and/or wherein the light chain (LC) immunoglobulin variable domain sequence comprises, or consists essentially of, or yet further consists of an amino acid sequence of aa 21 to aa 132 of SEQ ID NO: 7 or an equivalent thereof.

Embodiment 17. The composition of embodiment 1, wherein the heavy chain (HC) immunoglobulin variable domain sequence comprises, or consists essentially of, or yet further consists of an amino acid sequence of aa 25 to aa 144 of SEQ ID NO: 1 or an equivalent thereof, and/or wherein the light chain (LC) immunoglobulin variable domain sequence comprises, or consists essentially of, or yet further consists of an amino acid sequence of aa 21 to aa 132 of SEQ ID NO: 8 or an equivalent thereof.

Embodiment 18. The composition of embodiment 1, wherein the heavy chain (HC) immunoglobulin variable domain sequence comprises, or consists essentially of, or yet further consists of an amino acid sequence of aa 25 to aa 144 of SEQ ID NO: 1 or an equivalent thereof, and/or wherein the light chain (LC) immunoglobulin variable domain sequence comprises, or consists essentially of, or yet further consists of an amino acid sequence of aa 21 to aa 132 of SEQ ID NO: 9 or an equivalent thereof.

Embodiment 19. The composition of embodiment 1, wherein the heavy chain (HC) immunoglobulin variable domain sequence comprises, or consists essentially of, or yet further consists of an amino acid sequence of aa 25 to aa 144 of SEQ ID NO: 2 or an equivalent thereof, and/or wherein the light chain (LC) immunoglobulin variable domain sequence comprises, or consists essentially of, or yet further consists of an amino acid sequence aa 21 to aa 132 of SEQ ID of NO: 7 or an equivalent thereof.

Embodiment 20. The composition of embodiment 1, wherein the heavy chain (HC) immunoglobulin variable domain sequence comprises, or consists essentially of, or yet further consists of an amino acid sequence of aa 25 to aa 144 of SEQ ID NO: 2 or an equivalent thereof, and/or wherein the light chain (LC) immunoglobulin variable domain sequence comprises, or consists essentially of, or yet further consists of an amino acid sequence of aa 21 to aa 132 of SEQ ID NO: 8 or an equivalent thereof.

Embodiment 21. The composition of embodiment 1, wherein the heavy chain (HC) immunoglobulin variable domain sequence comprises, or consists essentially of, or yet further consists of an amino acid sequence of aa 25 to aa 144 of SEQ ID NO: 2 or an equivalent thereof, and/or wherein the light chain (LC) immunoglobulin variable domain sequence comprises, or consists essentially of, or yet further consists of an amino acid sequence of aa 21 to aa 132 of SEQ ID NO: 9 or an equivalent thereof.

Embodiment 22. The composition of embodiment 1, wherein the heavy chain (HC) immunoglobulin variable domain sequence comprises, or consists essentially of, or yet further consists of an amino acid sequence of aa 25 to aa 144 of SEQ ID NO: 3 or an equivalent thereof, and/or wherein the light chain (LC) immunoglobulin variable domain sequence comprises, or consists essentially of, or yet further consists of an amino acid sequence of aa 21 to aa 132 of SEQ ID NO: 7 or an equivalent thereof.

Embodiment 23. The composition of embodiment 1, wherein the heavy chain (HC) immunoglobulin variable domain sequence comprises, or consists essentially of, or yet further consists of an amino acid sequence of aa 25 to aa 144 of SEQ ID NO: 3 or an equivalent thereof, and/or wherein the light chain (LC) immunoglobulin variable domain sequence comprises, or consists essentially of, or yet further consists of an amino acid sequence of aa 21 to aa 132 of SEQ ID NO: 8 or an equivalent thereof.

Embodiment 24. The composition of embodiment 1, wherein the heavy chain (HC) immunoglobulin variable domain sequence comprises, or consists essentially of, or yet further consists of an amino acid sequence of aa 25 to aa 144 of SEQ ID NO: 3 or an equivalent thereof, and/or wherein the light chain (LC) immunoglobulin variable domain sequence comprises, or consists essentially of, or yet further consists of an amino acid sequence of aa 21 to aa 132 of SEQ ID NO: 9 or an equivalent thereof.

Embodiment 25. The composition of embodiment 1, wherein the heavy chain (HC) immunoglobulin variable domain sequence comprises, or consists essentially of, or yet further consists of an amino acid sequence of aa 25 to aa 144 of SEQ ID NO: 4 or an equivalent thereof, and/or wherein the light chain (LC) immunoglobulin variable domain sequence comprises, or consists essentially of, or yet further consists of an amino acid sequence of aa 21 to aa 126 of SEQ ID NO: 10 or an equivalent thereof.

Embodiment 26. The composition of embodiment 1, wherein the heavy chain (HC) immunoglobulin variable domain sequence comprises, or consists essentially of, or yet further consists of an amino acid sequence of aa 25 to aa 144 of SEQ ID NO: 4 or an equivalent thereof, and/or wherein the light chain (LC) immunoglobulin variable domain sequence comprises, or consists essentially of, or yet further consists of an amino acid sequence of aa 21 to aa 126 of SEQ ID NO: 11 or an equivalent thereof.

Embodiment 27. The composition of embodiment 1, wherein the heavy chain (HC) immunoglobulin variable domain sequence comprises, or consists essentially of, or yet further consists of an amino acid sequence of aa 25 to aa 144 of SEQ ID NO: 4 or an equivalent thereof, and/or wherein the light chain (LC) immunoglobulin variable domain sequence comprises, or consists essentially of, or yet further consists of an amino acid sequence of aa 21 to aa 126 of SEQ ID NO: 12 or an equivalent thereof.

Embodiment 28. The composition of embodiment 1, wherein the heavy chain (HC) immunoglobulin variable domain sequence comprises, or consists essentially of, or yet further consists of an amino acid sequence of aa 25 to aa 144 of SEQ ID NO: 5 or an equivalent thereof, and/or wherein the light chain (LC) immunoglobulin variable domain sequence comprises, or consists essentially of, or yet further consists of an amino acid sequence of aa 21 to aa 126 of SEQ ID NO: 10 or an equivalent thereof.

Embodiment 29. The composition of embodiment 1, wherein the heavy chain (HC) immunoglobulin variable domain sequence comprises, or consists essentially of, or yet further consists of an amino acid sequence of aa 25 to aa 144 of SEQ ID NO: 5 or an equivalent thereof, and/or wherein the light chain (LC) immunoglobulin variable domain sequence comprises, or consists essentially of, or yet further consists of an amino acid sequence of aa 21 to aa 126 of SEQ ID NO: 11 or an equivalent thereof.

Embodiment 30. The composition of embodiment 1, wherein the heavy chain (HC) immunoglobulin variable domain sequence comprises, or consists essentially of, or yet further consists of an amino acid sequence of aa 25 to aa 144 of SEQ ID NO: 5 or an equivalent thereof, and/or wherein the light chain (LC) immunoglobulin variable domain sequence comprises, or consists essentially of, or yet further consists of an amino acid sequence of aa 21 to aa 126 of SEQ ID NO: 12 or an equivalent thereof.

Embodiment 31. The composition of embodiment 1, wherein the heavy chain (HC) immunoglobulin variable domain sequence comprises, or consists essentially of, or yet further consists of an amino acid sequence of aa 25 to aa 144 of SEQ ID NO: 6 or an equivalent thereof, and/or wherein the light chain (LC) immunoglobulin variable domain sequence comprises, or consists essentially of, or yet further consists of an amino acid sequence of aa 21 to aa 126 of SEQ ID NO: 10 or an equivalent thereof.

Embodiment 32. The composition of embodiment 1, wherein the heavy chain (HC) immunoglobulin variable domain sequence comprises, or consists essentially of, or yet further consists of an amino acid sequence of aa 25 to aa 144 of SEQ ID NO: 6 or an equivalent thereof, and/or wherein the light chain (LC) immunoglobulin variable domain sequence comprises, or consists essentially of, or yet further consists of an amino acid sequence of aa 21 to aa 126 of SEQ ID NO: 11 or an equivalent thereof.

Embodiment 33. The composition of embodiment 1, wherein the heavy chain (HC) immunoglobulin variable domain sequence comprises, or consists essentially of, or yet further consists of an amino acid sequence of aa 25 to aa 144 of SEQ ID NO: 6 or an equivalent thereof, and/or wherein the light chain (LC) immunoglobulin variable domain sequence comprises, or consists essentially of, or yet further consists of an amino acid sequence of aa 21 to aa 126 of SEQ ID NO: 12 or an equivalent thereof.

Embodiment 34. The composition of embodiment 1, wherein the antibody or a fragment thereof comprising, or alternatively consisting essentially of, or yet further consisting of:
(i) a heavy chain (HC) immunoglobulin variable domain sequence comprising, or alternatively consisting essentially of, or yet further consisting of a sequence selected from the group of: amino acid (aa) 25 to aa 144 of SEQ ID NOs: 13, 24 or 26; and/or
(ii) a light chain (LC) immunoglobulin variable domain sequence comprising, or alternatively consisting essentially of, or yet further consisting of a sequence selected from the group of: aa 21 to aa 132 of SEQ ID NOs: 14 or 25, aa 21 to aa 126 of SEQ ID NO: 27.

Embodiment 35. The composition of embodiment 34, wherein the antibody comprises, or consists essentially of, or yet further consists of:
(i) a heavy chain (HC) immunoglobulin variable domain sequence comprising, or alternatively consisting essentially of, or yet further consisting of a sequence of aa 25 to aa 144 of SEQ ID NO: 24; and/or
(ii) a light chain (LC) immunoglobulin variable domain sequence comprising, or alternatively consisting essentially of, or yet further consisting of a sequence of aa 21 to aa 132 of SEQ ID NO: 25.

Embodiment 36. The composition of embodiment 34, wherein the antibody comprises, or consists essentially of, or yet further consists of:
(i) a heavy chain (HC) immunoglobulin variable domain sequence comprising, or alternatively consisting essentially of, or yet further consisting of a sequence selected from the group of: aa 25 to aa 144 of SEQ ID NOs: 1-6, 13, 24 or 26; and/or
(ii) a light chain (LC) immunoglobulin variable domain sequence comprising, or alternatively consisting essentially of, or yet further consisting of a sequence selected from the group of: aa 21 to aa 132 of SEQ ID NOs: 7-9, 14 or 25, aa 21 to aa 126 of SEQ ID NOs: 10-12 or 27.

Embodiment 37. The composition of embodiment 34, wherein the heavy chain (HC) immunoglobulin variable domain sequence comprises, or consists essentially of, or yet further consists of an amino acid sequence of aa 25 to aa 144 of SEQ ID NO: 1, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 38. The composition of embodiment 34, wherein the heavy chain (HC) immunoglobulin variable domain sequence comprises, or consists essentially of, or yet further consists of an amino acid sequence of aa 25 to aa 144 of SEQ ID NO: 2, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 39. The composition of embodiment 34, wherein the heavy chain (HC) immunoglobulin variable domain sequence comprises, or consists essentially of, or yet further consists of an amino acid sequence of aa 25 to aa 144 of SEQ ID NO: 3, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 40. The composition of embodiment 34, wherein the heavy chain (HC) immunoglobulin variable domain sequence comprises, or consists essentially of, or yet further consists of an amino acid sequence of aa 25 to aa 144 of SEQ ID NO: 4, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 41. The composition of embodiment 34, wherein the heavy chain (HC) immunoglobulin variable domain sequence comprises, or consists essentially of, or yet further consists of an amino acid sequence of aa 25 to aa 144 of SEQ ID NO: 5, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 42. The composition of embodiment 34, wherein the heavy chain (HC) immunoglobulin variable domain sequence comprises, or consists essentially of, or yet further consists of an amino acid sequence of aa 25 to aa 144 of SEQ ID NO: 6, and/or wherein the light chain (LC) immunoglobulin variable domain sequence comprises, or consists essentially of, or yet further consists of an amino acid sequence selected from the group of: aa 21 to aa 132 of SEQ ID NOs: 7-9, 14 or 25, aa 21 to aa 126 of SEQ ID NOs: 10-12 or 27.

Embodiment 43. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 132 of SEQ ID NO: 7.

Embodiment 44. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 132 of SEQ ID NO: 8.

Embodiment 45. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 132 of SEQ ID NO: 9.

Embodiment 46. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 126 of SEQ ID NO: 10.

Embodiment 47. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 126 of SEQ ID NO: 11.

Embodiment 48. The composition of embodiment 34, wherein the heavy chain (HC) immunoglobulin variable domain sequence comprises, or consists essentially of, or yet further consists of an amino acid sequence selected from the group of: aa 25 to aa 144 of SEQ ID NOs: 1-6, 13, 24, or 26, and/or wherein the light chain (LC) immunoglobulin variable domain sequence comprises, or consists essentially of, or yet further consists of an amino acid sequence of aa 21 to aa 126 of SEQ ID NO: 12.

Embodiment 49. The composition of embodiment 34, wherein the heavy chain (HC) immunoglobulin variable domain sequence comprises, or consists essentially of, or yet further consists of an amino acid sequence of aa 25 to aa 144 of SEQ ID NO: 1, and/or wherein the light chain (LC) immunoglobulin variable domain sequence comprises, or consists essentially of, or yet further consists of an amino acid sequence of aa 21 to aa 132 of SEQ ID NO: 7.

Embodiment 50. The composition of embodiment 34, wherein the heavy chain (HC) immunoglobulin variable domain sequence comprises, or consists essentially of, or yet further consists of an amino acid sequence of aa 25 to aa 144 of SEQ ID NO: 1, and/or wherein the light chain (LC) immunoglobulin variable domain sequence comprises, or consists essentially of, or yet further consists of an amino acid sequence of aa 21 to aa 132 of SEQ ID NO: 8.

Embodiment 51. The composition of embodiment 34, wherein the heavy chain (HC) immunoglobulin variable domain sequence comprises, or consists essentially of, or yet further consists of an amino acid sequence of aa 25 to aa 144 of SEQ ID NO: 1, and/or wherein the light chain (LC) immunoglobulin variable domain sequence comprises, or consists essentially of, or yet further consists of an amino acid sequence of aa 21 to aa 132 of SEQ ID NO: 9.

Embodiment 52. The composition of embodiment 34, wherein the heavy chain (HC) immunoglobulin variable domain sequence comprises, or consists essentially of, or yet further consists of an amino acid sequence of aa 25 to aa 144 of SEQ ID NO: 2, and/or wherein the light chain (LC) immunoglobulin variable domain sequence comprises, or consists essentially of, or yet further consists of an amino acid sequence aa 21 to aa 132 of SEQ ID of NO: 7.

Embodiment 53. The composition of embodiment 34, wherein the heavy chain (HC) immunoglobulin variable domain sequence comprises, or consists essentially of, or yet further consists of an amino acid sequence of aa 25 to aa 144 of SEQ ID NO: 2, and/or wherein the light chain (LC) immunoglobulin variable domain sequence comprises, or consists essentially of, or yet further consists of an amino acid sequence of aa 21 to aa 132 of SEQ ID NO: 8.

Embodiment 54. The composition of embodiment 34, wherein the heavy chain (HC) immunoglobulin variable domain sequence comprises, or consists essentially of, or yet further consists of an amino acid sequence of aa 25 to aa 144 of SEQ ID NO: 2, and/or wherein the light chain (LC) immunoglobulin variable domain sequence comprises, or consists essentially of, or yet further consists of an amino acid sequence of aa 21 to aa 132 of SEQ ID NO: 9.

Embodiment 55. The composition of embodiment 34, wherein the heavy chain (HC) immunoglobulin variable domain sequence comprises, or consists essentially of, or yet further consists of an amino acid sequence of aa 25 to aa 144 of SEQ ID NO: 3, and/or wherein the light chain (LC) immunoglobulin variable domain sequence comprises, or consists essentially of, or yet further consists of an amino acid sequence of aa 21 to aa 132 of SEQ ID NO: 7.

Embodiment 56. The composition of embodiment 34, wherein the heavy chain (HC) immunoglobulin variable domain sequence comprises, or consists essentially of, or yet further consists of an amino acid sequence of aa 25 to aa 144 of SEQ ID NO: 3, and/or wherein the light chain (LC) immunoglobulin variable domain sequence comprises, or consists essentially of, or yet further consists of an amino acid sequence of aa 21 to aa 132 of SEQ ID NO: 8.

Embodiment 57. The composition of embodiment 34, wherein the heavy chain (HC) immunoglobulin variable domain sequence comprises, or consists essentially of, or yet further consists of an amino acid sequence of aa 25 to aa 144 of SEQ ID NO: 3, and/or wherein the light chain (LC) immunoglobulin variable domain sequence comprises, or consists essentially of, or yet further consists of an amino acid sequence of aa 21 to aa 132 of SEQ ID NO: 9.

Embodiment 58. The composition of embodiment 34, wherein the heavy chain (HC) immunoglobulin variable domain sequence comprises, or consists essentially of, or yet further consists of an amino acid sequence of aa 25 to aa 144 of SEQ ID NO: 4, and/or wherein the light chain (LC) immunoglobulin variable domain sequence comprises, or consists essentially of, or yet further consists of an amino acid sequence of aa 21 to aa 126 of SEQ ID NO: 10.

Embodiment 59. The composition of embodiment 34, wherein the heavy chain (HC) immunoglobulin variable domain sequence comprises, or consists essentially of, or yet further consists of an amino acid sequence of aa 25 to aa 144 of SEQ ID NO: 4, and/or wherein the light chain (LC) immunoglobulin variable domain sequence comprises, or consists essentially of, or yet further consists of an amino acid sequence of aa 21 to aa 126 of SEQ ID NO: 11.

Embodiment 60. The composition of embodiment 34, wherein the heavy chain (HC) immunoglobulin variable domain sequence comprises, or consists essentially of, or yet further consists of an amino acid sequence of aa 25 to aa 144 of SEQ ID NO: 4, and/or wherein the light chain (LC) immunoglobulin variable domain sequence comprises, or consists essentially of, or yet further consists of an amino acid sequence of aa 21 to aa 126 of SEQ ID NO: 12.

Embodiment 61. The composition of embodiment 34, wherein the heavy chain (HC) immunoglobulin variable domain sequence comprises, or consists essentially of, or yet further consists of an amino acid sequence of aa 25 to aa 144 of SEQ ID NO: 5, and/or wherein the light chain (LC) immunoglobulin variable domain sequence comprises, or consists essentially of, or yet further consists of an amino acid sequence of aa 21 to aa 126 of SEQ ID NO: 10.

Embodiment 62. The composition of embodiment 34, wherein the heavy chain (HC) immunoglobulin variable domain sequence comprises, or consists essentially of, or yet further consists of an amino acid sequence of aa 25 to aa 144 of SEQ ID NO: 5, and/or wherein the light chain (LC) immunoglobulin variable domain sequence comprises, or consists essentially of, or yet further consists of an amino acid sequence of aa 21 to aa 126 of SEQ ID NO: 11.

Embodiment 63. The composition of embodiment 34, wherein the heavy chain (HC) immunoglobulin variable domain sequence comprises, or consists essentially of, or yet further consists of an amino acid sequence of aa 25 to aa 144 of SEQ ID NO: 5, and/or wherein the light chain (LC) immunoglobulin variable domain sequence comprises, or consists essentially of, or yet further consists of an amino acid sequence of aa 21 to aa 126 of SEQ ID NO: 12.

Embodiment 64. The composition of embodiment 34, wherein the heavy chain (HC) immunoglobulin variable domain sequence comprises, or consists essentially of, or yet further consists of an amino acid sequence of aa 25 to aa 144 of SEQ ID NO: 6, and/or wherein the light chain (LC) immunoglobulin variable domain sequence comprises, or consists essentially of, or yet further consists of an amino acid sequence of aa 21 to aa 126 of SEQ ID NO: 10.

Embodiment 65. The composition of embodiment 34, wherein the heavy chain (HC) immunoglobulin variable domain sequence comprises, or consists essentially of, or yet further consists of an amino acid sequence of aa 25 to aa 144 of SEQ ID NO: 6, and/or wherein the light chain (LC) immunoglobulin variable domain sequence comprises, or consists essentially of, or yet further consists of an amino acid sequence of aa 21 to aa 126 of SEQ ID NO: 11.

Embodiment 66. The composition of embodiment 34, wherein the heavy chain (HC) immunoglobulin variable domain sequence comprises, or consists essentially of, or yet further consists of an amino acid sequence of aa 25 to aa 144 of SEQ ID NO: 6, and/or wherein the light chain (LC) immunoglobulin variable domain sequence comprises, or consists essentially of, or yet further consists of an amino acid sequence of aa 21 to aa 126 of SEQ ID NO: 12.

Embodiment 67. The composition of embodiment 1, wherein the antibody or a fragment thereof comprising, or alternatively consisting essentially of, or yet further consisting of:
(i) a heavy chain (HC) comprising, or alternatively consisting essentially of, or yet further consisting of a sequence selected from the group of: amino acid (aa) 25 to aa 473 of SEQ ID NOs: 13, 24 or 26 or an equivalent of each thereof; and/or
(ii) a light chain (LC) comprising, or alternatively consisting essentially of, or yet further consisting of a sequence selected from the group of: aa 21 to aa 239 of SEQ ID NOs: 14 or 25, aa 21 to aa 233 of SEQ ID NO: 27 or an equivalent of each thereof.

Embodiment 68. The composition of embodiment 67, wherein the antibody comprises, or consists essentially of, or yet further consists of:
(i) a heavy chain (HC) comprising, or alternatively consisting essentially of, or yet further consisting of a sequence of aa 25 to aa 473 of SEQ ID NO: 24 or an equivalent thereof; and/or
(ii) a light chain (LC) comprising, or alternatively consisting essentially of, or yet further consisting of a sequence of aa 21 to aa 239 of SEQ ID NO: 25 or an equivalent thereof.

Embodiment 69. The composition of embodiment 67, wherein the antibody comprises, or consists essentially of, or yet further consists of:
(i) a heavy chain (HC) comprising, or alternatively consisting essentially of, or yet further consisting of a sequence selected from the group of: aa 25 to aa 473 of SEQ ID NOs: 1-6, 13, 24 or 26 or an equivalent of each thereof, and/or
(ii) a light chain (LC) comprising, or alternatively consisting essentially of, or yet further consisting of a sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 70. The composition of embodiment 67, wherein the heavy chain (HC) comprises, or consists essentially of, or yet further consists of an amino acid sequence of aa 25 to aa 473 of SEQ ID NO: 1 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 71. The composition of embodiment 67, wherein the heavy chain (HC) comprises, or consists essentially of, or yet further consists of an amino acid sequence of aa 25 to aa 473 of SEQ ID NO: 2 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 72. The composition of embodiment 67, wherein the heavy chain (HC) comprises, or consists essentially of, or yet further consists of an amino acid sequence of aa 25 to aa 473 of SEQ ID NO: 3 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 73. The composition of embodiment 67, wherein the heavy chain (HC) comprises, or consists essentially of, or yet further consists of an amino acid sequence of aa 25 to aa 473 of SEQ ID NO: 4 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 74. The composition of embodiment 67, wherein the heavy chain (HC) comprises, or consists essentially of, or yet further consists of an amino acid sequence of aa 25 to aa 473 of SEQ ID NO: 5 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 75. The composition of embodiment 67, wherein the heavy chain (HC) comprises, or consists essentially of, or yet further consists of an amino acid sequence of aa 25 to aa 473 of SEQ ID NO: 6 or an equivalent thereof, and/or wherein the light chain (LC) comprises, or consists essentially of, or yet further consists of an amino acid sequence selected from the group of: aa 21 to aa 239 of SEQ ID NOs: 7-9, 14 or 25, aa 21 to aa 233 of SEQ ID NOs: 10-12 or 27, or an equivalent of each thereof.

Embodiment 76. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 239 of SEQ ID NO: 7 or an equivalent thereof.

Embodiment 77. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 239 of SEQ ID NO: 8 or an equivalent thereof.

Embodiment 78. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 239 of SEQ ID NO: 9 or an equivalent thereof.

Embodiment 79. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 233 of SEQ ID NO: 10 or an equivalent thereof.

Embodiment 80. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 233 of SEQ ID NO: 11 or an equivalent thereof.

Embodiment 81. The composition of embodiment 67, wherein the heavy chain (HC) comprises, or consists essentially of, or yet further consists of an amino acid sequence selected from the group of: aa 25 to aa 473 of SEQ ID NOs: 1-6, 13, 24, or 26 or an equivalent of each thereof, and/or wherein the light chain (LC) comprises, or consists essentially of, or yet further consists of an amino acid sequence of aa 21 to aa 233 of SEQ ID NO: 12 or an equivalent thereof.

Embodiment 82. The composition of embodiment 67, wherein the heavy chain (HC) comprises, or consists essentially of, or yet further consists of an amino acid sequence of aa 25 to aa 473 of SEQ ID NO: 1 or an equivalent thereof, and/or wherein the light chain (LC) comprises, or consists essentially of, or yet further consists of an amino acid sequence of aa 21 to aa 239 of SEQ ID NO: 7 or an equivalent thereof.

Embodiment 83. The composition of embodiment 67, wherein the heavy chain (HC) comprises, or consists essentially of, or yet further consists of an amino acid sequence of aa 25 to aa 473 of SEQ ID NO: 1 or an equivalent thereof, and/or wherein the light chain (LC) comprises, or consists essentially of, or yet further consists of an amino acid sequence of aa 21 to aa 239 of SEQ ID NO: 8 or an equivalent thereof.

Embodiment 84. The composition of embodiment 67, wherein the heavy chain (HC) comprises, or consists essentially of, or yet further consists of an amino acid sequence of aa 25 to aa 473 of SEQ ID NO: 1 or an equivalent thereof, and/or wherein the light chain (LC) comprises, or consists essentially of, or yet further consists of an amino acid sequence of aa 21 to aa 239 of SEQ ID NO: 9 or an equivalent thereof.

Embodiment 85. The composition of embodiment 67, wherein the heavy chain (HC) comprises, or consists essentially of, or yet further consists of an amino acid sequence of aa 25 to aa 473 of SEQ ID NO: 2 or an equivalent thereof, and/or wherein the light chain (LC) comprises, or consists essentially of, or yet further consists of an amino acid sequence aa 21 to aa 239 of SEQ ID of NO: 7 or an equivalent thereof.

Embodiment 86. The composition of embodiment 67, wherein the heavy chain (HC) comprises, or consists essentially of, or yet further consists of an amino acid sequence of aa 25 to aa 473 of SEQ ID NO: 2 or an equivalent thereof, and/or wherein the light chain (LC) comprises, or consists essentially of, or yet further consists of an amino acid sequence of aa 21 to aa 239 of SEQ ID NO: 8 or an equivalent thereof.

Embodiment 87. The composition of embodiment 67, wherein the heavy chain (HC) comprises, or consists essentially of, or yet further consists of an amino acid sequence of aa 25 to aa 473 of SEQ ID NO: 2 or an equivalent thereof, and/or wherein the light chain (LC) comprises, or consists essentially of, or yet further consists of an amino acid sequence of aa 21 to aa 239 of SEQ ID NO: 9 or an equivalent thereof.

Embodiment 88. The composition of embodiment 67, wherein the heavy chain (HC) comprises, or consists essentially of, or yet further consists of an amino acid sequence of aa 25 to aa 473 of SEQ ID NO: 3 or an equivalent thereof, and/or wherein the light chain (LC) comprises, or consists essentially of, or yet further consists of an amino acid sequence of aa 21 to aa 239 of SEQ ID NO: 7 or an equivalent thereof.

Embodiment 89. The composition of embodiment 67, wherein the heavy chain (HC) comprises, or consists essentially of, or yet further consists of an amino acid sequence of aa 25 to aa 473 of SEQ ID NO: 3 or an equivalent thereof, and/or wherein the light chain (LC) comprises, or consists essentially of, or yet further consists of an amino acid sequence of aa 21 to aa 239 of SEQ ID NO: 8 or an equivalent thereof.

Embodiment 90. The composition of embodiment 67, wherein the heavy chain (HC) comprises, or consists essentially of, or yet further consists of an amino acid sequence of aa 25 to aa 473 of SEQ ID NO: 3 or an equivalent thereof, and/or wherein the light chain (LC) comprises, or consists essentially of, or yet further consists of an amino acid sequence of aa 21 to aa 239 of SEQ ID NO: 9 or an equivalent thereof.

Embodiment 91. The composition of embodiment 67, wherein the heavy chain (HC) comprises, or consists essentially of, or yet further consists of an amino acid sequence of aa 25 to aa 473 of SEQ ID NO: 4 or an equivalent thereof, and/or wherein the light chain (LC) comprises, or consists essentially of, or yet further consists of an amino acid sequence of aa 21 to aa 233 of SEQ ID NO: 10 or an equivalent thereof.

Embodiment 92. The composition of embodiment 67, wherein the heavy chain (HC) comprises, or consists essentially of, or yet further consists of an amino acid sequence of aa 25 to aa 473 of SEQ ID NO: 4 or an equivalent thereof, and/or wherein the light chain (LC) comprises, or consists essentially of, or yet further consists of an amino acid sequence of aa 21 to aa 233 of SEQ ID NO: 11 or an equivalent thereof.

Embodiment 93. The composition of embodiment 67, wherein the heavy chain (HC) comprises, or consists essentially of, or yet further consists of an amino acid sequence of aa 25 to aa 473 of SEQ ID NO: 4 or an equivalent thereof, and/or wherein the light chain (LC) comprises, or consists essentially of, or yet further consists of an amino acid sequence of aa 21 to aa 233 of SEQ ID NO: 12 or an equivalent thereof.

Embodiment 94. The composition of embodiment 67, wherein the heavy chain (HC) comprises, or consists essentially of, or yet further consists of an amino acid sequence of aa 25 to aa 473 of SEQ ID NO: 5 or an equivalent thereof, and/or wherein the light chain (LC) comprises, or consists essentially of, or yet further consists of an amino acid sequence of aa 21 to aa 233 of SEQ ID NO: 10 or an equivalent thereof.

Embodiment 95. The composition of embodiment 67, wherein the heavy chain (HC) comprises, or consists essentially of, or yet further consists of an amino acid sequence of aa 25 to aa 473 of SEQ ID NO: 5 or an equivalent thereof, and/or wherein the light chain (LC) comprises, or consists essentially of, or yet further consists of an amino acid sequence of aa 21 to aa 233 of SEQ ID NO: 11 or an equivalent thereof.

Embodiment 96. The composition of embodiment 67, wherein the heavy chain (HC) comprises, or consists essentially of, or yet further consists of an amino acid sequence of aa 25 to aa 473 of SEQ ID NO: 5 or an equivalent thereof, and/or wherein the light chain (LC) comprises, or consists essentially of, or yet further consists of an amino acid sequence of aa 21 to aa 233 of SEQ ID NO: 12 or an equivalent thereof.

Embodiment 97. The composition of embodiment 67, wherein the heavy chain (HC) comprises, or consists essentially of, or yet further consists of an amino acid sequence of aa 25 to aa 473 of SEQ ID NO: 6 or an equivalent thereof, and/or wherein the light chain (LC) comprises, or consists essentially of, or yet further consists of an amino acid sequence of aa 21 to aa 233 of SEQ ID NO: 10 or an equivalent thereof.

Embodiment 98. The composition of embodiment 67, wherein the heavy chain (HC) comprises, or consists essentially of, or yet further consists of an amino acid sequence of aa 25 to aa 473 of SEQ ID NO: 6 or an equivalent thereof, and/or wherein the light chain (LC) comprises, or consists essentially of, or yet further consists of an amino acid sequence of aa 21 to aa 233 of SEQ ID NO: 11 or an equivalent thereof.

Embodiment 99. The composition of embodiment 67, wherein the heavy chain (HC) comprises, or consists essentially of, or yet further consists of an amino acid sequence of aa 25 to aa 473 of SEQ ID NO: 6 or an equivalent thereof, and/or wherein the light chain (LC) comprises, or consists essentially of, or yet further consists of an amino acid sequence of aa 21 to aa 233 of SEQ ID NO: 12 or an equivalent thereof.

Embodiment 100. The composition of any one of embodiments 1-99, wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, and optionally wherein the HMGB1 is a human HMGB1 or a murine HMGB1.

Embodiment 101. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an anti-DNABII antibody or a fragment thereof comprising, or alternatively consisting essentially of, or yet further consisting of:
   (i) a heavy chain (HC) comprising, or alternatively consisting essentially of, or yet further consisting of a sequence selected from the group of: SEQ ID NOs: 13, 24 or 26 or an equivalent of each thereof, and/or
   (ii) a light chain (LC) comprising, or alternatively consisting essentially of, or yet further consisting of a sequence selected from the group of: SEQ ID NOs: 14, 25, or 27 or an equivalent of each thereof.

Embodiment 102. The composition of embodiment 101, wherein the antibody comprises, or consists essentially of, or yet further consists of:
(i) a heavy chain (HC) comprising, or alternatively consisting essentially of, or yet further consisting of a sequence of SEQ ID NO: 24 or an equivalent thereof; and/or
(ii) a light chain (LC) comprising, or alternatively consisting essentially of, or yet further consisting of a sequence of SEQ ID NO: 25 or an equivalent thereof.

Embodiment 103. The composition of embodiment 101, wherein the antibody comprises, or consists essentially of, or yet further consists of:
(i) a heavy chain (HC) comprising, or alternatively consisting essentially of, or yet further consisting of a sequence selected from the group of: SEQ ID NOs: 1-6, 13, 24 or 26 or an equivalent of each thereof, and/or
(ii) a light chain (LC) comprising, or alternatively consisting essentially of, or yet further consisting of a sequence selected from the group of: SEQ ID NOs: 7-12, 14, 25, or 27, or an equivalent of each thereof.

Embodiment 104. The composition of embodiment 101, wherein the heavy chain (HC) comprises, or consists essentially of, or yet further consists of an amino acid sequence of SEQ ID NO: 1 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 105. The composition of embodiment 101, wherein the heavy chain (HC) comprises, or consists essentially of, or yet further consists of an amino acid sequence of SEQ ID NO: 2 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 106. The composition of embodiment 101, wherein the heavy chain (HC) comprises, or consists essentially of, or yet further consists of an amino acid sequence of SEQ ID NO: 3 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 107. The composition of embodiment 101, wherein the heavy chain (HC) comprises, or consists essentially of, or yet further consists of an amino acid sequence of SEQ ID NO: 4 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 108. The composition of embodiment 101, wherein the heavy chain (HC) comprises, or consists essentially of, or yet further consists of an amino acid sequence of SEQ ID NO: 5 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 109. The composition of embodiment 101, wherein the heavy chain (HC) comprises, or consists essentially of, or yet further consists of an amino acid sequence of SEQ ID NO: 6 or an equivalent thereof, and/or wherein the light chain (LC) comprises, or consists essentially of, or yet further consists of an amino acid sequence selected from the group of: SEQ ID NOs: 7-12, 14, 25, or 27, or an equivalent of each thereof.

Embodiment 110. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 7 or an equivalent thereof.

Embodiment 111. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 8 or an equivalent thereof.

Embodiment 112. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 9 or an equivalent thereof.

Embodiment 113. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 10 or an equivalent thereof.

Embodiment 114. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 11 or an equivalent thereof.

Embodiment 115. The composition of embodiment 101, wherein the heavy chain (HC) comprises, or consists essentially of, or yet further consists of an amino acid sequence selected from the group of: SEQ ID NOs: 1-6, 13, 24, or 26 or an equivalent of each thereof, and/or wherein the light chain (LC) comprises, or consists essentially of, or yet further consists of an amino acid sequence of SEQ ID NO: 12 or an equivalent thereof.

Embodiment 116. The composition of embodiment 101, wherein the heavy chain (HC) comprises, or consists essentially of, or yet further consists of an amino acid sequence of SEQ ID NO: 1 or an equivalent thereof, and/or wherein the light chain (LC) comprises, or consists essentially of, or yet further consists of an amino acid sequence of SEQ ID NO: 7 or an equivalent thereof.

Embodiment 117. The composition of embodiment 101, wherein the heavy chain (HC) comprises, or consists essentially of, or yet further consists of an amino acid sequence of SEQ ID NO: 1 or an equivalent thereof, and/or wherein the light chain (LC) comprises, or consists essentially of, or yet further consists of an amino acid sequence of SEQ ID NO: 8 or an equivalent thereof.

Embodiment 118. The composition of embodiment 101, wherein the heavy chain (HC) comprises, or consists essentially of, or yet further consists of an amino acid sequence of SEQ ID NO: 1 or an equivalent thereof, and/or wherein the light chain (LC) comprises, or consists essentially of, or yet further consists of an amino acid sequence of SEQ ID NO: 9 or an equivalent thereof.

Embodiment 119. The composition of embodiment 101, wherein the heavy chain (HC) comprises, or consists essentially of, or yet further consists of an amino acid sequence of SEQ ID NO: 2 or an equivalent thereof, and/or wherein the light chain (LC) comprises, or consists essentially of, or yet further consists of an amino acid sequence of SEQ ID NO: 7 or an equivalent thereof.

Embodiment 120. The composition of embodiment 101, wherein the heavy chain (HC) comprises, or consists essentially of, or yet further consists of an amino acid sequence of SEQ ID NO: 2 or an equivalent thereof, and/or wherein the light chain (LC) comprises, or consists essentially of, or yet further consists of an amino acid sequence of SEQ ID NO: 8 or an equivalent thereof.

Embodiment 121. The composition of embodiment 101, wherein the heavy chain (HC) comprises, or consists essentially of, or yet further consists of an amino acid sequence of SEQ ID NO: 2 or an equivalent thereof, and/or wherein the light chain (LC) comprises, or consists essentially of, or yet further consists of an amino acid sequence of SEQ ID NO: 9 or an equivalent thereof.

Embodiment 122. The composition of embodiment 101, wherein the heavy chain (HC) comprises, or consists essentially of, or yet further consists of an amino acid sequence of SEQ ID NO: 3 or an equivalent thereof, and/or wherein the light chain (LC) comprises, or consists essentially of, or yet further consists of an amino acid sequence of SEQ ID NO: 7 or an equivalent thereof.

Embodiment 123. The composition of embodiment 101, wherein the heavy chain (HC) comprises, or consists essentially of, or yet further consists of an amino acid sequence of SEQ ID NO: 3 or an equivalent thereof, and/or wherein the light chain (LC) comprises, or consists essentially of, or yet further consists of an amino acid sequence of SEQ ID NO: 8 or an equivalent thereof.

Embodiment 124. The composition of embodiment 101, wherein the heavy chain (HC) comprises, or consists essentially of, or yet further consists of an amino acid sequence of SEQ ID NO: 3 or an equivalent thereof, and/or wherein the light chain (LC) comprises, or consists essentially of, or yet further consists of an amino acid sequence of SEQ ID NO: 9 or an equivalent thereof.

Embodiment 125. The composition of embodiment 101, wherein the heavy chain (HC) comprises, or consists essentially of, or yet further consists of an amino acid sequence of SEQ ID NO: 4 or an equivalent thereof, and/or wherein the light chain (LC) comprises, or consists essentially of, or yet further consists of an amino acid sequence of SEQ ID NO: 10 or an equivalent thereof.

Embodiment 126. The composition of embodiment 101, wherein the heavy chain (HC) comprises, or consists essentially of, or yet further consists of an amino acid sequence of SEQ ID NO: 4 or an equivalent thereof, and/or wherein the light chain (LC) comprises, or consists essentially of, or yet further consists of an amino acid sequence of SEQ ID NO: 11 or an equivalent thereof.

Embodiment 127. The composition of embodiment 101, wherein the heavy chain (HC) comprises, or consists essentially of, or yet further consists of an amino acid sequence of SEQ ID NO: 4 or an equivalent thereof, and/or wherein the light chain (LC) comprises, or consists essentially of, or yet further consists of an amino acid sequence of SEQ ID NO: 12 or an equivalent thereof.

Embodiment 128. The composition of embodiment 101, wherein the heavy chain (HC) comprises, or consists essentially of, or yet further consists of an amino acid sequence of SEQ ID NO: 5 or an equivalent thereof, and/or wherein the light chain (LC) comprises, or consists essentially of, or yet further consists of an amino acid sequence of SEQ ID NO: 10 or an equivalent thereof.

Embodiment 129. The composition of embodiment 101, wherein the heavy chain (HC) comprises, or consists essentially of, or yet further consists of an amino acid sequence of SEQ ID NO: 5 or an equivalent thereof, and/or wherein the light chain (LC) comprises, or consists essentially of, or yet further consists of an amino acid sequence of SEQ ID NO: 11 or an equivalent thereof.

Embodiment 130. The composition of embodiment 101, wherein the heavy chain (HC) comprises, or consists essentially of, or yet further consists of an amino acid sequence of SEQ ID NO: 5 or an equivalent thereof, and/or wherein the light chain (LC) comprises, or consists essentially of, or yet further consists of an amino acid sequence of SEQ ID NO: 12 or an equivalent thereof.

Embodiment 131. The composition of embodiment 101, wherein the heavy chain (HC) comprises, or consists essentially of, or yet further consists of an amino acid sequence of SEQ ID NO: 6 or an equivalent thereof, and/or wherein the light chain (LC) comprises, or consists essentially of, or yet further consists of an amino acid sequence of SEQ ID NO: 10 or an equivalent thereof.

Embodiment 132. The composition of embodiment 101, wherein the heavy chain (HC) comprises, or consists essentially of, or yet further consists of an amino acid sequence of SEQ ID NO: 6 or an equivalent thereof, and/or wherein the light chain (LC) comprises, or consists essentially of, or yet further consists of an amino acid sequence of SEQ ID NO: 11 or an equivalent thereof.

Embodiment 133. The composition of embodiment 101, wherein the heavy chain (HC) comprises, or consists essentially of, or yet further consists of an amino acid sequence of SEQ ID NO: 6 or an equivalent thereof, and/or wherein the light chain (LC) comprises, or consists essentially of, or yet further consists of an amino acid sequence of SEQ ID NO: 12 or an equivalent thereof.

Embodiment 134. The composition of any one of embodiments 101-133, wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 135. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an anti-DNABII antibody or a fragment thereof comprising, or alternatively consisting essentially of, or yet further consisting of one or two or three or four or five or all six of the following (i) to (vi):
   (iii) a heavy chain complementarity-determining region 1 (CDRH1) comprising, or alternatively consisting essentially of, or yet further consisting of a sequence selected from the group of: GFTFXXY (amino acid (aa) 50 to aa 56 of SEQ ID NO: 13), GFTFRTY (aa 50 to aa 56 of SEQ ID NO: 1 or 2 or 3 or 24), or GFTFSRY (aa 50 to aa 56 of SEQ ID NO: 4 or 5 or 6 or 26), wherein X is any amino acid or an amino acid at the aligned aa position of a sequence selected from SEQ ID NOs: 1-6;
   (iv) a heavy chain complementarity-determining region 2 (CDRH2) comprising, or alternatively consisting essentially of, or yet further consisting of a sequence selected from the group of: XSXXXX (amino acid (aa) 76 to aa 81 of SEQ ID NO: 13), GSDRRH (aa 76 to aa 81 of SEQ ID NO: 1 or 2 or 3 or 24), or SSGGSY (aa 76 to aa 81 of SEQ ID NO: 4 or 5 or 6 or 26), wherein X is any amino acid or an amino acid at the aligned aa position of a sequence selected from SEQ ID NOs: 1-6;
   (v) a heavy chain complementarity-determining region 3 (CDRH3) comprising, or alternatively consisting essentially of, or yet further consisting of a sequence selected from the group of: XXXXXXXYXXFDX (amino acid (aa) 121 to aa 133 of SEQ ID NO: 13), VGPYDGYYGEFDY (aa 121 to aa 133 of SEQ ID NO: 1 or 2 or 3 or 24), or ERHGGDGYWYFDV (aa 121 to aa 133 of SEQ ID NO: 4 or 5 or 6 or 26), wherein X is any amino acid or an amino acid at the aligned aa position of a sequence selected from SEQ ID NOs: 1-6;
   (vi) a light chain complementarity-determining region 1 (CDRL1) comprising, or alternatively consisting essentially of, or yet further consisting of a sequence selected from the group of: QXXXXXXXXXX (aa 47 to aa 57 of SEQ ID NO: 14), QXXXXX (aa 47 to aa 52 of SEQ ID NO: 14), QSLLDSDGKTF (aa 47 to aa 57 of SEQ ID NO: 7 or 8 or 9 or 25), or QDISNY (aa 47 to aa 52 of SEQ ID NO: 10 or 11 or 12 or 27), wherein X is any amino acid or an amino acid at the aligned aa position of a sequence selected from SEQ ID NOs: 7-12;
   (vii) a light chain complementarity-determining region 2 (CDRL2) comprising, or alternatively consisting essentially of, or yet further consisting of a sequence selected from the group of: XXS (aa 75 to aa 77 of SEQ ID NO: 14), LVS (aa 75 to aa 77 of SEQ ID NO: 7 or 8 or 9 or 25), or YTS (aa 70 to aa 72 of SEQ ID NO: 10 or 11 or 12 or 27), wherein X is any amino acid or an amino acid at the aligned aa position of a sequence selected from SEQ ID NOs: 7-12; and
   (viii) a light chain complementarity-determining region 3 (CDRL3) comprising, or alternatively consisting essentially of, or yet further consisting of a sequence selected from the group of: XQGXXXXXT (aa 114 to aa 122 of SEQ ID NO: 14), WQGTHFPYT (aa 114 to aa 122 of SEQ ID NO: 7 or 8 or 9 or 25), or QQGNPLRT (aa 109 to aa 116 of SEQ ID NO: 10 or 11 or 12 or 27), wherein X is any amino acid or an amino acid at the aligned aa position of a sequence selected from SEQ ID NOs: 7-12,
   optionally wherein the antibody or fragment thereof comprises, or alternatively consists essentially of, or yet further consists of a light chain and a heavy chain, further optionally wherein the light chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof, and further optionally wherein the heavy chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof.

Embodiment 136. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an anti-DNABII antibody or a fragment thereof comprising, or alternatively consisting essentially of, or yet further consisting of one or two or three or four or five or all six of the following (i) to (vi):
   (i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
   (ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
   (iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
   (iv) a light chain complementarity-determining region 1 (CDRL1) comprising, or alternatively consisting essentially of, or yet further consisting of a sequence of QSLLDSDGKTF (aa 47 to aa 57 of SEQ ID NO: 7 or 8 or 9 or 25);
   (v) a light chain complementarity-determining region 2 (CDRL2) comprising, or alternatively consisting essentially of, or yet further consisting of a sequence of LVS (aa 75 to aa 77 of SEQ ID NO: 7 or 8 or 9 or 25); and
   (vi) a light chain complementarity-determining region 3 (CDRL3) comprising, or alternatively consisting essentially of, or yet further consisting of a sequence of WQGTHFP (aa 114 to aa 120 of SEQ ID NO: 7 or 8 or 9 or 25),
   optionally wherein the antibody or fragment thereof comprises, or alternatively consists essentially of, or yet further consists of a light chain and a heavy chain, further optionally wherein the light chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof, and further optionally wherein the heavy chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof.

Embodiment 137. The composition of any one of embodiments 135-136, wherein the CDRH1 comprises, or consists essentially of, or yet further consists of a sequence of GFTFRTYA (aa 50 to aa 57 of SEQ ID NO: 1 or 2 or 3 or 24).

Embodiment 138. The composition of any one of embodiments 135-137, wherein the CDRH1 comprises, or consists essentially of, or yet further consists of a sequence of aASGFTFRTYAMS (aa 47 to aa 59 of SEQ ID NO: 24), wherein the small letter a is A (aa 47 to aa 59 of SEQ ID NO: 1 or 2) or wherein the small letter a is K (aa 47 to aa 59 of SEQ ID NO: 3).

Embodiment 139. The composition of any one of embodiments 135-138, wherein the CDRH2 comprises, or consists essentially of, or yet further consists of a sequence of IGSDRRHT (aa 75 to aa 82 of SEQ ID NO: 1 or 2 or 3 or 24).

Embodiment 140. The composition of any one of embodiments 135-139, wherein the CDRH2 comprises, or consists essentially of, or yet further consists of a sequence of IGSDRRHTY (aa 75 to aa 83 of SEQ ID NO: 1 or 2 or 3 or 24).

Embodiment 141. The composition of any one of embodiments 135-140, wherein the CDRH2 comprises, or consists essentially of, or yet further consists of a sequence of TIGSDRRHTY (aa 74 to aa 83 of SEQ ID NO: 1 or 2 or 3 or 24).

Embodiment 142. The composition of any one of embodiments 135-141, wherein the CDRH2 comprises, or consists essentially of, or yet further consists of a sequence of WVATIGSDRRHTYYP (aa 71 to aa 85 of SEQ ID NO: 1 or 2 or 3 or 24).

Embodiment 143. The composition of any one of embodiments 135-142, wherein the CDRL1 comprises, or consists essentially of, or yet further consists of a sequence of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9).

Embodiment 144. The composition of any one of embodiments 135-143, wherein the CDRL2 comprises, or consists essentially of, or yet further consists of a sequence of LVSKlDS (aa 75 to aa 81 of SEQ ID NO: 25), wherein the smaller letter l is L (aa 75 to aa 81 of SEQ ID NO: 7 or 9) or wherein the smaller letter l is R (aa 75 to aa 81 of SEQ ID NO: 8).

Embodiment 145. The composition of any one of embodiments 135-144, wherein the CDRL2 comprises, or consists essentially of, or yet further consists of a sequence of YLVSKlDS (aa 74 to aa 81 of SEQ ID NO: 25), wherein the smaller letter l is L (aa 74 to aa 81 of SEQ ID NO: 7 or 9) or wherein the smaller letter l is R (aa 74 to aa 81 of SEQ ID NO: 8).

Embodiment 146. The composition of any one of embodiments 135-144, wherein the CDRL2 comprises, or consists essentially of, or yet further consists of a sequence of LVSKlDSG (aa 75 to aa 82 of SEQ ID NO: 25), wherein the smaller letter l is L (aa 75 to aa 82 of SEQ ID NO: 7 or 9) or wherein the smaller letter l is R (aa 75 to aa 82 of SEQ ID NO: 8).

Embodiment 147. The composition of any one of embodiments 135-146, wherein the CDRL2 comprises, or consists essentially of, or yet further consists of a sequence of YLVSKlDSGV (aa 74 to aa 83 of SEQ ID NO: 25), wherein the smaller letter l is L (aa 74 to aa 83 of SEQ ID NO: 7 or 9) or wherein the smaller letter l is R (aa 74 to aa 83 of SEQ ID NO: 8).

Embodiment 148. The composition of any one of embodiments 135-147, wherein the CDRL2 comprises, or consists essentially of, or yet further consists of a sequence of RLIYLVSKlDSGVPD (aa 71 to aa 85 of SEQ ID NO: 25), wherein the smaller letter l is L (aa 71 to aa 85 of SEQ ID NO: 7 or 9) or wherein the smaller letter l is R (aa 71 to aa 85 of SEQ ID NO: 8).

Embodiment 149. The composition of any one of embodiments 135-148, wherein the CDRL3 comprises, or consists essentially of, or yet further consists of a sequence of WQGTHFPY (aa 114 to aa 121 of SEQ ID NO: 7 or 8 or 9 or 25).

Embodiment 150. The composition of any one of embodiments 135-149, wherein the CDRL3 comprises, or consists essentially of, or yet further consists of a sequence of WQGTHFPYT (aa 114 to aa 122 of SEQ ID NO: 7 or 8 or 9 or 25).

Embodiment 151. The composition of embodiment 135 or 136, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of a sequence of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 152. The composition of any one of embodiments 135, 136, and 151, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTYA (aa 50 to aa 57 of SEQ ID NO: 1 or 2 or 3 or 24);
(ii) a heavy chain complementarity-determining region 2 (CDRH2) comprising, or alternatively consisting essentially of, or yet further consisting of a sequence of IGSDRRHT (aa 75 to aa 82 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of a sequence of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 153. The composition of any one of embodiments 135, 136, and 151, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of aASGFTFRTYAMS (aa 47 to aa 59 of SEQ ID NO: 24), wherein the small letter a is A (aa 47 to aa 59 of SEQ ID NO: 1 or 2) or wherein the small letter a is K (aa 47 to aa 59 of SEQ ID NO: 3);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9);
(v) a light chain complementarity-determining region 2 (CDRL2) comprising, or alternatively consisting essentially of, or yet further consisting of a sequence of YLVSKlDS (aa 74 to aa 81 of SEQ ID NO: 25), wherein the smaller letter l is L (aa 74 to aa 81 of SEQ ID NO: 7 or 9) or wherein the smaller letter l is R (aa 74 to aa 81 of SEQ ID NO: 8); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 154. The composition of any one of embodiments 135, 136, 151 and 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of a sequence of YLVSKLDS (aa 74 to aa 81 of SEQ ID NO: 7); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 155. The composition of any one of embodiments 135, 136, 151, and 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of a sequence of YLVSKRDS (aa 74 to aa 81 of SEQ ID NO: 8); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 156. The composition of any one of embodiments 135, 136, 151, and 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of AASGFTFRTYAMS (aa 47 to aa 59 of SEQ ID NO: 1 or 2);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of a sequence of YLVSKLDS (aa 74 to aa 81 of SEQ ID NO: 9); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 157. The composition of any one of embodiments 135, 136, 151, and 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of: RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of a sequence of YLVSKLDS (aa 74 to aa 81 of SEQ ID NO: 7); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 158. The composition of any one of embodiments 135, 136, 151, and 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of a sequence of YLVSKRDS (aa 74 to aa 81 of SEQ ID NO: 8); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 159. The composition of any one of embodiments 135, 136, 151, an 153, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of KASGFTFRTYAMS (aa 47 to aa 59 of SEQ ID NO: 3);
(ii) a heavy chain complementarity-determining region 2 (CDRH2) comprising, or alternatively consisting essentially of, or yet further consisting of a sequence of TIGSDRRHTY (aa 74 to aa 83 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of a sequence of YLVSKLDS (aa 74 to aa 81 of SEQ ID NO: 9); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 160. The composition of any one of embodiments 135, 136, or 151, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9);
(v) a light chain complementarity-determining region 2 (CDRL2) comprising, or alternatively consisting essentially of, or yet further consisting of a sequence of LVSKlDS (aa 75 to aa 81 of SEQ ID NO: 25), wherein the smaller letter l is L (aa 75 to aa 81 of SEQ ID NO: 7 or 9) or wherein the smaller letter l is R (aa 75 to aa 81 of SEQ ID NO: 8); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 161. The composition of any one of embodiments 135, 136, 151, or 160, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of RSSQSLLDSDGKTFLN (aa 44 to aa 59 of SEQ ID NO: 7);
(v) a light chain complementarity-determining region 2 (CDRL2) comprising, or alternatively consisting essentially of, or yet further consisting of a sequence of LVSKLDS (aa 75 to aa 81 of SEQ ID NO: 7); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 162. The composition of any one of embodiments 135, 136, 151, or 160, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of RSSQSLLDSDGKTFLN (aa 44 to aa 59 of SEQ ID NO: 8);
(v) a light chain complementarity-determining region 2 (CDRL2) comprising, or alternatively consisting essentially of, or yet further consisting of a sequence of LVSKRDS (aa 75 to aa 81 of SEQ ID NO: 8); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 163. The composition of any one of embodiments 135, 136, 151, or 160, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTY (aa 50 to aa 56 of SEQ ID NO: 1 or 2 or 3 or 24);
(ii) a heavy chain complementarity-determining region 2 (CDRH2) comprising, or alternatively consisting essentially of, or yet further consisting of a sequence of GSDRRH (aa 76 to aa 81 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of KSSQSLLDSDGKTFLN (aa 44 to aa 59 of SEQ ID NO: 9);
(v) a light chain complementarity-determining region 2 (CDRL2) comprising, or alternatively consisting essentially of, or yet further consisting of a sequence of LVSKLDS (aa 75 to aa 81 of SEQ ID NO: 9); and
(vi) a light chain complementarity-determining region 3 (CDRL3) comprising, or alternatively consisting essentially of, or yet further consisting of a sequence of WQGTHFPYT (aa 114 to aa 122 of SEQ ID NO: 7 or 8 or 9 or 25).

Embodiment 164. The composition of embodiment 135 or 136, wherein the antibody comprises, or consists essentially of, or yet further consists of one or two or three or four or five or all six of the following (i) to (vi):
(i) a heavy chain complementarity-determining region 1 (CDRH1) comprising, or alternatively consisting essentially of, or yet further consisting of a sequence of GFTFRTYA (aa 50 to aa 57 of SEQ ID NO: 1 or 2 or 3 or 24);
(ii) a heavy chain complementarity-determining region 2 (CDRH2) comprising, or alternatively consisting essentially of, or yet further consisting of a sequence of IGSDRRHT (aa 75 to aa 82 of SEQ ID NO: 1 or 2 or 3 or 24);
(iii) a heavy chain complementarity-determining region 3 (CDRH3) comprising, or alternatively consisting essentially of, or yet further consisting of a sequence of VGPYDGYYGEFDY (aa 121 to aa 133 of SEQ ID NO: 1 or 2 or 3 or 24);
(iv) a light chain complementarity-determining region 1 (CDRL1) comprising, or alternatively consisting essentially of, or yet further consisting of a sequence of QSLLDSDGKTF (aa 47 to aa 57 of SEQ ID NO: 7 or 8 or 9 or 25);
(v) a light chain complementarity-determining region 2 (CDRL2) comprising, or alternatively consisting essentially of, or yet further consisting of a sequence of LVS (aa 75 to aa 77 of SEQ ID NO: 7 or 8 or 9 or 25); and
(vi) a light chain complementarity-determining region 3 (CDRL3) comprising, or alternatively consisting essentially of, or yet further consisting of a sequence of WQGTHFP (aa 114 to aa 120 of SEQ ID NO: 7 or 8 or 9 or 25).

Embodiment 165. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an anti-DNABII antibody or a fragment thereof comprising, or alternatively consisting essentially of, or yet further consisting of one or two or three or four or five or all six of the following (i) to (vi):
   (i) a heavy chain complementarity-determining region 1 (CDRH1) comprising, or alternatively consisting essentially of, or yet further consisting of a sequence of GFTFSRY (aa 50 to aa 56 of SEQ ID NO: 4 or 5 or 6 or 26);
   (ii) a heavy chain complementarity-determining region 2 (CDRH2) comprising, or alternatively consisting essentially of, or yet further consisting of a sequence of SSGGSY (aa 76 to aa 81 of SEQ ID NO: 4 or 5 or 6 or 26);
   (iii) a heavy chain complementarity-determining region 3 (CDRH3) comprising, or alternatively consisting essentially of, or yet further consisting of a sequence of ER (aa 121 to aa 122 of SEQ ID NO: 4 or 5 or 6 or 26);
   (iv) a light chain complementarity-determining region 1 (CDRL1) comprising, or alternatively consisting essentially of, or yet further consisting of a sequence of QDISNY (aa 47 to aa 52 of SEQ ID NO: 10 or 11 or 12 or 27);
   (v) a light chain complementarity-determining region 2 (CDRL2) comprising, or alternatively consisting essentially of, or yet further consisting of a sequence of YTS (aa 70 to aa 72 of SEQ ID NO: 10 or 11 or 12 or 27); and
   (vi) a light chain complementarity-determining region 3 (CDRL3) comprising, or alternatively consisting essentially of, or yet further consisting of a sequence of QQ (aa 109 to aa 110 of SEQ ID NO: 10 or 11 or 12 or 27),
   optionally wherein the antibody or fragment thereof comprises, or alternatively consists essentially of, or yet further consists of a light chain and a heavy chain, further optionally wherein the light chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof, and further optionally wherein the heavy chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof.

Embodiment 166. The composition of embodiment 135 or 165, wherein the CDRH1 comprises, or consists essentially of, or yet further consists of a sequence of GFTFSRYG (aa 50 to aa 57 of SEQ ID NO: 4 or 5 or 6 or 26).

Embodiment 167. The composition of any one of embodiments 135 or 165-166, wherein the CDRH1 comprises, or consists essentially of, or yet further consists of a sequence of aASGFTFSRYGMS (aa 47 to aa 59 of SEQ ID NO:26), wherein the small letter a is A (aa 47 to aa 59 of SEQ ID NO: 4 or 5) or wherein the small letter a is T (aa 47 to aa 59 of SEQ ID NO: 6).

Embodiment 168. The composition of any one of embodiments 135 or 165-167, wherein the CDRH2 comprises, or consists essentially of, or yet further consists of a sequence of ISSGGSYT (aa 75 to aa 82 of SEQ ID NO: 4 or 5 or 6 or 26).

Embodiment 169. The composition of any one of embodiments 135 or 165-168, wherein the CDRH2 comprises, or consists essentially of, or yet further consists of a sequence of TISSGGSYTY (aa 74 to aa 83 of SEQ ID NO: 4 or 5 or 6 or 26).

Embodiment 170. The composition of any one of embodiments 135 or 165-169, wherein the CDRH3 comprises, or consists essentially of, or yet further consists of a sequence of ERHGGDGYWYFDV (aa 121 to aa 133 of SEQ ID NO: 4 or 5 or 6 or 26).

Embodiment 171. The composition of any one of embodiments 135 or 165-170, wherein the CDRL1 comprises, or consists essentially of, or yet further consists of a sequence of RASQDISNYLN (aa 44 to aa 54 of SEQ ID NO: 10 or 11 or 12 or 27).

Embodiment 172. The composition of any one of embodiments 135 or 165-171, wherein the CDRL2 comprises, or consists essentially of, or yet further consists of a sequence of YTSRLHS (aa 70 to aa 76 of SEQ ID NO: 10 or 11 or 12 or 27).

Embodiment 173. The composition of any one of embodiments 135 or 165-172, wherein the CDRL2 comprises, or consists essentially of, or yet further consists of a sequence of YYTSRLHS (aa 69 to aa 76 of SEQ ID NO: 10 or 11 or 12 or 27).

Embodiment 174. The composition of any one of embodiments 135 or 165-173, wherein the CDRL3 comprises, or consists essentially of, or yet further consists of a sequence of QQGNPLRT (aa 109 to aa 116 of SEQ ID NO: 10 or 11 or 12 or 27).

Embodiment 175. The composition of embodiment 135 or 165, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of GFTFSRY (aa 50 to aa 56 of SEQ ID NO: 4 or 5 or 6 or 26);
(ii) a heavy chain complementarity-determining region 2 (CDRH2) comprising, or alternatively consisting essentially of, or yet further consisting of a sequence of SSGGSY (aa 76 to aa 81 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of a sequence of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 176. The composition of any one of embodiments 135, 165, or 175, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of GFTFSRYG (aa 50 to aa 57 of SEQ ID NO: 4 or 5 or 6 or 26);
(ii) a heavy chain complementarity-determining region 2 (CDRH2) comprising, or alternatively consisting essentially of, or yet further consisting of a sequence of ISSGGSYT (aa 75 to aa 82 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of a sequence of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 177. The composition of any one of embodiments 135, 165, or 175, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of aASGFTFSRYGMS (aa 47 to aa 59 of SEQ ID NO:26), wherein the small letter a is A (aa 47 to aa 59 of SEQ ID NO: 4 or 5) or wherein the small letter a is T (aa 47 to aa 59 of SEQ ID NO: 6);
(ii) a heavy chain complementarity-determining region 2 (CDRH2) comprising, or alternatively consisting essentially of, or yet further consisting of a sequence of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of a sequence of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 178. The composition of any one of embodiments 135, 165, 175, or 177, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of AASGFTFSRYGMS (aa 47 to aa 59 of SEQ ID NO: 4 or 5);
(ii) a heavy chain complementarity-determining region 2 (CDRH2) comprising, or alternatively consisting essentially of, or yet further consisting of a sequence of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of a sequence of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 179. The composition of any one of embodiments 135, 165, 175, or 177 comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of TASGFTFSRYGMS (aa 47 to aa 59 of SEQ ID NO: 6);
(ii) a heavy chain complementarity-determining region 2 (CDRH2) comprising, or alternatively consisting essentially of, or yet further consisting of a sequence of TISSGGSYTY (aa 74 to aa 83 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of a sequence of YYTSRLHS (aa 69 to aa 76 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 180. The composition of any one of embodiments 135, 165, or 175, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of GFTFSRY (aa 50 to aa 56 of SEQ ID NO: 4 or 5 or 6 or 26);
(ii) a heavy chain complementarity-determining region 2 (CDRH2) comprising, or alternatively consisting essentially of, or yet further consisting of a sequence of SSGGSY (aa 76 to aa 81 of SEQ ID NO: 4 or 5 or 6 or 26);
(iii) a heavy chain complementarity-determining region 3 (CDRH3) comprising, or alternatively consisting essentially of, or yet further consisting of a sequence of ERHGGDGYWYFDV (aa 121 to aa 133 of SEQ ID NO: 4 or 5 or 6 or 26);
(iv) a light chain complementarity-determining region 1 (CDRL1) comprising, or alternatively consisting essentially of, or yet further consisting of a sequence of RASQDISNYLN (aa 44 to aa 54 of SEQ ID NO: 10 or 11 or 12 or 27);
(v) a light chain complementarity-determining region 2 (CDRL2) comprising, or alternatively consisting essentially of, or yet further consisting of a sequence of YTSRLHS (aa 70 to aa 76 of SEQ ID NO: 10 or 11 or 12 or 27); and
(vi) a light chain complementarity-determining region 3 (CDRL3) comprising, or alternatively consisting essentially of, or yet further consisting of a sequence of QQGNPLRT (aa 109 to aa 116 of SEQ ID NO: 10 or 11 or 12 or 27).

Embodiment 181. The composition of any one of embodiments 135 or 165, comprising, or alternatively consisting essentially of, or yet further consisting of one or two or three or four or five or all six of the following (i) to (vi):
(i) a heavy chain complementarity-determining region 1 (CDRH1) comprising, or alternatively consisting essentially of, or yet further consisting of a sequence of GFTFSRYG (aa 50 to aa 57 of SEQ ID NO: 4 or 5 or 6 or 26);
(ii) a heavy chain complementarity-determining region 2 (CDRH2) comprising, or alternatively consisting essentially of, or yet further consisting of a sequence of ISSGGSYT (aa 75 to aa 82 of SEQ ID NO: 4 or 5 or 6 or 26);
(iii) a heavy chain complementarity-determining region 3 (CDRH3) comprising, or alternatively consisting essentially of, or yet further consisting of a sequence ER (aa 121 to aa 122 of SEQ ID NO: 4 or 5 or 6 or 26);
(iv) a light chain complementarity-determining region 1 (CDRL1) comprising, or alternatively consisting essentially of, or yet further consisting of a sequence of QDISNY (aa 47 to aa 52 of SEQ ID NO: 10 or 11 or 12 or 27);
(v) a light chain complementarity-determining region 2 (CDRL2) comprising, or alternatively consisting essentially of, or yet further consisting of a sequence of YTS (aa 70 to aa 72 of SEQ ID NO: 10 or 11 or 12 or 27); and
(vi) a light chain complementarity-determining region 3 (CDRL3) comprising, or alternatively consisting essentially of, or yet further consisting of a sequence of QQ (aa 109 to aa 110 of SEQ ID NO: 10 or 11 or 12 or 27).

Embodiment 182. The composition of any one of embodiments 135-181, wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S.

Embodiment 183. The composition of any one of embodiments 1-182, wherein the antibody comprises, or alternatively consists essentially of, or yet further consists of:
(i) CDRs 1-3 of a sequence selected from the group of: SEQ ID NOs: 1-6, 13, 24 or 26, or an equivalent of each thereof, and/or
(ii) CDRs 1-3 of a sequence selected from the group of: SEQ ID NOs: 7-12, 14, 25 or 27, or an equivalent of each thereof.

Embodiment 184. The composition of any one of embodiments 1-183, wherein the antibody comprises, or alternatively consists essentially of, or yet further consists of:
(i) CDRs 1-3 of a sequence selected from the group of: SEQ ID NOs: 1-6, 13, 24 or 26; and/or
(ii) CDRs 1-3 of a sequence selected from the group of: SEQ ID NOs: 7-12, 14, 25 or 27.

Embodiment 185. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
   (i) (b) an anti-DNABII antibody or a fragment thereof comprising, or alternatively consisting essentially of, or yet further consisting of: CDRs 1-3 of a sequence selected from the group of: SEQ ID NOs: 1-6, 13, 24 or 26, or an equivalent of each thereof, and/or
   (ii) CDRs 1-3 of a sequence selected from the group of: SEQ ID NOs: 7-12, 14, 25 or 27, or an equivalent of each thereof,
   optionally wherein the antibody or fragment thereof comprises, or alternatively consists essentially of, or yet further consists of a light chain and a heavy chain, further optionally wherein the light chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof, and further optionally wherein the heavy chain is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical to an amino acid sequence of any one or more of SEQ ID NOs: 7-12, 14, 25 or 27 or optionally a SEQ ID NO selected therefrom whose sequence comprises one or two or three CDR(s) of the antibody or a fragment thereof.

Embodiment 186. The composition of any one of embodiments 1-185, wherein the antibody is selected from the group of: a bispecific antibody, a trispecific antibody, a tetraspecific antibody, or a pentaspecific antibody.

Embodiment 187. The composition of any one of embodiments 1-186, wherein the antibody is selected from the group of an IgA, an IgD, an IgE, an IgG, or an IgM antibody.

Embodiment 188. The composition of any one of embodiments 1-187, wherein the antibody further comprises a constant region selected from the group of: an IgA constant region, an IgD constant region, an IgE constant region, an IgG constant region or an IgM constant region.

Embodiment 189. The composition of embodiment 188, wherein the constant region is an IgG1 constant region.

Embodiment 190. The composition of any one of embodiments 1-188, wherein the antibody further comprises a heavy chain (HC) constant region of SEQ ID NOs: 1-6, 13, 24 or 26, and/or a light chain (LC) constant region of SEQ ID NOs: 7-12, 14, 25 or 27.

Embodiment 191. The composition of embodiment 190, wherein the HC constant region comprises, or alternatively consists essentially of, or yet further consists of a constant region of SEQ ID NOs: 1-6, 13, 24 or 26 (optionally a sequence selected from aa 145 to aa 473 of SEQ ID NOs: 1-6, 13, 24 or 26), or wherein the HC constant regions comprises, or alternatively consists essentially of, or yet further consists of a constant region of any one of SEQ ID NOs: 15-22.

Embodiment 192. The composition of embodiment 190 or 191, wherein the LC constant region comprises, or alternatively consists essentially of, or yet further consists of a constant region of SEQ ID NOs: 7-12 or 27 (optionally a sequence selected from aa 133 to aa 239 of SEQ ID NOs: 7-9, 14 or 25, and/or aa 127 to aa 233 of SEQ ID NOs: 10-12 or 27), or wherein the LC constant regions comprises, or alternatively consists essentially of, or yet further consists of a constant region of SEQ ID NOs: 23.

Embodiment 193. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an anti-DNABII antibody or a fragment thereof that competes for binding to an epitope with an antibody or a fragment thereof as disclosed in any one of embodiments 1-192.

Embodiment 194. A composition of any one of embodiment 1-24, 34-57, 67-90, 100-124, 134-164 and 182-192, wherein the antibody or a fragment thereof competes for binding to a tip chimeric peptide IhfA5-mIhfB4_{NTHI}.

Embodiment 195. The composition of embodiment 194, wherein the tip chimeric peptide IhfA5-mIhfB4_{NTHI} comprises, or alternatively consists essentially of, or yet further consists of RPGRNPX1TGDVVPVSARRVV-X-FSLHHRQPRLGRNPX1TGDSV (SEQ ID NO: 38), wherein “X” is an optional amino acid linker sequence, optionally comprising, or consisting essentially of, or yet further consisting of between 1 to 20 amino acids, and wherein “X1” is any amino acid or alternatively “X1” is selected from the amino acids Q, R, K, S, or T.

Embodiment 196. The composition of embodiment 194 or 195, wherein the tip chimeric peptide IhfA5-mIhfB4_{NTHI} comprises, or alternatively consists essentially of, or yet further consists of RPGRNPKTGDVVPVSARRVV-X-FSLHHRQPRLGRNPKTGDSV (SEQ ID NO: 39), wherein “X” is an optional amino acid linker sequence, optionally comprising, or consisting essentially of, or yet further consisting of between 1 to 20 amino acids.

Embodiment 197. The composition of any one of embodiments 194-196, wherein the tip chimeric peptide IhfA5-mIhfB4_{NTHI} comprises, or alternatively consists essentially of, or yet further consists of RPGRNPKTGDVVPVSARRVVGPSLFSLHHRQPRLGRNPKTGDSV (SEQ ID NO: 40).

Embodiment 198. A composition of any one of embodiments 1, 3-15, 25-34, 36-47, 58-67, 69-81, 91-101, 103-115, 125-135, and 166-192, wherein the antibody or a fragment thereof competes for binding to a tail chimeric peptide IhfA3-IhfB2_{NTHI}.

Embodiment 199. The composition of embodiment 198, wherein the tail chimeric peptide IhfA3-IhfB2_{NTHI} comprises, or alternatively consists essentially of, or yet further consists of FLEEIRLSLESGQDVKLSGF-X-TLSAKEIENMVKDILEFISQ (SEQ ID NO: 41), wherein “X” is an optional amino acid linker sequence, optionally comprising, or consisting essentially of, or yet further consisting of between 1 to 20 amino acids, and/or wherein the tail-chimeric peptide IhfA3-IhfB2_{NTHI} comprises, or consists essentially of, or yet further consists of FLEEIRLSLESGQDVKLSGFGPSLTLSAKEIENMVKDILEFISQ (SEQ ID NO: 50).

Embodiment 200. The composition of any one of embodiments 195, 196, or 199, wherein the amino acid linker is selected from the group of: GGSGGS (SEQ ID NO: 42), GPSLKL (SEQ ID NO: 43), GGG (SEQ ID NO: 44), GPSL (SEQ ID NO: 45), GPS (SEQ ID NO: 46), PSLK (SEQ ID NO: 47), GPSLK (SEQ ID NO: 48), or SLKL (SEQ ID NO: 49).

Embodiment 201. The composition of any one of embodiments 193-200, wherein the antibody is a polyclonal, a monoclonal or a humanized antibody.

Embodiment 202. The composition of any one of embodiments 1-201, wherein the fragment is an antigen binding fragment.

Embodiment 203. The composition of embodiment 202, wherein the antigen binding fragment is selected from the group of Fab, F(ab′)2, Fab′, scFv, or Fv.

Embodiment 204. The composition of any one of embodiments 1-33, 67-134, or 183-203, wherein an equivalent to an amino acid sequence comprises a polypeptide having at least 80% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 205. The composition of any one of embodiments 1-33, 67-134, or 183-203, wherein an equivalent to an amino acid sequence comprises a polypeptide having at least 90% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 206. The composition of any one of embodiments 1-33, 67-134, or 183-203, wherein an equivalent to an amino acid sequence comprises a polypeptide having at least 95% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 207. The composition of any one of embodiments 1-33, 67-134, or 183-203, wherein an equivalent to an amino acid sequence comprises a polypeptide having at least 96% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 208. The composition of any one of embodiments 1-33, 67-134, or 183-203, wherein an equivalent to an amino acid sequence comprises a polypeptide having at least 97% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 209. The composition of any one of embodiments 1-33, 67-134, or 183-203, wherein an equivalent to an amino acid sequence comprises a polypeptide having at least 98% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 210. The composition of any one of embodiments 1-33, 67-134, or 183-203, wherein an equivalent to an amino acid sequence comprises a polypeptide having at least 99% amino acid identity to the amino acid sequence, and/or wherein an equivalent to the amino acid sequence comprises a polypeptide that is encoded by a polynucleotide that hybridizes under conditions of high stringency to the complement of the polynucleotide encoding the amino acid sequence.

Embodiment 211. The composition of any one of embodiments 1-210, wherein the antibody or fragment thereof is modified, and optionally wherein the modification is selected from the group of PEGylation, a PEG mimetic, polysialyation, HESylation or glycosylation.

Embodiment 212. The composition of any one of embodiments 1-211, wherein either or both of the HMGB polypeptide or a fragment thereof, or the anti-DNABII antibody or a fragment thereof further comprises a detectable marker or a purification marker.

Embodiment 213. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) a complementarity-determining region (CDR) of the compositions of any one of embodiments 1-212, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 214. The composition of embodiment 213, wherein the CDR is selected from the group consisting of CDR1, CDR2 or CDR3.

Embodiment 215. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) a complementarity-determining region (CDR) comprising, or alternatively consisting essentially of, or yet further consisting of any one or more of the following:
   (i) A complementarity-determining region (CDR) a heavy chain complementarity-determining region 1 (CDRH1) comprising, or alternatively consisting essentially of, or yet further consisting of a sequence selected from the group of: GFTFXXY (amino acid (aa) 50 to aa 56 of SEQ ID NO: 13), GFTFRTY (aa 50 to aa 56 of SEQ ID NO: 1 or 2 or 3 or 24), or GFTFSRY (aa 50 to aa 56 of SEQ ID NO: 4 or 5 or 6 or 26), wherein X is any amino acid or an amino acid at the aligned aa position of a sequence selected from SEQ ID NOs: 1-6; GFTFRTYA (aa 50 to aa 57 of SEQ ID NO: 1 or 2 or 3 or 24); aASGFTFRTYAMS (aa 47 to aa 59 of SEQ ID NO: 24), wherein the small letter a is A (aa 47 to aa 59 of SEQ ID NO: 1 or 2) or wherein the small letter a is K (aa 47 to aa 59 of SEQ ID NO: 3); GFTFSRYG (aa 50 to aa 57 of SEQ ID NO: 4 or 5 or 6 or 26); or aASGFTFSRYGMS (aa 47 to aa 59 of SEQ ID NO:26), wherein the small letter a is A (aa 47 to aa 59 of SEQ ID NO: 4 or 5) or wherein the small letter a is T (aa 47 to aa 59 of SEQ ID NO: 6);
   (ii) a heavy chain complementarity-determining region 2 (CDRH2) comprising, or alternatively consisting essentially of, or yet further consisting of a sequence selected from the group of: XSXXXX (amino acid (aa) 76 to aa 81 of SEQ ID NO: 13), GSDRRH (aa 76 to aa 81 of SEQ ID NO: 1 or 2 or 3 or 24), or SSGGSY (aa 76 to aa 81 of SEQ ID NO: 4 or 5 or 6 or 26), wherein X is any amino acid or an amino acid at the aligned aa position of a sequence selected from SEQ ID NOs: 1-6; IGSDRRHT (aa 75 to aa 82 of SEQ ID NO: 1 or 2 or 3 or 24); IGSDRRHTY (aa 75 to aa 83 of SEQ ID NO: 1 or 2 or 3 or 24); TIGSDRRHTY (aa 74 to aa 83 of SEQ ID NO: 1 or 2 or 3 or 24); WVATIGSDRRHTYYP (aa 71 to aa 85 of SEQ ID NO: 1 or 2 or 3 or 24); ISSGGSYT (aa 75 to aa 82 of SEQ ID NO: 4 or 5 or 6 or 26); or TISSGGSYTY (aa 74 to aa 83 of SEQ ID NO: 4 or 5 or 6 or 26);
   (iii) a heavy chain complementarity-determining region 3 (CDRH3) comprising, or alternatively consisting essentially of, or yet further consisting of a sequence selected from the group of: XXXXXXXYXXFDX (amino acid (aa) 121 to aa 133 of SEQ ID NO: 13), VGPYDGYYGEFDY (aa 121 to aa 133 of SEQ ID NO: 1 or 2 or 3 or 24), or ERHGGDGYWYFDV (aa 121 to aa 133 of SEQ ID NO: 4 or 5 or 6 or 26), wherein X is any amino acid or an amino acid at the aligned aa position of a sequence selected from SEQ ID NOs: 1-6; VGPYDGYYGEFDY (aa 121 to aa 133 of SEQ ID NO: 1 or 2 or 3 or 24); or ER (aa 121 to aa 122 of SEQ ID NO: 4 or 5 or 6 or 26);
   (iv) a light chain complementarity-determining region 1 (CDRL1) comprising, or alternatively consisting essentially of, or yet further consisting of a sequence selected from the group of: QXXXXXXXXXX (aa 47 to aa 57 of SEQ ID NO: 14), QXXXXX (aa 47 to aa 52 of SEQ ID NO: 14), QSLLDSDGKTF (aa 47 to aa 57 of SEQ ID NO: 7 or 8 or 9 or 25), or QDISNY (aa 47 to aa 52 of SEQ ID NO: 10 or 11 or 12 or 27), wherein X is any amino acid or an amino acid at the aligned aa position of a sequence selected from SEQ ID NOs: 7-12; rSSQSLLDSDGKTFLN (aa 44 to aa 59 of SEQ ID NO: 25), wherein the smaller letter r is R (aa 44 to aa 59 of SEQ ID NO: 7 or 8) or wherein the smaller letter r is K (aa 44 to aa 59 of SEQ ID NO: 9); or RASQDISNYLN (aa 44 to aa 54 of SEQ ID NO: 10 or 11 or 12 or 27);
   (v) a light chain complementarity-determining region 2 (CDRL2) comprising, or alternatively consisting essentially of, or yet further consisting of a sequence selected from the group of: XXS (aa 75 to aa 77 of SEQ ID NO: 14), LVS (aa 75 to aa 77 of SEQ ID NO: 7 or 8 or 9 or 25), or YTS (aa 70 to aa 72 of SEQ ID NO: 10 or 11 or 12 or 27), wherein X is any amino acid or an amino acid at the aligned aa position of a sequence selected from SEQ ID NOs: 7-12; LVSKlDS (aa 75 to aa 81 of SEQ ID NO: 25), wherein the smaller letter l is L (aa 75 to aa 81 of SEQ ID NO: 7 or 9) or wherein the smaller letter l is R (aa 75 to aa 81 of SEQ ID NO: 8); YLVSKlDS (aa 74 to aa 81 of SEQ ID NO: 25), wherein the smaller letter l is L (aa 74 to aa 81 of SEQ ID NO: 7 or 9) or wherein the smaller letter l is R (aa 74 to aa 81 of SEQ ID NO: 8); LVSKlDSG (aa 75 to aa 82 of SEQ ID NO: 25), wherein the smaller letter l is L (aa 75 to aa 82 of SEQ ID NO: 7 or 9) or wherein the smaller letter l is R (aa 75 to aa 82 of SEQ ID NO: 8); YLVSKlDSGV (aa 74 to aa 83 of SEQ ID NO: 25), wherein the smaller letter l is L (aa 74 to aa 83 of SEQ ID NO: 7 or 9) or wherein the smaller letter l is R (aa 74 to aa 83 of SEQ ID NO: 8); RLIYLVSKlDSGVPD (aa 71 to aa 85 of SEQ ID NO: 25), wherein the smaller letter l is L (aa 71 to aa 85 of SEQ ID NO: 7 or 9) or wherein the smaller letter l is R (aa 71 to aa 85 of SEQ ID NO: 8); YTSRLHS (aa 70 to aa 76 of SEQ ID NO: 10 or 11 or 12 or 27); or YYTSRLHS (aa 69 to aa 76 of SEQ ID NO: 10 or 11 or 12 or 27); and
   (vi) a light chain complementarity-determining region 3 (CDRL3) comprising, or alternatively consisting essentially of, or yet further consisting of a sequence selected from the group of: XQGXXXXXT (aa 114 to aa 122 of SEQ ID NO: 14), WQGTHFPYT (aa 114 to aa 122 of SEQ ID NO: 7 or 8 or 9 or 25), or QQGNPLRT (aa 109 to aa 116 of SEQ ID NO: 10 or 11 or 12 or 27), wherein X is any amino acid or an amino acid at the aligned aa position of a sequence selected from SEQ ID NOs: 7-12; WQGTHFP (aa 114 to aa 120 of SEQ ID NO: 7 or 8 or 9 or 25); WQGTHFPY (aa 114 to aa 121 of SEQ ID NO: 7 or 8 or 9 or 25); WQGTHFPYT (aa 114 to aa 122 of SEQ ID NO: 7 or 8 or 9 or 25); QQ (aa 109 to aa 110 of SEQ ID NO: 10 or 11 or 12 or 27), optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 216. A composition comprising
(a) a high mobility group box 1 protein (HMGB1) polypeptide optionally comprising one or more mutations (a mutant HMGB1 (mHMGB1) polypeptide), or a fragment thereof optionally comprising or consisting of B box or A box or the AB box of HMGB1 polypeptide, optionally wherein the polypeptide or a fragment thereof is isolated and/or engineered; and
(b) an isolated polypeptide comprising an amino acid sequence selected from the group of SEQ ID NOs: 1-14 or 24-27, or an equivalent of each thereof, Optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 217. The composition of embodiment 216, wherein the polypeptide further comprises a detectable or purification marker.

Embodiment 218. A composition comprising
an isolated polynucleotide encoding the HMGB1 polypeptide or a fragment thereof of (a) in the composition of any one of embodiments 1-217, or an equivalent thereof, and optionally operatively linked to a promoter and enhancer element; and
an isolated polynucleotide encoding the antibody or a fragment thereof of (b) in the composition of any one of embodiments 1-217, or an equivalent of each thereof, and optionally operatively linked to a promoter and enhancer element,
wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 219. The composition of embodiment 218, wherein either or both of the polynucleotides further comprises a signal peptide coding polynucleotide sequence located upstream of the variable domain, chain, the HMGB1 polypeptide or a fragment thereof, or CDR.

Embodiment 220. The composition of any one of embodiments 1-217, wherein either or both of the HMGB1 polypeptide or a fragment thereof of (a) or the anti-DNABII antibody or a fragment thereof of (b) further comprise a signal peptide located upstream of the variable domain, chains, CDRs, HMGB1 polypeptide or a fragment thereof.

Embodiment 221. A vector comprising one or both isolated polynucleotide(s) in the composition of embodiment 218 or 219.

Embodiment 222. The vector of embodiment 221, wherein the vector is a plasmid or a viral vector.

Embodiment 223. The vector of embodiment 222, wherein the viral vector is selected from a group consisting of a retroviral vector, a lentiviral vector, an adenoviral vector, and an adeno-associated viral vector.

Embodiment 224. A host cell comprising the composition of embodiment 218 or 219, or the vector of any one of embodiments 221-223.

Embodiment 225. A method for inhibiting or competing with the binding of a DNABII polypeptide or protein to a microbial DNA, comprising contacting the DNABII polypeptide or protein with an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, wherein the antibody or fragment thereof binds a tip region of a DNABII peptide, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 226. A method to disrupt a biofilm, comprising contacting the biofilm with an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, wherein the antibody or fragment thereof binds a tip region of a DNABII peptide, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 227. A method to prevent formation of or to disrupt a biofilm on a surface comprising treating the surface susceptible to or containing a biofilm with an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, wherein the antibody or fragment thereof binds a tip region of a DNABII peptide, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 228. A method to prevent or disrupt a biofilm in a subject, comprising administering to the subject an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 229. A method for inhibiting, preventing or treating a microbial infection that produces a biofilm in a subject, comprising administering to the subject an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, wherein the antibody or fragment thereof binds to a tip region of a DNABII peptide, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 230. A method to treat a condition characterized by the formation of a biofilm in a subject, comprising administering to the subject an HMGB1 polypeptide or a fragment thereof of (a) of a composition of any one of embodiments 1-24, 34-57, 67-90, 100-124, 134-164 and 182-201, 204-212 and 220 and an anti-DNABII antibody or a fragment thereof of (b) of the composition, wherein the antibody or fragment thereof binds to a tip region of a DNABII peptide, optionally wherein the one or more of the HMGB1 mutations are selected from the group of mutations at K12, C23, C45, C106, or K114, optionally to serine, glycine, alanine, valine, isoleucine or threonine, and optionally wherein the mHMGB1 comprises one or more mutations selected from C23S, C45S, and C106S, optionally wherein the HMGB1 is a human HMGB1 or a murine HMGB1.

Embodiment 231. The method of any one of embodiments 228 to 230, wherein the antibody or a fragment thereof reduce one or more of pro-inflammatory cytokines and increase one or more of anti-inflammatory cytokines in the subject.

Embodiment 232. The method of embodiment 230 or 231, wherein the condition is selected from the group consisting of: chronic non-healing wounds, Burkholderia, venous ulcers, diabetic foot ulcers, ear infections, sinus infections, urinary tract infections, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease.

Embodiment 233. The method of any one of embodiments 225 to 232, wherein contacting or administering with (a) of the composition and (b) of the composition are performed concurrently or sequentially.

Embodiment 234. The method of any one of embodiments 225 to 233, further comprising detecting a biofilm by contacting an antibody that binds a DNABII polypeptide or an antigen binding fragment of the antibody with a sample suspected of containing a biofilm, and detecting the binding of the biofilm and the antibody or fragment thereof.

Embodiment 235. A method for screening subjects for use of a composition or method as described herein, comprising contacting a composition of any one of embodiments 1-217 and 220 with a biological sample comprising the biofilm and isolated from the subject, and detecting the binding of the antibody or fragment thereof to any biofilm in the sample.

Embodiment 236. The composition of any one of embodiments 1-24, 34-57, 67-90, 100-124, 134-164 and 182-201, 204-212 and 220, wherein the fragment of the antibody is selected from the group of Fab, F(ab′)2, Fab′, scFv, or Fv, and wherein the antibody fragment specifically binds the tip region of a DNABII peptide.

Embodiment 237. The composition of embodiment 236 or the method of any one of embodiments 225-227 and 229-230, wherein the DNABII peptide is an IHF peptide.

1. A composition or combination comprising:
(a) a high mobility group box protein (HMGB) polypeptide, or a fragment thereof comprising a B box or an A box or an AB box thereof, and
(b) an anti-DNABII antibody or an antigen-binding fragment thereof comprising:
(i) a heavy chain complementarity-determining region 1 (CDRH1) comprising GFTFRTY (aa 50 to aa 56 of SEQ ID NO: 1 or 2 or 3 or 24);
(ii) a heavy chain complementarity-determining region 2 (CDRH2) comprising GSDRRH (aa 76 to aa 81 of SEQ ID NO: 1 or 2 or 3 or 24);
(iii) a heavy chain complementarity-determining region 3 (CDRH3) comprising VGPYDGYYGEFDY (aa 121 to aa 133 of SEQ ID NO: 1 or 2 or 3 or 24);
(iv) a light chain complementarity-determining region 1 (CDRL1) comprising QSLLDSDGKTF (aa 47 to aa 57 of SEQ ID NO: 7 or 8 or 9 or 25);
(v) a light chain complementarity-determining region 2 (CDRL2) comprising LVS (aa 75 to aa 77 of SEQ ID NO: 7 or 8 or 9 or 25); and
(vi) a light chain complementarity-determining region 3 (CDRL3) comprising WQGTHFPYT (aa 114 to aa 122 of SEQ ID NO: 7 or 8 or 9 or 25).

2. The composition or combination of claim 1, wherein the antibody or antigen-binding fragment thereof comprises a heavy chain (HC) immunoglobulin variable domain comprising amino acids 25 to 144 of any one of SEQ ID NO: 1, SEQ ID NO: 2, or SEQ ID NO: 3.

3. The composition or combination of claim 2, wherein the antibody or antigen-binding fragment thereof comprises a light chain (LC) immunoglobulin variable domain comprising amino acids 21 to 132 of any one of SEQ ID NO: 7, SEQ ID NO: 8, or SEQ ID NO: 9.

4. The composition or combination of claim 3, wherein the antibody or antigen-binding fragment thereof comprises a heavy chain (HC) immunoglobulin variable domain comprising amino acids 25 to 144 of any one of SEQ ID NO: 1, and wherein the LC immunoglobulin variable domain comprises amino acids 21 to 132 of SEQ ID NO: 7.

5. The composition or combination of claim 3, wherein the antibody or antigen-binding fragment thereof comprises a heavy chain (HC) immunoglobulin variable domain comprising amino acids 25 to 144 of any one of SEQ ID NO: 1, and wherein the LC immunoglobulin variable domain comprises amino acids 21 to 132 of SEQ ID NO: 8.

6. The composition or combination of claim 3, wherein the antibody or antigen-binding fragment thereof comprises a heavy chain (HC) immunoglobulin variable domain comprising amino acids 25 to 144 of any one of SEQ ID NO: 1, and wherein the LC immunoglobulin variable domain comprises amino acids 21 to 132 of SEQ ID NO: 9.

7. The composition or combination of claim 1, wherein the antibody comprises a constant region selected from an IgA constant region, an IgD constant region, an IgE constant region, an IgG constant region, or an IgM constant region.

8. The composition or combination of claim 1, wherein the antigen-binding fragment thereof comprises a Fab, F(ab′)2, Fab′, scFv, or Fv.

9. The composition or combination of claim 1, wherein the antibody or antigen-binding fragment thereof is modified.

10. The composition or combination of claim 9, wherein the antibody or antigen-binding fragment thereof is modified by a process selected from PEGylation, polysialyation, HESylation or glycosylation.

11. The composition or combination of claim 1, wherein the antibody or antigen-binding fragment thereof is a monoclonal antibody or an antigen-binding fragment of the monoclonal antibody.

12. (canceled)

13. The composition or combination of claim 1, wherein the HMGB polypeptide comprises the HMGB1 polypeptide that optionally further comprises one or more mutations selected from the group of mutations at K12, C23, C45, C106, or K114, or a HMGB polypeptide selected from the group of HMGB2, HMGB3, or HMGB4 polypeptide with corresponding mutations to the HMGB1 polypeptide that comprises one or more mutations selected from the group of mutations at K12, C23, C45, C106, or K114.

14. The composition or combination of claim 1, wherein the A box of the HMGB polypeptide, optionally the HMGB11 polypeptide that further comprises one or more mutations selected from the group of mutations at K12, C23, or C45 or corresponding mutations when the HMGB polypeptide selected from the group of HMGB2, HMGB3, or HMGB4 polypeptide with corresponding mutations to the mutated HMGB1 polypeptide.

15. The composition or combination of claim 1, wherein the B box of the HMGB, optionally the HMGB1 polypeptide further comprises one or both mutations at C106 or K114 or a corresponding mutation to the mutated HMGB1 polypeptide when the HMGB polypeptide selected from the group of HMGB2, HMGB3, or HMGB4 polypeptide.

16. The composition or combination of claim 13, wherein the one or more mutations are to serine, glycine, alanine, valine, isoleucine or threonine.

17. The composition or combination of claim 1, wherein the HMGB polypeptide, optionally the HMGB1 polypeptide further comprises one or more mutations selected from C23S, C45S, and C106S or the HMGB polypeptide selected from the group of HMGB2, HMGB3, or HMGB4 polypeptide with one or mutations corresponding to the mutated HMGB1 polypeptide.

18. The composition or combination of claim 1, wherein the HMGB polypeptide, optionally the HMGB1 polypeptide, further comprises the mutation of C45S or a HMGB polypeptide selected from the group of HMGB2, HMGB3, or HMGB4 polypeptide with corresponding mutations to the mutated HMGB1 polypeptide.

19. The composition or combination of claim 1, comprising:
(a) the HMGB polypeptide, optionally the HMGB1 polypeptide, further comprising the mutation of C45S; and
(b) the anti-DNABII antibody or antigen-binding fragment thereof comprising the heavy chain (HC) immunoglobulin variable domain that comprises amino acids 25 to 144 of SEQ ID NO: 1 and the light chain (LC) immunoglobulin variable domain that comprises amino acids 21 to 132 of SEQ ID NO: 7, SEQ ID NO: 8, or SEQ ID NO: 9.

20. A composition or combination comprising:
(a) a high mobility group box 1 protein (HMGB1) polypeptide, or a fragment thereof comprising a B box or an A box or an AB box thereof, and
(b) an antibody or an antigen-binding fragment thereof specifically recognizing and binding a tip domain of a DNABII protein,
with the proviso that (i) the composition or combination does not comprise SEQ ID NO: 52, or (ii) the antigen-binding fragment that does not comprise an Fab optionally an Fab of polyclonal antibodies or the antibody that does not comprise polyclonal antibodies, or both (i) and (ii).

21. (canceled)

22. A polypeptide comprising:
(a) a high mobility group box protein (HMGB) polypeptide, optionally the HMGB1 polypeptide or a fragment thereof comprising a B box or an A box or an AB box thereof; and
(b) an anti-DNABII antibody or an antigen-binding fragment thereof comprising:
(i) a heavy chain complementarity-determining region 1 (CDRH1) comprising GFTFRTY (aa 50 to aa 56 of SEQ ID NO: 1 or 2 or 3 or 24);
(ii) a heavy chain complementarity-determining region 2 (CDRH2) comprising GSDRRH (aa 76 to aa 81 of SEQ ID NO: 1 or 2 or 3 or 24);
(iii) a heavy chain complementarity-determining region 3 (CDRH3) comprising VGPYDGYYGEFDY (aa 121 to aa 133 of SEQ ID NO: 1 or 2 or 3 or 24);
(iv) a light chain complementarity-determining region 1 (CDRL1) comprising QSLLDSDGKTF (aa 47 to aa 57 of SEQ ID NO: 7 or 8 or 9 or 25);
(v) a light chain complementarity-determining region 2 (CDRL2) comprising LVS (aa 75 to aa 77 of SEQ ID NO: 7 or 8 or 9 or 25); and
(vi) a light chain complementarity-determining region 3 (CDRL3) comprising WQGTHFPYT (aa 114 to aa 122 of SEQ ID NO: 7 or 8 or 9 or 25).

23. A polypeptide comprising:
(a) a high mobility group box protein (HMGB), optionally the HMGB1 polypeptide, or a fragment thereof comprising a B box or an A box or an AB box thereof, and
(b) an antibody or an antigen-binding fragment thereof specifically recognizing and binding a tip domain of a DNABII protein,
with the proviso that (i) the polypeptide does not comprise SEQ ID NO: 52, or (ii) the antigen-binding fragment that does not comprise an Fab optionally an Fab of polyclonal antibodies or the antibody that does not comprise polyclonal antibodies, or both (i) and (ii).

24. The polypeptide of claim 23, wherein the tip domain comprises one or more amino acid sequence(s) selected from: NFELRDKSSRPGRNPKTGDVV (SEQ ID NO: 31); SLHHRQPRLGRNPKTGDSVNL (SEQ ID NO: 32); RPGRNPX₁TGDVVPVSARRVV-X-FSLHHRQPRLGRNPX₁TGDSV, wherein “X” is an optional amino acid linker sequence and wherein “X₁” is any amino acid (SEQ ID NO: 38); RPGRNPKTGDVVPVSARRVV-X-FSLHHRQPRLGRNPKTGDSV wherein “X₁” is any amino acid (SEQ ID NO: 39); or RPGRNPKTGDVVPVSARRVVGPSLFSLHHRQPRLGRNPKTGDSV (SEQ ID NO: 40).

25. (canceled)

26. A polynucleotide-encoding:
(a) a high mobility group box protein (HMGB) polypeptide, optionally the HMGB1 polypeptide, or a fragment thereof comprising a B box or an A box or an AB box thereof; and
(b) an anti-DNABII antibody or an antigen-binding fragment thereof comprising:
(i) a heavy chain complementarity-determining region 1 (CDRH1) comprising GFTFRTY (aa 50 to aa 56 of SEQ ID NO: 1 or 2 or 3 or 24);
(ii) a heavy chain complementarity-determining region 2 (CDRH2) comprising GSDRRH (aa 76 to aa 81 of SEQ ID NO: 1 or 2 or 3 or 24);
(iii) a heavy chain complementarity-determining region 3 (CDRH3) comprising VGPYDGYYGEFDY (aa 121 to aa 133 of SEQ ID NO: 1 or 2 or 3 or 24);
(iv) a light chain complementarity-determining region 1 (CDRL1) comprising QSLLDSDGKTF (aa 47 to aa 57 of SEQ ID NO: 7 or 8 or 9 or 25);
(v) a light chain complementarity-determining region 2 (CDRL2) comprising LVS (aa 75 to aa 77 of SEQ ID NO: 7 or 8 or 9 or 25); and
(vi) a light chain complementarity-determining region 3 (CDRL3) comprising WQGTHFPYT (aa 114 to aa 122 of SEQ ID NO: 7 or 8 or 9 or 25),
or a polynucleotide complementary thereto.

27. A polynucleotide encoding:
(a) a high mobility group box protein (HMGB), optionally the HMGB1 polypeptide, or a fragment thereof comprising a B box or an A box or an AB box thereof, and
(b) an antibody or an antigen-binding fragment thereof specifically recognizing and binding a tip domain of a DNABII protein,
or a polynucleotide complementary thereto,
with the proviso that (i) the polynucleotide does not encode SEQ ID NO: 52, or (ii) the antigen-binding fragment that does not comprise an Fab optionally an Fab of polyclonal antibodies or the antibody that does not comprise polyclonal antibodies, or both (i) and (ii).

28. (canceled)

29. A vector comprising the polynucleotide of claim 26.

30. A host cell comprising the composition or combination of claim 1.

31. (canceled)

32. A method for one or more of the following:
(A) preventing the formation of or disrupting a biofilm in vitro or ex vivo,
(B) preventing the formation of or disrupting a biofilm in a subject,
(C) inhibiting, preventing or treating a microbial infection that produces a biofilm in a subject, or
(D) treating a condition characterized by the formation of a biofilm in a subject, the method comprising administering to the subject:
(a) a high mobility group box protein (HMGB) polypeptide, optionally the HMGB1 polypeptide or a fragment thereof comprising a B box or an A box or an AB box thereof; and
(b) an anti-DNABII antibody or an antigen-binding fragment thereof comprising:
(i) a heavy chain complementarity-determining region 1 (CDRH1) comprising GFTFRTY (aa 50 to aa 56 of SEQ ID NO: 1 or 2 or 3 or 24);
(ii) a heavy chain complementarity-determining region 2 (CDRH2) comprising GSDRRH (aa 76 to aa 81 of SEQ ID NO: 1 or 2 or 3 or 24);
(iii) a heavy chain complementarity-determining region 3 (CDRH3) comprising VGPYDGYYGEFDY (aa 121 to aa 133 of SEQ ID NO: 1 or 2 or 3 or 24);
(iv) a light chain complementarity-determining region 1 (CDRL1) comprising QSLLDSDGKTF (aa 47 to aa 57 of SEQ ID NO: 7 or 8 or 9 or 25);
(v) a light chain complementarity-determining region 2 (CDRL2) comprising LVS (aa 75 to aa 77 of SEQ ID NO: 7 or 8 or 9 or 25); and
(vi) a light chain complementarity-determining region 3 (CDRL3) comprising WQGTHFPYT (aa 114 to aa 122 of SEQ ID NO: 7 or 8 or 9 or 25).

33. (canceled)

34. A method for inducing or increasing the formation of a neutrophil extracellular trap (NET) immediately adjacent to a biofilm in a subject, and disrupting the biofilm optionally without inducing a pro-inflammatory response, the method comprising administering to the subject:
(a) a high mobility group box 1 protein (HMGB1) polypeptide comprising the amino acid sequence of SEQ ID NO: 52, or a fragment thereof comprising, or consisting essentially of, or yet further consisting of a B box or an A box or an AB box thereof, and
(b) an anti-DNABII antibody or an antigen-binding fragment thereof comprising:
(i) a heavy chain complementarity-determining region 1 (CDRH1) comprising GFTFRTY (aa 50 to aa 56 of SEQ ID NO: 1 or 2 or 3 or 24);
(ii) a heavy chain complementarity-determining region 2 (CDRH2) comprising GSDRRH (aa 76 to aa 81 of SEQ ID NO: 1 or 2 or 3 or 24);
(iii) a heavy chain complementarity-determining region 3 (CDRH3) comprising VGPYDGYYGEFDY (aa 121 to aa 133 of SEQ ID NO: 1 or 2 or 3 or 24);
(iv) a light chain complementarity-determining region 1 (CDRL1) comprising QSLLDSDGKTF (aa 47 to aa 57 of SEQ ID NO: 7 or 8 or 9 or 25);
(v) a light chain complementarity-determining region 2 (CDRL2) comprising LVS (aa 75 to aa 77 of SEQ ID NO: 7 or 8 or 9 or 25); and
(vi) a light chain complementarity-determining region 3 (CDRL3) comprising WQGTHFPYT (aa 114 to aa 122 of SEQ ID NO: 7 or 8 or 9 or 25).

35. (canceled)

36. (canceled)

37. A method for one or more of the following:
(A) preventing the formation of or disrupting a biofilm in vitro or ex vivo,
(B) preventing the formation of or disrupting a biofilm in a subject,
(C) inhibiting, preventing or treating a microbial infection that produces a biofilm in a subject, or
(D) treating a condition characterized by the formation of a biofilm in a subject,
the method comprising administering to the subject the composition or combination of claim 1.

38. A method for inducing or increasing the formation of a neutrophil extracellular trap (NET) immediately adjacent to a biofilm in a subject, and disrupting the biofilm optionally without inducing a pro-inflammatory response, the method comprising administering to the subject the composition or combination of claim 1, wherein the HMGB1 polypeptide comprises the amino acid sequence of SEQ ID NO: 52, or a fragment thereof comprising, or consisting essentially of, or yet further consisting of a B box or an A box or an AB box thereof.

39. (canceled)

40. (canceled)

41. A kit comprising instructions for use and the composition or combination of claim 1.
